(19) United States
(12) Patent Application Publication
(10) Pub. No.: US 2023/0406838 A1
(43) Pub. Date: Dec. 21, 2023

(54) MUTANT SELECTIVE EGFR INHIBITORS AND METHODS OF USE THEREOF

(71) Applicant: Dana-Farber Cancer Institute, Inc., Boston, MA (US)

(72) Inventors: David Heppner, Brookline, MA (US); Michael Eck, Brookline, MA (US); Stefan Laufer, Tubingen (DE); Florian Wittlinger, Tubingen (DE); Marcel Guenther, Tubingen (DE)

(21) Appl. No.: 18/189,092

(22) Filed: Mar. 23, 2023

Related U.S. Application Data
(63) Continuation of application No. PCT/US2021/051989, filed on Sep. 24, 2021.
(60) Provisional application No. 63/083,567, filed on Sep. 25, 2020.

(51) Int. Cl. C07D 401/04; C07D 409/14; C07D 401/14; C07D 405/14; C07D 403/12; C07D 233/84; A61P 35/00

(52) U.S. Cl. CPC C07D 401/04; C07D 409/14; C07D 401/14; C07D 405/14; C07D 403/12; C07D 233/84; A61P 35/00

(57) ABSTRACT

The disclosure relates to compounds that act as inhibitors of epidermal growth factor receptor (EGFR); pharmaceutical compositions comprising the compounds; and methods of treating or preventing kinase-mediated disorders, including cancer and other proliferation diseases.

### RELATED APPLICATIONS

This application claims priority to U.S. Provisional Application No. 63/083,567, filed Sep. 25, 2020, the entire content of which is hereby incorporated by reference in its entirety.

### STATEMENT REGARDING FEDERALLY SPONSORED RESEARCH OR DEVELOPMENT

This invention was made with government support under Grant No. R01 CA201049 awarded by The National Institutes of Health. The government has certain rights in the invention.

### BACKGROUND

The epidermal growth factor receptor (EGFR, Erb-B1) belongs to a family of receptor tyrosine kinases that mediate the proliferation, differentiation, and survival of normal and malignant cells (Arteaga, C. L., J. Clin. Oncol. 19, 2001, 32-40). Deregulation of EGFR has been implicated in many types of human cancer, with overexpression of the receptor present in at least 70% of human cancers (Seymour, L. K., Curr. Drug Targets 2, 2001, 117-133), including non-small lung cell carcinomas, breast cancers, gliomas, squamous cell carcinomas of the head and neck, and prostate cancer (Raymond, E., et al., Drugs 60 (Suppl. 1), 2000, 15-23, discussion 41-2; Salomon, D. S., et al., Crit. Rev. Oncol. Hematol. 19, 1995, 183-232; Voldborg B. R., et al., Ann. Oncol. 8, 1997, 1197-1206), EGFR has, therefore, emerged as an attractive target for the design and development of diagnostic and therapeutic agents that can specifically bind and inhibit the receptor's tyrosine kinase activity and signal transduction pathway in cancer cells. For example, the EGFR tyrosine kinase (EGFR-TK) reversible inhibitor TARCEVA® is approved by the FDA for treatment of NSCLC and advanced pancreatic cancer. Other anti-EGFR targeted molecules have also been approved, including Lapatinib and IRESSA®.

The epidermal growth factor receptor (EGFR) is one of the most investigated receptor protein tyrosine kinases and its link to non-small-cell lung cancer (NSCLC) is well established (A. Russo, T. et al., Oncotarget 2015, 6, 26814). However, over 75% of patients die five years after their NSCLC diagnosis. Tumors driven by activating mutations within the EGFR tyrosine kinase domain, e.g., point-mutation L858R or in-frame exon-19 deletions (ex19del) are initially sensitive to EGFR tyrosine kinase inhibitors (TKIs), such as gefitinib and erlotinib, (Paez. J. G., et al., Science (New York, N.Y.) 2004, 304, 1497; Lynch, T. J., et al., The New England Journal of Medicine 2004, 350, 2129), but these inhibitors are rendered resistant due to the acquisition of the secondary ‘gatekeeper’ T790M mutation (Pao, W., et al., PLoS Medicine 2005, 2, e73; Yu, H. A., et al., Clinical Cancer Research 2013, 19, 2240). Efforts to overcome 1st generation TKI drug resistance resulted in the discovery and optimization of T790M-targeting irreversible inhibitors, which are rendered effective due to the ability to form covalent bonds with C797 (D. A. E. Cross, et al., Cancer Discovery 2014, 4, 1046; E. L. Kwak, et al., Proceedings of the National Academy of Sciences of the United States of America 2005, 102, 7665). Patients harboring T790M positive tumors respond well to treatment with AZD9291, and more recently this drug has been shown to be a superior treatment as a front-line therapy in untreated EGFR mutant NSCLC patients (J.-C. Soria, et al., The New England Journal of Medicine 2018, 378, 113). However, despite these successes, patients can acquire resistance to AZD9291 through the acquisition of the C797S mutation that precludes the ability for the drug to form their essential covalent bonds (K. S. Thress, et al., Nature Medicine 2015, 21, 560).

Thus, there is a need for potent small molecule EGFR inhibitors with alternative mechanisms of action targeting mutant EGFR.

### SUMMARY

In an aspect, provided herein is a compound of Formula I:

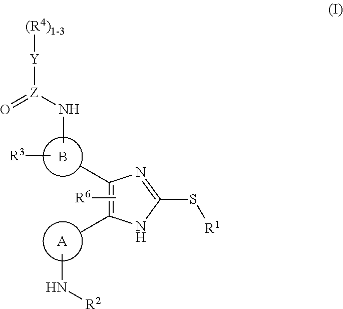

or a pharmaceutically acceptable salt thereof;
   wherein the variables are defined herein.

In an embodiment, the compound of Formula I is a compound of Formula IIa:

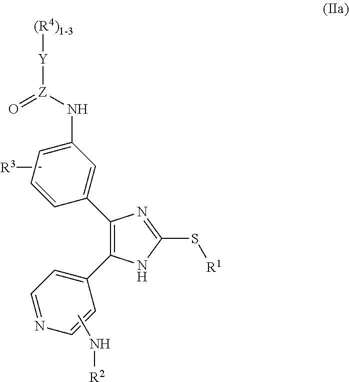

or a pharmaceutically acceptable salt thereof.

In another embodiment, the compound of Formula I is a compound of Formula IIb:

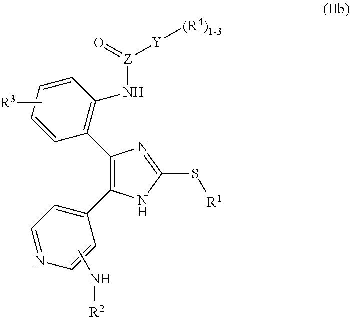

or a pharmaceutically acceptable salt thereof.

In another embodiment, the compound of Formula I is a compound of Formula III:

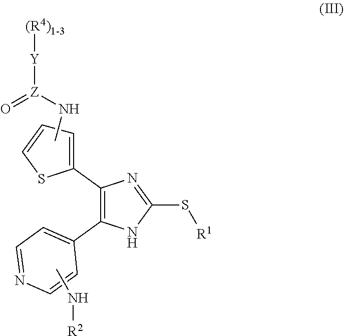

or a pharmaceutically acceptable salt thereof.

In yet another embodiment, the compound of Formula I is a compound of Formula IV:

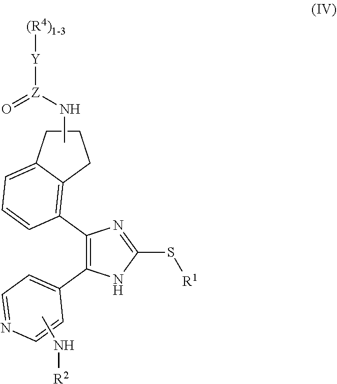

or a pharmaceutically acceptable salt thereof.

In an aspect, provided herein is a method of treating cancer or a proliferation disease, comprising administering to a subject in need thereof an effective amount of a compound of disclosed herein or a pharmaceutical composition comprising a compound disclosed herein and a pharmaceutically acceptable carrier. In one embodiment, the cancer is lung cancer, breast cancer, glioma, squamous cell carcinoma, or prostate cancer. In another embodiment, the cancer is non-small cell lung cancer (NSCLC).

In another aspect, provided herein is a method of inhibiting the activity of EGFR, comprising administering to a subject in need thereof an effective amount of a compound of disclosed herein or a pharmaceutical composition comprising a compound disclosed herein and a pharmaceutically acceptable carrier. In an embodiment, the compound targets Cys775 on EGFR.

The disclosure also provides a kit comprising a compound capable of inhibiting EGFR activity selected from a compound of the present disclosure, or a pharmaceutically acceptable salt thereof, and instructions for use in treating cancer. In one embodiment, the kit further comprises components for performing a test to determine whether a subject has an activating mutation in EGFR or a resistance mutation in EGFR

### DETAILED DESCRIPTION

### Definitions

Listed below are definitions of various terms used to describe the compounds and compositions disclosed herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

The term “administration” or the like as used herein refers to the providing a therapeutic agent to a subject. Multiple techniques of administering a therapeutic agent exist in the art including, but not limited to, intravenous, oral, aerosol, parenteral, ophthalmic, pulmonary, and topical administration.

The term “treat,” “treated,” “treating,” or “treatment” includes the diminishment or alleviation of at least one symptom associated or caused by the state, disorder or disease being treated. In certain embodiments, the treatment comprises bringing into contact with wild-type or mutant EGFR an effective amount of a compound disclosed herein for conditions related to cancer.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

The terms “combination” or “pharmaceutical combination” as used herein refer to either a fixed combination in one dosage unit form, or non-fixed combination in separate dosage forms, or a kit of parts for the combined administration where two or more therapeutic agents may be administered independently, at the same time or separately within time intervals. As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringers solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure, and are physiologically acceptable to the patient. Supplementary active compounds may also be incorporated into the compositions. The “pharmaceutically acceptable carrier” may further include a pharmaceutically acceptable salt of the compound disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

As used herein, the term “EGFR” refers to epidermal growth factor receptor (alternately referred to as ErbB-1 or HER1) and may refer to the wild-type receptor or to a receptor containing one or more mutations.

As used herein, the term “HER” or Her″ refers to members of the ErbB receptor tyrosine kinase family, including EGFR, ERBB2, HER3, and HER4.

As used herein, the term “allosteric site” refers to a site on EGFR other than the ATP binding site, such as that characterized in a crystal structure of EGFR. An “alosteric site” can be a site that is close to the ATP binding site, such as that characterized in a crystal structure of EGFR. For example, one allosteric site includes one or more of the following amino acid residues of epidermal growth factor receptor (EGFR): Lys745, Leu788, Ala743, Cys755, Leu777, Phe856, Asp855, Met766, Ile759, Glu762, and/or Ala763.

As used herein, the term “agent that prevents EGFR dimer formation,” or iterations thereof, refers to an agent that prevents dimer formation in which the C-lobe of the “activator” subunit impinges on the N-lobe of the “receiver” subunit. Examples of agents that prevent EGFR dimer formation include, but are not limited to, cetuximab, trastuzumab, panitumumab, and Mig6.

As used herein, the term “alkyl,” by itself or as part of another substituent means, unless otherwise stated, a straight or branched chain hydrocarbon having the number of carbon atoms designated (i.e., C₁-C₆ alkyl means an alkyl having one to six carbon atoms) and includes straight and branched chains. Examples include methyl, ethyl, propyl, isopropyl, butyl, isobutyl, tert butyl, pentyl, neopentyl, and hexyl. Other examples of C₁-C₆ alkyl include ethyl, methyl, isopropyl, isobutyl, n-pentyl, and n-hexyl.

As used herein, the term “haloalkyl” refers to an alkyl group, as defined above, substituted with one or more halo substituents, wherein alkyl and halo are as defined herein.

Haloalkyl includes, byway of example, chloromethyl, trifluoromethyl, bromoethyl, chlorofluoroethyl, and the like.

As used herein, the term “alkoxy” refers to the group —O-alkyl, wherein alkyl is as defined herein. Alkoxy includes, by way of example, methoxy, ethoxy, n-propoxy, isopropoxy, n-butoxy, sec-butoxy, t-butoxy and the like.

As used herein, the term “alkenyl” refers to a monovalent group derived from a hydrocarbon moiety containing, in certain embodiments, from two to six, or two to eight carbon atoms having at least one carbon-carbon double bond. The alkenyl group may or may not be the point of attachment to another group. The term “alkenyl” includes, but is not limited to, ethenyl, 1-propenyl, 1-butenyl, heptenyl, octenyl and the like.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” means a non-aromatic carbocyclic system that is fully saturated having 1, 2 or 3 rings wherein such rings may be fused. The term “fused” means that a second ring is present (i.e., attached or formed) by having two adjacent atoms in common (i.e., shared) with the first ring. Cycloalkyl also includes bicyclic structures that may be bridged or spirocyclic in nature with each individual ring within the bicycle varying from 3-8 atoms. The term “cycloalkyl” includes, but is not limited to, cyclopropyl, cyclobutyl, cyclopentyl, cyclohexyl, bicyclo[3.1.0]hexyl, spiro[3.3]heptanyl, and bicyclo[1.1.1]pentyl.

As used herein, the term “bicyclic ring” means a fused ring system comprising two rings, wherein the first ring is aryl or heteroaryl and the second ring is cycloalkyl or heterocycloalkyl. The term “bicyclic ring” Includes, but Is not limited to, isoindoline-1,3-dione, isoindolin-1-one, and dihydro-indene.

As used herein, the term “heterocyclyl” or “heterocycloalkyl” means a non-aromatic carbocyclic system containing 1, 2, 3 or 4 heteroatoms selected independently from N, O, and S and having 1, 2 or 3 rings wherein such rings may be fused, wherein fused is defined above. Heterocyclyl also includes bicyclic structures that may be bridged or spirocyclic in nature with each individual ring within the bicycle varying from 3-8 atoms, and containing 0, 1, or 2 N, O, or S atoms. The term “heterocyclyl” includes cyclic esters (i.e., lactones) and cyclic amides (i.e., lactams) and also specifically includes, but is not limited to, epoxidyl, oxetanyl, tetrahydro-furanyl, tetrahydropyranyl (i.e., oxanyl), pyranyl, dioxanyl, aziridinyl, azetidinyl, pyrrolidinyl, 2,5-dihydro-1H-pyrrolyl, oxazolidinyl, thiazolidinyl, piperidinyl, morpholinyl, piperazinyl, thiomorpholinyl, 1,3-oxazinanyl, 1,3-thiazinanyl, 2-azabicyclo[2.1.1]hexanyl, 5-azabicyclo-[2.1.1]hexanyl, 6-azabicyclo[3.1.1] heptanyl, 2-azabicyclo[2.2.1]heptanyl, 3-azabicyclo[3.1.1]-heptanyl, 2-azabicyclo[3.1.1]heptanyl, 3-azabicyclo[3.1.0]hexanyl, 2-azabicyclo[3.1.0]hexanyl, 3-azabicyclo[3.2.1]octanyl, 8-azabicyclo[3.2.1]octanyl, 3-oxa-7-azabicyclo[3.3.1]nonanyl, 3-oxa-9-azabicyclo[3.3.1]nonanyl, 2-oxa-5-azabicyclo[2.2.1]heptanyl, 6-oxa-3-azabicyclo[3.1.1]-heptanyl, 2-azaspiro[3.3]heptanyl, 2-oxa-6-azaspiro[3.3]heptanyl, 2-oxaspiro[3.3]heptanyl, 2-oxaspiro[3.5]nonanyl, 3-oxaspiro[5.3]nonanyl, and 8-oxabicyclo[3.2.1]octanyl.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “ary” means an aromatic carbocyclic system containing 1, 2 or 3 rings, wherein such rings may be fused, wherein fused is defined above. If the rings are fused, one of the rings must be fully unsaturated and the fused ring(s) may be fully saturated, partially unsaturated or fully unsaturated. The term “aryl” includes, but is not limited to, phenyl, naphthyl, indanyl, and 1,2,3,4-tetrahydronaphthalenyl. In some embodiments, aryl groups have 6 carbon atoms. In some embodiments, aryl groups have from six to ten carbon atoms. In some embodiments, aryl groups have from six to sixteen carbon atoms.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. The term “heteroaryl” includes, but is not limited to, furanyl, thienyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, imidazo[1,2-a]pyridinyl, pyrazolo[1,5-a]pyridinyl, 5,6,7,8-tetrahydroisoquinolinyl, 5,6,7,8-tetrahydroquinolinyl, 6,7-dihydro-5H-cyclopenta[b]pyridinyl, 6,7-dihydro-5H-cyclopenta-[c]pyridinyl, 1,4,5,6-tetrahydrocyclopenta[c]pyrazolyl, 2,4,5,6-tetrahydrocyclopenta[c]pyrazolyl, 5,6-dihydro-4H-pyrrolo[1,2-b]pyrazolyl, 6,7-dihydro-5H-pyrrolo[1,2-b][1,2,4]triazolyl, 5,6,7,8-tetrahydro-[1,2,4]triazolo[1,5-a]pyridinyl, 4,5,6,7-tetrahydropyrazolo[1,5-a]pyridinyl, 4,5,6,7-tetrahydro-1H-indazolyl and 4,5,6,7-tetrahydro-2H-indazolyl.

It is to be understood that if an aryl, heteroaryl, cycloalkyl, bicyclic ring, or heterocyclyl moiety may be bonded or otherwise attached to a designated moiety through differing ring atoms (i.e., shown or described without denotation of a specific point of attachment), then all possible points are intended, whether through a carbon atom or, for example, a trivalent nitrogen atom. For example, the term “pyridinyl” means 2-, 3- or 4-pyridinyl, the term “thienyl” means 2- or 3-thienyl, and so forth.

As used herein, the term “substituted” means that an atom or group of atoms has replaced hydrogen as the substituent attached to another group.

As used herein, the term “optionally substituted” means that the referenced group may be substituted or unsubstituted.

### Compounds

Provided herein are compounds that are inhibitors of epidermal growth factor receptor (EGFR) useful in the treatment of kinase-mediated disorders, including cancer and other proliferation diseases. In an embodiment, the compounds provided herein are mutant selective EGFR inhibitors.

In an aspect, provided herein is a compound of Formula I:

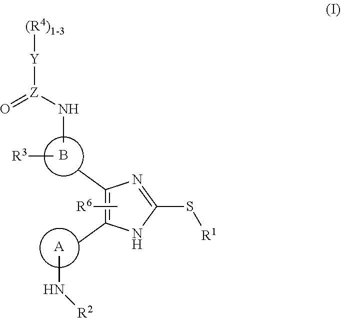

or a pharmaceutically acceptable salt thereof;
   wherein:
Z is C or S═O;
Y is selected from the group consisting of NH, C₁-C₆ alkyl, C₆-C₁₀ aryl, 5-10 membered heteroaryl, C₃-C₁₀ cycloalkyl, and 3-10 membered heterocycloalkyl;
alternatively, Z═O and Y are absent;
A is 5-10 membered heteroaryl containing at least one nitrogen atom;
alternatively, A and NHR² are absent;
B is selected from the group consisting of C₅-C₁₀ aryl, 5-10 membered heteroaryl, C₃-C₁₀ cycloalkyl, 3-10 membered heterocycloalkyl, and 5-10 membered bicyclic ring;
R¹ is C₁-C₆ alkyl;
R² is H, CO(C₁-C₆ alkyl), or C₆-C₁₀ aryl, wherein aryl is optionally substituted one or two times with R⁵;
R³ is selected from the group consisting of H, halo, CN, OH, NH₂, and CF₃;
each R⁴ is independently selected from the group consisting of H, OH, halo, C₁-C₆ alkyl, C₁-C₆ alkoxy, C₆-C₁₀ aryl, 5-10 membered heteroaryl, CH₂-(5-10 membered bicyclic ring), and CH₂NHC(O)(C₆-C₁₀ aryl), wherein aryl is optionally independently substituted one, two, or three times with halo, CO₂H, or C₁-C₆ haloalkyl;
each R⁵ is independently selected from the group consisting of halo, OH, C₁-C₆ alkoxy, and NHC(O)C₂-C₆ alkenyl and
R⁶ is H or C₁-C₆ alkyl.

In an embodiment,
Z is C or S═O;
Y is selected from the group consisting of NH, C₁-C₆ alkyl, C₆-C₁₀ aryl, 5-10 membered heteroaryl, C₃-C₁₀ cycloalkyl, and 3-10 membered heterocycloalkyl;
alternatively, Z═O and Y are absent;
A is pyridine;
alternatively, A and NHR² are absent;
B is selected from the group consisting of phenyl, thiophenyl, and 6-9 membered bicyclic ring;
R¹ is C₁-C₆ alkyl;
R² is CO(C₁-C₆ alkyl) or C₆-C₁₀ aryl, wherein aryl is optionally substituted one or two times with R⁵;
R³ is H or halo;
each R⁴ is independently selected from the group consisting of H, OH, halo, C₁-C₆ alkyl, C₁-C₆ alkoxy, C₆-C₁₀ aryl, 5-10 membered heteroaryl, CH₂-(5-10 membered bicyclic ring), and CH₂NHC(O)(C₆-C₁₀ aryl), wherein aryl is optionally independently substituted one, two, or three times with halo, CO₂H, or C₁-C₆ haloalkyl, and wherein bicyclic ring is optionally substituted one or two times with ═O;
each R⁵ is independently selected from the group consisting of C₁-C₆ alkoxy and NHC(O)C₁-C₆ alkenyl; and
R⁵ is H.

In an embodiment, A is pyridine. In another embodiment, B is phenyl, thiophene, or dihydro-indene.

In yet another embodiment, R¹ is C₁-C₃ alkyl. In still another embodiment, R² is CO(C₁-C₃ alkyl) or phenyl, wherein phenyl is optionally substituted one or two times with R⁵.

In an embodiment, Z is C. In another embodiment, Z is C═O.

In yet another embodiment, Y is selected from the group consisting of NH, C₁-C₃ alkyl, phenyl, naphthalene, pyridine, indole, thiophene, furan, C₃-C₅ cycloalkyl, and 3-5 membered heterocycloalkyl. In still another embodiment, Y is NH. In an embodiment Y is C₁-C₃ alkyl. In another embodiment, Y is phenyl or naphthalene. In yet another embodiment, Y is pyridine, indole, thiophene, or furan. In still another embodiment, Y is C₃-C₅ cycloalkyl or 3-5 membered heterocycloalkyl.

In an embodiment, Y is substituted with R4 once. In another embodiment, Y is independently substituted with R⁴ two times. In still another embodiment, Y is independently substituted with R⁴ three times.

In an embodiment, R³ is H. In another embodiment, R³ is halo.

In yet another embodiment, each R⁴ is independently selected from the group consisting of H, OH, halo, C₁-C₃ alkyl, C₁-C₃ alkoxy, phenyl, thiophene, indole, CH₂-(5-10 membered bicyclic ring), and CH₂NHC(O)phenyl, wherein phenyl is optionally substituted one, two, or three times with halo, CO₂H, or C₁-C₃ haloalkyl.

In still another embodiment, R⁴ is H. In an embodiment, R⁴ is OH. In another embodiment, R⁴ is halo. In yet another embodiment, R⁴ is C₁-C₃ alkyl or C₁-C₃ alkoxy. In still another embodiment, R⁴ is phenyl, thiophene, or indole, wherein phenyl is optionally substituted one, two, or three times with halo, CO₂H, or C₁-C₃ haloalkyl. In an embodiment, R⁴ is CH₂-(5-10 membered bicyclic ring) or CH₂NHC(O)phenyl, wherein phenyl is optionally substituted one, two, or three times with halo, CO₂H, or C₁-C₃ haloalkyl.

In an embodiment, each R⁴ is independently selected from the group consisting of H, OH, halo, C₁-C₃ alkyl, C₁-C₃ alkoxy, phenyl, thiophene, indole,

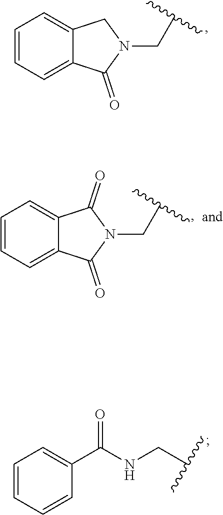 [SMILES: *CN1Cc2ccccc2C1=O ; *CN1C(=O)c2ccccc2C1=O ; *CNC(=O)c1ccccc1]

wherein phenyl is optionally substituted with halo, CO₂H, or C₁-C₃ haloalkyl.

In another embodiment, R⁴ is

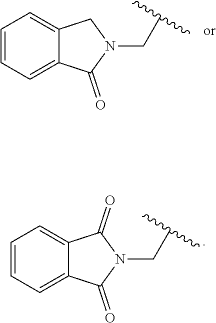 [SMILES: *CN1Cc2ccccc2C1=O ; *CN1C(=O)c2ccccc2C1=O]

In yet another embodiment, R⁵ is H.

In still another embodiment, the compound of Formula I is a compound of Formula

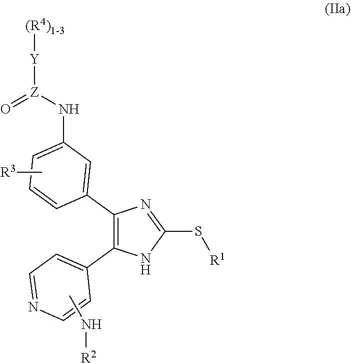

or a pharmaceutically acceptable salt thereof.

In an embodiment, the compound of Formula I is a compound of Formula IIb:

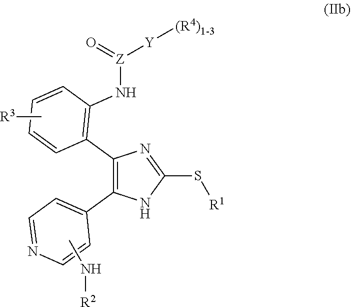

or a pharmaceutically acceptable salt thereof.

In another embodiment, the compound of Formula I is a compound of Formula III:

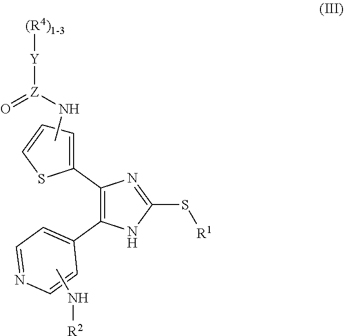

or a pharmaceutically acceptable salt thereof.

In yet another embodiment, the compound of Formula I is a compound of Formula IV:

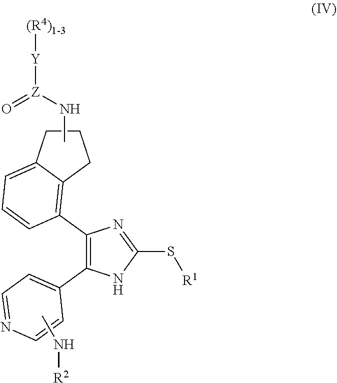

or a pharmaceutically acceptable salt thereof.

In an embodiment, Y is substituted with R⁴ once. In another embodiment, Y is independently substituted with R⁴ two times. In still another embodiment, Y is independently substituted with R⁴ three times.

In an embodiment, the compound of Formula I is selected from the group consisting of a compound in Table 1.

TABLE 1

| Com- | |
|---|---|
| pound | |
| No. | Structure |
| 001 | 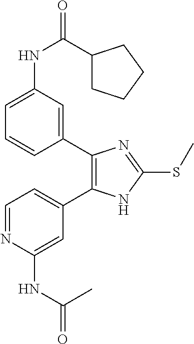 [SMILES: CSc1nc(-c2cccc(NC(=O)C3CCCC3)c2)c(-c2ccnc(NC(C)=O)c2)[nH]1] |
| 002 | 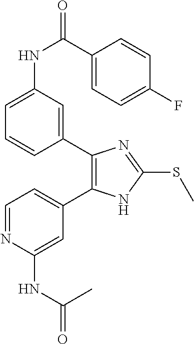 [SMILES: CSc1nc(-c2cccc(NC(=O)c3ccc(F)cc3)c2)c(-c2ccnc(NC(C)=O)c2)[nH]1] |
| 003 | 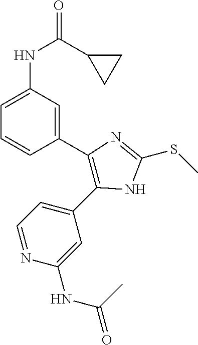 [SMILES: CSc1nc(-c2cccc(NC(=O)C3CC3)c2)c(-c2ccnc(NC(C)=O)c2)[nH]1] |
| 004 | 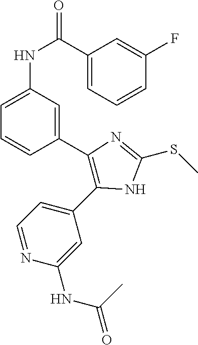 [SMILES: CSc1nc(-c2cccc(NC(=O)c3cccc(F)c3)c2)c(-c2ccnc(NC(C)=O)c2)[nH]1] |
| 005 | 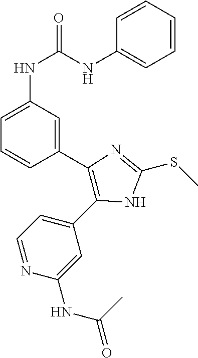 [SMILES: CSc1nc(-c2cccc(NC(=O)Nc3ccccc3)c2)c(-c2ccnc(NC(C)=O)c2)[nH]1] |
| 006 | 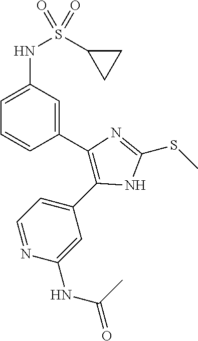 [SMILES: CSc1nc(-c2cccc(NS(=O)(=O)C3CC3)c2)c(-c2ccnc(NC(C)=O)c2)[nH]1] |
| 007 | 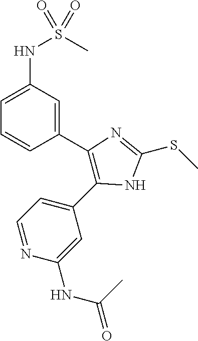 [SMILES: CSc1nc(-c2cccc(NS(C)(=O)=O)c2)c(-c2ccnc(NC(C)=O)c2)[nH]1] |
| 008 | 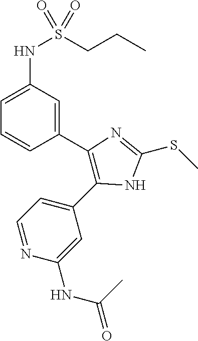 [SMILES: CCCS(=O)(=O)Nc1cccc(-c2nc(SC)[nH]c2-c2ccnc(NC(C)=O)c2)c1] |
| 009 | 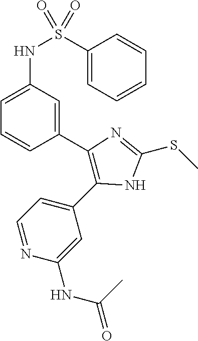 [SMILES: CSc1nc(-c2cccc(NS(=O)(=O)c3ccccc3)c2)c(-c2ccnc(NC(C)=O)c2)[nH]1] |
| 010 | 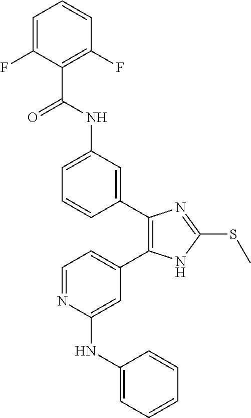 [SMILES: CSc1nc(-c2cccc(NC(=O)c3c(F)cccc3F)c2)c(-c2ccnc(Nc3ccccc3)c2)[nH]1] |
| 011 | 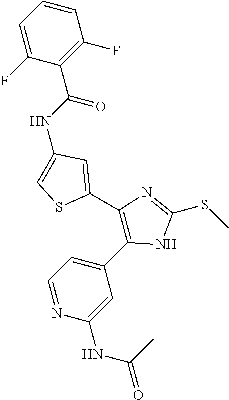 [SMILES: CSc1nc(-c2cc(NC(=O)c3c(F)cccc3F)cs2)c(-c2ccnc(NC(C)=O)c2)[nH]1] |
| 012 | 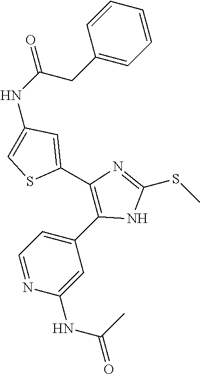 [SMILES: CSc1nc(-c2cc(NC(=O)Cc3ccccc3)cs2)c(-c2ccnc(NC(C)=O)c2)[nH]1] |
| 013 | 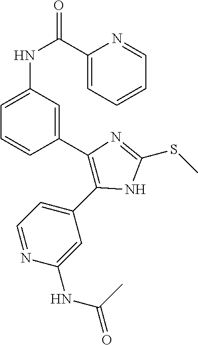 [SMILES: CSc1nc(-c2cccc(NC(=O)c3ccccn3)c2)c(-c2ccnc(NC(C)=O)c2)[nH]1] |
| 014 | 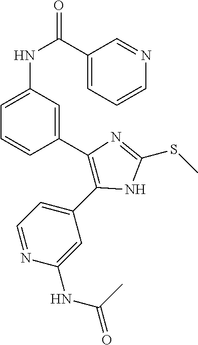 [SMILES: CSc1nc(-c2cccc(NC(=O)c3cccnc3)c2)c(-c2ccnc(NC(C)=O)c2)[nH]1] |
| 015 | 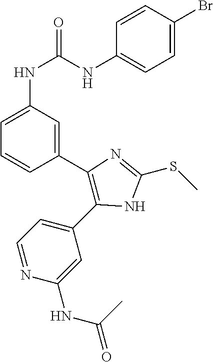 [SMILES: CSc1nc(-c2cccc(NC(=O)Nc3ccc(Br)cc3)c2)c(-c2ccnc(NC(C)=O)c2)[nH]1] |
| 016 | 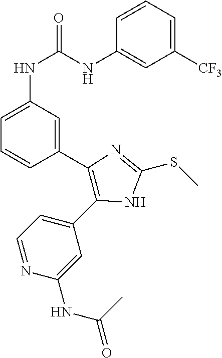 [SMILES: CSc1nc(-c2cccc(NC(=O)Nc3cccc(C(F)(F)F)c3)c2)c(-c2ccnc(NC(C)=O)c2)[nH]1] |
| 017 | 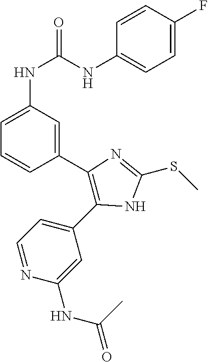 [SMILES: CSc1nc(-c2cccc(NC(=O)Nc3ccc(F)cc3)c2)c(-c2ccnc(NC(C)=O)c2)[nH]1] |
| 018 | 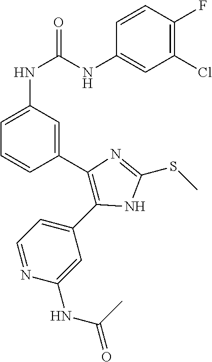 [SMILES: CSc1nc(-c2cccc(NC(=O)Nc3ccc(F)c(Cl)c3)c2)c(-c2ccnc(NC(C)=O)c2)[nH]1] |
| 019 | 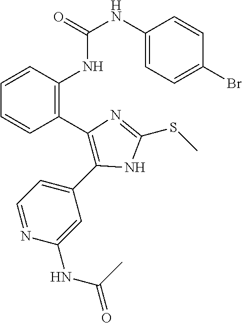 [SMILES: CSc1nc(-c2ccccc2NC(=O)Nc2ccc(Br)cc2)c(-c2ccnc(NC(C)=O)c2)[nH]1] |
| 020 | 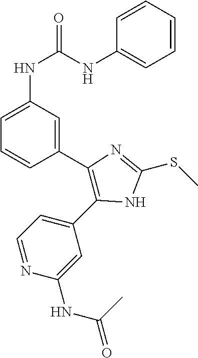 [SMILES: CSc1nc(-c2cccc(NC(=O)Nc3ccccc3)c2)c(-c2ccnc(NC(C)=O)c2)[nH]1] |
| 021 | 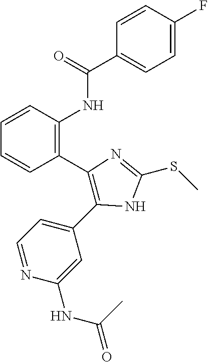 [SMILES: CSc1nc(-c2ccccc2NC(=O)c2ccc(F)cc2)c(-c2ccnc(NC(C)=O)c2)[nH]1] |
| 022 | 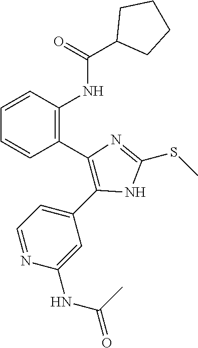 [SMILES: CSc1nc(-c2ccccc2NC(=O)C2CCCC2)c(-c2ccnc(NC(C)=O)c2)[nH]1] |
| 023 | 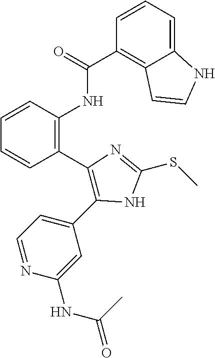 [SMILES: CSc1nc(-c2ccccc2NC(=O)c2cccc3[nH]ccc23)c(-c2ccnc(NC(C)=O)c2)[nH]1] |
| 024 | 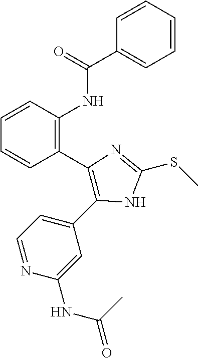 [SMILES: CSc1nc(-c2ccccc2NC(=O)c2ccccc2)c(-c2ccnc(NC(C)=O)c2)[nH]1] |
| 025 | 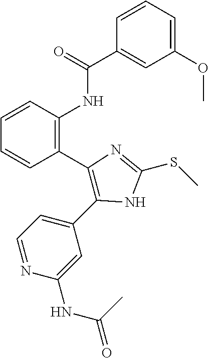 [SMILES: COc1cccc(C(=O)Nc2ccccc2-c2nc(SC)[nH]c2-c2ccnc(NC(C)=O)c2)c1] |
| 026 | 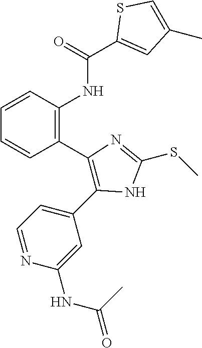 [SMILES: CSc1nc(-c2ccccc2NC(=O)c2cc(C)cs2)c(-c2ccnc(NC(C)=O)c2)[nH]1] |
| 027 | 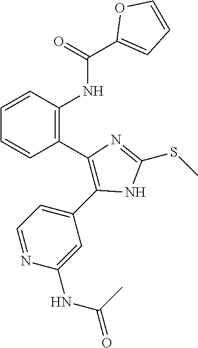 [SMILES: CSc1nc(-c2ccccc2NC(=O)c2ccco2)c(-c2ccnc(NC(C)=O)c2)[nH]1] |
| 028 | 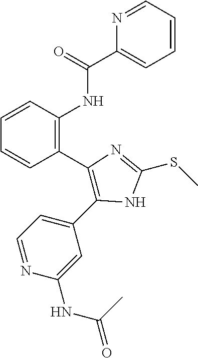 [SMILES: CSc1nc(-c2ccccc2NC(=O)c2ccccn2)c(-c2ccnc(NC(C)=O)c2)[nH]1] |
| 029 | 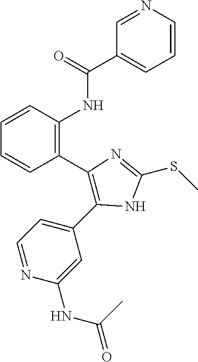 [SMILES: CSc1nc(-c2ccccc2NC(=O)c2cccnc2)c(-c2ccnc(NC(C)=O)c2)[nH]1] |
| 030 | 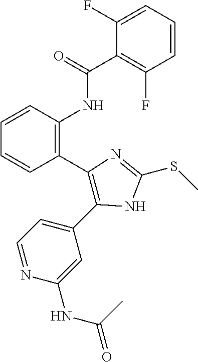 [SMILES: CSc1nc(-c2ccccc2NC(=O)c2c(F)cccc2F)c(-c2ccnc(NC(C)=O)c2)[nH]1] |
| 031 | 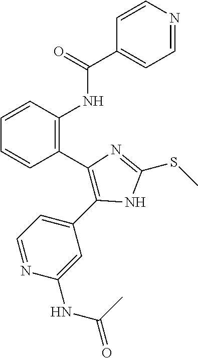 [SMILES: CSc1nc(-c2ccccc2NC(=O)c2ccncc2)c(-c2ccnc(NC(C)=O)c2)[nH]1] |
| 032 | 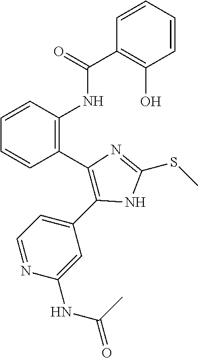 [SMILES: CSc1nc(-c2ccccc2NC(=O)c2ccccc2O)c(-c2ccnc(NC(C)=O)c2)[nH]1] |
| 033 | 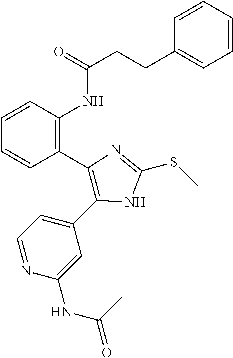 [SMILES: CSc1nc(-c2ccccc2NC(=O)CCc2ccccc2)c(-c2ccnc(NC(C)=O)c2)[nH]1] |
| 034 | 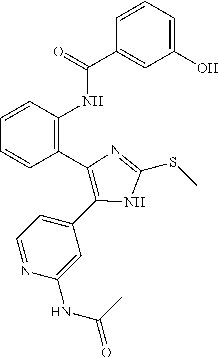 [SMILES: CSc1nc(-c2ccccc2NC(=O)c2cccc(O)c2)c(-c2ccnc(NC(C)=O)c2)[nH]1] |
| 035 | 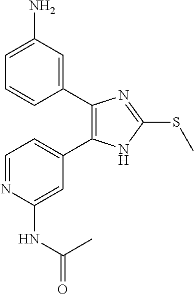 [SMILES: CSc1nc(-c2cccc(N)c2)c(-c2ccnc(NC(C)=O)c2)[nH]1] |
| 036 | 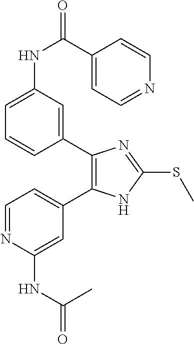 [SMILES: CSc1nc(-c2cccc(NC(=O)c3ccncc3)c2)c(-c2ccnc(NC(C)=O)c2)[nH]1] |
| 037 | 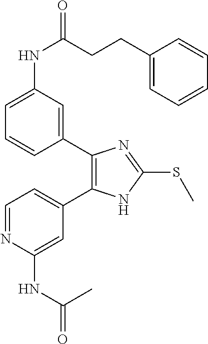 [SMILES: CSc1nc(-c2cccc(NC(=O)CCc3ccccc3)c2)c(-c2ccnc(NC(C)=O)c2)[nH]1] |
| 038 | 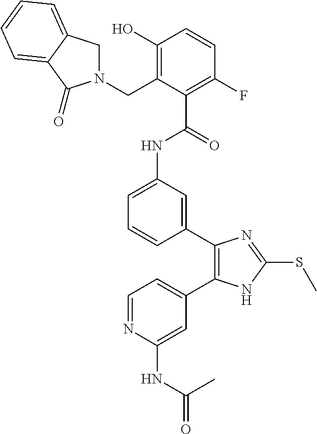 [SMILES: CSc1nc(-c2cccc(NC(=O)c3c(F)ccc(O)c3CN3Cc4ccccc4C3=O)c2)c(-c2ccnc(NC(C)=O)c2)[nH]1] |
| 039 | 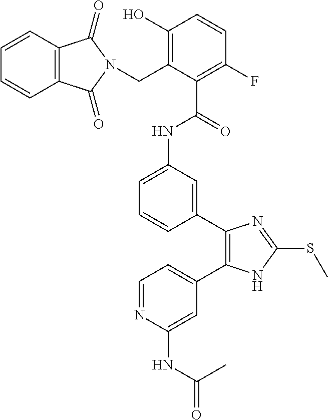 [SMILES: CSc1nc(-c2cccc(NC(=O)c3c(F)ccc(O)c3CN3C(=O)c4ccccc4C3=O)c2)c(-c2ccnc(NC(C)=O)c2)[nH]1] |
| 040 | 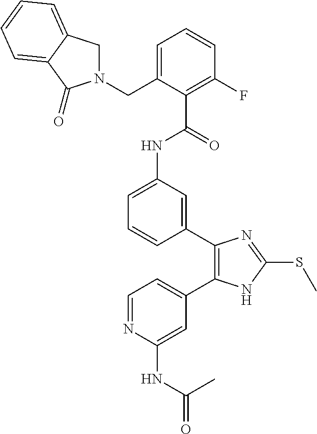 [SMILES: CSc1nc(-c2cccc(NC(=O)c3c(F)cccc3CN3Cc4ccccc4C3=O)c2)c(-c2ccnc(NC(C)=O)c2)[nH]1] |
| 041 | 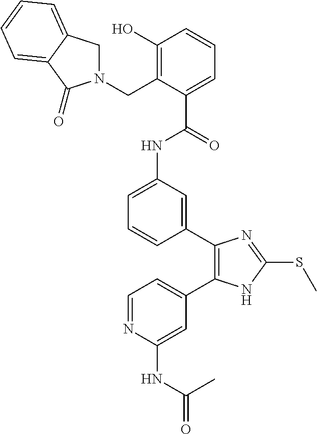 [SMILES: CSc1nc(-c2cccc(NC(=O)c3cccc(O)c3CN3Cc4ccccc4C3=O)c2)c(-c2ccnc(NC(C)=O)c2)[nH]1] |
| 042 | 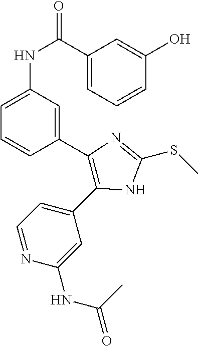 [SMILES: CSc1nc(-c2cccc(NC(=O)c3cccc(O)c3)c2)c(-c2ccnc(NC(C)=O)c2)[nH]1] |
| 043 | 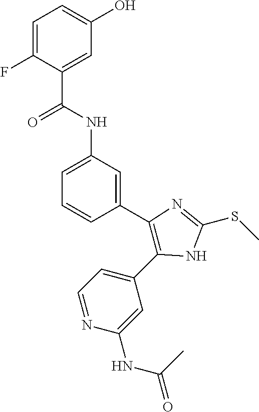 [SMILES: CSc1nc(-c2cccc(NC(=O)c3cc(O)ccc3F)c2)c(-c2ccnc(NC(C)=O)c2)[nH]1] |
| 044 | 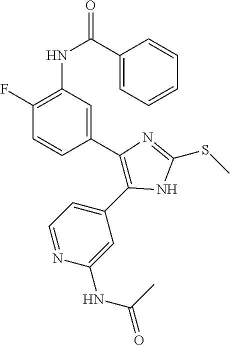 [SMILES: CSc1nc(-c2ccc(F)c(NC(=O)c3ccccc3)c2)c(-c2ccnc(NC(C)=O)c2)[nH]1] |
| 045 | 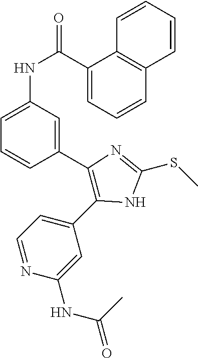 [SMILES: CSc1nc(-c2cccc(NC(=O)c3cccc4ccccc34)c2)c(-c2ccnc(NC(C)=O)c2)[nH]1] |
| 046 | 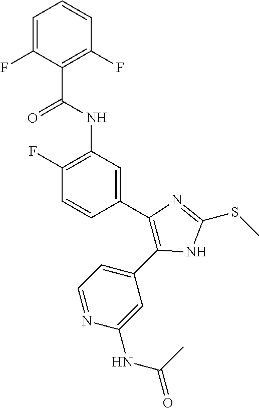 [SMILES: CSc1nc(-c2ccc(F)c(NC(=O)c3c(F)cccc3F)c2)c(-c2ccnc(NC(C)=O)c2)[nH]1] |
| 047 | 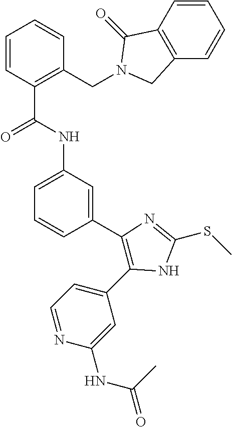 [SMILES: CSc1nc(-c2cccc(NC(=O)c3ccccc3CN3Cc4ccccc4C3=O)c2)c(-c2ccnc(NC(C)=O)c2)[nH]1] |
| 048 | 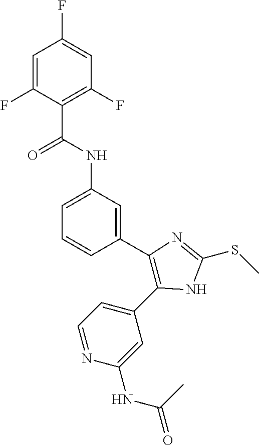 [SMILES: CSc1nc(-c2cccc(NC(=O)c3c(F)cc(F)cc3F)c2)c(-c2ccnc(NC(C)=O)c2)[nH]1] |
| 049 | 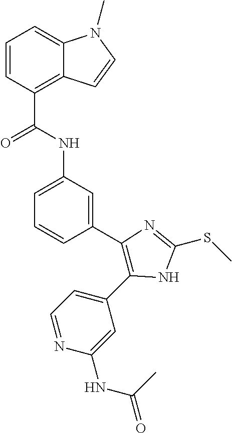 [SMILES: CSc1nc(-c2cccc(NC(=O)c3cccc4c3ccn4C)c2)c(-c2ccnc(NC(C)=O)c2)[nH]1] |
| 050 | 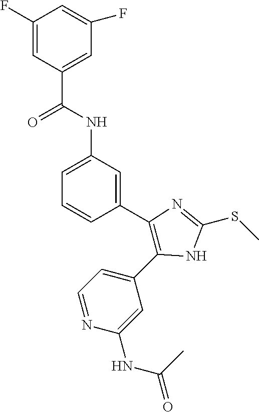 [SMILES: CSc1nc(-c2cccc(NC(=O)c3cc(F)cc(F)c3)c2)c(-c2ccnc(NC(C)=O)c2)[nH]1] |
| 051 | 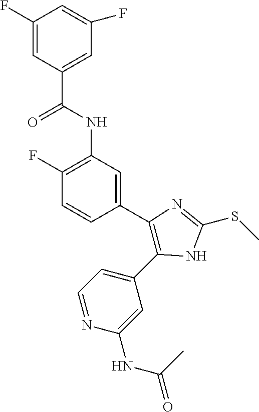 [SMILES: CSc1nc(-c2ccc(F)c(NC(=O)c3cc(F)cc(F)c3)c2)c(-c2ccnc(NC(C)=O)c2)[nH]1] |
| 052 | 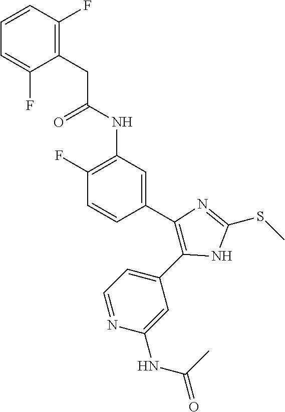 [SMILES: CSc1nc(-c2ccc(F)c(NC(=O)Cc3c(F)cccc3F)c2)c(-c2ccnc(NC(C)=O)c2)[nH]1] |
| 053 | 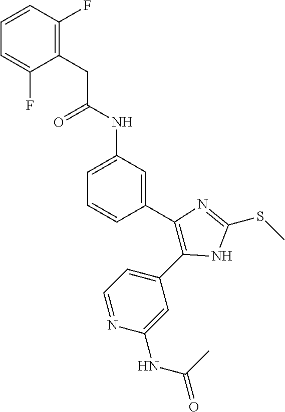 [SMILES: CSc1nc(-c2cccc(NC(=O)Cc3c(F)cccc3F)c2)c(-c2ccnc(NC(C)=O)c2)[nH]1] |
| 054 | 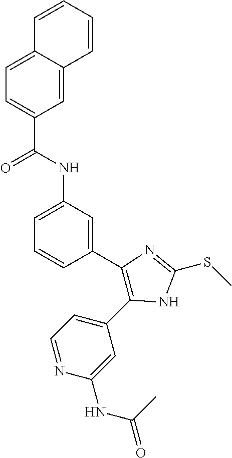 [SMILES: CSc1nc(-c2cccc(NC(=O)c3ccc4ccccc4c3)c2)c(-c2ccnc(NC(C)=O)c2)[nH]1] |
| 055 | 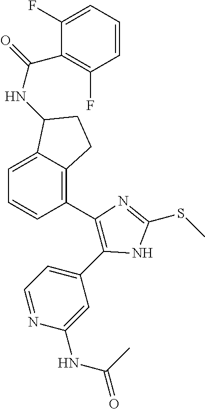 [SMILES: CSc1nc(-c2cccc3c2CCC3NC(=O)c2c(F)cccc2F)c(-c2ccnc(NC(C)=O)c2)[nH]1] |
| 056 | 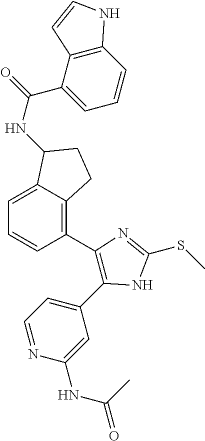 [SMILES: CSc1nc(-c2cccc3c2CCC3NC(=O)c2cccc3[nH]ccc23)c(-c2ccnc(NC(C)=O)c2)[nH]1] |
| 057 | 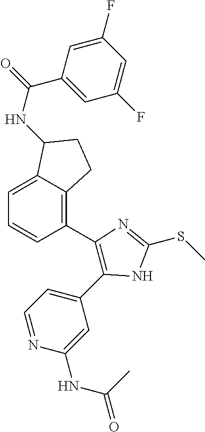 [SMILES: CSc1nc(-c2cccc3c2CCC3NC(=O)c2cc(F)cc(F)c2)c(-c2ccnc(NC(C)=O)c2)[nH]1] |
| 058 | 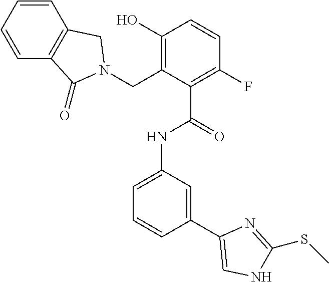 [SMILES: CSc1nc(-c2cccc(NC(=O)c3c(F)ccc(O)c3CN3Cc4ccccc4C3=O)c2)c[nH]1] |
| 059 | 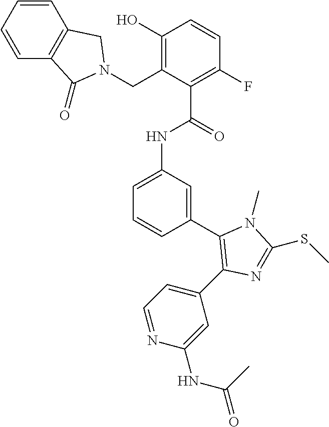 [SMILES: CSc1nc(-c2ccnc(NC(C)=O)c2)c(-c2cccc(NC(=O)c3c(F)ccc(O)c3CN3Cc4ccccc4C3=O)c2)n1C] |
| 060 | 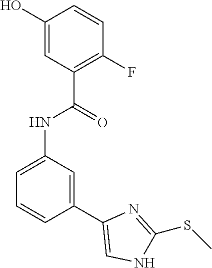 [SMILES: CSc1nc(-c2cccc(NC(=O)c3cc(O)ccc3F)c2)c[nH]1] |
| 061 | 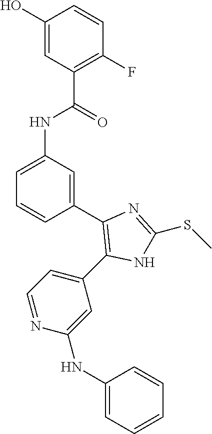 [SMILES: CSc1nc(-c2cccc(NC(=O)c3cc(O)ccc3F)c2)c(-c2ccnc(Nc3ccccc3)c2)[nH]1] |
| 062 | 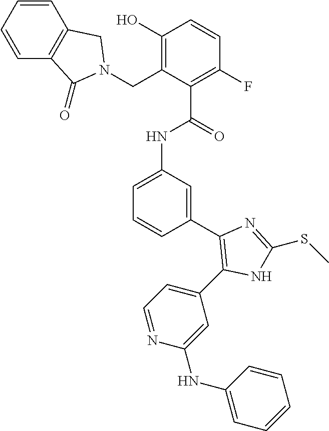 [SMILES: CSc1nc(-c2cccc(NC(=O)c3c(F)ccc(O)c3CN3Cc4ccccc4C3=O)c2)c(-c2ccnc(Nc3ccccc3)c2)[nH]1] |
| 063 | 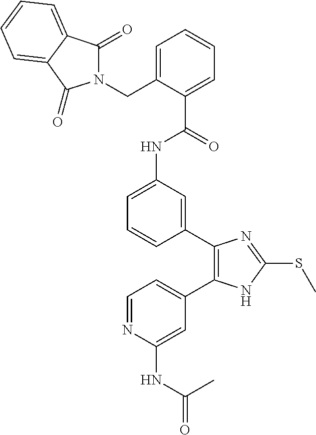 [SMILES: CSc1nc(-c2cccc(NC(=O)c3ccccc3CN3C(=O)c4ccccc4C3=O)c2)c(-c2ccnc(NC(C)=O)c2)[nH]1] |
| 064 | 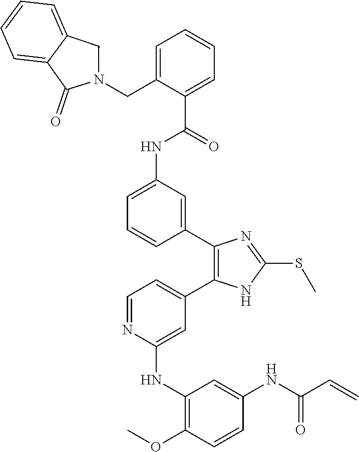 [SMILES: C=CC(=O)Nc1ccc(OC)c(Nc2cc(-c3[nH]c(SC)nc3-c3cccc(NC(=O)c4ccccc4CN4Cc5ccccc5C4=O)c3)ccn2)c1] |
| 065 | 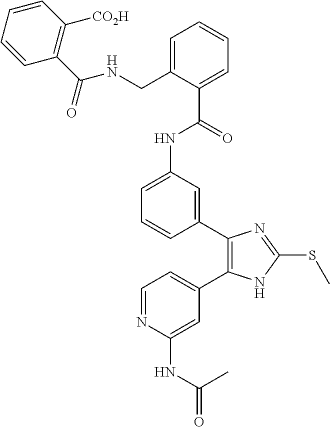 [SMILES: CSc1nc(-c2cccc(NC(=O)c3ccccc3CNC(=O)c3ccccc3C(=O)O)c2)c(-c2ccnc(NC(C)=O)c2)[nH]1] |
| 066 | 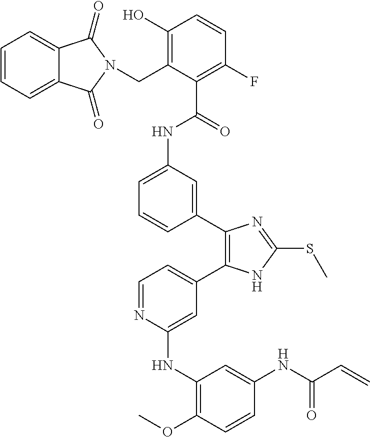 [SMILES: C=CC(=O)Nc1ccc(OC)c(Nc2cc(-c3[nH]c(SC)nc3-c3cccc(NC(=O)c4c(F)ccc(O)c4CN4C(=O)c5ccccc5C4=O)c3)ccn2)c1] |
| 067 | 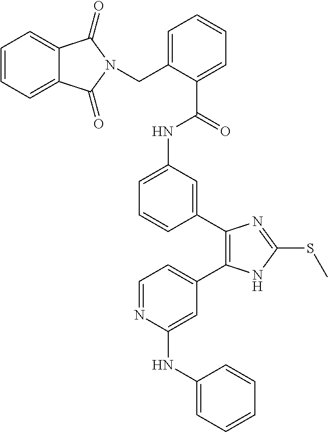 [SMILES: CSc1nc(-c2cccc(NC(=O)c3ccccc3CN3C(=O)c4ccccc4C3=O)c2)c(-c2ccnc(Nc3ccccc3)c2)[nH]1] |
| 068 | 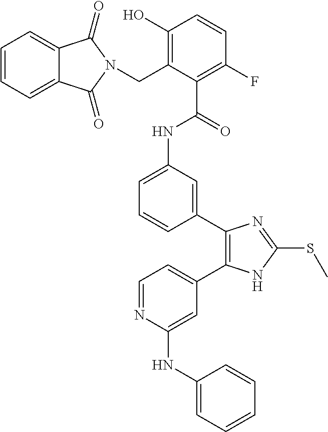 [SMILES: CSc1nc(-c2cccc(NC(=O)c3c(F)ccc(O)c3CN3C(=O)c4ccccc4C3=O)c2)c(-c2ccnc(Nc3ccccc3)c2)[nH]1] |
| 069 | 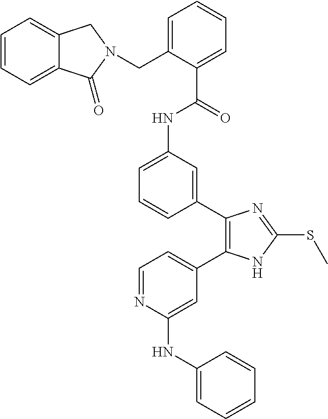 [SMILES: CSc1nc(-c2cccc(NC(=O)c3ccccc3CN3Cc4ccccc4C3=O)c2)c(-c2ccnc(Nc3ccccc3)c2)[nH]1] |

or a pharmaceutically acceptable salt thereof.

In another embodiment,
Z is C or S═O;
Y is selected from the group consisting of NH, C₁-C₃ alkyl, phenyl, naphthalene, pyridine, indole, thiophene, furan, C₁-C₅ cycloalkyl, and 3-5 membered heterocycloalkyl;
A is pyridine;
B is phenyl, thiophene, or dihydro-indene;
R¹ is C₁-C₃ alkyl;
R² is CO(C₁-C₃ alkyl) or phenyl, wherein phenyl is optionally substituted one or two times with R⁵;
R³ is H or halo;
each R⁴ is independently selected from the group consisting of H, OH, halo, C₁-C₃ alkyl, C₁-C₃ alkoxy, phenyl, thiophene, indole, CH₂-(5-10 membered bicyclic ring), and CH₂NHC(O)phenyl, wherein phenyl is optionally substituted one, two, or three times with halo, CO₂H, or C₁-C₃ haloalkyl;
each R⁵ is independently selected from the group consisting of halo, OH, C₁-C₃ alkoxy, and NHC(O)C₂-C₃ alkenyl; and
R⁶ is H.

In an embodiment, when Y is thiophene, R⁴ is not H.

In another embodiment, when Y is phenyl, R⁴ is not H.

In yet another embodiment, when Y is C₁ alkyl and R⁴ is unsubstituted phenyl, B is not phenyl.

In another embodiment, the compound of Formula I is not

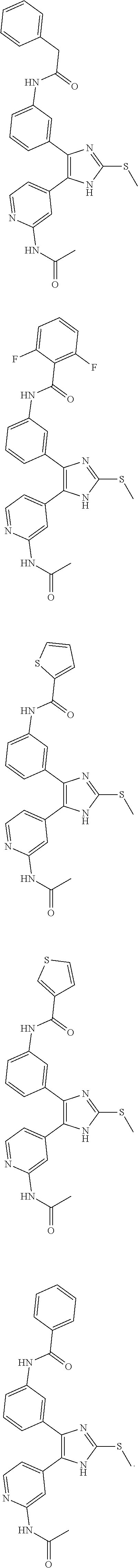 [SMILES: CSc1nc(-c2cccc(NC(=O)Cc3ccccc3)c2)c(-c2ccnc(NC(C)=O)c2)[nH]1 ; CSc1nc(-c2cccc(NC(=O)c3c(F)cccc3F)c2)c(-c2ccnc(NC(C)=O)c2)[nH]1 ; CSc1nc(-c2cccc(NC(=O)c3cccs3)c2)c(-c2ccnc(NC(C)=O)c2)[nH]1 ; CSc1nc(-c2cccc(NC(=O)c3ccsc3)c2)c(-c2ccnc(NC(C)=O)c2)[nH]1 ; CSc1nc(-c2cccc(NC(=O)c3ccccc3)c2)c(-c2ccnc(NC(C)=O)c2)[nH]1]

The compounds disclosed herein may exist as tautomers and optical isomers (e.g., enantiomers, diastereomers, diastereomeric mixtures, racemic mixtures, and the like).

It is generally well known in the art that any compound that will be converted in vivo to provide a compound disclosed herein is a prodrug within the scope of the present disclosure.

Compounds provided herein can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

In the compounds provided herein, any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Also, unless otherwise stated, when a position is designated specifically as “D” or “deuterium”, the position is understood to have deuterium at an abundance that is at least 3000 times greater than the natural abundance of deuterium, which is 0.015% (i.e., at least 45% incorporation of deuterium).

In embodiments, the compounds provided herein have an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In an aspect, provided herein is a pharmaceutical composition comprising any one of the compounds disclosed herein, or a pharmaceutically acceptable salt thereof, and at least one pharmaceutically acceptable carrier.

In an embodiment, the composition further comprises a second active agent. In another embodiment, the second active agent is selected from the group consisting of a MEK inhibitor, a PI3K inhibitor, and an mTor inhibitor. In yet another embodiment, the second active agent prevents EGFR dimer formation in a subject. In still another embodiment, the second active agent is selected from the group consisting of cetuximab, trastuzumab, and panitumumab. In an embodiment, the second active agent is an ATP competitive EGFR inhibitor. In another embodiment, the ATP competitive EGFR inhibitor is osimertinib, gefitinib, or erlotinib. In another embodiment, the ATP competitive EGFR inhibitor is osimertinib.

In another aspect, provided herein are pharmaceutical compositions comprising a compound of the present disclosure, or a pharmaceutically acceptable salt thereof, and a pharmaceutically acceptable carrier.

In another aspect, provided herein is a method of inhibiting the activity of EGFR, comprising administering to a subject in need thereof an effective amount of a compound of disclosed herein or a pharmaceutical composition comprising a compound disclosed herein and a pharmaceutically acceptable carrier. In an embodiment, the compound targets Cys775 on EGFR.

In another aspect, the pharmaceutical composition further comprises a second active agent, wherein said second active agent prevents EGFR dimer formation, and a pharmaceutically acceptable carrier. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab.

A compound that binds to an allosteric site in EGFR, such as the compounds of the present disclosure (e.g., the compounds of the formulae disclosed herein), optionally in combination with a second active agent, wherein said second active agent prevents EGFR dimer formation, are capable of modulating EGFR activity. In some embodiments, the compounds of the present disclosure are capable of inhibiting or decreasing EGFR activity without a second active agent (e.g., an antibody such as cetuximab, trastuzumab, or panitumumab). In other embodiments, the compounds of the present disclosure in combination with a second active agent. In an embodiment, the second active agent prevents EGFR dimer formation and/or are capable of inhibiting or decreasing EGFR activity. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

### Methods of Treatment

In an aspect, provided herein is a method of treating cancer in an individual in need thereof, comprising administering to the individual a therapeutically effective amount of a compound disclosed herein. In an embodiment, the cancer is selected from the group consisting of lung cancer, colon cancer, breast cancer, endometrial cancer, thyroid cancer, glioma, squamous cell carcinoma, and prostate cancer. In another embodiment, the cancer is non-small cell lung cancer (NSCLC).

In another aspect, provided herein is a method of inhibiting the activity of a kinase in a subject in need thereof, comprising administering to the subject a therapeutically effective amount of a compound provided herein. In an embodiment, the kinase is EGFR. In another embodiment, the EGFR is characterized by a mutation selected from the group consisting of L858R, T790M, and C797S, or any combination thereof.

In yet another aspect, provided herein is a method of treating or preventing a kinase-mediated disorder in an individual in need thereof, comprising administering to the individual a therapeutically effective amount of a compound of the present disclosure. In an embodiment, the kinase-mediated disorder is resistant to an EGFR-targeted therapy. In another embodiment, the EGFR-treated therapy is selected from the group consisting of gefitinib, erlotinib, osimertinib, CO-1686, and WZ4002.

In some embodiments, the compounds of the present disclosure are capable of modulating (e.g., inhibiting or decreasing) the activity of EGFR containing one or more mutations. In some embodiments, the mutant EGFR contains one or more mutations selected from T790M, L718Q, L844V, V948R, L858R, I941R, C797S, and Del. In other embodiments, the mutant EGFR contains a combination of mutations, wherein the combination is selected from Del/L718Q, Del/L844V, Del/T790M, Del/T790M/L718Q, Del/T790M/L844V, L858R/L718Q, L858R/L844V, L858R/T790M, L858R/T790M/I941R, Del/T790M, Del/T790M/C797S, L858R/T790M/C797S, and L858R/T790M/L718Q. In other embodiments, the mutant EGFR contains a combination of mutations, wherein the combination is selected from Del/L844V, L858R/L844V, L858R/T790M, L858R/T790M/I941R, L858R/T790M/C797S, Del/T790M, Del/T790M, Del/T790M/C797S, and L858R/T790M. In other embodiments, the mutant EGFR contains a combination of mutations, wherein the combination is selected from L858R/T790M, L858R/T790M/I941R, L858R/T790M/C797S, Del/T790M, Del/T790M/C797S, and L858R/T790M.

In some embodiments, the compounds of the present disclosure in combination with a second active agent, wherein said second active agent prevents EGFR dimer formation, are capable of modulating (e.g., inhibiting or decreasing) the activity of EGFR containing one or more mutations. In some embodiments, the mutant EGFR contains one or more mutations selected from T790M, L718Q, L844V, V948R, L858R, I941R, C797S, and Del. In other embodiments, the mutant EGFR contains a combination of mutations, wherein the combination is selected from Del/L718Q, Del/L844V, Del/T790M, Del/T790M/L718Q, Del/T790M/L844V, L858R/L7180, L858R/L844V, L858R/T790M, L858R/T790M/I941R, Del/T790M, Del/T790M/C797S, L858R/T790M/C797S, and L858R/T790M/L718Q. In other embodiments, the mutant EGFR contains a combination of mutations, wherein the combination is selected from Del/L844V, L858R/L844V, L858R/T790M, L858R/T790M/I941R, L858R/T790M/C797S, Del/T790M, Del/T790M1C797S, and L858R/T790M. In other embodiments, the mutant EGFR contains a combination of mutations, wherein the combination is selected from L858R/T790M, L858R/T790M/I941R, L858R/T790M/C797S, Del/T790M, Del/T790M/C797S, and L858R/T790M. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib.

In some embodiments, the compounds of the present disclosure are capable of modulating (e.g., inhibiting or decreasing) the activity of EGFR containing one or more mutations, but do not affect the activity of a wild-type EGFR.

In other embodiments, the compounds of the present disclosure in combination with a second active agent, wherein said second active agent prevents EGFR dimer formation, are capable of modulating (e.g., inhibiting or decreasing) the activity of EGFR containing one or more mutations, but do not affect the activity of a wild-type EGFR. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

Modulation of EGFR containing one or more mutations, such as those described herein, but not a wild-type EGFR, provides an approach to the treatment, prevention, or amelioration of diseases including, but not limited to, cancer and metastasis, inflammation, arthritis, systemic lupus erythematosus, skin-related disorders, pulmonary disorders, cardiovascular disease, ischemia, neurodegenerative disorders, liver disease, gastrointestinal disorders, viral and bacterial infections, central nervous system disorders, Alzheimer's disease, Parkinson's disease, Huntington's disease, amyotrophic lateral sclerosis, spinal cord injury, and peripheral neuropathy.

In some embodiments, the compounds of the disclosure exhibit greater inhibition of EGFR containing one or more mutations as described herein relative to a wild-type EGFR. In certain embodiments, the compounds of the disclosure exhibit at least 2-fold, 3-fold, 5-fold, 10-fold, 25-fold, 50-fold or 100-fold greater inhibition of EGFR containing one or more mutations as described herein relative to a wild-type EGFR. In various embodiments, the compounds of the disclosure exhibit up to 1000-fold greater inhibition of EGFR containing one or more mutations as described herein relative to a wild-type EGFR. In various embodiments, the compounds of the disclosure exhibit up to 10000-fold greater inhibition of EGFR having a combination of mutations described herein (e.g., L858R/T790M, L858R/T790M/I941R, L858R/T790M/C797S, Del/T790M, Del/T790M/C797S, and L858R/T790M) relative to a wild-type EGFR.

In other embodiments, the compounds of the disclosure in combination with a second active agent, wherein said second active agent prevents EGFR dimer formation, exhibit greater inhibition of EGFR containing one or more mutations as described herein relative to a wild-type EGFR. In certain embodiments, the compounds of the disclosure in combination with a second active agent, wherein said second active agent prevents EGFR dimer formation, exhibit at least 2-fold, 3-fold, 5-fold, 10-fold, 25-fold, 50-fold or 100-fold greater inhibition of EGFR containing one or more mutations as described herein relative to a wild-type EGFR. In various embodiments, the compounds of the disclosure in combination with a second active agent, wherein said second active agent prevents EGFR dimer formation, exhibit up to 1000-fold greater inhibition of EGFR containing one or more mutations as described herein relative to a wild-type EGFR. In various embodiments, the compounds of the disclosure in combination with a second active agent, wherein said second active agent prevents EGFR dimer formation, exhibit up to 10000-fold greater inhibition of EGFR having a combination of mutations described herein (e.g., L858R/T790M, L858R/T790M/I941R, L858R/T790M/C797S, Del/T790M, Del/T790M/C797S, and L858R/T790M) relative to a wild-type EGFR. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In some embodiments, the compounds of the disclosure exhibit from about 2-fold to about 10-fold greater inhibition of EGFR containing one or more mutations as described herein relative to a wild-type EGFR. In various embodiments, the compounds of the disclosure exhibit from about 10-fold to about 100-fold greater inhibition of EGFR containing one or more mutations as described herein relative to a wild-type EGFR. In various embodiments, the compounds of the disclosure exhibit from about 100-fold to about 1000-fold greater inhibition of EGFR containing one or more mutations as described herein relative to a wild-type EGFR. In various embodiments, the compounds of the disclosure exhibit from about 1000-fold to about 10000-fold greater inhibition of EGFR containing one or more mutations as described herein relative to a wild-type EGFR.

In other embodiments, the compounds of the disclosure in combination with a second active agent, wherein said second active agent prevents EGFR dimer formation, exhibit from about 2-fold to about 10-fold greater inhibition of EGFR containing one or more mutations as described herein relative to a wild-type EGFR. In other embodiments, the compounds of the disclosure in combination with a second active agent, wherein said second active agent prevents EGFR dimer formation, exhibit from about 10-fold to about 100-fold greater inhibition of EGFR containing one or more mutations as described herein relative to a wild-type EGFR. In other embodiments, the compounds of the disclosure in combination with a second active agent wherein said second active agent prevents EGFR dimer formation exhibit from about 100-fold to about 1000-fold greater inhibition of EGFR containing one or more mutations as described herein relative to a wild-type EGFR. In other embodiments, the compounds of the disclosure in combination with a second active agent, wherein said second active agent prevents EGFR dimer formation, exhibit from about 1000-fold to about 10000-fold greater inhibition of EGFR containing one or more mutations as described herein relative to a wild-type EGFR. In other embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In certain embodiments, the compounds of the disclosure exhibit at least 2-fold greater inhibition of EGFR having a combination of mutations selected from L858R/T790M, L858R/T790M/I941R, L858R/T790M/C797S, Del/T790M, De/T790M/C797S, and L858R/T790M relative to a wild-type EGFR. In certain embodiments, the compounds of the disclosure exhibit at least 3-fold greater inhibition of EGFR having a combination of mutations selected from L858R/T790M, L858R/T790M/I941R, L858R/T790M/C797S, Del/T790M, Del/T790M/C797S, and L858R/T790M relative to a wild-type EGFR. In certain embodiments, the compounds of the disclosure exhibit at least 5-fold greater inhibition of EGFR having a combination of mutations selected from L858R/T790M, L858R/T790M/I941R, L858R/T790M/C797S, Del/T790M, Del/T790M/C797S, and L858R/T790M relative to a wild-type EGFR.

In certain embodiments, the compounds of the disclosure exhibit at least 10-fold greater inhibition of EGFR having a combination of mutations selected from L858R/T790M, L858R/T790M/I941R, L858R/T790M/C797S, Del/T790M, Del/T790M/C797S, and L858R/T790M relative to a wild-type EGFR. In certain embodiments, the compounds of the disclosure exhibit at least 25-fold greater inhibition of EGFR having a combination of mutations selected from L858R/T790M, L858R/T790M/I941R, L858R/T790M/C797S, Del/T790M, Del/T790M/C797S, and L858R/T790M relative to a wild-type EGFR. In certain embodiments, the compounds of the disclosure exhibit at least 50-fold greater inhibition of EGFR having a combination of mutations selected from L L858R/T790M, L858R/T790M/I941R, L858R/T790M/C797S, Del/T790M, Del/T790M/C797S, and L858R/T790M relative to a wild-type EGFR. In certain embodiments, the compounds of the disclosure exhibit at least 100-fold greater inhibition of EGFR having a combination of mutations selected from L858R/T790M, L858R/T790M/I941R, L858R/T790M/C797S, Del/T790M, Del/T790M/C797S, and L858R/T790M relative to a wild-type EGFR.

In certain embodiments, the compounds of the disclosure in combination with a second active agent, wherein said second active agent prevents EGFR dimer formation, exhibit at least 2-fold greater inhibition of EGFR having a combination of mutations selected from L858R/T790M, L858R/T790M/I941R, L858R/T790M/C797S, Del/T790M, Del/T790M/C797S, and L858R/T790M relative to a wild-type EGFR. In certain embodiments, the compounds of the disclosure in combination with a second active agent, wherein said second active agent prevents EGFR dimer formation, exhibit at least 3-fold greater inhibition of EGFR having a combination of mutations selected from L858R/T790M, L858R/T790M/I941R, L858R/T790M/C797S, Del/T790M, Del/T790M/C797S, and L858R/T790M relative to a wild-type EGFR. In certain embodiments, the compounds of the disclosure in combination with a second active agent, wherein said second active agent prevents EGFR dimer formation, exhibit at least 5-fold greater inhibition of EGFR having a combination of mutations selected from L858R/T790M, L858R/T790M/I941R, L858R/T790M/C797S, Del/T790M, De/T790M/C797S, and L858R/T790M relative to a wild-type EGFR.

In certain embodiments, the compounds of the disclosure in combination with a second active agent, wherein said second active agent prevents EGFR dimer formation, exhibit at least 10-fold greater inhibition of EGFR having a combination of mutations selected from L858R/T790M, L858R/T790M/I941R, L858R/T790M/C797S, Del/T790M, Del/T790M/C797S, and L858R/T790M relative to a wild-type EGFR. In certain embodiments, the compounds of the disclosure in combination with a second active agent, wherein said second active agent prevents EGFR dimer formation, exhibit at least 25-fold greater inhibition of EGFR having a combination of mutations selected from L858R/T790M, L858R/T1790M/I941R, L858R/T790M/C797S, Del/T790M, Del/T790M/C797S, and L858R/T790M relative to a wild-type EGFR. In certain embodiments, the compounds of the disclosure in combination with a second active agent, wherein said second active agent prevents EGFR dimer formation, exhibit at least 50-fold greater inhibition of EGFR having a combination of mutations selected from L L858R/T790M, L858R/T790M/I941R, L858R/T790M/C797S, Del/T790M, Del/T790M/C797S, and L858R/T790M relative to a wild-type EGFR. In certain embodiments, the compounds of the disclosure in combination with a second active agent, wherein said second active agent prevents EGFR dimer formation, exhibit at least 100-fold greater inhibition of EGFR having a combination of mutations selected from L858R/T790M, L858R/T790M/I941R, L858R/T790M/C797S, Del/T790M, Del/T790M/C797S, and L858R/T790M relative to a wild-type EGFR.

In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib.

In some embodiments, the inhibition of EGFR activity is measured by IC₅₀.

In some embodiments, the inhibition of EGFR activity is measured by EC₅₀.

In some embodiments, the inhibition of EGFR by a compound of the disclosure can be measured via a biochemical assay. By illustrative and non-limiting example, a homogenous time-resolved fluorescence (HTRF) assay may be used to determine inhibition of EGFR activity using conditions and experimental parameters disclosed herein. The HTRF assay may, for example, employ concentrations of substrate (e.g., biotin-Lck-peptide substrate) of about 1 μM; concentrations of EGFR (mutant or WT) from about 0.2 nM to about 40 nM; and concentrations of inhibitor from about 0.000282 μM to about 50 μM. A compound of the disclosure screened under these conditions may, for example, exhibit an ICs value from about 1 nM to >1 μM; from about 1 nM to about 400 nM; from about 1 nM to about 150 nM; from about 1 nM to about 75 nM; from about 1 nM to about 40 nM; from about 1 nM to about 25 nM; from about 1 nM to about 15 nM; or from about 1 nM to about 10 nM. In certain embodiments, a compound of the disclosure screened under the above conditions for inhibition of EGFR having a mutation or combination of mutations selected from L858R/T790M, L858R, and T790M may, for example, exhibit an IC₅₀ value from about 1 nM to >1 μM; from about 1 nM to about 400 nM; from about 1 nM to about 150 nM; from about 1 nM to about 75 nM; from about 1 nM to about 40 nM; from about 1 nM to about 25 nM; from about 1 nM to about 15 nM; or from about 1 nM to about 10 nM.

In some embodiments, the compounds of the disclosure bind to an allosteric site in EGFR. In some embodiments, the compounds of the disclosure interact with at least one amino acid residue of epidermal growth factor receptor (EGFR) selected from Lys745, Leu788, and Ala 743. In other embodiments, the compounds of the disclosure interact with at least one amino acid residue of epidermal growth factor receptor (EGFR) selected from Cys755, Leu777, Phe856, and Asp855. In other embodiments, the compounds of the disclosure interact with at least one amino acid residue of epidermal growth factor receptor (EGFR) selected from Met766, Ile759, Glu762, and Ala763. In other embodiments, the compounds of the disclosure interact with at least one amino acid residue of epidermal growth factor receptor (EGFR) selected from Lys745, Leu788, and Ala 743; at least one amino acid residue of epidermal growth factor receptor (EGFR) selected from Cys755, Leu777, Phe856, and Asp855; and at least one amino acid residue of epidermal growth factor receptor (EGFR) selected from Met766, Ile759, Glu762, and Ala763. In other embodiments, the compounds of the disclosure do not interact with any of the amino acid residues of epidermal growth factor receptor (EGFR) selected from Met793, Gly796, and Cys797.

In some embodiments, the disclosure provides a compound comprising an allosteric kinase inhibitor, wherein the compound is a more potent inhibitor of a drug-resistant EGFR mutant relative to a wild type EGFR. For example, the compound can be at least about 2-fold, 3-fold, 5-fold, 10-fold, 25-fold, 50-fold or about 100-fold more potent at inhibiting the kinase activity of the drug-resistant EGFR mutant relative to a wild-type EGFR. In some embodiments, the drug-resistant EGFR mutant is resistant to one or more known EGFR inhibitors, including but not limited to gefitinib, erlotinib, lapatinib, WZ4002, HKI-272, CL-387785, and osimertinib.

In some embodiments, the drug-resistant EGFR mutant comprises a sensitizing mutation, such as Del and L858R.

In some embodiments, the disclosure provides a compound comprising an allosteric kinase inhibitor in combination with a second active agent, wherein said second active agent prevents EGFR dimer formation, wherein the compound is a more potent inhibitor of a drug-resistant EGFR mutant relative to a wild type EGFR. For example, the compound in combination with a second active agent, wherein said second active agent prevents EGFR dimer formation, can be at least about 2-fold, 3-fold, 5-fold, 10-fold, 25-fold, 50-fold or about 100-fold more potent at inhibiting the kinase activity of the drug-resistant EGFR mutant relative to a wild-type EGFR. In some embodiments, the drug-resistant EGFR mutant is resistant to one or more known EGFR inhibitors, including but not limited to gefitinib, erlotinib, lapatinib, WZ4002, HKI-272, CL-387785, and osimertinib. In some embodiments, the drug-resistant EGFR mutant comprises a sensitizing mutation, such as Del and L858R. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib.

In some embodiments, the disclosure provides a compound comprising an allosteric kinase inhibitor, wherein the compound inhibits kinase activity of a drug-resistant EGFR mutant harboring a sensitizing mutation (e.g., Del and L858R) and a drug-resistance mutation (e.g., T790M, L718Q, C797S, and L844V) with less than a 10-fold difference in potency (e.g., as measured by IC₅₀) relative to an EGFR mutant harboring the sensitizing mutation but not the drug-resistance mutation. In some embodiments, the difference in potency is less than about 9-fold, 8-fold, 7-fold, 6-fold, 5-fold, 4-fold, 3-fold, or 2-fold.

In other embodiments, the disclosure provides a compound comprising an allosteric kinase inhibitor in combination with a second active agent, wherein said second active agent prevents EGFR dimer formation, wherein the compound in combination with the second active agent inhibits kinase activity of a drug-resistant EGFR mutant harboring a sensitizing mutation (e.g., Del and L858R) and a drug-resistance mutation (e.g., T790M, L718Q, C797S, and L844V) with less than a 10-fold difference in potency (e.g., as measured by IC₅₀) relative to an EGFR mutant harboring the sensitizing mutation but not the drug-resistance mutation. In some embodiments, the difference in potency is less than about 9-fold, 8-fold, 7-fold, 6-fold, 5-fold, 4-fold, 3-fold, or 2-fold. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In some embodiments, the disclosure provides a compound comprising an allosteric kinase inhibitor, wherein the compound is more potent than one or more known EGFR inhibitors, including but not limited to gefitinib, erlotinib, lapatinib, WZ4002, HKI-272, CL-387785, and osimertinib, at inhibiting the activity of EGFR containing one or more mutations as described herein, such as T790M, L718Q, L844V, L858R, C797S, and Del. For example, the compound can be at least about 2-fold, 3-fold, 5-fold, 10-fold, 25-fold, 50-fold or about 100-fold more potent (e.g., as measured by IC₅₀) than gefitinib, erlotinib, lapatinib, WZ4002, HKI-272, CL-387785, and osimertinib at inhibiting the activity of the EGFR containing one or more mutations as described herein.

In other embodiments, the disclosure provides a compound comprising an allosteric kinase inhibitor in combination with a second active agent, wherein said second active agent prevents EGFR dimer formation, wherein the compound in combination with the second active agent is more potent than one or more known EGFR inhibitors, including but not limited to gefitinib, erlotinib, lapatinib, WZ4002, HKI-272, CL-387785, and osimertinib, at inhibiting the activity of EGFR containing one or more mutations as described herein, such as T790M, L718Q, L844V, L858R, C797S, and Del. For example, the compound in combination with a second active agent, wherein said second active agent prevents EGFR dimer formation, can be at least about 2-fold, 3-fold, 5-fold, 10-fold, 25-fold, 50-fold or about 100-fold more potent (e.g., as measured by IC₅₀) than gefitinib, erlotinib, lapatinib, WZ4002, HKI-272, CL-387785, and osimertinib at inhibiting the activity of the EGFR containing one or more mutations as described herein. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In some embodiments, the disclosure provides a compound comprising an allosteric kinase inhibitor, wherein the compound is less potent than one or more known EGFR inhibitors, including but not limited to gefitinib, erlotinib, lapatinib, WZ4002, HKI-272, CL-387785, and osimertinib, at inhibiting the activity of a wild-type EGFR. For example, the compound can be at least about 2-fold, 3-fold, 5-fold, 10-fold, 25-fold, 50-fold or about 100-fold less potent (e.g., as measured by IC₅₀) than gefitinib, erlotinib, lapatinib, WZ4002, HKI-272, CL-387785, and osimertinib, at inhibiting the activity of a wild-type EGFR.

In other embodiments, the disclosure provides a compound comprising an allosteric kinase inhibitor in combination with a second active agent, wherein said second active agent prevents EGFR dimer formation, wherein the compound in combination with the second active agent is less potent than one or more known EGFR inhibitors, including but not limited to gefitinib, erlotinib, lapatinib, WZ4002, HKI-272, CL-387785, and osimertinib, at inhibiting the activity of a wild-type EGFR. For example, the compound in combination with a second active agent, wherein said second active agent prevents EGFR dimer formation can be at least about 2-fold, 3-fold, 5-fold, 10-fold, 25-fold, 50-fold or about 100-fold less potent (e.g., as measured by IC₅₀) than gefitinib, erlotinib, lapatinib, WZ4002, HKI-272, CL-387785, and osimertinib, at inhibiting the activity of a wild-type EGFR. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

Potency of the inhibitor can be determined by EC₅₀ value. A compound with a lower EC₅₀ value, as determined under substantially similar conditions, is a more potent inhibitor relative to a compound with a higher EC₅₀ value. In some embodiments, the substantially similar conditions comprise determining an EGFR-dependent phosphorylation level, in vitro or in vivo (e.g., in 3T3 cells expressing a wild type EGFR, a mutant EGFR, or a fragment of any thereof).

Potency of the inhibitor can also be determined by IC₅₀ value. A compound with a lower IC₅₀ value, as determined under substantially similar conditions, is a more potent inhibitor relative to a compound with a higher IC₅₀ value. In some embodiments, the substantially similar conditions comprise determining an EGFR-dependent phosphorylation level, in vitro or in vivo (e.g., in 3T3 cells expressing a wild type EGFR, a mutant EGFR, or a fragment of any thereof).

An EGFR sensitizing mutation comprises without limitation L858R, G719S, G719C, G719A, L861Q, a deletion in exon 19 and/or an insertion in exon 20. A drug-resistant EGFR mutant can have without limitation a drug resistance mutation comprising T790M, T854A, L718Q, C797S, or D761Y.

The selectivity between wild-type EGFR and EGFR containing one or more mutations as described herein can also be measured using cellular proliferation assays where cell proliferation is dependent on kinase activity. For example, murine Ba/F3 cells transfected with a suitable version of wild-type EGFR (such as VIII; containing a WT EGFR kinase domain), or Ba/F3 cells transfected with L858R/T790M, Del/T790M/L718Q, L858R/T790M/L718Q, L858R/T790M/C797S, Del/T790M/C797S, L858R/T790M/I941R, or Exon 19 deletion/T790M can be used. Proliferation assays are performed at a range of inhibitor concentrations (10 μM, 3 μM, 1.1 μM, 330 nM, 110 nM, 33 nM, 11 nM, 3 nM, I nM) and an EC₅₀ is calculated.

An alternative method to measure effects on EGFR activity is to assay EGFR phosphorylation. Wild type or mutant (L858R/T790M, Del/T790M, Del/T790M/L7180, L858R/T790M/C797S, Del/T790M/C797S, L858R/T790M/I941R, or L858R/T790M/L718Q) EGFR can be transfected into NIH-3T3 cells (which do not normally express endogenous EGFR) and the ability of the inhibitor (using concentrations as above) to inhibit EGFR phosphorylation can be assayed. Cells are exposed to increasing concentrations of inhibitor for 6 hours and stimulated with EGF for 10 minutes. The effects on EGFR phosphorylation are assayed by Westem Blotting using phospho-specific (Y1068) EGFR antibodies.

In another aspect, the present disclosure relates to a compound that binds to an allosteric site in EGFR, wherein the compound exhibits greater than 2-fold, 3-fold, 5-fold, 10-fold, 25-fold, 50-fold, 100-fold, or 1000-fold inhibition of EGFR containing one or more mutations as described herein (e.g., L858R/T790M, Del/T790M, Del/T790M/L718Q, L858R/T790M/C797S, Del/T790M/C797S, L858R/T790M/I941R, or L858R/T790M/L718Q) relative to a wild-type EGFR.

In other embodiments, the disclosure provides a compound that binds to an allosteric site in EGFR in combination with a second active agent, wherein said second active agent prevents EGFR dimer formation, wherein the compound in combination with the second active agent greater than 2-fold, 3-fold, 5-fold, 10-fold, 25-fold, 50-fold, 100-fold, or 1000-fold inhibition of EGFR containing one or more mutations as described herein (e.g., L858R/T790M, Del/T790M, Del/T790M/L718Q, Del/T790M/C797S, L858R/T790M/C797S, L858R/T790M/I941R, or L858R/T790M/L7180) relative to a wild-type EGFR. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In still another aspect, the disclosure provides a method of inhibiting epidermal growth factor receptor (EGFR), the method comprising administering to a subject in need thereof an effective amount of a compound of disclosed herein, or a pharmaceutically acceptable salt thereof. In some embodiments, the method further comprises administering a second active agent, wherein said second active agent prevents EGFR dimer formation. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In another aspect, provided herein is a method of treating or preventing a disease, the method comprising administering to a subject in need thereof an effective amount of a compound of disclosed herein, or a pharmaceutically acceptable salt thereof. In some embodiments, the disease is mediated by a kinase. In further embodiments, the kinase comprises a mutated cysteine residue. In further embodiments, the mutated cysteine residue is located in or near the position equivalent to Cys 797 in EGFR, including such positions in Jak3, Blk, Bmx, Btk, HER2 (ErbB2), HER4 (ErbB4), Itk, Tec, and Txk. In some embodiments, the method further comprises administering a second active agent, wherein said second active agent prevents dimer formation of the kinase. In some embodiments, the second active agent that prevents kinase dimer formation is an antibody. In further embodiments, the second active agent prevents EGFR dimer formation. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In some embodiments, the disease is mediated by EGFR (e.g., EGFR plays a role in the initiation or development of the disease). In some embodiments, the disease is mediated by a Her-kinase. In further embodiments, the Her-kinase is HER1, HER2, or HER4.

In certain embodiments, the disease is resistant to a known EGFR inhibitor, including but not limited to, gefitinib, erlotinib, osimertinib, CO-1686, or WZ4002. In certain embodiments, a diagnostic test is performed to determine if the disease is associated with an activating mutation in EGFR. In certain embodiments, a diagnostic test is performed to determine if the disease is associated with an EGFR harboring an activating mutation and/or a drug resistance mutation. Activating mutations comprise without limitation L858R, G719S, G719C, G719A, L718Q, L8610, a deletion in exon 19 and/or an insertion in exon 20. Drug resistant EGFR mutants can have without limitation a drug resistance mutation comprising T790M, T854A, L718Q, C797S, or D761Y. The diagnostic test can comprise sequencing, pyrosequencing, PCR, RT-PCR, or similar analysis techniques known to those of skill in the art that can detect nucleotide sequences.

In certain embodiments, the disease is cancer or a proliferation disease.

In further embodiments, the disease is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, or solid tumors. In further embodiments, the disease is lung cancer, breast cancer, glioma, squamous cell carcinoma, or prostate cancer. In still further embodiments, the disease is non-small cell lung cancer.

In certain embodiments, the disease is resistant to a known EGFR inhibitor, including but not limited to, gefitinib, erlotinib, osimertinib, CO-1686, or WZ4002. In certain embodiments, a diagnostic test is performed to determine if the disease is associated with an activating mutation in EGFR. In certain embodiments, a diagnostic test is performed to determine if the disease is associated with an EGFR harboring an activating mutation and/or a drug resistance mutation. Activating mutations comprise without limitation L858R, G719S, G719C, G719A, L718Q, L861Q, a deletion in exon 19 and/or an insertion in exon 20. Drug resistant EGFR mutants can have without limitation a drug resistance mutation comprising T790M, T854A, L718Q, C797S, or D761Y. The diagnostic test can comprise sequencing, pyrosequencing, PCR, RT-PCR, or similar analysis techniques known to those of skill in the art that can detect nucleotide sequences.

In yet another aspect, provided herein is a method of treating a kinase-mediated disorder comprising administering to a subject in need thereof an effective amount of a compound disclosed herein, or a pharmaceutically acceptable salt thereof. In some embodiments, the compound is an inhibitor of HER1, HER2, or HER4. In other embodiments, the subject is administered an additional therapeutic agent. In other embodiments, the compound and the additional therapeutic agent are administered simultaneously or sequentially.

In another aspect, the disclosure provides a method of treating a kinase mediated disorder, the method comprising administering to a subject in need thereof an effective amount of a compound of disclosed herein, or a pharmaceutically acceptable salt thereof, and a second active agent, wherein said second active agent prevents EGFR dimer formation. In some embodiments, the compound is an inhibitor of HER1, HER2, or HER4. In other embodiments, the subject is administered an additional therapeutic agent. In other embodiments, the compound, the second active agent that prevents EGFR dimer formation, and the additional therapeutic agent are administered simultaneously or sequentially. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In other embodiments, the disease is cancer. In further embodiments, the cancer is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, or solid tumors. In further embodiments, the disease is lung cancer, breast cancer, glioma, squamous cell carcinoma, or prostate cancer. In still further embodiments, the disease is non-small cell lung cancer.

In another aspect, provided herein is a method of treating cancer, wherein the cancer cell comprises activated EGFR, comprising administering to a subject in need thereof an effective amount of a compound of disclosed herein, or a pharmaceutically acceptable salt thereof.

In another aspect, provided herein is a method of treating cancer, wherein the cancer cell comprises activated EGFR, comprising administering to a subject in need thereof an effective amount of a compound of disclosed herein, or a pharmaceutically acceptable salt thereof and a second active agent, wherein said second active agent prevents EGFR dimer formation. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In certain embodiments, the EGFR activation is selected from mutation of EGFR, amplification of EGFR, expression of EGFR, and ligand mediated activation of EGFR.

In further embodiments, the mutation of EGFR is selected from G719S, G719C, G719A, L858R, L861Q, an exon 19 deletion mutation, and an exon 20 insertion mutation.

In still another aspect, provided herein is a method of treating cancer in a subject, wherein the subject is identified as being in need of EGFR inhibition for the treatment of cancer, comprising administering to the subject an effective amount of a compound disclosed herein, or a pharmaceutically acceptable salt thereof.

In certain embodiments, the subject identified as being in need of EGFR inhibition is resistant to a known EGFR inhibitor, including but not limited to, gefitinib, erlotinib, osimertinib, CO-1686, or WZ4002. In certain embodiments, a diagnostic test is performed to determine if the subject has an activating mutation in EGFR. In certain embodiments, a diagnostic test is performed to determine if the subject has an EGFR harboring an activating mutation and/or a drug resistance mutation. Activating mutations comprise without limitation L858R, G719S, G719C, G719A, L718Q, L861Q, a deletion in exon 19 and/or an insertion in exon 20. Drug resistant EGFR mutants can have without limitation a drug resistance mutation comprising T790M, T854A, L718Q, C797S, or D761Y. The diagnostic test can comprise sequencing, pyrosequencing, PCR, RT-PCR, or similar analysis techniques known to those of skill in the art that can detect nucleotide sequences.

In an aspect, provided herein is a method of preventing resistance to a known EGFR inhibitor (including but not limited to gefitinib, erlotinib, osimertinib, CO-1686, or WZ4002) in a subject, comprising administering to a subject in need thereof an effective amount of a compound disclosed herein, or a pharmaceutically acceptable salt thereof.

In another aspect, provided herein is a method of preventing resistance to a known EGFR inhibitor (including but not limited to gefitinib, erlotinib, osimertinib, CO-1686, or WZ4002) in a disease, comprising administering to a subject in need thereof an effective amount of a compound disclosed herein, or a pharmaceutically acceptable salt thereof, and a second active agent, wherein said second active agent prevents EGFR dimer formation. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab.

In an embodiment of the methods disclosed herein, the subject is a human.

In another aspect, the disclosure provides a compound disclosed herein, or a pharmaceutically acceptable salt thereof, for use in the manufacture of a medicament for treating or preventing a disease in which EGFR plays a role.

In an aspect, provided herein is a method of treating or preventing a condition selected from the group consisting of autoimmune diseases, inflammatory diseases, proliferative and hyperproliferative diseases, immunologically-mediated diseases, bone diseases, metabolic diseases, neurological and neurodegenerative diseases, cardiovascular diseases, hormone related diseases, allergies, asthma, and Alzheimer's disease. In other embodiments, said condition is selected from a proliferative disorder and a neurodegenerative disorder.

One aspect of this disclosure provides compounds that are useful for the treatment of diseases, disorders, and conditions characterized by excessive or abnormal cell proliferation. Such diseases include, but are not limited to, a proliferative or hyperproliferative disease, and a neurodegenerative disease. Examples of proliferative and hyperproliferative diseases include, without limitation, cancer. The term “cancer” includes, but is not limited to, the following cancers: breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, colorectal, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon, rectum, large intestine, rectum, brain and central nervous system, chronic myeloid leukemia (CML), and leukemia. The term “cancer” includes, but is not limited to, the following cancers: myeloma, lymphoma, or a cancer selected from gastric, renal, head and neck, oropharyngeal, non-small cell lung cancer (NSCLC), endometrial, hepatocarcinoma, non-Hodgkin's lymphoma, and pulmonary.

The term “cancer” refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL). B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodysplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in preventing, treating and studying are, for example, colon carcinoma, familial adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma. In one aspect of the disclosure, the present disclosure provides for the use of one or more compounds of the disclosure in the manufacture of a medicament for the treatment of cancer, including without limitation the various types of cancer disclosed herein.

In some embodiments, the compounds of this disclosure are useful for treating cancer, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In some embodiments, the compounds of this disclosure are useful for treating hematopoietic disorders, in particular, acute-myelogenous leukemia (AML), chronic-myelogenous leukemia (CML), acute-promyelocytic leukemia, and acute lymphocytic leukemia (ALL).

The term “cancerous cell” as provided herein, includes a cell afflicted by any one of the above-identified conditions.

The disclosure further provides a method for the treatment or prevention of cell proliferative disorders such as hyperplasias, dysplasias and pre-cancerous lesions. Dysplasia is the earliest form of pre-cancerous lesion recognizable in a biopsy by a pathologist. The subject compounds may be administered for the purpose of preventing said hyperplasias, dysplasias, or pre-cancerous lesions from continuing to expand or from becoming cancerous. Examples of pre-cancerous lesions may occur in skin, esophageal tissue, breast and cervical intra-epithelial tissue.

Examples of neurodegenerative diseases include, without limitation, adrenoleukodystrophy (ALD), Alexander's disease, Alper's disease, Alzheimers disease, amyotrophic lateral sclerosis (Lou Gehrig's Disease), ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, Creutzfeldt-Jakob disease, familial fatal insomnia, frontotemporal lobar degeneration, Huntington's disease, HIV-associated dementia, Kennedy's disease, Krabbe's disease, Lewy body dementia, neuroborreliosis, Machado-Joseph disease (spinocerebellar ataxia type 3), multiple system atrophy, multiple sclerosis, narcolepsy, Niemann Pick disease, Parkinson's disease, Pelizaeus-Merzbacher disease, Pick's disease, primary lateral sclerosis, prion diseases, progressive supranuclear palsy, Refsum's disease, Sandhoff disease, Schilders disease, subacute combined degeneration of spinal cord secondary to pernicious anaemia, Spielmeyer-Vogt-Sjogren-Batten disease (also known as Batten disease), spinocerebellar ataxia (multiple types with varying characteristics), spinal muscular atrophy, Steele-Richardson-Olszewski disease, tabes dorsalis, and toxic encephalopathy.

Another aspect of this disclosure provides a method for the treatment or lessening the severity of a disease selected from a proliferative or hyperproliferative disease, or a neurodegenerative disease, comprising administering an effective amount of a compound, or a pharmaceutically acceptable composition comprising a compound, to a subject in need thereof. In other embodiments, the method further comprises administering a second active agent, wherein said second active agent prevents EGFR dimer formation. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

The activity of the compounds and compositions of the present disclosure as EGFR kinase inhibitors may be assayed in vitro, in vivo, or in a cell line. In vitro assays include assays that determine inhibition of either the kinase activity or ATPase activity of the activated kinase. Alternate in vitro assays quantitate the ability of the inhibitor to bind to the protein kinase and may be measured either by radio labelling the inhibitor prior to binding, isolating the inhibitor/kinase complex and determining the amount of radio label bound, or by running a competition experiment where new inhibitors are incubated with the kinase bound to known radioligands. Detailed conditions for assaying a compound utilized in this disclosure as an inhibitor of various kinases are set forth in the Examples below.

In accordance with the foregoing, the present disclosure further provides a method for preventing or treating any of the diseases or disorders described above in a subject in need of such treatment, which method comprises administering to said subject a therapeutically effective amount of a compound of the disclosure, or a pharmaceutically acceptable salt thereof, and optionally a second active agent, wherein said second active agent prevents EGFR dimer formation. For any of the above uses, the required dosage will vary depending on the mode of administration, the particular condition to be treated and the effect desired.

In other embodiments, the compound and the second active agent that prevents EGFR dimer formation are administered simultaneously or sequentially.

### Administration/Dosages/Formulations

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations (for example, sterile injectable aqueous or oleaginous suspensions) may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringers solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an of vehicle.

Compositions for rectal or vaginal administration are preferably suppositories which can be prepared by mixing the compounds of this disclosure with suitable non-irritating excipients or carriers such as cocoa butter, polyethylene glycol, or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this disclosure.

The ointments, pastes, creams and gels may contain, in addition to an active compound of this disclosure, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this disclosure, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present disclosure, disorders are treated or prevented in a subject, such as a human or other animal, by administering to the subject a therapeutically effective amount of a compound of the disclosure, in such amounts and for such time as is necessary to achieve the desired result. The term “therapeutically effective amount” of a compound of the disclosure, as used herein, means a sufficient amount of the compound so as to decrease the symptoms of a disorder in a subject. As is well understood in the medical arts a therapeutically effective amount of a compound of this disclosure will be at a reasonable benefit/risk ratio applicable to any medical treatment.

In general, compounds of the disclosure will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g., humans, is in the range from about 0.5 mg to about 100 mg, conveniently administered, e.g., in divided doses up to four times a day or in retard form. Suitable unit dosage forms for oral administration comprise from ca. 1 to 50 mg active ingredient.

In certain embodiments, a therapeutic amount or dose of the compounds of the present disclosure may range from about 0.1 mg/Kg to about 500 mg/Kg, alternatively from about 1 to about 50 mg/Kg. In general, treatment regimens according to the present disclosure comprise administration to a patient in need of such treatment from about 10 mg to about 1000 mg of the compound(s) of this disclosure per day in single or multiple doses. Therapeutic amounts or doses will also vary depending on route of administration, as well as the possibility of co-usage with other agents.

Upon improvement of a subject's condition, a maintenance dose of a compound, composition or combination of this disclosure may be administered, if necessary. Subsequently, the dosage or frequency of administration, or both, may be reduced, as a function of the symptoms, to a level at which the improved condition is retained; when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present disclosure will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The disclosure also provides for a pharmaceutical combination, e.g., a kit, comprising a) a first agent which is a compound of the disclosure as disclosed herein, in free form or in pharmaceutically acceptable salt form, and b) at least one co-agent. The kit can comprise instructions for its administration.

In certain embodiments, these compositions optionally further comprise one or more additional therapeutic agents. For example, an agent that prevents EGFR dimer formation, chemotherapeutic agents or other antiproliferative agents may be combined with the compounds of this disclosure to treat proliferative diseases and cancer.

Some examples of materials which can serve as pharmaceutically acceptable carriers include, but are not limited to, ion exchangers; alumina; aluminum stearate; lecithin; serum proteins, such as human serum albumin; buffer substances such as phosphates, glycine, sorbic acid, or potassium sorbate; partial glyceride mixtures of saturated vegetable fatty acids; water; salts or electrolytes, such as protamine sulfate; disodium hydrogen phosphate; potassium hydrogen phosphate; sodium chloride; zinc salts; colloidal silica; magnesium trisilicate; polyvinyl pyrrolidone; polyacrylates; waxes; polyethylenepolyoxypropylene-block polymers; wool fat sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols, such a propylene glycol or polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; and phosphate buffer solutions. Further, non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The protein kinase inhibitors or pharmaceutical salts thereof may be formulated into pharmaceutical compositions for administration to animals or humans. These pharmaceutical compositions, which comprise an amount of the protein inhibitor effective to treat or prevent a protein kinase-mediated condition and a pharmaceutically acceptable carrier, are other embodiments of the present disclosure.

### Kits

In an aspect, provided herein is a kit comprising a compound capable of inhibiting kinase activity selected from one or more compounds of disclosed herein, or pharmaceutically acceptable salts thereof, and instructions for use in treating cancer. In certain embodiments, the kit further comprises components for performing a test to determine whether a subject has activating and/or drug resistance mutations in EGFR.

In another aspect, the disclosure provides a kit comprising a compound capable of inhibiting EGFR activity selected from a compound disclosed herein, or a pharmaceutically acceptable salt thereof.

In another aspect, the disclosure provides a kit comprising a compound capable of inhibiting kinase activity selected from one or more compounds of disclosed herein, or pharmaceutically acceptable salts thereof, a second active agent, wherein said second active agent prevents EGFR dimer formation; and instructions for use in treating cancer. In certain embodiments, the kit further comprises components for performing a test to determine whether a subject has activating and/or drug resistance mutations in EGFR. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab.

In another aspect, the disclosure provides a kit comprising a compound capable of inhibiting EGFR activity selected from a compound of disclosed herein, or a pharmaceutically acceptable salt thereof and a second active agent, wherein said second active agent prevents EGFR dimer formation. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### EXAMPLES

The application is further illustrated by the following examples, which should not be construed as further limiting. The practice of the present disclosure will employ, unless otherwise indicated, conventional techniques of organic synthesis, cell biology, cell culture, and molecular biology, which are within the skill of the art.

### General:

Chemical synthesis was carried out using commonly applied techniques and general procedures. All starting materials and reagents were of commercial quality and were used without further purification unless otherwise stated. Thin layer chromatography (TLC) was carried out on Merck 60 F254 and Macherey Nager ALUGRAM® Xtra SIL G/UV254 silica plates and were visualized under UV light (254 nm and 366 nm) or developed with an appropriate staining reagent. Preparative column chromatography was carried out with an Interchim PuriFlash 430 or PuriFlash XS420 automated flash chromatography system on Grace Davison Davisil LC60A 20-45 micron or Merck Geduran Si60 63-200 micron silica.

Analytical Methods and Instrumentation:

NMR: ¹H and ¹³C spectra were recorded on Bruker Avance 200, Bruker Avance 400 or Bruker Avance 600 instruments. The samples were dissolved in deuterated solvents and chemical shifts are given in relation to tetramethylsilane (TMS). Spectra were calibrated using the residual peaks of the used solvent.

MS: Mass spectra were obtained using a Advion TLC-MS interface with electron spray ionization (ESI) in positive and/or negative mode. Instrument settings as follows: ESI voltage 3.50 kV, capillary voltage 187 V, source voltage 44 V, capillary temperature 250° C., desolvation gas temperature 250° C., gas flow 5 l/min nitrogen.

HPLC: Purity of final compounds was determined using an Agilent 1100 Series LC with Phenomenex Luna C8 columns (150×4.6 mm, 5 μm) and detection was performed with a UV DAD at 254 nm and 230 nm wavelength. Elution was carried out with the following gradient 0.01 M KH₂PO₄, pH 2.30 (solvent A), MeOH (solvent B), 40% B to 85% B in 8 min, 85% B for 5 min, 85% to 40% B in 1 min, 40% B for 2 min, stop time 16 min, flow 1.5 ml/min. Unless otherwise state all final compounds showed a purity above 95% in the means of area percent at the two different wavelengths.

### Example 1: Synthetic Protocol

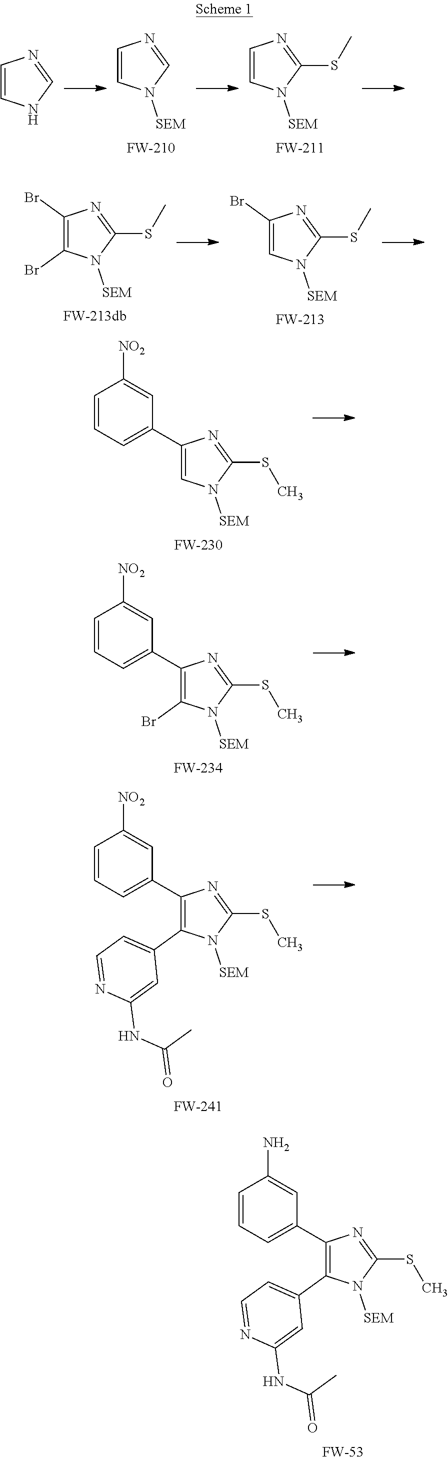

### 1-((2-(Trimethylsilyl)ethoxy)methyl)-1H-Imidazole (FW-210)

10.02 g (147.2 mmol) of imidazole was dissolved in 250 mL of THF under nitrogen atmosphere and cooled down to −5° C. 6.77 g (169.3 mmol) of a 60% dispersion in oil of sodium hydride was added portion wise to the solution maintaining temperature under 0° C. 27.20 mL (154.5 mmol) SEM-Cl was added dropwise to the stirred reaction mixture maintaining the temperature under 10° C. After full addition, the mixture was warmed up to room temperature and stirred there for 1 h until complete conversion. 200 mL of brine was added and the organic layer was separated. The aqueous phase was extracted with EtOAc twice and the combined organic layers were dried over Na₂SO₄. After evaporation of the solvent the residue was distilled to obtain a colorless liquid (bp: 85° C., p=8×10⁻³ mbar) in 91% yield (26.47 g, 133.9 mmol). ¹H NMR (200 MHz, CDCl₃) δ 7.5 (s, 1H), 7.06-7.00 (m, 1H), 7.00-6.95 (m, 1H), 5.21 (s, 2H), 3.46-3.35 (m, 2H), 0.89-0.78 (m, 2H), −0.09 (s, 9H). ¹³C NMR (50 MHz, CDCl₃) δ 137.4, 129.9, 118.9, 75.9, 66.4, 17.7, −1.4.

### 2-(Methylthio)-1-((2-(trimethylsilyl)ethoxy)methyl)-1H-Imidazole (FW-211)

19.73 g (99.5 mmol) FW-210 was dissolved in 500 mL THF under argon atmosphere and cooled down to −75° C. 39.8 mL (99.5 mmol) of a 2.5 M n-BuLi in n-hexane solution was added via dropping funnel over 20 min maintaining the temperature at −75° C. The solution was quenched with 8.84 mL (99.5 mmol) of dimethyl disulfide and was then slowly warmed up to room temperature. 200 ml of brine was added and the organic layer was separated. The aqueous phase was extracted with EtOAc three times. The combined organic layers were dried over Na₂SO₄ and the solvent was removed in vacuo. The product was obtained as a yellow oil (99%, 24.15 g, 98.5 mmol) and was used without further purification in the next step. ¹H NMR (200 MHz, CDCl₃) δ 7.07 (d, J=1.0 Hz, 1H), 7.04 (d, J=0.9 Hz, 1H), 5.25 (s, 2H), 3.54-3.45 (m, 2H), 2.60 (s, 3H), 0.95-0.85 (m, 2H), −0.02 (s, 9H). ¹³C NMR (50 MHz, CDCl₃) δ 143.9, 129.3, 121.2, 75.1, 66.6, 17.9, 16.5, −1.3.

### 4,5-Dibromo-2-(methylthio)-1-((2-(trimethylsilyl)ethoxy)methyl)-1H-imidazole (FW-213db)

10.30 g (42.1 mmol) FW-211 was dissolved in 85 mL chloroform and cooled down to −5° C. 15.0 g (84.3 mmol) of N-Bromosuccinimide was added portion wise maintaining the temperature under 5° C. After complete conversion, the precipitating succinimide was filtered off and the filtrate was quenched with saturated sodium sulfite solution. After vigorous stirring for 10 min the organic layer was separated, and the aqueous layer was extracted with DCM twice. Combined organic layers were dried over Na₂SO₄ and solvent was removed in vacuo. The residue was purified via flash chromatography (SiO₂; isocratic:n-hexane/EtOAc 95:5). The product was obtained as yellow oil in 73% yield (12.40 g, 30.8 mmol). ¹H NMR (200 MHz, CDCl₃) δ 5.28 (s, 2H), 3.62-3.51 (m, 2H), 2.62 (s, 3H), 0.97-0.88 (m, 2H), −0.00 (s, 9H). ¹³C NMR (50 MHz, CDCl₃) δ 145.5, 117.5, 1042, 74.6, 67.1, 17.9, 16.1, −1.3.

### 4-Bromo-2-(methylthio)-1-((2-(trimethylsilyl)ethoxy)methyl)-1H-Imidazole (FW-213)

12.40 g (30.8 mmol) FW-213db was dissolved in 155 ml THF under argon atmosphere and cooled down to −75° C. 12.33 ml (30.8 mmol) of a 2.5 M n-BuLi in n-hexane solution was added via dropping funnel maintaining the temperature at −75° C. After complete addition, the reaction mixture was quenched with 50 ml MeOH and warmed to room temperature. Organic layer was separated and the aqueous layer was extracted twice with EtOAc. Combined organic layers were dried over Na₂SO₄ and solvent was removed in vacuo. The product was obtained as yellow oil in 95% yield (9.54 g, 29.5 mmol) and was used without further purification in the next step. ¹H NMR (200 MHz, CDCl₃) δ 6.99 (s, 1H), 5.18 (s, 2H), 3.55-3.41 (m, 2H), 2.59 (s, 3H), 0.95-0.84 (m, 2H), −0.03 (s, 9H). ¹³C NMR (50 MHz, CDCl₃) δ 144.1, 120.1, 115.4, 75.1, 66.7, 17.8, 16.3, −1.4.

### 2-(Methylthio)-4-(3-nitrophenyl)-1-((2-(trimethylsilyl)ethoxy)methyl)-1H-imidazole (FW-230)

3.00 g (9.3 mmol) FW-213, 2.01 g (12.1 mmol) 3-Nitrophenylboronic acid and 5.91 g (27.8 mmol) of K₃PO₄ were dissolved in 90 ml 1,4-dioxane and 20 ml demineralized water. The solution was degassed with three cycles of evacuation and backfilling with argon. 130 mg (2.5 mol %) P(t-Bu)₃ Pd G3 were added to the solution and another three cycles of evacuation and argon backfilling were carried out. The reaction mixture was warmed up to 50° C. and stirred overnight. After cooling to room temperature, the mixture was diluted with DCM, washed once with brine, dried over Na₂SO₄, filtered and evaporated to dryness. The crude product was purified via flash chromatography (SiO₂; n-hexane/EtOAc 6:4) obtaining a yellow oil in 88% yield (2.99 g, 8.2 mmol). ¹H NMR (400 MHz, CDCl₃) δ 8.58 (t, J=1.9 Hz, 1H), 8.12-8.08 (m, 1H), 8.05 (ddd, J=8.2, 2.3, 1.0 Hz, 1H), 7.50 (t, J=8.0 Hz, 1H), 7.45 (s, 1H), 5.29 (s, 2H), 3.60-3.52 (m, 2H), 2.69 (s, 3H), 0.96-0.90 (m, 2H), −0.01 (s, 9H). ¹³C NMR (101 MHz, CDCl₃) δ 148.9, 145.2, 140.2, 135.8, 130.6, 129.5, 121.5, 119.6, 117.8, 75.3, 66.8, 17.9, 16.5, −1.3. TLC-MS (ESI+): calcd. m/z 365.12 for C₁₆H₂₃N₃O₃SSi. Found 366.2 [M+H]⁺.

### 5-Bromo-2-(methylthio)-4-(3-nitrophenyl)-1-((2-(trimethylsilyl)ethoxy)methyl)-1H-imidazole (FW-234)

2.93 g (8.0 mmol) FW-230 was dissolved in 80 ml ACN under argon atmosphere. The solution was cooled to −30° C. 1.50 g (8.4 mmol) N-bromosuccinimide dissolved in 40 ml ACN was added dropwise under vigorous stirring maintaining −30° C. The reaction mixture was stirred for 1 h at −30° C. and was then slowly warmed to room temperature. The reaction was quenched by the addition of an aqueous, saturated Na₂SO₃ solution. The product was partitioned between water and DCM. The organic layer was washed with brine, dried over Na₂SO₄, filtered and evaporated to dryness. The crude product was purified via flash chromatography (SiO₂; n-hexane/EtOAc 6:4) to obtain a yellow oil in 94% yield (3.35 g, 7.5 mmol). ¹H NMR (200 MHz, CDCl₃) δ 8.89 (t, J=1.8 Hz, 1H), 8.35 (ddd, J=7.9, 1.6, 1.1 Hz, 1H), 8.14 (ddd, J=8.2, 2.3, 1.0 Hz, 1H), 7.56 (t, J=8.0 Hz, 1H), 5.37 (s, 2H), 3.70-3.58 (m, 2H), 2.70 (s, 3H), 1.01-0.91 (m, 2H), 0.01 (s, 9H). ¹³C NMR (50 MHz, CDCl₃) δ 148.6, 146.3, 137.1, 134.8, 132.3, 129.4, 122.0, 121.5, 101.6, 74.0, 67.0, 18.0, 16.0, −1.3. TLC-MS (ESI+): calcd. m/z 443.03 for C₁₆H₂₂BrN₃O₃SSi. Found 498.4/500.3 [M+MeOH+Na]⁺.

### N-(4(2-(Methylthio)-4-(3-nitrophenyl)-1-((2-(trimethylsilyl)ethoxy)methyl)-1H-imidazol-5-yl)pyridin-2-yl)acetamide (FW-241)

2.60 g (5.9 mmol) FW-234, 2.30 g (8.8 mmol) (N-(4-(4,4,5,5-tetramethyl-1,3,2-dioxaborolan-2-yl)pyridin-2-yl)acetamide), and 3.73 g (17.0 mmol) of K₃PO₄ were suspended in 58 ml 1,4-dioxane and 12 ml demineralized water. The solution was degassed with three cycles of evacuation and backfilling with argon. 83 mg (2.5 mol %) P(t-Bu)₃ Pd G3 were added to the solution and another three cycles of evacuation and argon backfilling were carried out. The reaction mixture was warmed up to 50° C. and stirred overnight. After cooling to room temperature, the mixture was diluted with EtOAc, washed once with brine, dried over Na₂SO₄, filtered and evaporated to dryness. The crude product was purified via flash chromatography (SiO₂; n-hexane/EtOAc/MeOH 35:60:5) obtaining a yellow oil in 90% yield (2.65 g, 5.3 mmol) with residues of pinacol. ¹H NMR (400 MHz, CDCl₃) δ 9.06 (s, 1H), 8.39-8.35 (m, 1H), 8.34-8.25 (m, 2H), 8.03-7.97 (m, 1H), 7.78-7.72 (m, 1H), 7.36 (t, J=8.0 Hz, 1H), 7.06 (dd, J=5.2, 1.2 Hz, 1H), 5.18 (s, 2H), 3.55-3.46 (m, 2H), 2.73 (s, 3H), 2.17 (s, 3H), 0.93-0.86 (m, 2H), −0.05 (s, 9H). ¹³C NMR (101 MHz, CDCl₃) δ 169.0, 152.6, 148.5, 148.5, 146.8, 140.4, 137.8, 135.6, 133.0, 129.2, 129.0, 122.1, 121.8, 121.1, 115.3, 73.2, 66.7, 24.6, 17.9, 16.2, −1.4. TLC-MS (ESI+): calcd. m/z 499.17 for C₂₃H₂₉N₅O₄SSi. Found 522.2 [M+Na]⁺.

### N-(4-(4-(3-Aminophenyl)-2-(methylthio)-1-((2-(trimethylsilyl)ethoxy)methyl)-1H-imidazol-5-yl)pyridin-2-yl)acetamide (FW-53/GM-719)

2.10 g (4.2 mmol) FW-241 was dissolved in MeOH and 1.37 g (21.0 mmol) zinc powder was added. 1.32 g (21.0 mmol) ammonium formate were added portion wise over 15 min to the suspension. After TLC indicated complete conversion the crude mixture was filtered over celite and washed with MeOH. The filtrate was evaporated to dryness. The thereby obtained yellowish oily residue was solved in MeOH and precipitated in iced water. Filtration and washing with iced water led to a white solid with 85% yield (1.68 g, 3.5 mmol) after drying. ¹H NMR (200 MHz, DMSO-d₆) δ 10.61 (s, 1H), 8.47-8.25 (m, 1H), 8.11 (s, 1H), 7.09-6.97 (m, 1H), 6.91-6.76 (m, 2H), 6.47-6.31 (m, 2H), 5.09 (s, 2H), 5.01 (s, 2H), 3.49-3.37 (m, 2H), 2.65 (s, 3H), 2.08 (s, 3H), 0.78 (t, J=7.7 Hz, 2H), −0.08 (s, 9H). ¹³C NMR (50 MHz, DMSO-d₆) δ 169.3, 152.6, 148.5, 148.3, 144.4, 140.0, 139.3, 134.1, 128.5, 127.4, 120.8, 114.7, 114.5, 1128, 112.6, 72.5, 65.5, 23.9, 17.2, 15.6, −1.5. TLC-MS (ESI+): calcd. m/z 469.20 for C₂₃H₃₁N₅O₂SSi. Found 470.1 [M+H]⁺.

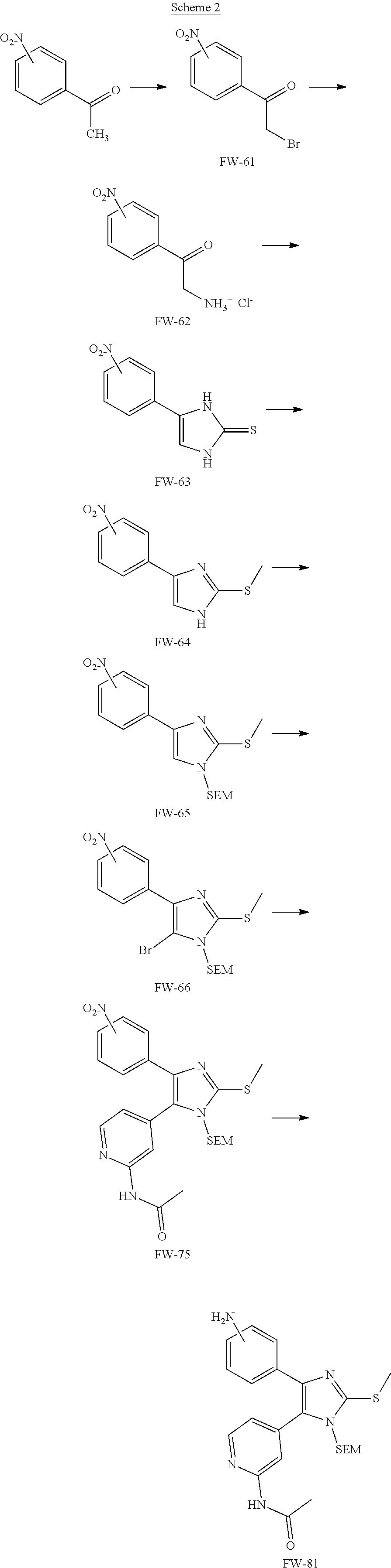

### 2-Bromo-1-(2-nitrophenyl)ethan-1-one (FW-61)

10.1 g (61.2 mmol) 2-Nitroacetophenone was dissolved in CHCl₃. Bromine (1 equiv) was added dropwise at room temperature under vigorous stirring. After complete addition, the mixture was stirred for additional 30 min at room temperature until decolorization. After addition of water and sodium sulfite, the mixture was extracted three times with DCM. The combined organic layers were dried over Na₂SO₄, filtered, and the solvent removed by rotary evaporation. Yield: 15.4 g (quantitative yield) as yellow oil. The crude product was used directly in the next step without further purification. ¹H NMR (200 MHz, CDCl₃) δ 8.28-8.16 (m, J=8.9 Hz, 1H), 7.83-7.73 (m, 1H), 7.71-7.61 (in, 1H), 7.48 (dd, J=7.3, 1.1 Hz, 1H), 4.28 (s, 2H). ¹³C NMR (50 MHz, CDCl₃) δ 194.40, 135.05, 134.85, 131.85, 131.39, 129.23, 124.55, 33.93.

### 2-Amino-1-(2-nitrophenyl)ethan-1-one hydrochlorid (FW-62)

15 g (61.5 mmol) FW-61 was dissolved in 120 ml chloroform. Urotropine (8.6 g, 61.5 mmol, 1.0 eq.) was added portionwise and the suspension was stirred for overnight at 70° C. After cooling to ambient temperature, the solids were collected by filtration and washed with chloroform. The dry solid was resuspended in 100 ml EtOH and treated with 50 ml concentrated hydrochloric acid. The suspension was stirred for overnight at ambient temperature. The mixture was cooled to 0° C., filtered and solids were washed with EtOH. Yield: 9.4 g (43.4 mmol, 70%) as white solid. The crude product was used directly in the next step without further purification. ¹H NMR (200 MHz, MeOH) δ 8.28-8.18 (m, 1H), 7.97-7.75 (m, 3H), 4.48 (s, 2H). ¹³C NMR (50 MHz, MeOH) δ 196.03, 147.50, 135.76, 134.25, 133.60, 129.49, 125.81.

### 4-(2-Nitrophenyl)-1,3-dihydro-2H-imidazole-2-thione (FW-63)

9.4 g (43.4 mmol) FW-62 was suspended in 150 ml glacial acetic acid. 4.7 g (47.7 mmol, 1.1 eq.) KSCN was added in one portion to the well stirred suspension. The mixture was refluxed for 1 hour, a color change from orange to a red suspension was observed. After complete reaction, the mixture was cooled to 0° C. and the solids collected by filtration. The filtrate was poured on water and extracted with DCM. The solid was combined with the organic phase of the extraction and solvents were evaporated to dryness. Yield: 2.8 g (12.75 mmol, 29%) as red solid. ¹H NMR (200 MHz, DMSO) δ 12.47 (s, 1H), 12.29 (a, 1H), 8.12-7.98 (m, J=7.7 Hz, 1H), 7.82-7.71 (m, 1H), 7.67-7.56 (m, J=6.7, 3.5 Hz, 2H), 7.07 (s, 1H). ¹³C NMR (50 MHz, DMSO) δ 161.85, 147.14, 133.53, 131.65, 129.68, 124.80, 124.65, 122.64, 114.61.

### 2-(Methylthio)-4-(2-nitrophenyl)-1H-imidazole (FW-64)

2.8 g (12.65 mmol) FW-63 was suspended with 2.1 g (15.19 mmol, 1.2 eq.) K₂CO₃ in 75 ml MeOH. The suspension was vigorously stirred for 30 minutes at ambient temperature. Methyl iodide (827 μl, 13.29 mmol, 1.05 eq.) was added dropwise to the well stirred solution at ambient temperature and stirred overnight. After complete consumption of the starting material, the mixture was poured on water and the aqueous phase was extracted with EtOAc. The combined organic layers were dried over Na₂SO₄, filtered and solvents were removed in vacuo. The crude product was purified via flash chromatography (SiO₂, DCM→DCM/MeOH 95:5; SiO₂, Hex→Hex/EA 1:1). Yield: 1.7 g (7.34 mmol, 58%) of the pure product as a white solid. ESI-MS: 235.7 [M+H]⁺. ¹H NMR (200 MHz, DMSO) δ 12.45 (s, 1H), 7.80-7.50 (m, 4H), 7.45-7.32 (m, 1H), 2.47 (s, 3H). ¹³C NMR (50 MHz, DMSO) δ 147.95, 141.97, 136.11, 131.54, 128.97, 127.36, 126.75, 123.27, 116.88, 15.21.

### 2-(Methylthio)-4-(2-nitrophenyl)-1-((2-(trimethylsilyl)ethoxy)methyl)-1H-Imidazole (FW-65)

1.7 g (7.22 mmol) FW-64 was dissolved in 60 ml dry THF and cooled to −15° C. under argon atmosphere. Sodium hydride (60% disp.) (346 mg, 8.67 mmol, 1.2 eq.) was added portionwise under vigorous stirring. The mixture was stirred for 10 minutes at −10° C. and SEM-Cl (1343 μl, 7.58 mmol, 1.05 eq.) dissolved in 30 ml dry THF was added dropwise. The mixture was stirred for 2 hours, quenched by the addition of a saturated aqueous NH₄Cl solution and the aqueous phase was extracted with EtOAc. The organic layer was separated, dried over Na₂SO₄, filtered and evaporated. The regioisomeric mixture was dissolved in 12 ml of MeCN and cat amounts of SEM-Cl (65 μl, 0.05 eq.) were added under an argon atmosphere. The flask was sealed and stirred at 80° C. for 1 h until complete conversion. The crude product was purified via flash chromatography (SiO₂, Hex→Hex/EA 7:3) to give 2 g of the pure product as a colorless oil in 90% (5.48 mmol) yield. ESI-MS: 387.9 [M+Na]⁺. ¹H NMR (200 MHz, CDCl₃) δ 7.95-7.86 (m, 1H), 7.67-7.50 (m, 2H), 7.42-7.28 (m, 2H), 5.26 (s, 2H), 3.55 (t, 2H), 2.65 (s, 3H), 0.93 (t, 2H), −0.00 (s, 9H). ¹³C NMR (50 MHz, CDCl₃) δ 148.61, 144.84, 136.47, 131.76, 130.24, 127.68, 127.33, 123.59, 119.74, 75.32, 66.74, 17.85, 16.36, −1.31.

### 5-Bromo-2-(methylthio)-4-(2-nitrophenyl)-1-((2-(trimethylsilyl)ethoxy)methyl)-1H-imidazole (FW-66)

2 g (5.47 mmol) FW-65 was dissolved in 55 ml MeCN and the solution was cooled to −30° C. under argon atmosphere. 1022 mg (5.75 mmol, 1.05 eq.) NBS dissolved in 27 ml MeCN was added dropwise and the mixture was stirred at −30° C. for 2 hours. After addition of water and sodium sulfite, the mixture was extracted three times with EtOAc. The separated organic layers were dried over Na₂SO₄, filtered and evaporated. The crude product was purified via flash chromatography (SiO₂, Hex→Hex/EA 7:3) to give 2.2 g of the pure product as a pale yellow oil in 89% (4.87 mmol) yield. ESI-MS: 466.0, 468.0 [M+Na]⁺. ¹H NMR (200 MHz, CDCl₃) δ 7.87 (dd, J=8.0, 0.9 Hz, 1H), 7.74-7.55 (m, 2H), 7.53-7.42 (m, 1H), 5.35 (s, 2H), 3.62 (t, 2H), 2.62 (s, 3H), 0.95 (t, 2H), 0.01 (s, 9H). ¹³C NMR (50 MHz, CDCl₃) δ 149.34, 146.17, 136.28, 132.26, 131.77, 128.88, 127.41, 124.56, 102.42, 74.21, 66.95, 17.95, 16.05, −1.30.

### N-(4-(2-(Methylthio)-4-(2-nitrophenyl)-1-((2-(trimethylsilyl)ethoxy)methyl)-1H-imidazol-5-yl)pyridin-2-yl)acetamide (FW-75)

500 mg (1.12 mmol, 1.0 eq.) FW-66, 442 mg (1.69 mmol, 1.5 eq.) (N-(4-(4,4,5,5-tetramethyl-1,3,2-dioxaborolan-2-yl)pyridin-2-yl)acetamide) was dissolved in 11 ml degassed 1,4-dioxane. An aqueous solution of K₃PO₄ (1.5 M, 3.38 mmol, 3 eq.) was added and the biphasic mixture was degassed several times. 32 mg (0.056 mmol, 0.05 eq.) P(tBu)₃ Pd G3 was added under an atmosphere of argon. The mixture was stirred at 55° C. overnight and additional 2 hours at 60° C. The mixture was then cooled to ambient temperature, Brine was added and the aqueous phase was extracted with DCM. The combined organic layers were dried over Na₂SO₄, filtered and the volatiles evaporated. The crude product was purified via flash chromatography (SiO₂, Hex→Hex/EA 3:7) to give 370 mg of the pure product as a pale yellow solid in 65% (0.73 mmol) yield. ESI-MS: 521.7 [M+Na]⁺. ¹H NMR (200 MHz, DMSO) δ 10.60 (s, 1H), 8.30 (d, J=5.2 Hz, 1H), 8.05 (s, 1H), 7.93-7.82 (m, 1H), 7.62-7.46 (m, 2H), 7.32-7.20 (m, 1H), 7.04-6.94 (m, 1H), 5.24 (s, 2H), 3.44 (t, 2H), 2.57 (s, 3H), 2.04 (s, 3H), 0.82 (t, 2H), −0.07 (s, 9H). ¹³C NMR (50 MHz, DMSO) δ 169.23, 152.56, 149.36, 148.24, 146.11, 138.22, 135.72, 132.53, 131.53, 129.15, 128.96, 127.68, 124.21, 119.40, 113.53, 73.51, 65.64, 23.84, 17.18, 15.38, −1.52.

### N-(4(4-(4-(2-Aminophenyl)-2-(methylthio)-1-((2-(trimethylsilyl)ethoxy)methyl)-1H-imidazol-5-yl)pyridin-2-yl)acetamide (FW-81)

370 mg (0.75 mmol) FW-75 and 242 mg (3.70 mmol, 5.0 eq.) zinc dust were suspended in 6 ml of Methanol. Under vigorous stirring 233 mg (3.70 mmol, 5.0 eq.) of ammonium formate was added in one portion. After 1 h stirring at room temperature, the reaction mixture was diluted with DCM and filtrated over celite. The filtrate was washed with sat aq. NH₄Cl solution and the separated organic layer was dried over Na₂SO₄ and solvents were removed in vacuo. Yield: 327 mg (0.70 mmol, 94%) of a pale yellow solid. ESI-MS: 492.1 [M+Na]⁺. ¹H NMR (200 MHz, DMSO) δ 10.55 (a, 1H), 8.27 (d, J=4.7 Hz, 1H), 8.08 (s, 1H), 7.03-6.84 (m, 2H), 6.77-6.57 (m, 2H), 6.41-6.23 (m, 1H), 5.57 (s, 2H), 5.16 (s, 2H), 3.38 (t, 3H), 2.63 (s, 3H), 2.05 (s, 3H), 0.82 (t, 2H), −0.08 (s, 9-1). ¹³C NMR (50 MHz, DMSO) δ 169.15, 152.57, 148.18, 146.81, 144.56, 139.68, 138.89, 129.42, 128.15, 128.00, 120.29, 116.40, 115.53, 114.02, 72.66, 65.58, 23.85, 17.16, 15.45, −1.50.

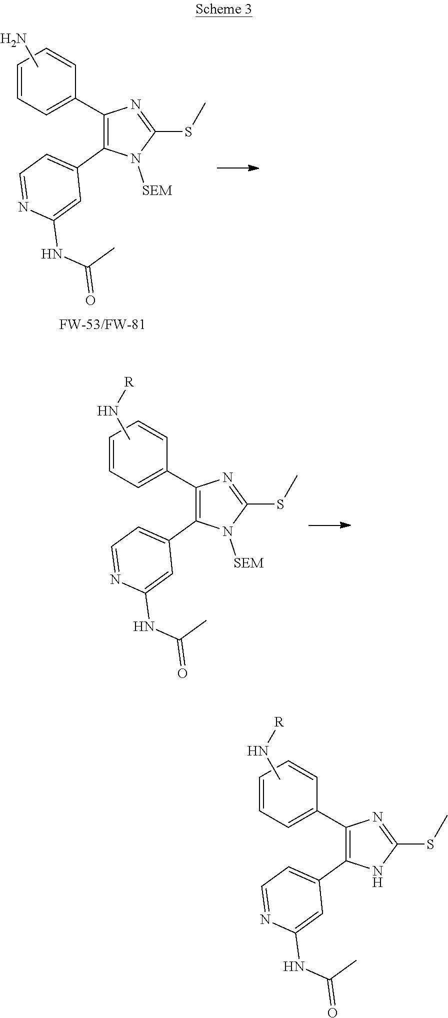

Final compounds of the 2-/3-anilino derivatives were synthesized by at first either amide coupling, coupling with a corresponding sulfonylchloride or urea formation with application of a corresponding isocyanate and subsequent removal of SEM protection group.

General Procedure 1: Amide and Urea Formation.

1A) A corresponding amine (FW-53/GM-719, FW-81, GM-790, GM-795, FW-226, FW-240), the corresponding carboxylic acid and an appropriate coupling reagent (PyBOP, TBTU, EDC-HCl) were dissolved in dry DMF (0.05 M) and a base (triethylamine, diisopropylamine) was added to the solution in the mentioned amounts. The reaction mixture was stirred overnight at ambient temperature. After addition of brine, the aqueous phase was extracted three times with DCM, the combined organic layers were dried over Na₂SO₄, filtered and volatiles were removed in vacuo. The crude product was purified via gradient silica flash chromatography.

1B) A corresponding amine (FW-53/GM-719, FW-81) was dissolved in dry DCM (0.1 M) and the corresponding isocyanate was added in one portion to the solution. After complete conversion of the starting material (3-12 h), brine was added and the aqueous phase was extracted three times with DCM. Combined organic layers were dried over Na₂SO₄, filtered and volatiles were removed in vacuo. The crude product was purified via gradient silica flash chromatography.

General Procedure 2: SEM Deprotection:

2A) The starting material was dissolved in dry DCM (10 ml per mmol). Trifluoroacetic acid (5 ml per mmol) was added dropwise under vigorous stirring. The mixture was stirred at ambient temperature overnight. After complete consumption of the starting material, the volatiles were removed by rotary evaporation and the oily residue suspended in DCM. A saturated NaHCO₃ solution was added and the product extracted with DCM or EtOAc three times. The combined organic layers were dried over Na₂SO₄, filtered and the solvents removed by rotary evaporation. The crude product was purified via flash chromatography. 2B) The starting material was dissolved in 2.5 M HCl in Ethanol (5 ml per 0.1 mmol) and stirred until complete consumption of the starting material (usually 24-72 hours). After complete reaction the solvents were evaporated and the oily residue suspended in saturated aqueous NaHCO₃ solution. The product was extracted three times with EtOAc or DCM. The combined organic layers were dried over Na₂SO₄, filtered and the solvents removed by rotary evaporation. The crude product was purified by flash chromatography.

### N-(3-(5-(2-acetamidopyridin-4-yl)-2-(methylthio)-1-((2-(trimethylsilyl)ethoxy)methyl)-1H-imidazol-4-yl)phenyl)cyclopropanecarboxamide (FW-55)

The title compound was synthesized according to general procedure 1A) from 70 mg (0.15 mmol) FW-53, 26 μl (0.33 mmol 2.2 eq.) cyclopropanecarboxylic acid, 171 mg (0.33 mmol, 2.2 eq.) PyBOP and 78 μl (0.45 mmol, 3.0 eq.) DIPEA. Flash chromatography (SiO₂, n-hex->n-hex/EtOAc 40:60). Yield: 66 mg (82%) as white solid. ESI-MS: 559.9 [M+Na]⁺. ¹H NMR (200 MHz, CDCl₃+MeOD) δ 9.46-9.20 (m, 1H), 8.21-8.09 (m, 2H), 7.75-7.62 (m, 1H), 7.41-7.34 (m, 1H), 7.08-6.92 (m, 2H), 6.89-6.77 (m, 1H), 5.17 (s, 2H), 3.49-3.38 (m, 2H), 2.60 (s, 3H), 2.10 (s, 3H), 1.60-1.47 (m, 1H), 0.96-0.88 (m, 2H), 0.88-0.78 (m, 3H), 0.75-0.67 (m, 2H), −0.11 (s, 9H).

### N-(3-(5-(2-acetamidopyridin-4-yl)-2-(methylthio)-1H-imidazol-4-yl)phenyl)cyclopropane-carboxamide (003)

The title compound was synthesized according to general procedure 2B) from 60 mg (0.11 mmol) FW-55. Flash chromatography (SiO₂, DCM->DCM/MeOH 10%) Yield: 29 mg (63%) as white solid. ESI-MS: 429.8 [M+Na]⁺. ¹H NMR (200 MHz, DMSO) δ 12.71 (s, 1H), 10.54-10.13 (m, 1H), 8.43-7.99 (m, 1H), 7.72-7.60 (m, 1H), 7.33 (s, 1H), 7.10-6.96 (m, 1H), 2.61 (s, 1H), 2.05 (s, 1H), 1.81-1.71 (m, 1H), 0.81-0.73 (m, 2H).

### N-(3-(5-(2-acetamidopyridin-4-yl)-2-(methylthio)-1-((2-(trimethylsilyl)ethoxy)methyl)-1H-imidazol-4-yl)phenyl)cyclopentanecarboxamide (FW-56)

The title compound was synthesized according to general procedure 1A) from 70 mg (0.15 mmol) FW-53, 21 μl (0.19 mmol 1.3 eq.) cyclopentanecarboxylic acid, 100 mg (0.19 mmol, 1.3 eq.) PyBOP and 77 μl (0.45 mmol, 3.0 eq.) DIPEA. Flash chromatography (SiO₂, n-hex->n-hex/EtOAc 30:70). Yield: 52 mg (62%) as white solid. ESI-MS: 587.9 [M+Na]⁺. ¹H NMR (200 MHz, CDCl₃+MeOD) δ 8.22-8.01 (m, 2H), 7.78-7.58 (m, 1H), 7.37 (s, 1H), 7.12-6.90 (m, 2H), 6.90-6.75 (m, 1H), 5.18 (s, 2H), 3.49-3.37 (m, 2H), 2.61 (s, 3H), 2.11 (s, 3H), 1.90-1.43 (m, 9H), 0.79 (s, 3H), −0.07-−0.18 (m, 9H).

### N-(3-(5-(2-acetamidopyridin-4-yl)-2-(methylthio)-1H-imidazol-4-yl)phenyl)cyclopentane-carboxamide (001)

The title compound was synthesized according to general procedure 26) from 50 mg (0.09 mmol) FW-56. Flash chromatography (SiO₂, DCM->DCM/MeOH 10%) Yield: 25 mg (64%) as off-white solid. ESI-MS: 458.0 [M+Na]⁺. As mixture of tautomers ¹H NMR (200 MHz, DMSO) δ 12.88-12.55 (m, 1H), 10.55-10.26 (m, 11H), 10.04-9.79 (m, 11H), 8.41-8.06 (m, 2H), 7.79-7.57 (m, 2H), 7.41-7.14 (m, 11H), 7.13-6.96 (m, 2H), 2.82-2.69 (m, 1H), 2.61 (s, 3H), 2.05 (s, 3H), 1.90-1.46 (m, 8H).

### N-(3-(5-(2-acetamidopyridin-4-yl)-2-(methylthio)-1-((2-(trimethylsilyl)ethoxy)methyl)-1H-imidazol-4-yl)phenyl)-4-fluorobenzamide (FW-59)

The title compound was synthesized according to general procedure 1A) from 70 mg (0.15 mmol) FW-53, 27 mg (0.19 mmol 1.3 eq.) 4-fluorobenzoic acid, 101 mg (0.19 mmol, 1.3 eq.) PyBOP and 77 μl (0.45 mmol, 3.0 eq.) DIPEA. Flash chromatography (SiO₂, n-hex->n-hex/EtOAc 40:60). Yield: 68 mg (77%) as white solid. ESI-MS: 613.8 [M+Na]⁺. ¹H NMR (200 MHz, CDCl₃+MeOD) δ 9.81-9.62 (m, 2H), 8.36-8.23 (m, 2H), 7.99 (dd, J=8.6, 5.4 Hz, 2H), 7.88 (d, J=8.3 Hz, 1H), 7.67 (s, 11H), 7.26-7.08 (m, 4H), 7.07-6.98 (m, 1H), 5.31 (s, 2H), 3.57 (t, 2H), 2.73 (s, 3H), 2.21 (s, 3H), 0.97 (t, 2H), 0.02 (s, 9H).

### N-(3-(5-(2-acetamidopyridin-4-yl)-2-(methylthio)-1H-imidazol-4-yl)phenyl)-4-fluorobenzamide (002)

The title compound was synthesized according to general procedure 2B) from 68 mg (0.11 mmol) FW-59. Flash chromatography (SiO₂, DCM->DCM/MeOH 10%) Yield: 32 mg (60%) as off-white solid. ESI-MS: 483.9 [M+Na]⁺. 1H NMR (200 MHz, DMSO) δ 12.87-12.65 (m, 11H), 10.54-10.24 (m, 2H), 8.38 (s, 11H), 8.24-7.98 (m, 3H), 7.94-7.71 (m, 2H), 7.49-7.29 (m, 3H), 7.25-7.11 (m, 11H), 7.10-6.97 (m, 11H), 2.62 (s, 3H), 2.12-1.98 (m, 3H).

### N-(3-(5-(2-acetamidopyridin-4-yl)-2-(methylthio)-1-((2-(trimethylsilyl)ethoxy)methyl)-1H-imidazol-4-yl)phenyl)-3-fluorobenzamide (FW-60)

The title compound was synthesized according to general procedure 1A) from 70 mg (0.15 mmol) FW-53, 27 mg (0.19 mmol 1.3 eq.) 3-fluorobenzoic acid, 101 mg (0.19 mmol, 1.3 eq.) PyBOP and 77 μl (0.45 mmol, 3.0 eq.) DIPEA. Flash chromatography (SiO₂, n-hex->n-hex/EtOAc 40:60). Yield: 82 mg (92%) as white solid. ESI-MS: 613.8 [M+Na]⁺. ¹H NMR (200 MHz, CDCl₃+MeOD) δ 9.78 (s, 1H), 9.60 (s, 1H), 8.37-8.18 (m, 2H), 7.94-7.84 (m, 1H), 7.80-7.61 (m, 3H), 7.52-7.38 (m, 1H), 7.28-7.14 (m, 2H), 7.11-6.96 (m, 2H), 5.28 (s, 2H), 3.66-3.45 (m, 2H), 2.71 (s, 3H), 2.20 (s, 3H), 1.00-0.86 (m, 2H), −0.00 (s, 9H).

### N-(3-(5-(2-acetamidopyridin-4-yl)-2-(methylthio)-1H-imidazol-4-yl)phenyl)-3-fluorobenzamide (004)

The title compound was synthesized according to general procedure 2A) from 80 mg (0.14 mmol) FW-60. Flash chromatography (SiO₂, DCM->DCM/MeOH 10%) Yield: 26 mg (41%) as off-white solid. ESI-MS: 484.0 [M+Na]⁺. ¹H NMR (200 MHz, DMSO) δ 12.76 (s, 1H), 10.49-10.26 (m, 2H), 8.42-8.23 (m, 1H), 8.22-8.07 (m, 1H), 7.93 (s, 1H), 7.87-7.71 (m, 3H), 7.66-7.39 (m, 3H), 7.22-7.14 (m, 1H), 7.10-7.01 (m, 1H), 2.63 (s, 3H), 2.05 (s, 3H).

### N-(4-(2-(methylthio)-4-(3-(3-phenylureido)phenyl)-1-((2-(trimethylsilyl)ethoxy)methyl)-1H-imidazol-5-yl)pyridin-2-yl)acetamide (FW-76)

The title compound was synthesized according to general procedure 1B) from 50 mg (0.11 mmol) FW-53 and 14 mg (0.12 mmol 1.1 eq.) phenyl isocyanate. Flash chromatography (SiO₂, n-hex->n-hex/EtOAc 30:70). Yield: 49 mg (78%) as white solid. ESI-MS: 610.8 [M+Na]⁺. ¹H NMR (200 MHz, DMSO) δ 10.65 (s, 1H), 8.72-8.51 (m, 2H), 8.45-8.31 (m, 1H), 8.14 (s, 1H), 7.56 (s, 1H), 7.49-7.35 (m, 3H), 7.34-7.21 (m, 2H), 7.18-7.03 (m, 2H), 7.01-6.84 (m, 2H), 5.11 (s, 2H), 3.51-3.37 (m, 2H), 2.68 (s, 3H), 2.07 (s, 3H), 0.85-0.73 (m, 2H), −0.08 (s, 9H).

### N-(4-(2-(methylthio)-4-(3-(3-phenylureido)phenyl)-1H-imidazol-5-yl)pyridin-2-yl)acetamide (005)

The title compound was synthesized according to general procedure 2A) from 49 mg (0.14 mmol) FW-76. Flash chromatography (SiO₂, n-hex->EtOAc) Yield: 22 mg (55%) as off-white solid. ESI-MS: 480.9 [M+Na]⁺. As mixture of tautomers: ¹H NMR (200 MHz, DMSO) δ 12.83-12.55 (m, 1H), 10.57-10.18 (m, 1H), 8.88-8.51 (m, 2H), 8.40-8.07 (m, 2H), 7.58-7.21 (m, 7H), 7.11-6.91 (m, 3H), 2.62 (s, 3H), 2.05 (s, 3H).

### N-(4-(4-(3-(3-(4-bromophenyl)ureido)phenyl)-2-(methylthio)-1-((2-(trimethylsilyl)-ethoxy)methyl)-1H-Imidazol-5-yl)pyridin-2-yl)acetamide (FW-86)

The title compound was synthesized according to general procedure 1B) from 60 mg (0.13 mmol) FW-53 and 30 mg (0.15 mmol 1.2 eq.) 4-bromophenyl isocyanate. Flash chromatography (SiO₂, n-hex->n-hex/EtOAc 30:70). Yield: 68 mg (79%) as white solid. ESI-MS: 691.0 [M+Na]⁺. ¹H NMR (200 MHz, CDCl₃) δ 9.16 (s, 1H), 8.26-8.07 (m, 2H), 7.94 (s, 1H), 7.74 (s, 1H), 7.44 (s, 1H), 7.24-6.92 (m, 8H), 5.17 (s, 2H), 3.61-3.50 (m, 2H), 2.64 (s, 3H), 2.13 (s, 3H), 1.00-0.88 (m, 2H), 0.02-−0.06 (m, 9H).

### N-(4-(4-(3-(3-(4-bromophenyl)ureido)phenyl)-2-(methylthio)-1H-imidazol-5-yl)pyridin-2-yl)acetamide (FW-92/015)

The title compound was synthesized according to general procedure 2B) from 68 mg (0.10 mmol) FW-86. Flash chromatography (SO₂, n-hex->EtOAc) Yield: 20 mg (36%) as off-white solid. ESI-MS: 559.0, 561.0 [M+Na]⁺. ¹H NMR (200 MHz, DMSO) δ 12.71 (s, 1H), 10.41 (s, 1H), 8.97-8.58 (m, 2H), 8.30 (s, 1H), 8.14 (s, 1H), 7.72-7.28 (m, 7H), 7.14-6.96 (m, 2H), 2.62 (s, 3H), 2.06 (s, 3H).

### N-(4-(4-(3-(3-(4-fluorophenyl)ureido)phenyl)-2-(methylthio)-1-((2-(trimethylsilyl)ethoxy)-methyl)-1H-imidazol-5-yl)pyridin-2-yl)acetamide (FW-94)

The title compound was synthesized according to general procedure 1B) from 50 mg (0.11 mmol) FW-53 and 16 mg (0.12 mmol 1.1 eq.) 4-fluorophenyl isocyanate. Flash chromatography (SiO₂, n-hex->n-hex/EtOAc 30:70). Yield: 60 mg (91%) as white solid. ESI-MS: no mass detected. ¹H NMR (200 MHz, CDCl₃+MeOD) δ 9.23 (s, 1H), 8.31-8.12 (m, 2H), 8.04-7.86 (m, 1H), 7.53-7.24 (m, 4H), 7.14-6.85 (m, 5H), 5.24 (s, 2H), 3.59-3.49 (m, 2H), 2.68 (s, 3H), 2.17 (s, 3H), 1.01-0.89 (m, 2H), −0.01 (s, 9H).

### N-(4-(4-(3-(3-(4-fluorophenyl)ureido)phenyl)-2-(methylthio)-1H-imidazol-5-yl)pyridin-2-yl)acetamide (017)

The title compound was synthesized according to general procedure 26) from 50 mg (0.08 mml) FW-94. Flash chromatography (SiO₂, n-hex->EtOAc) Yield: 20 mg (50%) as off-white solid. ESI-MS: 499.2 [M+Na]⁺. As mixture of tautomers: ¹H NMR (200 MHz, DMSO) δ 12.89-12.44 (m, 1H), 10.57-10.21 (m, 1H), 8.81-8.53 (m, 2H), 8.39-8.05 (m, 2H), 7.56-6.98 (m, 9H), 2.62 (s, 3H), 2.05 (s, 3H).

### N-(4(4-3(3-(3-chloro-4-fluorophenyl)ureido)phenyl)-2-(methylthio)-1-((2-(trimethylsilyl)ethoxy)methyl)-1H-imidazol-5-yl)pyridin-2-yl)acetamide (FW-95)

The title compound was synthesized according to general procedure 16) from 50 mg (0.11 mmol) FW-53 and 22 mg (0.13 mmol 1.2 eq.) 3-chloro-4-fluorophenyl isocyanate. Flash chromatography (SiO₂, n-hex->n-hex/EtOAc 30:70). Yield: 52 mg (76%) as white solid. ESI-MS: 662.9 [M+Na]⁺. ¹H NMR (200 MHz, CDCl₃) δ 9.23 (s, 1H), 8.39-8.06 (m, 2H), 8.04-7.67 (m, 2H), 7.48 (s, 1H), 7.41-7.28 (m, 1H), 7.21-6.76 (m, 6H), 5.19 (s, 2H), 3.61-3.50 (m, 2H), 2.63 (s, 3H), 2.13 (s, 3H), 0.99-0.87 (m, 2H), −0.03 (s, 9H).

### N-(4-(3-(3-(3-chloro-4-fluorophenyl)ureido)phenyl)-2-(methylthio)-1H-imidazol-5-yl)pyridin-2-yl)acetamide (018)

The title compound was synthesized according to general procedure 26) from 50 mg (0.08 mmol) FW-95. Flash chromatography (SiO₂, DCM/MeOH 2%->8%) Yield: 20 mg (50%) as off-white solid. ESI-MS: 533.1 [M+Na]⁺. As mixture of tautomers: ¹H NMR (400 MHz, DMSO) δ 12.82-12.59 (m, 1H), 10.52-10.24 (m, 1H), 9.01-8.73 (m, 2H), 8.42-8.04 (m, 2H), 7.78 (d, J=5.6 Hz, 1H), 7.57-7.43 (m, 2H), 7.38-7.19 (m, 3H), 7.08-6.99 (m, 2H), 2.62 (s, 3H), 2.12-2.01 (m, 3H).

### N-(4-(2-(methylthio)-4-(3-(3-(3-(trifluoromethyl)phenyl)ureido)phenyl)-1-((2-(trimethylsilyl)ethoxy)methyl)-1H-imidazol-5-yl)pyridin-2-yl)acetamide (FW-90)

The title compound was synthesized according to general procedure 1B) from 50 mg (0.11 mmol) FW-53 and 24 mg (0.13 mmol 1.2 eq.) 3-(trifluoromethyl)phenyl isocyanate. Flash chromatography (SiO₂, n-hex->n-hex/EtOAc 40:60). Yield: 59 mg (84%) as white solid. ESI-MS: 679.0 [M+Na]⁺. ¹H NMR (200 MHz, CDCl₃) δ 9.43 (s, 1H), 8.47-7.84 (m, 4H), 7.62-7.01 (m, 9H), 5.22 (s, 2H), 3.68-3.50 (m, 2H), 2.65 (s, 3H), 2.17 (s, 3H), 1.03-0.91 (m, 2H), 0.01 (s, 9H).

### N-(4(2-(methylthio)-4-(3-(3-(3-(trifluoromethyl)phenyl)ureido)phenyl)-1H-imidazol-5-yl)pyridin-2-yl)acetamide (FW-93/016)

The title compound was synthesized according to general procedure 2B) from 55 mg (0.08 mmol) FW-90. Flash chromatography (SiO₂, DCM/MeOH 2%->8%) Yield: 13 mg (30%) as off-white solid. ESI-MS: 549.0 [M+Na]⁺. As mixture of tautomers: ¹H NMR (200 MHz, DMSO) δ 12.91-12.56 (m, 1H), 10.63-10.20 (m, 1H), 9.13-8.77 (m, 2H), 8.40-7.95 (m, 3H), 7.61-7.19 (m, 6H), 7.12-6.97 (m, 2H), 2.62 (s, 3H), 2.14-1.96 (m, 3H).

### N-(4-(4-(3-aminophenyl)-2-(methylthio)-1H-imidazol-5-yl)pyridin-2-yl)acetamide (035)

The title compound was synthesized according to general procedure 2B) from 40 mg (0.09 mmol) FW-53. Flash chromatography (SiO₂, DCM/MeOH 1%->10%) Yield: 20 mg (69%) as off-white solid. ESI-MS: 361.9 [M+Na]⁺. ¹H NMR (400 MHz, DMSO) δ 12.81-12.39 (m, 1H), 10.59-10.14 (m, 1H), 8.50-8.00 (m, 2H), 7.15-6.90 (m, 2H), 6.78-6.45 (m, 3H), 5.40-4.94 (m, 2H), 2.59 (s, 3H), 2.15-2.01 (m, 3H).

### N-(3(5-(2-acetamidopyridin-4-yl)-2-(methylthio)-1-((2-(trimethylsilyl)ethoxy)methyl)-1H-Imidazol-4-yl)phenyl)-1-naphthamide (FW-253a)

The title compound was synthesized according to general procedure 1A) from 60 mg (0.13 mmol) FW-53, 33 mg (0.19 mmol 1.5 eq.) 1-naphthoic acid, 82 mg (0.26 mmol, 2.0 eq.) TBTU and 53 μl (0.45 mmol, 3.0 eq.) TEA. Flash chromatography (SiO₂, DCM/MeOH 1%->10%). The product was directly used in the next step without further characterization. ESI-MS: 646.1 [M+Na]⁺.

### N-(3-(5-(2-acetamidopyridin-4-yl)-2-(methylthio)-1H-Imidazol-4-yl)phenyl)-1-naphthamide (045)

The title compound was synthesized according to general procedure 2A) from the product of FW-253a. Flash chromatography (SiO₂, DCM/MeOH 1%->10%) Yield: 40 mg (62%) over two steps as off-white solid. ESI-MS: 516.3 [M+Na]⁺. ¹H NMR (200 MHz, DMSO) δ 12.76 (s, 1H), 10.75-10.25 (m, 2H), 8.43-7.95 (m, 6H), 7.90-7.69 (m, 2H), 7.66-7.55 (m, 3H), 7.53-7.27 (m, 1H), 7.27-7.05 (m, 2H), 2.63 (s, 3H), 2.05 (s, 3H).

### N-(3-(5-(2-acetamidopyridin-4-yl)-2-(methylthio)-1-((2-(trimethylsilyl)ethoxy)methyl)-1H-imidazol-4-yl)phenyl)-2-naphthamide (FW-254a)

The title compound was synthesized according to general procedure 1A) from 60 mg (0.13 mmol) FW-53, 33 mg (0.19 mmol 1.5 eq.) 2-naphthoic acid, 82 mg (0.26 mmol, 2.0 eq.) TBTU and 53 μl (0.45 mmol, 3.0 eq.) TEA. Flash chromatography (SiO₂, DCM/MeOH 1%->10). The product was directly used in the next step without further characterization. ESI-MS: 646.1 [M+Na]⁺.

### N-(3-(5-(2-acetamidopyridin-4-yl)-2-(methylthio)-1H-imidazol-4-yl)phenyl)-2-naphthamide (054)

The title compound was synthesized according to general procedure 2A) from the product of FW-254a. Flash chromatography (SiO₂, DCM/MeOH 1%->10%) Yield: 45 mg (70%) over two steps as off-white solid. ESI-MS: 516.3 [M+Na]⁺. As mixture of tautomers: ¹H NMR (400 MHz, DMSO) δ 12.90-12.62 (m, 1H), 10.57-10.25 (m, 2H), 8.57 (s, 1H), 8.41-8.11 (m, 2H), 8.10-7.97 (m, 5H), 7.93-7.81 (m, 1H), 7.72-7.57 (m, 2H), 7.53-7.27 (m, 1H), 7.22-7.03 (m, 2H), 2.63 (s, 3H), 2.05 (s, 3H).

### 3(5-(2-acetamidopyridin-4-yl)-2-(methylthio)-1-((2-(trimethylsilyl)ethoxy)methyl)-1H-imidazol-4-yl)phenyl)carbamoyl)phenyl acetate (FW-255a)

The title compound was synthesized according to general procedure 1A) from 60 mg (0.13 mmol) FW-53, 35 mg (0.19 mmol 1.5 eq.) FW-179, 82 mg (0.25 mmol, 2.0 eq.) TBTU and 53 μl (0.38 mmol, 3.0 eq.) TEA. Flash chromatography (SiO₂, DCM/MeOH 1%->10%). The product was directly used in the next step without further characterization. ESI-MS: 654.1 [M+Na]⁺.

### N-(3-(5-(2-acetamidopyridin-4-yl)-2-(methylthio)-1H-imidazol-4-yl)phenyl)-3-hydroxybenzamide (042)

The product of FW-255a was dissolved in 2 ml DCM and 1 ml of TFA was added dropwise under vigorous stirring. The mixture was stirred at ambient temperature overnight. After complete consumption of the starting material, the volatiles were removed by rotary evaporation and the oily residue suspended in DCM. A saturated NaHCO₃ solution was added and the product extracted with EtOAc three times. The combined organic layers were dried over Na₂SO₄, filtered and the solvents removed by rotary evaporation. ESI-MS: 524.3 [M+Na]⁺. The residue was dissolved in MeOH, sat aqueous NaHCO₃ was added until precipitation was observable, and the mixture was stirred at ambient temperature for 1 hour. Aqueous phase was extracted with DCM three times and the combined organic layers were dried over Na₂SO₄, filtered and volatiles removed in vacuo. Purification via Flash chromatography (SiO₂, DCM/MeOH 1%->10%) Yield: 27 mg (42%) over two steps as off-white sold. ESI-MS: 482.2 [M+Na]⁺. ¹H NMR (400 MHz, DMSO) δ 12.73 (s, 1H), 10.55-10.14 (m, 2H), 9.82 (br s, 1H), 8.48-8.05 (m, 2H), 7.92 (s, 1H), 7.87-7.74 (m, 1H), 7.46-7.26 (m, 4H), 7.19-7.12 (m, 1H), 7.10-7.02 (m, 1H), 6.99-6.94 (m, 1H), 2.62 (s, 3H), 2.05 (s, 3H).

### N-(3-(5-(2-acetamidopyridin-4-yl)-2-(methylthio)-1-((2-(trimethylsilyl)ethoxy)methyl)-1H-imidazol-4-yl)phenyl)-2,4,6-trifluorobenzamide (FW-266a)

50 mg (0.11 mmol) FW-63 was dissolved in 1 ml dry THF and an aqueous 1 M NaHCO₃ solution (0.21 mmol, 2.0 eq.) was added. The mixture was cooled down to 0° C. and 17 μl (0.133 mmol, 1.25 eq.) 2,4,6-trifluorobenzoyl chloride was added dropwise. After stirring for 30 minutes at 0° C. TLC indicated complete conversion. Brine was added and the aqueous phase was extracted three times with DCM. The combined organic layers were dried over Na₂SO₄, filtered and volatiles were removed in vacuo. The crude product was purified via flash chromatography (SiO₂, DCM/MeOH 1%->10%) and was directly used in the next step without further characterization. ESI-MS: 650.3 [M+Na]⁺.

### N-(3-(5-(2-acetamidopyridin-4-yl)-2-(methylthio)-1H-imidazol-4-yl)phenyl)-2,4,6-trifluorobenzamide (048)

The title compound was synthesized according to general procedure 2A) from the product of FW-266a. Flash chromatography (SiO₂, DCM/MeOH 1%->10%) Yield: 41 mg (77%) over two steps as off-white solid. ESI-MS: 520.1 [M+Na]⁺. ¹H NMR (200 MHz, DMSO) δ 12.77 (s, 1H), 11.10-10.16 (m, 2H), 8.45-8.05 (m, 2H), 7.89-7.60 (m, 2H), 7.53-7.27 (m, 3H), 7.24-7.15 (m, 1H), 7.10-6.99 (m, 1H), 2.62 (s, 3H), 2.05 (s, 3H).

### N-(3(5-(2-acetamidopyridin-4-yl)-2-(methylthio)-1-((2-(trimethylsilyl)ethoxy)methyl)-1H-imidazol-4-yl)phenyl)-3,5-difluorobenzamide (FW-272a)

The title compound was synthesized according to general procedure 1A) from 80 mg (0.17 mmol) FW-53, 32 mg (0.20 mmol, 1.2 eq.) 3,5-difluorobenzoic acid, 106 mg (0.20 mmol, 1.2 eq.) PyBOP and 45 μl (0.26 mmol, 1.5 eq.) DIPEA. Flash chromatography (SiO₂, n-hex->n-hex/EtOAc 50:50). Yield: 87 mg (84%) as white solid. ¹H NMR (200 MHz, CDCl₃) δ 9.57 (s, 1H), 9.01 (s, 1H), 8.29-8.20 (m, 2H), 7.94-7.82 (m, 1H), 7.71 (s, 1H), 7.42-7.34 (m, 2H), 7.23-7.11 (m, 3H), 6.88 (t, J=8.6 Hz, 1H), 5.15 (s, 2H), 3.59-3.48 (m, 2H), 2.66 (s, 3H), 2.14 (s, 3H), 0.96-0.86 (m, 2H), −0.03 (s, 9H).

### N-(3-(5-(2-acetamidopyridin-4-yl)-2-(methylthio)-1H-imidazol-4-yl)phenyl)-3,5-difluorobenzamide (050)

The title compound was synthesized according to general procedure 2B) from 87 mg (0.14 mmol) of FW-272a. Flash chromatography (SiO₂, DCM/MeOH 1%->10%) Yield: 41 mg (77%) as off-white solid. ESI-MS: 478.0 [M−H]⁻. ¹H NMR (200 MHz, DMSO) δ 12.75 (s, 1H), 10.61-10.14 (m, 2H), 8.49-8.25 (m, 1H), 8.23-8.07 (m, 1H), 8.00-7.77 (m, 2H), 7.76-7.61 (m, 2H), 7.59-7.35 (m, 2H), 7.31-7.13 (m, 1H), 7.13-6.99 (m, 1H), 2.63 (s, 3H), 2.05 (s, 3H).

### N-(3-(5-(2-acetamidopyridin-4-yl)-2-(methylthio)-1-((2-(trimethylsilyl)ethoxy)methyl)-1H-imidazol-4-yl)phenyl)-2-(2,6-difluorophenyl)acetamide (FW-273a)

The title compound was synthesized according to general procedure 1A) from 80 mg (0.17 mmol) FW-53, 32 mg (0.20 mmol, 1.2 eq.) 2-(2,6-difluorophenyl)acetic acid, 106 mg (0.20 mmol, 1.2 eq.) PyBOP and 35 μl (0.26 mmol, 1.5 eq.) TEA. Flash chromatography (SiO₂, n-hex->n-hex/EtOAc 50:50). Yield: 106 mg (99%) as white solid. ¹H NMR (200 MHz, CDCl₃) δ 9.33 (s, 1H), 8.28-8.17 (m, 3H), 7.73-7.62 (m, 1H), 7.51 (s, 1H), 7.23-7.01 (m, 4H), 6.89-6.79 (m, 2H), 5.19 (s, 2H), 3.70-3.61 (m, 3H), 3.57-3.45 (m, 2H), 2.67 (s, 3H), 2.12 (s, 3H), 0.95-0.85 (m, 2H), −0.05 (s, 9H).

### N-(3-(5-(2-acetamidopyridin-4-yl)-2-(methylthio)-1H-Imidazol-4-yl)phenyl)-2-(2,6-difluorophenyl)acetamide (053)

The title compound was synthesized according to general procedure 2B) from 106 mg (0.17 mmol) of FW-273a. Flash chromatography (SiO₂, DCM/MeOH 1%->10%) Yield: 60 mg (84%) as off-white solid. ESI-MS: 492.0 [M−H]⁻. ¹H NMR (200 MHz, CDCl₃ δ 12.71 (s, 1H), 10.55-10.23 (m, 2H), 8.46-8.04 (m, 2H), 7.83-7.56 (m, 2H), 7.48-7.27 (m, 2H), 7.16-7.00 (m, 4H), 3.77 (s, 2H), 2.61 (s, 3H), 2.06 (s, 3H).

### N-(3-(5-(2-acetamidopyridin-4-yl)-2-(methylthio)-1-((2-(trimethylsilyl)ethoxy)methyl)-1H-imidazol-4-yl)phenyl)-1-methyl-1H-indole-4-carboxamide (FW-274a)

The title compound was synthesized according to general procedure 1A) from 80 mg (0.17 mmol) FW-53, 36 mg (0.20 mmol, 1.2 eq.) 1-methyl-1H-indole-4-carboxylic acid, 106 mg (0.20 mmol, 1.2 eq.) PyBOP and 45 μl (0.26 mmol, 1.5 eq.) DIPEA. Flash chromatography (SiO₂, n-hex->n-hex/EtOAc 50:50). Yield: 85 mg (80%) as white solid. ¹H NMR (200 MHz, CDCl₃) δ 9.32 (s, 1H), 8.43 (s, 1H), 8.39-8.30 (m, 2H), 7.99 (d, J=7.6 Hz, 1H), 7.84 (s, 1H), 7.67 (d, J=7.1 Hz, 1H), 7.55 (d, J=8.2 Hz, 1H), 7.36 (d, J=7.4 Hz, 2H), 7.31-7.27 (m, 2H), 7.21-7.16 (m, 1H), 6.97 (d, J=3.0 Hz, 1H), 5.33 (s, 2H), 3.90 (s, 3H), 3.68-3.58 (m, 2H), 2.83 (s, 3H), 2.23 (s, 3H), 1.08-0.98 (m, 2H), 0.08 (s, 9H).

### N-(3-(5-(2-acetamidopyridin-4-yl)-2-(methylthio)-1H-Imidazol-4-yl)phenyl)-1-methyl-1H-Indole-4-carboxamide (049)

The title compound was synthesized according to general procedure 2B) from 85 mg (0.14 mmol) of FW-274a. Flash chromatography (SiO₂, DCM/MeOH 1%->10%) Yield: 32 mg (67%) as off-white solid. ESI-MS: 495.1 [M−H]⁻. As mixture of tautomers: ¹H NMR (200 MHz, DMSO) δ 12.90-12.59 (m, 1H), 10.52-10.13 (m, 2H), 8.44-8.09 (m, 2H), 8.03-7.77 (m, 2H), 7.66 (d, J=8.0 Hz, 1H), 7.57 (d, J=7.1 Hz, 1H), 7.51-7.35 (m, 2H), 7.27 (t, J=7.7 Hz, 1H), 7.19-7.03 (m, 2H), 6.85-6.75 (m, 1H), 3.85 (s, 3H), 2.63 (s, 3H), 2.05 (s, 3H).

### N-(3(5-(2-acetamidopyridin-4-yl)-2-(methylthio)-1H-imidazol-4-yl)phenyl)-2-fluoro-5-hydroxybenzamide (043)

60 mg (0.12 mmol) FW-53, 33 mg (0.16 mmol) FW-265 and 62 mg (0.19 mmol) TBTU were dissolved in 2 ml DMF and 53 μl (0.38 mmol) triethylamine was added. The reaction mixture was stirred overnight at 40° C. MeOH and a saturated aqueous NaHCO₃ were added and stirring proceeded for further 1.5 h [identification of intermediate via TLC-MS (ESI+): calcd. m/z 649.22 for C₃₂H₃₆FN₅O₅SSi. Found 672.2 [M+Na]⁺]. MeOH was evaporated and the aqueous layer was extracted with EtOAc. The combined organic layers were dried over Na₂SO₄ and solvents were removed in vacuo. The residue was purified via flash chromatography (SiO₂; DCM/MeOH 90:10) [identification of intermediate via TLC-MS (ESI+): calcd. m/z 607.21 for C₃₀H₃₄FN₅O₄SSi. Found 630.3 [M+Na]⁺] and then directly dissolved in a 25% TFA/DCM mixture. After stirring overnight at room temperature, a saturated aqueous NaHCO₃ solution was added and the aqueous layer was extracted with EtOAc. Combined organic layers were dried over Na₂SO₄ and solvents were removed in vacuo. The residue was again purified via flash chromatography (SiO₂; DCM/MeOH 90:10) yielding 57% (35 mg, 0.07 mmol) of a white solid. As mixture of tautomers: ¹H NMR (400 MHz. DMSO-d₆) δ 12.93-12.62 (m, 1H), 10.48-10.38 (m, 1H), 10.31 (s, 1H), 9.77 (s, 1H), 8.38-8.08 (m, 2H), 7.92-7.80 (m, 1H), 7.79-7.64 (m, 1H), 7.44-7.23 (m, 1H), 7.18-7.09 (m, 2H), 7.08-7.00 (m, 1H), 6.98-6.86 (m, 2H), 2.62 (s, 3H), 2.04 (s, 3H). ¹³C NMR (101 MHz, DMSO-d₆) δ 169.3, 169.1, 163.0, 162.8, 153.5, 152.5, 152.2 (d, J=239.2 Hz), 148.2, 148.2, 147.6, 143.8, 142.4, 139.5, 139.3, 139.0, 134.9, 134.4, 131.1, 130.7, 129.4, 128.7, 128.7, 126.1, 126.1, 125.4, 125.2, 125.1, 124.1, 119.7, 119.2, 118.7 (d, J=6.3 Hz), 117.3, 117.0 (d, J=24.0 Hz), 116.6, 115.3 (d, J=2.2 Hz), 111.0, 110.6, 23.9, 15.2. HRMS (ESI): exact mass calcd for C₂₄H₂₀FN₅O₃S [M+H]⁺: 478.13482. Found: 478.13482.

### N-(3-(5-(2-acetamidopyridin-4-yl)-2-(methylthio)-1-((2-(trimethylsilyl)ethoxy)methyl)-1H-imidazol-4-yl)phenyl)-2-((1-oxoisoindolin-2-yl)methyl)benzamide (FW-268a)

The title compound was synthesized according to general procedure 1A) from 75 mg (0.16 mmol) FW-53, 55 mg (0.20 mmol 1.3 eq.) FW-259, 76 mg (0.24 mmol, 1.5 eq.) TBTU and 67 μl (0.47 mmol, 3.0 eq.) TEA. Flash chromatography (SiO₂, DCM/MeOH 1%->10%). The product was directly used in the next step without further characterization. ESI-MS: 741.3 [M+Na]⁺.

### N-(3-(5-(2-acetamidopyridin-4-yl)-2-(methylthio)-1H-imidazol-4-yl)phenyl)-2-((1-oxoisoindolin-2-yl)methyl)benzamide (047)

The title compound was synthesized according to general procedure 2A) from the product of FW-268a. Flash chromatography (SiO₂, DCM/MeOH 1%->10%) Yield: 50 mg (63%) over two steps as off-white solid. ESI-MS: 611.0 [M+Na]⁺. As mixture of tautomers: ¹H NMR (400 MHz, DMSO) δ 12.82-12.66 (m, 1H), 10.59-10.27 (m, 2H), 8.39-8.09 (m, 2H), 7.98-7.86 (m, 1H), 7.80-7.67 (m, 2H), 7.62-7.53 (m, 3H), 7.52-7.37 (m, 4H), 7.27 (d, J=7.4 Hz, 1H), 7.17 (d, J=7.6 Hz, 1H), 7.11-7.02 (m, 1H), 4.89 (s, 2H), 4.41 (s, 2H), 2.63 (s, 3H), 2.03 (s, 3H).

### N-(3-(5-(2-acetamidopyridin-4-yl)-2-(methylthio)-1-((2-(trimethylsilyl)ethoxy)methyl)-1H-imidazol-4-yl)phenyl)-3-((tert-butyldimethylsilyl)oxy)-6-fluoro-2-((1-oxoisoindolin-2-yl)methyl)benzamide (FW-292)

86 mg (0.18 mmol) FW-291 was solved in 3 ml THF and 5 drops DMF were added. 19 μl (0.21 mmol) oxalyl chloride was added drop wise under gas formation and the mixture was stirred for 1 h at room temperature, whereupon the excess of oxalyl chloride was removed in vacuo. 75 mg (0.16 mmol) FW-53 was solved in 3 ml THF, 66 μl (0.48 mmol) triethylamine was added and the mixture was cooled down to 0° C. The beforehand prepared acid chloride solved in 2 ml THF was slowly added, whereupon the mixture was warmed to room temperature and stirred for 1 h. Brine was added and the aqueous layer was extracted with EtOAc. The combined organic layers were dried over Na₂SO₄ and solvents were removed in vacuo. Purification via flash chromatography (SiO₂; n-hexane/EtOAc 10:90) yielded 75% (105 mg, 0.12 mmol) of a white solid. ¹H NMR (200 MHz, CDCl₃) δ 9.00 (s, 1H), 8.55 (s, 1H), 8.33-8.20 (m, 2H), 7.84 (s, 2H), 7.60-7.52 (m, 1H), 7.45-7.27 (m, 5H), 7.12-7.06 (m, 1H), 6.98 (t, J=8.7 Hz, 1H), 6.88 (dd, J=8.7, 3.5 Hz, 1H), 5.22 (s, 2H), 4.92 (s, 2H), 4.42 (s, 2H), 3.59-3.48 (m, 2H), 2.75 (s, 3H), 2.15 (s, 3H), 0.99 (s, 9H), 0.96-0.89 (m, 2H), 0.22 (s, 6H), −0.00 (s, 9H). ¹³C NMR (50 MHz, CDCl₃) 169.2, 167.5, 166.5 (d, J=2.6 Hz), 153.5 (d, J=252.8 Hz), 152.0, 148.1, 146.8 (d, J=3.1 Hz), 146.1, 140.8, 140.0, 138.4, 136.5, 135.3, 134.4, 133.8, 133.7, 130.7, 129.1 (d, J=6.7 Hz), 128.0, 127.7, 123.8, 122.8 (d, J=7.4 Hz), 121.7, 121.0, 120.7, 119.3, 119.2, 116.3 (d, J=21.4 Hz), 115.9, 73.1, 66.5, 49.3, 43.9, 25.7 24.6, 18.2, 17.9, 16.4, −1.3, −4.2. TLC-MS (ESI+): calcd. m/z 866.35 for C₄₅H₅₅FN₅O₅SSi₂. Found 890.0 [M+Na]⁺.

### N-(3-(5-(2-acetamidopyridin-4-yl)-2-(methylthio)-1H-imidazol-4-yl)phenyl)-6-fluoro-3-hydroxy-2-((1-oxoisoindolin-2-yl)methyl)benzamide (FW-2931038)

90 mg (0.10 mmol) FW-292 was solved in a 33% TFA/DCM mixture and stirred overnight at room temperature [identification of intermediate via TLC-MS (ESI+): calcd. m/z 736.27 for C₃₉H₄₁FN₆O₄SSi Found 759.6 [M+Na]⁺]. A saturated aqueous NaHCO₃ solution was added and the aqueous layer was extracted with DCM. The combined organic layers were dried over Na₂SO₄ and solvents were removed in vacuo. The residue was solved in 3 ml THF and cooled down to 0° C. 205 μl (0.20 mmol) of a 1 M TBAF in THF solution were added and the mixture was stirred overnight at room temperature. Brine was added and the organic layer was extracted with EtOAc. The combined organic layers were dried over Na₂SO₄ and solvents were removed in vacuo. The crude product afforded two purification steps via flash chromatography (SiO₂; n-hexane/EtOAc/MeOH 0:95:5; DCM/MeOH 90:10) to yield 48% (31 mg, 0.05 mmol) of an off-white solid. As mixture of tautomers: ¹H NMR (400 MHz, DMSO-d₆) δ 12.87-12.60 (m, 1H), 10.62-10.27 (m, 2H), 9.96 (s, 1H), 8.40-8.07 (m, 2H), 8.00-7.86 (m, 1H), 7.82-7.68 (m, 1H), 7.63-7.54 (m, 1H), 7.51-7.25 (m, 4H), 7.20-7.13 (m, 1H), 7.12-7.01 (m, 2H), 6.98-6.88 (m, 1H), 4.84 (s, 2H), 4.27 (s, 2H), 2.63 (s, 3H), 2.12-1.97 (m, 3H). ¹³C NMR (101 MHz, DMSO-d₆) δ 169.1, 168.9, 167.2, 167.1, 164.9 (d, J=1.8 Hz), 152.4, 151.3 (d, J=259.3 Hz), 148.6 (d, J=2.1 Hz), 148.1, 147.5, 143.7, 143.3, 142.2, 139.6, 139.5, 139.4, 139.2, 136.4, 136.3, 135.2, 134.8, 134.3, 131.0, 130.6, 130.4, 130.3, 129.3, 129.2, 128.5, 128.1, 128.0, 127.7, 127.3, 127.2, 126.0, 123.8, 123.1, 119.7, 119.6, 119.6, 119.0 (d, J=7.0 Hz), 117.2, 116.5, 115.8 (d, J=21.2 Hz), 110.9, 110.5, 47.6, 43.0, 23.8, 15.1, 15.0. HRMS (ESI): exact mass calcd for C₃₃H₂₇FN₆O₄S [M+H]⁺: 623.18713. Found: 623.18759.

### N-(3-(5-(2-acetamidopyridin-4-yl)-2-(methylthio)-1-((2-(trimethylsilyl)ethoxy)methyl)-1H-imidazol-4-yl)phenyl)-3-((tert-butyldimethylsilyl)oxy)-2-((1,3-dioxoisoindolin-2-yl)methyl)-6-fluorobenzamide (FW-284)

131 mg (0.30 mmol) FW-283 was solved in 3 ml DCM and 3 drops DMF were added. 31 μl (0.42 mmol) oxalyl chloride was added drop wise under gas formation and the mixture was stirred for 1.5 h at room temperature, whereupon the excess of oxalyl chloride was removed in vacuo. 144 mg (0.30 mmol) FW-53 was solved in 3 ml THF, 128 μl (0.92 mmol) triethylamine was added and the mixture was cooled down to 0° C. The beforehand prepared acid chloride solved in 2 ml DCM was slowly added, whereupon the mixture was warmed to room temperature and stirred for 0.5 h. Solvents were removed in vacuo and purification via flash chromatography (SiO₂; DCM/MeOH 97:3) yielded 87% (235 mg, 0.27 mmol) of a white solid. ¹H NMR (400 MHz, CDCl₃) δ 9.56 (s, 1H), 8.71 (s, 1H), 8.40 (s, 1H), 7.92-7.82 (m, 1H), 7.79-7.73 (m, 2H), 7.66-7.61 (m, 1H), 7.54-7.49 (m, 2H), 7.47-7.41 (m, 1H), 7.24 (s, 1H), 7.06-7.00 (m, 1H), 6.99-6.95 (m, 1H), 6.73-6.59 (m, 2H), 5.12 (s, 2H), 4.94 (s, 2H), 3.58-3.50 (m, 2H), 2.73 (s, 3H), 2.20 (s, 3H), 0.95-0.88 (m, 11H), 0.23 (s, 6H), −0.03 (s, 9H). ¹³C NMR (101 MHz, CDCl₃) δ 169.4, 168.7, 162.4, 154.6, 151.1 (d, J=234.9 Hz), 152.0, 147.7, 145.9, 141.3, 139.3, 139.2, 138.3, 134.2, 133.9, 131.9, 129.3, 127.8, 123.9 (d, J=2.4 Hz), 123.6, 123.0, 120.8, 120.1 (d, J=8.1 Hz), 118.1, 116.5, 115.4, 115.2 (d, J=23.5 Hz), 73.0, 66.5, 36.4, 26.0, 24.7, 18.6, 18.0, 16.5, −1.3, −3.7. TLC-MS (ESI+): calcd. m/z 880.33 for C₄₅H₅₃FN₆O₆SSi₂. Found 903.0 [M+Na]⁺.

### N-(3-(5-(2-acetamidopyridin-4-yl)-2-(methylthio)-1H-imidazol-4-yl)phenyl)-2-((1,3-dioxoisoindolin-2-yl)methyl)-6-fluoro-3-hydroxybenzamide (039)

100 mg (0.11 mmol) FW-284 was solved in a 33% TFA/DCM mixture and stirred overnight at room temperature [identification of intermediate via TLC-MS (ESI+): calcd. m/z 750.25 for C₃₉H₃₉FN₆O₅SSi Found 773.9 [M+Na]⁺]. A saturated aqueous NaHCO₃ solution was added and the aqueous layer was extracted with DCM. The combined organic layers were dried over Na₂SO₄ and solvents were removed in vacuo. The residue was solved in 3 ml THF and cooled down to 0° C. 340 μl (0.34 mmol) of a 1 M TBAF in THF solution were added and the mixture was stirred overnight at room temperature. Brine was added and the organic layer was extracted with EtOAc. The combined organic layers were dried over Na₂SO₄ and the solvents removed in vacuo. The crude product afforded two purification steps via flash chromatography (SiO₂; DCM/MeOH 90:10, n-hexane/EtOAc/MeOH 35:55:10) to yield 49% (36 mg, 0.05 mmol) of an off-white solid. As mixture of tautomers: ¹H NMR (400 MHz, DMSO-d₆) δ 12.80-12.64 (m, 1H), 10.58-10.30 (m, 2H), 9.94-9.81 (m, 1H), 8.43-8.08 (m, 2H), 7.74-7.52 (m, 5H), 7.51-7.40 (m, 1H), 7.28-6.97 (m, 4H), 6.90-6.81 (m, 1H), 4.82 (s, 2H), 2.62 (s, 3H), 2.16-1.95 (m, 3H). ¹³C NMR (101 MHz, DMSO-d₆) δ 169.2, 168.9, 167.2, 162.1, 162.0, 152.6, 152.5, 151.8, 151.7, 151.7 (d, J=235.4 Hz), 148.0, 147.4, 143.7, 143.2, 1421, 139.6, 139.4, 139.1, 138.8, 134.3, 134.0, 131.6, 131.0, 130.4, 128.9, 128.2, 126.1, 125.9, 125.9, 123.7, 122.8, 122.8, 122.8, 120.5 (d, J=2.6 Hz), 119.2 (d, J=8.7 Hz), 118.8, 117.2, 116.4, 116.4, 115.0 (d, J=23.2 Hz), 110.8, 110.6, 34.4, 23.9, 15.2. HRMS (ESI): exact mass calcd for C₃₃H₂₅FN₆O₅S [M+H]⁺: 637.16639. Found: 637.16676.

### N-(3-(5-(2-acetamidopyridin-4-yl)-2-(methylthio)-1-((2-(trimethylsilyl)ethoxy)methyl)-1H-imidazol-4-yl)phenyl)-2-fluoro-6-((1-oxoisoindolin-2-yl)methyl)benzamide (FW-297a)

30 mg (0.10 mmol, 1.0 eq.) FW-296 was solved in 3.5 ml THF and 3 drops DMF were added. 14 μl (0.16 mmol) oxalyl chloride was added drop wise under gas formation and the mixture was stirred for 0.5 h at room temperature, whereupon the excess of oxalyl chloride was removed in vacuo. 50 mg (0.16 mmol, 1.0 eq.) FW-53 was solved in 3.5 ml THF, 45 μl (0.32 mmol) triethylamine was added and the mixture was cooled down to 0 T. The beforehand prepared acid chloride solved in 3.5 ml THF was slowly added, whereupon the mixture was warmed up to room temperature and stirred for 0.5 h. Volatiles were removed in vacuo and the crude product was purified via flash chromatography (SiO₂; DCM/MeOH 1%->1%). The product was directly used without further characterization in the next step. ESI-MS: 760.4 [M+Na]⁺.

### N-(3-(5-(2-acetamidopyridin-4-yl)-2-(methylthio)-1H-imidazol-4-yl)phenyl)-2-fluoro-6-((1-oxoisoindolin-2-yl)methyl)benzamide (040)

The title compound was synthesized according to general procedure 2A) from the product of FW-297a. Flash chromatography (SiO₂, n-hex->n-hex/EtOAc/MeOH 30:60:10) Yield: 16 mg (25%) over two steps as off-white solid. ESI-MS: 630.0 [M+Na]⁺. ¹H NMR (400 MHz, DMSO) δ 12.82-12.66 (m, 1H), 10.60-10.30 (m, 2H), 8.39-8.08 (m, 2H), 7.97-7.84 (m, 1H), 7.78-7.67 (m, 1H), 7.63-7.54 (m, 2H), 7.51-7.33 (m, 4H), 7.31-7.27 (m, 1H), 7.26-7.21 (m, 1H), 7.17 (d, J=7.7 Hz, 1H), 7.10-7.02 (m, 1H), 4.85 (s, 2H), 4.43 (s, 2H), 2.63 (s, 3H), 2.08-1.99 (m, 3H).

### 3-((3-(5-(2-acetamidopyridin-4-yl)-2-(methylthio)-1-((2-(trimethylsilyl)ethoxy)methyl)-1H-imidazol-4-yl)phenyl)carbamoyl)-2-((1-oxoisoindolin-2-yl)methyl)phenyl acetate (FW-304a)

The title compound was synthesized according to general procedure 1A) from 50 mg (0.11 mmol) FW-53, 38 mg (0.12 mmol 1.1 eq.) FW-300, 44 mg (0.14 mmol, 1.3 eq.) TBTU and 44 μl (0.32 mmol, 3.0 eq.) TEA. Flash chromatography (SiO₂, DCM/MeOH 1%->10%). The product was directly used in the next step without further characterization. ESI-MS: 799.9 [M+Na]⁺.

### N-(3-(5-(2-acetamidopyridin-4-yl)-2-(methylthio)-1H-imidazol-4-yl)phenyl)-3-hydroxy-2-((1-oxoisoindolin-2-yl)methyl)benzamide (041)

The product of FW-304a was dissolved in 2 ml DCM and 1 ml of TFA was added dropwise under vigorous stirring. The mixture was stirred at ambient temperature overnight. After complete consumption of the starting material, the volatiles were removed by rotary evaporation and the oily residue suspended in DCM. A sat. aqueous NaHCO₃ solution was added and the product extracted with EtOAc three times. The combined organic layers were dried over Na₂SO₄, filtered and the solvents removed by rotary evaporation. ESI-MS: 669.7 [M+Na]⁺. The residue was dissolved in MeOH, sat aqueous NaHCO₃ was added until precipitation was observable, and the mixture was stirred at ambient temperature for 1 hour. The aqueous phase was extracted with DCM three times and the combined organic layers were dried over Na₂SO₄, filtered and volatiles removed in vacuo. Purification via Flash chromatography (SiO₂, DCM/MeOH 1%->10%) Yield: 24 mg (39%) over two steps as off-white sold. ESI-MS: 603.8 [M−H]⁻. As mixture of tautomers: ¹H NMR (200 MHz, DMSO) δ 12.84-12.64 (m, 1H), 10.85-10.55 (m, 1H), 10.50-10.25 (m, 1H), 10.15-9.92 (m, 1H), 8.39-8.04 (m, 2H), 7.90-7.66 (m, 2H), 7.65-7.50 (m, 3H), 7.48-7.22 (m, 3H), 7.20-7.02 (m, 2H), 7.02-6.89 (m, 2H), 4.77 (s, 2H), 4.38 (s, 2H), 2.69-2.58 (m, 3H), 2.03 (s, 3H).

### N-(3-(5-(2-acetamidopyridin-4-yl)-2-(methylthio)-1-((2-(trimethylsilyl)ethoxy)methyl)-1H-imidazol-4-yl)phenyl)-2-((1,3-dioxoisoindolin-2-yl)methyl)benzamide (FW-307)

The title compound was synthesized according to general procedure 1A) from 135 mg (0.29 mmol) FW-53, 121 mg (0.43 mmol 1.5 eq.) FW-256, 138 mg (0.43 mmol, 1.5 eq.) TBTU and 120 μl (0.86 mmol, 3.0 eq.) TEA. Flash chromatography (SiO₂, n-hex->n-hex/EtOAc 15/85). The product was directly used in the next step without further characterization. ESI-MS: 755.6 [M+Na]⁺.

### N-(3-(5-(2-acetamidopyridin-4-yl)-2-(methylthio)-1H-imidazol-4-yl)phenyl)-2-((1,3-dioxoisoindolin-2-yl)methyl)benzamide (063)

The title compound was synthesized according to general procedure 2A) from the product of FW-307. Flash chromatography (SiO₂, DCM/MeOH 1%->10%) Yield: 112 mg (64%) over two steps as off-white solid. ESI-MS: 625.6 [M+Na]⁺. As mixture of tautomers: ¹H NMR (200 MHz, DMSO) δ 12.92-12.55 (m, 1H), 10.67-10.27 (m, 2H), 8.43-8.05 (m, 2H), 8.01-7.70 (m, 6H), 7.56 (s, 1H), 7.48-7.14 (m, 5H), 7.12-7.00 (m, 1H), 4.98 (s, 2H), 2.63 (s, 3H), 2.04 (s, 3H).

### 2-((2-((3-(5-(2-acetamidopyridin-4-yl)-2-(methylthio)-1H-imidazol-4-yl)phenyl)carbamoyl)-benzyl)carbamoyl)benzoic acid (065)

40 mg (0.06 mmol) of FW-308 was dissolved in 1.3 ml MeOH, 1.3 ml of a sat aqueous NaHCO₃ was added and the mixture was stirred for 5 h at ambient temperature. Demin. water was added and the pH was adjusted to 4-5 with diluted aqueous HCl. The aqueous phase was extracted multiple times with EtOAc. Combined organic layers were dried over Na₂SO₄, filtered and volatiles were removed in vacuo. EtOAc was added to the residue and the solution was filtered with a syringe filter. Yield: 35 mg (85%, 0.056 mmol) as yellow solid. ESI-MS: 619.3 [M−H]⁻. ¹H NMR (200 MHz, DMSO) δ 12.81 (s, 1H), 10.71-10.26 (m, 2H), 8.97 (s, 1H), 8.40-8.09 (m, 2H), 7.99-7.73 (m, 3H), 7.66-7.59 (m, 1H), 7.57-7.43 (m, 5H), 7.43-7.28 (m, 2H), 7.20-7.04 (m, 2H), 4.66-4.50 (m, 2H), 2.62 (s, 3H), 2.10-1.97 (m, 3H).

### N-(3-(5-(2-acetamidopyridin-4-yl)-2-(methylthio)-1-((2-(trimethylsilyl)ethoxy)methyl)-1H-imidazol-4-yl)phenyl)thiophene-2-carboxamide (GM-728)

GM-719 (41 mg, 0.087 mmol, 1.0 eq.) 2-thiophenecarboxylic acid (11 mg, 0.087 mmol, 1.0 eq.), HATU (49 mg, 0.130 mmol, 1.5 eq,) and DIPEA (44 μl, 0.26 mmol, 3 eq.) was dissolved in 4 ml DCM. The mixture was stirred at ambient temperature overnight. After complete consumption of the starting material, Celite was added and all volatiles were removed by rotary evaporation. The crude mixture was purified by flash chromatography (SiO₂, Hex→Hex/EA 1:2) to give 26 mg of the pure product as a colorless oil in 52% yield. ¹H NMR (200 MHz, Chloroform-d) δ 9.30 (s, 1H), 8.37 (d, J=7.1 Hz, 2H), 8.26 (d, J=5.2 Hz, 1H), 7.87 (d, J=6.8 Hz, 1H), 7.71 (d, J=3.8 Hz, 2H), 7.53 (dd, J=4.9, 1.0 Hz, 1H), 7.25-7.06 (m, 4H), 5.24 (s, 2H), 3.67-3.46 (m, 2H), 2.75 (s, 3H), 2.23 (s, 3H), 0.97-0.92 (m, 2H), 0.02 (s, 9H). ¹³C NMR (50 MHz, CDCl₃) δ 171.10, 169.37, 160.01, 151.68, 147.39, 146.10, 140.73, 140.15, 139.65, 137.83, 134.06, 130.64, 128.97, 128.35, 127.79, 127.53, 123.83, 121.34, 119.56, 119.37, 116.05, 66.43, 38.54, 24.43, 17.75, 16.30, −1.50.

### N-(3-(5-(2-acetamidopyridin-4-yl)-2-(methylthio)-1-((2-(trimethylsilyl)ethoxy)methyl)-1H-imidazol-4-yl)phenyl)isonicotinamide (GM-827)

FW-53 (80 mg, 0.168 mmol, 1.0 eq.) 4-pyridinylcarbonic acid (25 mg, 0.20 mmol, 1.2 eq.), PyBOP (104 mg, 0.20 mmol, 1.2 eq.) and DIPEA (44 μl, 0.25 mmol, 1.5 eq.) was dissolved in 4 ml DMF. The mixture was stirred at ambient temperature overnight. After complete consumption of the starting material, Ether was added and the organic layer washed three times with water. The organic layer was separated, dried over Na₂SO₄, filtered and all volatiles were removed by rotary evaporation. The crude mixture was purified by flash chromatography (SiO₂, DCM→DCM/MeOH 5%) to give 104 mg of the pure product as a pale yellow solid in quantitative yield. ¹H NMR (200 MHz, Chloroform-d) δ 9.84 (s, 1H), 9.62 (s, 1H), 8.61 (d, J=4.9 Hz, 2H), 8.40-8.05 (m, 2H), 7.95-7.62 (m, 4H), 7.23-7.01 (m, 3H), 5.17 (s, 2H), 3.60-3.43 (m, 2H), 2.66 (s, 3H), 2.11 (s, 3H), 0.99-0.83 (m, 2H), −0.04 (s, 9H). ¹³C NMR (50 MHz, CDCl₃) δ 169.47, 164.07, 151.95, 149.91, 147.75, 145.82, 142.30, 140.22, 139.96, 138.09, 134.09, 128.65, 127.83, 124.11, 121.55, 121.16, 119.96, 116.01, 72.90, 66.34, 24.29, −1.54.

### N-(3-(5-(2-acetamidopyridin-4-yl)-2-(methylthio)-1-((2-(trimethylsilyl)ethoxy)methyl)-1H-imidazol-4-yl)phenyl)nicotinamide (GM-830)

FW-53 (80 mg, 0.168 mmol, 1.0 eq.) 3-pyridinylcarbonic acid (25 mg, 0.20 mmol, 1.2 eq.), PyBOP (104 mg, 0.20 mmol, 1.2 eq.) and DIPEA (44 μl, 0.25 mmol, 1.5 eq.) was dissolved in 4 ml DMF. The mixture was stirred at ambient temperature overnight. After complete consumption of the starting material, Ether was added and the organic layer washed three times with water. The organic layer was separated, dried over Na₂SO₄, filtered and all volatiles were removed by rotary evaporation. The crude mixture was purified by flash chromatography (SiO₂, DCM→DCM/MeOH 5%) to give 82 mg of the pure product as a pale yellow solid in 85% yield. ¹H NMR (200 MHz, Chloroform-d) δ 9.56 (s, 1H), 9.42 (s, 1H), 9.06 (s, 1H), 8.79-8.56 (m, 1H), 8.39-8.07 (m, 3H), 8.02-7.74 (m, 2H), 7.50-7.01 (m, 4H), 5.17 (s, 2H), 3.53 (t, J=8.3 Hz, 2H), 2.69 (s, 3H), 2.15 (s, 3H), 1.00-0.84 (m, 2H), −0.02 (s, 9H). ¹³C NMR (50 MHz, CDCl₃) δ 169.58, 164.20, 151.82, 148.45, 147.73, 145.96, 140.30, 139.93, 138.09, 135.43, 134.04, 130.86, 128.85, 127.84, 124.01, 123.15, 121.27, 119.76, 119.60, 116.35, 72.91, 66.40, 46.23, 26.21, 16.16, −1.52.

### N-(3-(5-(2-acetamidopyridin-4-yl)-2-(methylthio)-1-((2-(trimethylsilyl)ethoxy)methyl)-1H-imidazol-4-yl)phenyl)picolinamide (GM-829)

FW-53 (80 mg, 0.168 mmol, 1.0 eq.) 2-pyridinylcarbonic acid (25 mg. 0.20 mmol, 1.2 eq.), PyBOP (104 mg, 0.20 mmol, 1.2 eq.) and DIPEA (44 μl, 0.25 mmol, 1.5 eq.) was dissolved in 4 ml DMF. The mixture was stirred at ambient temperature overnight. After complete consumption of the starting material, Ether was added and the organic layer washed three times with water. The organic layer was separated, dried over Na₂SO₄, filtered and all volatiles were removed by rotary evaporation. The crude mixture was purified by flash chromatography (SiO₂, DCM→DCM/MeOH 5%) to give 61 mg of the pure product as a pale yellow solid in 63% yield. ¹H NMR (200 MHz, Chloroform-d) δ 10.05 (s, 1H), 9.33 (s, 1H), 8.60 (d, J=4.7 Hz, 1H), 8.38 (s, 1H), 8.28 (d, J=6.5 Hz, 2H), 7.97-7.85 (m, 3H), 7.54-7.38 (m, 1H), 7.35-7.17 (m, 1H), 7.17-7.04 (m, 2H), 5.27 (s, 2H), 3.63-3.49 (m, 2H), 2.77 (s, 3H), 2.21 (s, 3H), 0.95 (dd, J=9.3, 7.4 Hz, 2H), −0.00 (s, 9H). ¹³C NMR (50 MHz, CDCl₃) δ 168.85, 161.86, 152.23, 149.76, 147.86, 147.77, 145.90, 140.84, 139.89, 137.83, 137.50, 134.41, 128.89, 127.84, 126.29, 123.42, 122.28, 121.39, 118.83, 118.65, 115.28, 72.93, 66.31, 24.52, 17.71, 16.36, −1.51, −1.55.

### N-(3-(5-(2-acetamidopyridin-4-yl)-2-(methylthio)-1H-imidazol-4-yl)phenyl)isonicotinamide (036)

The title compound was prepared from GM-827 (104 mg, 0.181 mmol) according to general procedure 2B. The crude product was purified by flash chromatography (SiO₂, DCM→DCM/MeOH 10%). 50 mg (0,112 mmol) of the pure product were obtained as a white solid in 63% yield. ¹H NMR (200 MHz, DMSO-d₆) δ 12.78 (s, 1H), 10.47 (d, J=43.4 Hz, 2H), 8.82 (s, 2H), 8.47-7.77 (m, 6H), 7.59-7.00 (m, 3H), 2.65 (s, 3H), 2.08 (s, 3H). ¹³C NMR (50 MHz, DMSO) δ 169.32, 164.50, 152.84, 150.65, 147.93, 144.09, 142.65, 142.24, 139.41, 134.76, 131.31, 131.08, 129.66, 124.76, 121.95, 120.66, 116.85, 110.90, 24.25, 15.48. ESI-MS: 445.5[M+H]⁺.

### N-(3-(5-(2-acetamidopyridin-4-yl)-2-(methylthio)-1H-imidazol-4-yl)phenyl)nicotinamide (014)

The title compound was prepared from GM-830 (82 mg, 0.143 mmol) according to general procedure 2B. The crude product was purified by flash chromatography (SiO2, DCM→DCM/MeOH 10%). 49 mg (0,110 mmol) of the pure product were obtained as a white solid in 77% yield. ¹H NMR (200 MHz, DMSO-d₆) δ 12.74 (s, 1H), 10.53 (s, 1H), 10.41 (s, 1H), 9.12 (s, 1H), 8.87-8.74 (m, 1H), 8.43-8.27 (m, 2H), 8.18 (d, J=5.3 Hz, 1H), 7.96 (s, 1H), 7.86 (d, J=8.3 Hz, 1H), 7.66-7.55 (m, 1H), 7.50-7.36 (m, 1H), 7.21 (d, J=7.5 Hz, 1H), 7.10 (d, J=5.2 Hz, 1H), 2.66 (s, 3H), 2.07 (s, 3H). ¹³C NMR (50 MHz, DMSO) δ 169.38, 164.52, 152.88, 152.52, 149.06, 148.09, 139.56, 135.84, 130.94, 129.42, 123.87, 120.38, 117.05, 111.00, 24.25, 15.45.

### N-(3-(5-(2-acetamidopyridin-4-yl)-2-(methylthio)-1H-Imidazol-4-yl)phenyl)picolinamide (013)

The title compound was prepared from GM-829 (61 mg, 0.106 mmol) according to general procedure 2B. The crude product was purified by flash chromatography (SiO₂, DCM→DCM/MeOH 10%). 38 mg (0,085 mmol) of the pure product were obtained as a white solid in 81% yield. ¹H NMR (400 MHz, DMSO-d₆) δ 12.84-12.57 (m, 1H), 10.84-10.26 (m, 2H), 8.88-8.61 (m, 1H), 8.40 (s, 1H), 8.26-8.03 (m, 4H), 8.01-7.80 (m, 1H), 7.72-7.63 (m, 1H), 7.49-7.28 (m, 1H), 7.24-7.16 (m, 1H), 7.12-7.02 (m, 1H), 2.64 (s, 3H), 2.05 (s, 3H). ¹³C NMR (101 MHz, DMSO) δ 169.38, 163.07, 152.92, 150.28, 148.89, 147.96, 144.27, 142.66, 139.18, 138.59, 134.92, 131.48, 131.18, 129.65, 127.43, 124.59, 122.85, 120.69, 120.59, 116.99, 111.10, 24.31, 15.62.

### N-(3-(5-(2-acetamidopyridin-4-yl)-2-(methylthio)-1-((2-(trimethylsilyl)ethoxy)methyl)-1H-imidazol-4-yl)phenyl)-3-phenylpropanamide (GM-853)

FW-53 (80 mg, 0.168 mmol, 1.0 eq.) phenylpropionic acid (31 mg, 0.20 mmol, 1.2 eq.), PyBOP (104 mg, 0.20 mmol, 1.2 eq.) and DIPEA (44 μl, 0.25 mmol, 1.5 eq.) was dissolved in 4 ml DMF. The mixture was stirred at ambient temperature overnight. After complete consumption of the starting material, Ether was added and the organic layer washed three times with water. The organic layer was separated, dried over Na₂SO₄, filtered and all volatiles were removed by rotary evaporation. The crude mixture was purified by flash chromatography (SiO₂, Hex→Hex/EA 1:2) to give 82 mg of the pure product as a pale yellow solid in 81% yield. ¹H NMR (200 MHz, Chloroform-d) δ 9.27 (s, 1H), 8.41-8.17 (m, 2H), 8.01 (s, 1H), 7.74 (s, 1H), 7.53 (s, 1H), 7.29-7.11 (m, 8H), 5.22 (s, 2H), 3.57 (t, J=8.3 Hz, 2H), 2.97 (t, J=7.8 Hz, 2H), 2.71 (s, 3H), 2.64-2.47 (m, 2H), 2.17 (s, 3H), 0.94 (s, 2H), 0.01 (s, 9H). ¹³C NMR (50 MHz, CDCl₃) δ 170.62, 169.29, 151.90, 147.63, 146.00, 140.78, 140.58, 140.04, 138.18, 133.87, 128.92, 128.42, 128.23, 127.81, 126.09, 123.36, 121.31, 119.10, 118.85, 115.82, 72.99, 66.45, 39.01, 31.36, 24.43, 17.75, 16.23, −1.51.

### N-(3-(5-(2-acetamidopyridin-4-yl)-2-(methylthio)-1H-imidazol-4-yl)phenyl)-3-phenylpropanamide (037)

The title compound was prepared from GM-853 (80 mg, 0.133 mmol) according to general procedure 2B. The crude product was purified by flash chromatography (SiO₂, n-hexane→EtOAc). 35 mg (0,074 mmol) of the pure product were obtained as a white solid in 56% yield. ¹H NMR (400 MHz, DMSO-d₆) δ 12.91-12.57 (m, 1H), 10.60-10.21 (m, 1H), 10.07-9.76 (m, 1H), 8.35 (s, 1H), 8.25-8.06 (m, 1H), 7.77-7.55 (m, 2H), 7.42-7.14 (m, 6H), 7.08 (d, J=7.5 Hz, 1H), 7.00 (d, J=5.3 Hz, 1H), 2.90 (t, J=7.6 Hz, 2H), 2.67-2.57 (m, 5H), 2.06 (s, 3H). ¹³C NMR (101 MHz, DMSO) δ 170.97, 169.33, 152.95, 147.90, 144.24, 141.61, 140.06, 134.80, 131.56, 131.13, 129.64, 128.76, 128.67, 126.38, 123.73, 119.45, 116.89, 111.10, 38.41, 31.26, 24.34, 15.59.

### N-(4-(2-(methylthio)-4-(3-(propylsulfonamido)phenyl)-1H-imidazol-5-yl)pyridin-2-yl)acetamide (008)

FW-53 (55 mg, 0.12 mmol, 1.0 eq.) was dissolved in 100 μl pyridine and stirred at ambient temperature. Propane sulfonylchloride (16 μl, 0.14 mmol, 1.2 eq.) was added to the well stirred solution. The mixture was stirred at ambient temperature until complete consumption of the starting material. Then dry DCM (4 ml) was added followed by 2 ml TFA. Stirring was continued for 24 hours. The mixture was quenched by the careful addition of saturated NaHCO₃ solution and the product extracted with EtOAc three times. The combined organic layers were dried over sodium sulfate, filtered and the solvents removed by rotary evaporation. The crude product was purified by flash chromatography (SiO₂, EtOAc) to yield the give the final compound in 37% yield (20 mg, 0.044 mmol) over two steps as an off-white sold. ¹H NMR (200 MHz, DMSO-d₆) δ 12.76 (s, 1H), 10.44 (s, 1H), 9.87 (s, 1H), 8.33-8.07 (m, 2H), 7.22 (ddd, J=37.8, 25.1, 6.5 Hz, 5H), 3.04 (t, J=7.6 Hz, 2H), 2.64 (s, 3H), 2.07 (s, 3H), 1.67 (h, J=7.4 Hz, 2H), 0.94 (t, J=7.4 Hz, 3H). ¹³C NMR (50 MHz, CDCl₃) δ 169.40, 152.84, 148.07, 142.98, 139.07, 130.15, 123.75, 119.06, 117.20, 111.09, 52.58, 24.28, 17.16, 15.48, 12.90.

### N-(4(4-(3-(methylsulfonamido)phenyl)-2-(methylthio)-1H-imidazol-5-yl)pyridin-2-yl)acetamide (007)

FW-53 (88 mg, 0.19 mmol, 1.0 eq.) and NaHCO₃ (32 mg, 0.376 mmol, 2.0 eq.) was dissolved in THF/water (400 μl 1:1) and stirred at ambient temperature. Methane sulfonylchloride (17 μl, 0.225 mmol, 1.2 eq.) was added to the well stirred solution. The mixture was stirred at ambient temperature until complete consumption of the starting material (four hours). Then brine was added followed by EtOAc. The organic layer was separated, dried over sodium sulfate, filtered and the solvents removed by rotary evaporation. The residue was dissolved in 5 ml DCM and 2.5 ml TFA. Stirring was continued for 24 hours. The mixture was quenched by the careful addition of saturated NaHCO₃ solution and the product extracted with EtOAc three times. The combined organic layers were dried over sodium sulfate, filtered and the solvents removed by rotary evaporation. The crude product was purified by flash chromatography (SiO₂, EtOAc) to yield the give the final compound in 51% yield (40 mg, 0.096 mmol) over two steps as an off-white solid. ¹H NMR (200 MHz, DMSO-d₆) δ 12.76 (s, 1H), 10.61-10.26 (m, 1H), 9.80 (s, 1H), 8.22 (d, J=26.0 Hz, 2H), 7.53-7.06 (m, 5H), 2.96 (s, 3H), 2.64 (s, 3H), 2.06 (s, 3H).

### N-(4-(4-(3-(cyclopropanesulfonamido)phenyl)-2-(methylthio)-1H-imidazol-5-yl)pyridin-2-yl)acetamide (006)

FW-53 (66 mg, 0.14 mmol, 1.0 eq.) was dissolved in 200 μl DCM followed by 17 μl pyridine (0.21 mmol, 1.5 eq.) and stirred at ambient temperature. Cyclopropane sulfonylchloride (17 μl, 0.17 mmol, 1.2 eq.) was added to the well stirred solution. The mixture was stirred at ambient temperature until complete consumption of the starting material. Then dry DCM (4 ml) was added followed by 2 ml TFA. Stirring was continued for 24 hours. The mixture was quenched by the careful addition of saturated NaHCO₃ solution and the product extracted with EtOAc three times. The combined organic layers were dried over sodium sulfate, filtered and the solvents removed by rotary evaporation. The crude product was purified by flash chromatography (SiO₂, EtOAc) to yield the give the final compound in 50% yield (31 mg, 0.07 mmol) over two steps as an off-white solid. ¹H NMR (400 MHz, DMSO-d₆) δ 12.75 (d, J=23.6 Hz, 1H), 10.41 (d, J=55.5 Hz, 1H), 9.71 (d, J=54.5 Hz, 1H), 8.40-8.03 (m, 2H), 7.48-7.10 (m, 4H), 7.02 (d, J=5.3 Hz, 1H), 2.62 (s, 3H), 2.04 (s, 3H), 1.09 (t, J=7.0 Hz, 1H), 0.98-0.84 (m, 4H). ¹³C NMR (101 MHz, DMSO) δ 169.46, 152.95, 148.04, 144.29, 142.82, 135.20, 131.59, 131.03, 130.20, 124.34, 120.42, 117.16, 111.29, 65.34, 29.94, 24.32, 15.61, 5.41.

### N-(4-(2-(methylthio)-4-(3-(phenylsulfonamido)phenyl)-1H-imidazol-5-yl)pyridin-2-yl)acetamide (009)

FW-53 (64 mg, 0.137 mmol, 1.0 eq.) and NaHCO₃ (23 mg, 0.274 mmol, 2.0 eq.) was dissolved in THF/water (400 μl 1:1) and stirred at ambient temperature. Benzene sulfonylchloride (21 μl, 0.164 mmol, 1.2 eq.) was added to the well stirred solution. The mixture was stirred at ambient temperature until complete consumption of the starting material (four hours). Then brine was added followed by EtOAc. The organic layer was separated, dried over sodium sulfate, filtered and the solvents removed by rotary evaporation. The residue was dissolved in 5 ml DCM and 2.5 ml TFA. Stirring was continued for 24 hours. The mixture was quenched by the careful addition of saturated NaHCO₃ solution and the product extracted with EtOAc three times. The combined organic layers were dried over sodium sulfate, filtered and the solvents removed by rotary evaporation. The crude product was purified by flash chromatography (SiO₂, EtOAc) to yield the give the final compound in 43% yield (28 mg, 0.058 mmol) over two steps as a white solid. ¹H NMR (200 MHz. DMSO-d₆) δ 12.72 (s, 1H), 10.41 (d, J=18.0 Hz, 2H), 8.41-8.03 (m, 2H), 7.73 (d, J=7.4 Hz, 2H), 7.69-7.47 (m, 3H), 7.37-7.00 (m, 4H), 6.87 (d, J=5.4 Hz, 1H), 2.62 (s, 3H), 2.10 (s, 3H). ¹³C NMR (50 MHz, DMSO) δ 169.44, 152.88, 147.99, 143.04, 139.95, 138.38, 133.29, 129.92, 129.61, 126.94, 124.13, 119.99, 117.09, 111.26, 24.30, 15.40.

### N-(4-(4-(2-(3-(4-bromophenyl)ureido)phenyl)-2-(methylthio)-1-((2-(trimethylsilyl)ethoxy)-methyl)-1H-imidazol-5-yl)pyridin-2-yl)acetamide (FW-97)

The title compound was synthesized according to general procedure 1B) from 49 mg (0.10 mmol) FW-81 and 25 mg (0.13 mmol 1.2 eq.) 4-bromophenyl isocyanate. Flash chromatography (SiO₂, n-hex->n-hex/EtOAc 40:60). Yield: 63 mg (90%) as white solid. ESI-MS: 690.3, 692.1 [M+Na]⁺. ¹H NMR (200 MHz, CDCl₃) δ 10.06 (s, 1H), 9.46 (s, 1H), 8.35 (s, 1H), 7.87-7.72 (m, 1H), 7.67-7.54 (m, 1H), 7.49-7.04 (m, 9H), 6.90-6.60 (m, 1H), 5.32 (s, 2H), 3.57-3.41 (m, J=7.4 Hz, 2H), 2.47 (s, 3H), 2.21 (s, 3H), 0.96-0.88 (m, 2H), −0.02 (s, 10H).

### N-(4-(4-(2(3-(4-bromophenyl)ureido)phenyl)-2-(methylthio)-1H-imidazol-5-yl)pyridin-2-yl)acetamide (019)

The title compound was synthesized according to general procedure 2B) from 63 mg (0.10 mmol) of FW-97. Flash chromatography (SiO₂, DCM/MeOH 2%->8%) Yield: 20 mg (40%) as off-white solid. ESI-MS: 558.9, 560.8 [M+Na]⁺. As mixture of tautomers: ¹H NMR (400 MHz, DMSO) δ 13.08-12.58 (m, 1H), 10.39-10.14 (m, 1H), 9.06 (s, 1H), 8.45-7.94 (m, 3H), 7.69 (s, 1H), 7.52-7.34 (m, 3H), 7.30-7.18 (m, 3H), 7.13-6.96 (m, 1H), 6.79-6.71 (m, 1H), 2.64 (s, 3H), 2.02 (s, 3H).

### N-(4-(2-(methylthio)-4-(2-(3-phenylureido)phenyl)-1-((2-(trimethylsilyl)ethoxy)methyl)-1H-imidazol-5-yl)pyridin-2-yl)acetamide (FW-101)

The title compound was synthesized according to general procedure 1B) from 50 mg (0.11 mmol) FW-81 and 14 mg (0.12 mmol 1.1 eq.) phenyl isocyanate. Flash chromatography (SiO₂, n-hex->n-hex/EtOAc 40:60). Yield: 56 mg (89%) as pale yellow solid. ESI-MS: 611.1 [M+Na]⁺. ¹H NMR (200 MHz, CDCl₃) δ 10.12 (s, 1H), 9.50 (s, 1H), 8.38 (s, 1H), 7.95-7.51 (m, 2H), 7.39-6.99 (m, 9H), 6.77 (s, 1H), 5.34 (s, 2H), 3.55-3.41 (m, 2H), 2.42 (s, 3H), 2.24 (s, 3H), 0.93 (t, J=8.2 Hz, 2H), −0.02 (s, 9H).

### N-(4-(2-(methylthio)-4-(3-(3-phenylureido)phenyl)-1H-imidazol-5-yl)pyridin-2-yl)acetamide (020)

The title compound was synthesized according to general procedure 2B) from 50 mg (0.09 mmol) of FW-101. Flash chromatography (SiO₂, DCM/MeOH 2%->8%) Yield: 22 mg (57%) as off-white solid. ESI-MS: 481.2 [M+Na]⁺. As mixture of tautomers: ¹H NMR (400 MHz, DMSO) δ 13.16-12.52 (m, 1H), 10.31-10.13 (m, 1H), 8.92 (s, 1H), 8.40-7.95 (m, 3H), 7.65 (s, 1H), 7.42 (t, J=7.4 Hz, 1H), 7.31-7.26 (m, 2H), 7.25-7.17 (m, 3H), 7.12-6.97 (m, 1H), 6.93 (t, J=7.2 Hz, 1H), 6.76 (dd, J=5.3, 1.3 Hz, 1H), 2.63 (s, 3H), 2.01 (s, 3H).

### N-(2-(5-(2-acetamidopyridin-4-yl)-2-(methylthio)-1-((2-(trimethylsilyl)ethoxy)methyl)-1H-imidazol-4-yl)phenyl)cyclopentanecarboxamide (FW-102)

The title compound was synthesized according to general procedure 1A) from 50 mg (0.11 mmol) FW-81, 15 μl (0.14 mmol 1.3 eq.) cyclopentanecarboxylic acid, 72 mg (0.14 mmol, 1.3 eq.) PyBOP and 55 μl (0.32 mmol, 3.0 eq.) DIPEA. Flash chromatography (SiO₂, n-hex->n-hex/EtOAc 35:65). Yield: 40 mg (66%) as off-white solid. ESI-MS: 588.1 [M+Na]⁺. ¹H NMR (200 MHz, CDCl₃) δ 10.69 (s, 1H), 9.02 (s, 1H), 8.57-8.38 (m, 1H), 8.34 (s, 1H), 8.26-8.08 (m, 1H), 7.19 (t, J=7.4 Hz, 1H), 6.94-6.82 (m, 2H), 6.80-6.67 (m, 1H), 5.26 (s, 2H), 3.58-3.42 (m, 2H), 2.73 (s, 3H), 2.19 (s, 3H), 1.97-1.53 (m, 9H), 0.96-0.86 (m, 2H), −0.04 (s, 9H).

### N-(2(5-(2-acetamidopyridin-4-yl)-2-(methylthio)-1H-imidazol-4-yl)phenyl)cyclopentane-carboxamide (022)

The title compound was synthesized according to general procedure 2B) from 40 mg (0.07 mmol) of FW-102. Flash chromatography (SiO₂, DCM/MeOH 1%->8%) Yield: 20 mg (64%) as off-white solid. ESI-MS: 458.2 [M+Na]⁺. As mixture of tautomers: ¹H NMR (400 MHz, DMSO) δ 13.33-12.38 (m, 1H), 10.33 (s, 1H), 9.42-8.16 (m, 2H), 8.09-7.67 (m, 2H), 7.44-7.07 (m, 3H), 6.87-6.62 (m, 1H), 2.63 (s, 3H), 2.05 (s, 3H).

### N-(2-(5-(2-acetamidopyridin-4-yl)-2-(methylthio)-1-((2-(trimethylsilyl)ethoxy)methyl)-1H-imidazol-4-yl)phenyl)-4-fluorobenzamide (FW-103)

The title compound was synthesized according to general procedure 1A) from 58 mg (0.13 mmol) FW-81, 22 mg (0.16 mmol 1.3 eq.) 4-fluorobenzoic acid, 83 mg (0.16 mmol, 1.3 eq.) PyBOP and 65 μl (0.37 mmol, 3.0 eq.) DIPEA. Flash chromatography (SiO₂, n-hex->n-hex/EtOAc 30:70). Yield: 63 mg (86%) as off-white solid. ESI-MS: 614.1 [M+Na]⁺. ¹H NMR (200 MHz, CDCl₃ δ 11.72 (s, 1H), 9.22-9.03 (m, 1H), 8.68-8.56 (m, 1H), 8.41 (s, 1H), 8.28-8.18 (m, 1H), 8.15-8.01 (m, 2H), 7.36-7.14 (m, 3H), 6.99-6.80 (m, 3H), 5.32 (s, 2H), 3.59-3.46 (m, 2H), 2.69 (s, 3H), 2.25 (s, 3H), 1.00-0.89 (m, 2H), −0.02 (s, 9H).

### N-(2-(5-(2-acetamidopyridin-4-yl)-2-(methylthio)-1H-imidazol-4-yl)phenyl)-4-fluorobenzamide (021)

The title compound was synthesized according to general procedure 2B) from 53 mg (0.09 mmol) of FW-103. Flash chromatography (SiO₂, DCM/MeOH 1%->6%) Yield: 25 mg (60%) as off-white solid. ESI-MS: 484.1 [M+Na]⁺. As mixture of tautomers: ¹H NMR (200 MHz, DMSO) δ 13.27-12.45 (m, 1H), 11.36-10.43 (m, 1H), 10.22-9.41 (m, 1H), 8.46-8.06 (m, 1H), 8.02-7.70 (m, 1H), 7.60-7.24 (m, 1H), 7.24-6.64 (m, 1H), 2.59 (s, 3H), 2.04 (s, 3H).

### N-(2-(5-(2-acetamidopyridin-4-yl)-2-(methylthio)-1-((2-(trimethylsilyl)ethoxy)methyl)-1H-imidazol-4-yl)phenyl)benzamide (FW-142)

The title compound was synthesized according to general procedure 1A) from 50 mg (0.13 mmol) FW-81, 17 mg (0.14 mmol 1.3 eq.) benzoic acid, 72 mg (0.14 mmol, 1.3 eq.) PyBOP and 45 μl (0.26 mmol, 3.0 eq.) DIPEA. Flash chromatography (SiO₂, n-hex->n-hex/EtOAc 30:70). Yield: 59 mg (96%) as off-white solid. ESI-MS: 596.6 [M+Na]⁺. The product was directly used in the next step without further characterization

### N-(2-(5-(2-acetamidopyridin-4-yl)-2-(methylthio)-1H-imidazol-4-yl)phenyl)benzamide (024)

The title compound was synthesized according to general procedure 2B) from 59 mg (0.13 mmol) of FW-142. Flash chromatography (SiO₂, DCM/MeOH 1%->8%) Yield: 40 mg (70%) as off-white solid. ESI-MS: 466.5 [M+Na]⁺. As mixture of tautomers: ¹H NMR (200 MHz, DMSO) δ 13.36-12.47 (m, 1H), 11.55-10.40 (m, 1H), 10.36-9.27 (m, 1H), 8.52-8.11 (m, 2H), 8.06-7.74 (m, 2H), 7.64-7.18 (m, 6H), 7.10-6.39 (m, 2H), 2.60 (s, 3H), 2.05 (s, 3H).

### N-(2-(5-(2-acetamidopyridin-4-yl)-2-(methylthio)-1-((2-(trimethylsilyl)ethoxy)methyl)-1H-imidazol-4-yl)phenyl)-2,6-difluorobenzamide (FW-163)

The title compound was synthesized according to general procedure 1A) from 40 mg (0.09 mmol) FW-81, 16 mg (0.10 mmol 1.2 eq.) 2,6-difluorobenzoic acid, 62 mg (0.12 mmol, 1.4 eq.) PyBOP and 40 μl (0.26 mmol, 3.0 eq.) TEA. Flash chromatography (SiO₂, n-hex->n-hex/EtOAc 35:65). Yield: 36 mg (69%) as pale yellow solid. ESI-MS: 632.7 [M+Na]⁺. ¹H NMR (200 MHz, CDCl₃) δ 11.70 (s, 1H), 9.10-8.89 (m, 1H), 8.72-8.61 (m, 1H), 8.48-8.34 (m, 1H), 8.29-8.18 (m, 1H), 7.51-7.24 (m, 2H), 7.06-6.82 (m, 5H), 5.23 (s, 2H), 3.60-3.46 (m, 2H), 2.35 (s, 3H), 2.25 (s, 3H), 1.00-0.89 (m, 2H), −0.01 (s, 9H).

### N-(2-(5-(2-acetamidopyridin-4-yl)-2-(methylthio)-1H-imidazol-4-yl)phenyl)-2,6-difluorobenzamide (030)

The title compound was synthesized according to general procedure 2B) from 32 mg (0.05 mmol) of FW-163. Flash chromatography (SiO₂, DCM/MeOH 1%->10%) Yield: 19 mg (75%) as off-white solid. ESI-MS: 502.6 [M+Na]⁺. ¹H NMR (200 MHz, DMSO) δ 13.77-12.96 (m, 1H), 11.35 (s, 1H), 10.45 (s, 1H), 8.22-7.85 (m, 3H), 7.65-7.31 (m, 4H), 7.23-7.00 (m, 3H), 2.62 (s, 3H), 2.15 (s, 3H).

### N-(2-(5-(2-acetamidopyridin-4-yl)-2-(methylthio)-1-((2-(trimethylsilyl)ethoxy)methyl)-1H-imidazol-4-yl)phenyl)-1H-indole-4-carboxamide (FW-128)

The title compound was synthesized according to general procedure 1A) from 65 mg (0.14 mmol) FW-81, 30 mg (0.19 mmol 1.35 eq.) 1H-indole-4-carboxylic acid, 97 mg (0.19 mmol, 1.35 eq.) PyBOP and 72 μl (0.26 mmol, 3.0 eq.) DIPEA. Flash chromatography (SiO₂, n-hex->n-hex/EtOAc 20:80). Yield: 80 mg (94%) as pale yellow solid. ESI-MS: 635.6 [M+Na]⁺. The product was directly used in the next step without further characterization

### N-(2-(5-(2-acetamidopyridin-4-yl)-2-(methylthio)-1H-imidazol-4-yl)phenyl)-1H-indole-4-carboxamide (023)

The title compound was synthesized according to general procedure 2B) from 75 mg (0.12 mmol) of FW-128. Flash chromatography (SiO₂, DCM/MeOH 2%->10%, n-hex->EtOAc) Yield: 20 mg (33%) as off-white solid. ESI-MS: 505.3 [M+Na]⁺. ¹H NMR (400 MHz, DMSO) δ 13.19-12.63 (m, 1H), 11.51-11.23 (m, 1H), 11.17-9.00 (m, 2H), 8.56-7.98 (m, 3H), 7.64-7.39 (m, 3H), 7.38-7.34 (m, 1H), 7.32-7.07 (m, 2H), 7.02-6.99 (m, 1H), 6.97-6.83 (m, 1H), 6.78-6.41 (m, 1H), 2.59-2.40 (m, 3H), 2.08-2.03 (m, 3H).

### N-(2-(5-(2-acetamidopyridin-4-yl)-2-(methylthio)-1-((2-(trimethylsilyl)ethoxy)methyl)-1H-imidazol-4-yl)phenyl)-4-methylthiophene-2-carboxamide (FW-145)

The title compound was synthesized according to general procedure 1A) from 50 mg (0.11 mmol) FW-81, 20 mg (0.14 mmol 1.3 eq.) 4-methylthiophene-2-carboxylic acid, 72 mg (0.14 mmol, 1.3 eq.) PyBOP and 55 μl (0.32 mmol, 3.0 eq.) DIPEA. Flash chromatography (SiO₂, n-hex->n-hex/EtOAc 25:75). Yield: 54 mg (85%) as pale yellow solid. ESI-MS: 616.4 [M+Na]⁺. ¹H NMR (200 MHz, CDCl₃) δ 11.61 (s, 1H), 9.26 (s, 1H), 8.56 (d, J=8.2 Hz, 1H), 8.42 (s, 1H), 8.22 (d, J=5.2 Hz, 1H), 7.61 (s, 1H), 7.33-7.22 (m, 1H), 7.17-7.12 (m, 1H), 6.97-6.90 (m, 2H), 6.87-6.78 (m, 1H), 5.36 (s, 2H), 3.58-3.45 (m, 2H), 2.78 (s, 3H), 2.34 (s, 3H), 2.25 (s, 3H), 0.98-0.89 (m, 2H), −0.03 (s, 9H).

### N-(2-(5-(2-acetamidopyridin-4-yl)-2-(methylthio)-1H-imidazol-4-yl)phenyl)-4-methyl-thiophene-2-carboxamide (026)

The title compound was synthesized according to general procedure 2B) from 50 mg (0.08 mmol) of FW-145. Flash chromatography (SiO₂, DCM/MeOH 1%->8%) Yield: 20 mg (51%) as off-white solid. ESI-MS: 486.5 [M+Na]⁺. ¹H NMR (200 MHz, CDCl₃+MeOD) δ 10.82 (s, 1H), 9.54 (s, 1H), 8.37 (d, J=7.8 Hz, 1H), 8.07-7.83 (m, 2H), 7.44-7.21 (m, 3H), 7.10-6.90 (m, 3H), 2.69 (s, 3H), 2.26 (s, 3H), 2.20 (s, 3H).

### N-(2-(5-(2-acetamidopyridin-4-yl)-2-(methylthio)-1-((2-(trimethylsilyl)ethoxy)methyl)-1H-imidazol-4-yl)phenyl)-3-methoxybenzamide (FW-147)

The title compound was synthesized according to general procedure 1A) from 50 mg (0.11 mmol) FW-81, 21 mg (0.14 mmol 1.3 eq.) 3-methoxybenzoic acid, 72 mg (0.14 mmol, 1.3 eq.) PyBOP and 55 μl (0.32 mmol, 3.0 eq.) DIPEA. Flash chromatography (SiO₂, n-hex->n-hex/EtOAc 25:75). Yield: 52 mg (81%) as white solid. ESI-MS: 626.5 [M+Na]⁺. ¹H NMR (200 MHz, CDCl₃) δ 11.60 (s, 1H), 9.23 (s, 1H), 8.62 (d, J=8.3 Hz, 1H), 8.41 (s, 1H), 8.22 (d, J=5.1 Hz, 1H), 7.70-7.52 (m, 2H), 7.46-7.36 (m, 1H), 7.35-7.24 (m, 1H), 7.14-7.04 (m, 1H), 7.03-6.91 (m, 2H), 6.90-6.80 (m, 1H), 5.43-5.18 (m, 2H), 4.00-3.81 (m, 3H), 3.60-3.45 (m, 2H), 2.78-2.60 (m, 3H), 2.24 (s, 3H), 1.00-0.88 (m, 2H), 0.04-−0.11 (m, 9H).

### N-(2-(5-(2-acetamidopyridin-4-yl)-2-(methylthio)-1H-imidazol-4-yl)phenyl)-3-methoxybenzamide (025)

The title compound was synthesized according to general procedure 2B) from 50 mg (0.08 mmol) of FW-147. Flash chromatography (SiO₂, DCM/MeOH 1%->10%) Yield: 30 mg (76%) as off-white solid. ESI-MS: 496.5 [M+Na]⁺. ¹H NMR (400 MHz, DMSO) δ 13.38-12.54 (m, 1H), 11.60-9.20 (m, 2H), 8.47-8.07 (m, 2H), 8.03-7.69 (m, 1H), 7.56-7.15 (m, 5H), 7.15-6.72 (m, 3H), 3.77 (s, 3H), 2.59 (s, 3H), 2.05 (s, 3H).

### N-(2-(5-(2-acetamidopyridin-4-yl)-2-(methylthio)-14(2-(trimethylsilyl)ethoxy)methyl)-1H-imidazol-4-yl)phenyl)furan-2-carboxamide (FW-149)

The title compound was synthesized according to general procedure 1A) from 55 mg (0.12 mmol) FW-81, 17 mg (0.15 mmol 1.3 eq.) furan-2-carboxylic acid, 79 mg (0.15 mmol, 1.3 eq.) PyBOP and 49 μl (0.35 mmol, 3.0 eq.) TEA. Flash chromatography (SiO₂, n-hex->n-hex/EtOAc 30:70). Yield: 60 mg (91%) as white solid. ESI-MS: no mass detected. ¹H NMR (200 MHz, CDCl₃) δ 11.45 (s, 1H), 9.24 (s, 1H), 8.56 (d, J=8.2 Hz, 1H), 8.41 (s, 1H), 8.20 (d, J=5.0 Hz, 1H), 7.50 (s, 1H), 7.33-7.22 (m, 2H), 7.02-6.80 (m, 3H), 6.63-6.53 (m, 1H), 5.31 (s, 2H), 3.62-3.51 (m, 2H), 2.88 (s, 3H), 2.23 (s, 3H), 1.02-0.91 (m, 2H), −0.02 (s, 9H).

### N-(2-(5-(2-acetamidopyridin-4-yl)-2-(methylthio)-1H-imidazol-4-yl)phenyl)furan-2-carboxamide (027)

The title compound was synthesized according to general procedure 2B) from 60 mg (0.11 mmol) of FW-149. Flash chromatography (SiO₂, DCM/MeOH 1%->8%) Yield: 34 mg (73%) as off-white solid. ESI-MS: 456.5 [M+Na]⁺. ¹H NMR (200 MHz, DMSO) δ 13.25-12.51 (m, 1H), 11.59-8.80 (m, 2H), 8.13 (t, J=50.0 Hz, 4H), 7.21 (t, J=24.4 Hz, 3H), 6.94 (s, 2H), 6.70 (s, 1H), 2.72 (s, 3H), 2.06 (s, 3H).

### N-(2(5-(2-acetamidopyridin-4-yl)-2-(methylthio)-1-((2-(trimethylsilyl)ethoxy)methyl)-1H-imidazol-4-yl)phenyl)nicotinamide (FW-154)

The title compound was synthesized according to general procedure 1A) from 50 mg (0.11 mmol) FW-81, 17 mg (0.14 mmol 1.3 eq.) nicotinic acid, 72 mg (0.14 mmol, 1.3 eq.) PyBOP and 45 μl (0.32 mmol, 3.0 eq.) TEA. Flash chromatography (SiO₂, n-hex->n-hex/EtOAc 20:80). Yield: 45 mg (73%) as white solid. ESI-MS: 597.3 [M+Na]⁺. ¹H NMR (200 MHz. CDCl₃) δ 11.74 (s, 1H), 9.32 (s, 1H). 9.10 (s, 1H), 8.73-8.58 (m, 1H), 8.54-8.13 (m, 4H), 7.89-7.28 (m, 2H), 7.19 (t, J=7.1 Hz, 1H), 6.96-6.70 (m, 3H), 5.17 (s, 2H), 3.48-3.35 (m, 2H), 2.55 (s, 3H), 2.10 (s, 3H), 0.86-0.73 (m, 2H), −0.16 (s, 9H).

### N-(2-(5-(2-acetamidopyridin-4-yl)-2-(methylthio)-1H-imidazol-4-yl)phenyl)nicotinamide (029)

The title compound was synthesized according to general procedure 2B) from 45 mg (0.08 mmol) of FW-154. Flash chromatography (SiO₂, n-hex->n-hex/EtOAc/MeOH 5:90:5) Yield: 15 mg (45%) as off-white solid. ESI-MS: 487.6 [M+Na]⁺. ¹H NMR (200 MHz, DMSO) δ 12.77 (s, 1H), 11.31-9.40 (m, 2H), 9.04-8.42 (m, 2H), 8.32-7.65 (m, 4H), 7.59-7.04 (m, 4H), 6.82 (s, 1H), 2.60 (s, 3H), 2.05 (s, 3H).

### N-(2-(5-(2-acetamidopyridin-4-yl)-2-(methylthio)-14(2-(trimethylsilyl)ethoxy) methyl)-1H-imidazol-4-yl)phenyl)isonicotinamide (FW-155)

The title compound was synthesized according to general procedure 1A) from 50 mg (0.11 mmol) FW-81, 17 mg (0.14 mmol 1.3 eq.) isonicotinic acid, 72 mg (0.14 mmol, 1.3 eq.) PyBOP and 45 μl (0.32 mmol, 3.0 eq.) TEA. Flash chromatography (SiO₂, n-hex->n-hex/EtOAc 20:80). Yield: 50 mg (82%) as white solid. ESI-MS: 597.3 [M+Na]⁺. ¹H NMR (200 MHz, CDCl₃) δ 11.87 (s, 1H), 9.38 (s, 1H), 8.63 (d, J=4.2 Hz, 2H), 8.35 (d, J=8.2 Hz, 1H), 8.15-8.10 (m, 1H), 7.87-7.74 (m, 3H), 7.15 (t, J=7.0 Hz, 1H), 6.96-6.61 (m, 3H), 5.15 (s, 2H), 3.43-3.29 (m, 2H), 2.44 (s, 3H), 2.05 (s, 3H), 0.81-0.70 (m, 2H), −0.19 (s, 9H).

### N-(2-(5-(2-acetamidopyridin-4-yl)-2-(methylthio)-1H-imidazol-4-yl)phenyl)isonicotinamide (031)

The title compound was synthesized according to general procedure 2B) from 50 mg (0.09 mmol) of FW-155. Flash chromatography (SiO₂, DCM/MeOH 1%->9%) Yield: 16 mg (41%) as off-white solid. ESI-MS: 443.6 [M−H]⁻. ¹H NMR (200 MHz, DMSO) δ 12.69 (s, 1H), 10.66-9.39 (m, 2H), 8.77-8.55 (m, 2H), 8.30-7.89 (m, 3H), 7.58-7.12 (m, 5H), 6.80 (s, 1H), 2.59 (s, 3H), 2.04 (s, 3H).

### N-(2-(5-(2-acetamidopyridin-4-yl)-2-(methylthio)-1-((2-(trimethylsilyl)ethoxy)methyl)-1H-imidazol-4-yl)phenyl)picolinamide (FW-160)

The title compound was synthesized according to general procedure 1A) from 50 mg (0.11 mmol) FW-81, 20 mg (0.16 mmol 1.5 eq.) picolinic acid, 83 mg (0.16 mmol, 1.5 eq.) PyBOP and 45 μl (0.32 mmol, 3.0 eq.) TEA. Flash chromatography (SiO₂, n-hex->n-hex/EtOAc 20:80). Yield: 50 mg (82%) as white solid. ESI-MS: 597.5 [M+Na]⁺. ¹H NMR (200 MHz, CDCl₃) δ 12.11 (s, 1H), 9.40 (s, 1H), 8.78-8.52 (m, 1H), 8.39 (s, 1H), 8.31 (d, J=7.8 Hz, 1H), 8.12 (d, J=5.1 Hz, 1H), 7.87 (t, J=7.5 Hz, 1H), 7.51-7.39 (m, 1H), 7.37-7.26 (m, 1H), 7.09-6.82 (m, 3H), 5.30 (s, 2H), 3.72-3.52 (m, 2H), 2.97 (s, 3H), 2.19 (s, 3H), 1.03-0.91 (m, 2H), 0.02 (s, 9H).

### N-(2-(5-(2-acetamidopyridin-4-yl)-2-(methylthio)-1H-imidazol-4-yl)phenyl)picolinamide (028)

The title compound was synthesized according to general procedure 26) from 50 mg (0.09 mmol) of FW-160. Flash chromatography (SiO₂, DCM/MeOH 1%->10%) Yield: 29 mg (75%) as off-white solid. ESI-MS: 467.6 [M+Na]⁺. ¹H NMR (200 MHz, DMSO) δ 13.07 (s, 1H), 11.92 (s, 1H), 10.40 (s, 1H), 8.70-8.38 (m, 2H), 8.29-7.91 (m, 4H), 7.68-7.55 (m, 1H), 7.51-7.03 (m, 3H), 6.96-6.77 (m, 1H), 2.76 (s, 3H), 2.04 (s, 3H).

### N-(2-(5-(2-acetamidopyridin-4-yl)-2-(methylthio)-1-((2-(trimethylsilyl)ethoxy)methyl)-1H-imidazol-4-yl)phenyl)-2-hydroxybenzamide (FW-164)

The title compound was synthesized according to general procedure 1A) from 55 mg (0.12 mmol) FW-81, 24 mg (0.18 mmol 1.5 eq.) salicylic acid, 91 mg (0.18 mmol, 1.5 eq.) PyBOP and 49 μl (0.35 mmol, 3.0 eq.) TEA. Flash chromatography (SiO₂, n-hex->n-hex/EtOAc 20:80). Yield: 30 mg (43%) as yellow solid. ESI-MS: 612.7 [M+Na]⁺. The product was directly used in the next step without further characterization

### N-(2-(5-(2-acetamidopyridin-4-yl)-2-(methylthio)-1H-imidazol-4-yl)phenyl)-2-hydroxybenzamide (032)

The title compound was synthesized according to general procedure 2B) from 30 mg (0.05 mmol) of FW-164. Flash chromatography (SiO₂, DCM/MeOH 1%->10%) Yield: 12 mg (51%) as off-white solid. ESI-MS: 482.6 [M+Na]⁺. ¹H NMR (200 MHz, DMSO) δ 12.75 (s, 1H), 11.25 (s, 1H), 11.02-10.14 (m, 2H), 8.74-8.19 (m, 2H), 7.99 (s, 1H), 7.86 (d, J=7.2 Hz, 1H), 7.62-7.12 (m, 4H), 7.06-6.63 (m, 3H), 2.61 (s, 3H), 2.03 (s, 3H).

### 3-((2-(5-(2-acetamidopyridin-4-yl)-2-(methylthio)-1-((2-(trimethylsilyl)ethoxy)methyl)-1H-imidazol-4-yl)phenyl)carbamoyl)phenyl acetate (FW-181)

The title compound was synthesized according to general procedure 1A) from 70 mg (0.15 mmol) FW-81, 40 mg (0.22 mmol 1.5 eq.) FW-179, 72 mg (0.22 mmol, 1.5 eq.) TBTU and 62 μl (0.45 mmol, 3.0 eq.) TEA. Flash chromatography (SiO₂, n-hex->n-hex/EtOAc 20:80). Yield: 50 mg (53%) as yellow solid. ESI-MS: 654.6 [M+Na]⁺. ¹H NMR (200 MHz, CDCl₃) δ 11.66 (s, 1H), 9.03-8.80 (m, 1H), 8.60 (d, J=8.2 Hz, 1H), 8.42 (s, 1H), 8.27-8.18 (m, 1H), 8.04-7.90 (m, 1H), 7.81-7.71 (m, 1H), 7.54 (t, J=7.9 Hz, 1H), 7.48-7.25 (m, 2H), 7.00-6.79 (m, 3H), 5.32 (s, 2H), 3.61-3.48 (m, 2H), 2.69 (s, 3H), 2.38 (s, 3H), 2.25 (s, 3H), 1.01-0.90 (m, 2H), −0.02 (s, 9H).

### N-(2(5-(2-acetamidopyridin-4-yl)-2-(methylthio)-1H-imidazol-4-yl)phenyl)-3-hydroxybenzamide (034)

45 mg (0.07 mmol) FW-181 was dissolved in 2 ml DCM and 1 ml of TFA was added dropwise under vigorous stirring. The mixture was stirred at ambient temperature overnight. After complete consumption of the starting material, the volatiles were removed by rotary evaporation and the oily residue suspended in DCM. A saturated NaHCO₃ solution was added and the product extracted with EtOAc three times. The combined organic layers were dried over Na₂SO₄, filtered and the solvents removed by rotary evaporation. ESI-MS: 424.6 [M+Na]⁺. The residue was dissolved in MeOH, sat. aqueous NaHCO₃ was added until precipitation was observable, and the mixture was stirred at ambient temperature for 1 hour. Aqueous phase was extracted with DCM three times and the combined organic layers were dried over Na₂SO₄, filtered and volatiles removed in vacuo. Purification via Flash chromatography (SiO₂, DCM/MeOH 1%->10%) Yield: 20 mg (61%) over two steps as off-white sold. ESI-MS: 482.5 [M+Na]⁺. As mixture of tautomers: ¹H NMR (200 MHz, DMSO) δ 13.45-12.30 (m, 1H), 11.31 (s, 1H), 10.45 (s, 1H), 9.72 (s, 1H), 8.50-7.71 (m, 3H), 7.45-7.06 (m, 5H), 7.02-6.73 (m, 3H), 2.61 (s, 3H), 2.06 (s, 3H).

### N-(2-(5-(2-acetamidopyridin-4-yl)-2-(methylthio)-14(2-(trimethylsilyl)ethoxy)methyl)-1H-imidazol-4-yl)phenyl)-3-phenylpropanamide (FW-171)

The title compound was synthesized according to general procedure 1A) from 55 mg (0.12 mmol) FW-81, 26 mg (0.18 mmol 1.5 eq.) 3-phenylpropanoic acid, 56 mg (0.18 mmol, 1.5 eq.) TBTU and 49 μl (0.35 mmol, 3.0 eq.) TEA. Flash chromatography (SiO₂, n-hex->n-hex/EtOAc 20:80). Yield: 50 mg (73%) as yellow solid. ESI-MS: 624.7 [M+Na]⁺. ¹H NMR (200 MHz, CDCl₃) δ 10.74 (s, 1H), 9.96 (s, 1H), 8.58-8.35 (m, 2H), 8.25-7.99 (m, 1H), 7.32-7.24 (m, 6H), 6.98-6.76 (m, 3H), 5.42-5.10 (m, 2H), 3.66-3.52 (m, 2H), 3.13-3.03 (m, 2H), 2.77-2.68 (m, 5H), 2.25 (s, 3H), 1.07-0.93 (m, 2H), 0.02 (s, 9H).

### N-(2(5-(2-acetamidopyridin-4-yl)-2-(methylthio)-1H-Imidazol-4-yl)phenyl)-3-phenylpropanamide (033)

The title compound was synthesized according to general procedure 2B) from 50 mg (0.08 mmol) of FW-171. Flash chromatography (SiO₂, DCM/MeOH 1%->10%) Yield: 32 mg (83%) as off-white solid. ESI-MS: 494.7 [M+Na]⁺. ¹H NMR (200 MHz, CDCl₃) δ 12.14 (s, 1H), 10.61-9.00 (m, 4H), 8.53-8.14 (m, 2H), 8.08-7.97 (m, 2H), 7.37-7.10 (m, 7H), 7.06-6.94 (m, 2H), 2.94 (t, 2H), 2.69-2.47 (m, 5H), 2.13 (s, 7H).

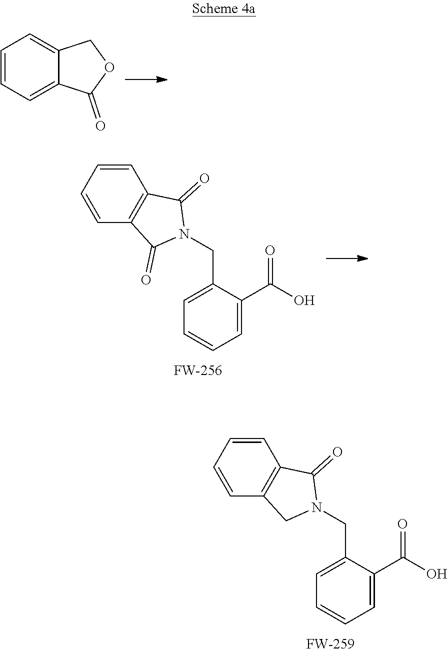

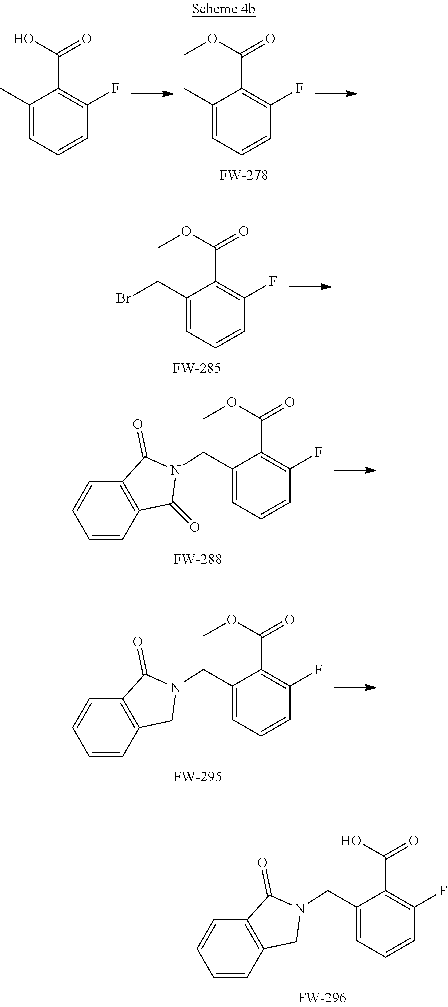 [SMILES: Cc1cccc(F)c1C(=O)O ; COC(=O)c1c(C)cccc1F ; COC(=O)c1c(F)cccc1CBr ; COC(=O)c1c(F)cccc1CN1C(=O)c2ccccc2C1=O ; COC(=O)c1c(F)cccc1CN1Cc2ccccc2C1=O ; O=C(O)c1c(F)cccc1CN1Cc2ccccc2C1=O]

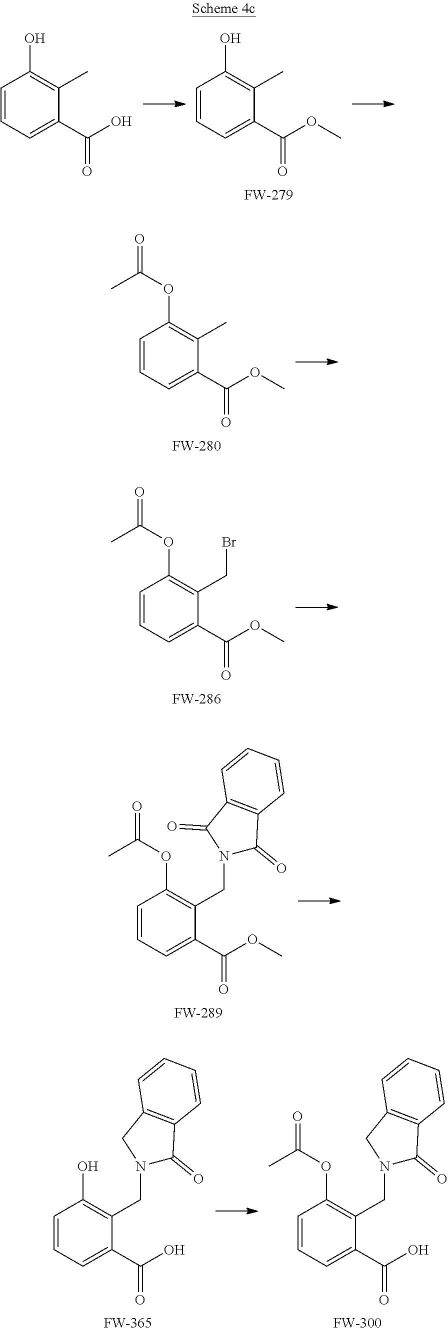

Benzoic acid precursors were prepared as shown in the synthetical schemes above.

### 2-((1,3-dioxoisoindolin-2-yl)methyl)benzoic acid (FW-256)

1.5 g (11.2 mmol) phthalide and 2.199 g (11.8 mmol) of potassium phthalimide were suspended in DMF and refluxed for 12 hours under vigorous stirring. The mixture was cooled to 0° C., iced water and aqueous 1N HCl were added under precipitation. After filtration, the residue was washed with iced water and EtOH to yield 65% (2.06 g, 7.28 mmol) of a white solid. ¹H NMR (200 MHz, DMSO-d₆) δ 513.22 (br s, 1H), 7.99-7.81 (m, 5H), 7.55-7.33 (m, 2H), 7.16 (d, J=7.5 Hz, 1H), 5.15 (s, 2H). ¹³C NMR (50 MHz, DMSO-d₆) δ 168.3, 168.0, 137.5, 134.6, 132.5, 131.8, 130.8, 129.2, 127.2, 126.2, 123.3. TLC-MS (ESI+): calcd. m/z 281.07 for C₁₆H₁₁NO₄. Found 304.9 [M+Na]⁺.

### 2-((1-oxoisoindolin-2-yl)methyl)benzoic acid (FW-259)

500 mg (1.77 mmol) FW-256 was suspended in 7 ml of a conc. HCl/AcOH (1+1) mixture. The mixture was warmed up to 60 C, 633 mg (5.33 mmol) of tin powder was added portion wise under vigorous stirring for 8 h. The mixture was cooled to room temperature and extracted with EtOAc. Drying the organic layers over Na₂SO₄ and evaporation of solvents yielded a yellowish residue. Recrystallisation in AcOH yielded 73% (350 mg, 1.29 mmol) of a white solid. ¹H NMR (200 MHz, DMSO-d₆) δ 13.13 (s, 1H), 7.92 (d, J=7.5 Hz, 1H), 7.74 (d, J=7.3 Hz, 1H), 7.55 (dt, J=10.8, 5.8 Hz, 4H), 7.38 (t, J=7.3 Hz, 1H), 7.14 (d, J=7.5 Hz, 1H), 5.10 (s, 2H), 4.44 (s, 2H). ¹³C NMR (50 MHz, DMSO-d₆) δ 168.4, 167.7, 142.0, 138.5, 132.3, 132.0, 131.4, 130.6, 129.9, 127.9, 127.6, 127.2, 123.6, 122.9, 49.9, 43.9. TLC-MS (ESI−): calcd. m/z 267.09 for C₁₆H₁₃NO₃. Found 265.9 [M−H]⁻.

### methyl 2-fluoro-6-methylbenzoate (FW-278)

750 mg (4.86 mmol) 2-Fluoro-6-methylbenzoic acid was dissolved in 10 ml of dry MeOH and five drops of conc. H₂SO₄ were added. Refluxing over 48 h led to complete conversion. After cooling to ambient temperature, sat. aq. NaHCO₃ was added and the aqueous phase was extracted with EtOAc. The combined organic layers were dried over Na₂SO₄, filtered and volatiles were evaporated. Yield: 630 mg (3.74 mmol, 77%) of an amorphic solid. ¹H NMR (200 MHz, DMSO) δ 7.43 (dd, J=14.2, 7.7 Hz, 1H), 7.21-7.05 (m, 2H), 3.87 (s, 3H), 2.32 (s, 3H). ¹³C NMR (50 MHz, DMSO) δ 165.42, 161.52, 156.59, 138.18, 138.13, 131.91, 131.73, 126.38, 126.32, 121.27, 120.95, 113.41, 112.98, 52.45, 19.05, 19.01.

### methyl 2-(bromomethyl)-6-fluorobenzoate (FW-285)

590 mg (3.51 mmol) FW-278 was dissolved in 8 ml of CCl₄ and the temperature adjusted to refluxing conditions. 655 mg (3.68 mmol, 1.05 eq.) NBS and 34 mg (0.35 mmol, 0.1 eq.) of AIBN were added in one portion and the mixture was refluxed overnight After cooling down to ambient temperature, the solvent was removed in vacuo and the residue was purified via flash chromatography (SiO₂, Hex>Hex/EtOAc 9:1). Yield: 620 mg (2.63 mmol, 75%) as colorless oil with 92% purity (HPLC indicated). ¹H NMR (200 MHz, DMSO) δ 7.63-7.50 (m, 1H), 7.46-7.40 (m, 1H), 7.38-7.27 (m, 1H), 4.78 (s, 2H), 3.91 (s, 3H). ¹³C NMR (50 MHz, DMSO) δ 164.32, 161.77, 156.79, 138.43, 138.38, 132.88, 132.69, 126.78, 126.72, 120.72, 120.40, 116.73, 116.29, 52.82, 30.29, 30.24.

### methyl 2-((1,3-dioxoisoindolin-2-yl)methyl)-6-fluorobenzoate (FW-288)

580 mg (2.4 mmol) FW-285 and 458 mg (2.47 mmol, 1.05 eq.) potassium phthalimide were suspended in DMF and stirred at ambient temperature for 48 h. Solvent was evaporated and the residue was precipitated in n-hexane and filtered. Brine was added to the residue and the aqueous phase was extracted with EtOAc multiple times. The combined organic layers were dried over Na₂SO₄ and solvents were evaporated to yield 530 mg (1.66 mmol, 71%) of a crude product as yellow solid. ESI-MS: 336.1 [M+Na]⁺. ¹H NMR (200 MHz, DMSO) δ 7.94-7.80 (m, 4H), 7.57-7.44 (m, 1H), 7.34-7.15 (m, 2H), 4.88 (s, 2H), 3.86 (s, 3H). ¹³C NMR (50 MHz, DMSO) δ 167.49, 164.66, 161.68, 156.72, 136.78, 136.74, 134.62, 132.64, 132.45, 131.49, 124.11, 124.06, 123.27, 120.05, 119.72, 115.42, 114.99, 52.79, 38.44.

### methyl 2-fluoro-6-((1-oxoisoindolin-2-yl)methyl)benzoate (FW-295)

200 mg (0.63 mmol) FW-288 was suspended in 3 ml acetic acid and 3 ml concentrated hydrochloric acid and warmed up to 40° C. 397 mg (3.35 mmol, 5.0 eq.) of tin powder was added portion wise over 4 h under stirring at 40° C. until complete conversion via TLC was indicated. The reaction mixture was cooled down to room temperature the aqueous layer was extracted with DCM. The combined organic layers were dried over Na₂SO₄, solvents were removed in vacuo and the residue was purified via flash chromatography (SiO₂; n-hexane/EtOAc 6:4). Resulting in a colorless oil in 95% yield (190 mg, 0.63 mmol). ESI-MS: 322.3 [M+Na]⁺. ¹H NMR (200 MHz, CDCl₃) δ 7.92-7.84 (m, 1H), 7.60-7.44 (m, 2H), 7.43-7.31 (m, 2H), 7.21-7.14 (m, 1H), 7.11-7.00 (m, 1H), 4.90 (s, 2H), 4.29 (s, 2H), 3.93 (s, 3H). ¹³C NMR (50 MHz, CDCl₃) δ 168.75, 165.70, 162.76, 157.73, 141.52, 138.16, 132.38, 132.35, 132.20, 131.71, 128.24, 125.06, 125.00, 124.06, 122.97, 121.40, 121.08, 115.74, 115.30, 52.94, 49.85, 43.72.

### 2-fluoro-6-((1-oxoisoindolin-2-yl)methyl)benzoic acid (FW-296)

180 mg (0.60 mmol) of FW-295 was dissolved in 3 ml THF and 6 ml of a aq. 3 N NaOH solution was added. The reaction mixture was stirred for 15 h at 50° C. After cooling down to ambient temperature, EtOAc was added and the layers were separated. The aqueous layer was acidified with 1 N HCl precipitating a white solid. The solid was filtered, washed with small amounts of an iced eq. 0.1 N HCl and dried in the oven. ESI-MS: 284.3 [M−H]⁻. ¹H NMR (200 MHz, DMSO) δ 13.71 (s, 1H), 7.82-7.38 (m, 5H), 7.34-7.18 (m, 1H), 7.16-6.99 (m, 1H), 4.82 (s, 2H), 4.36 (s, 2H).

### methyl 3-hydroxy-2-methylbenzoate (FW-279)

3.80 g (25.0 mmol) 3-hydroxy-2-methylbenzoic acid was dissolved in 50 ml of dry MeOH and five drops of conc. H₂SO₄ were added. Refluxing over 48 h led to complete conversion. After cooling to ambient temperature, the solvent was evaporated, sat aq. NaHCO₃ was added to the residue and the aqueous phase was extracted with EtOAc. The combined organic layers were dried over Na₂SO₄, filtered and volatiles were evaporated. Yield: 3.84 g (23.1 mmol, 92%) of an amorphic solid. ¹H NMR (200 MHz, CDCl₃) δ 7.41 (d, J=7.7 Hz, 1H), 7.10 (t, J=7.7 Hz, 1H), 6.94 (d, J=7.8 Hz, 1H), 5.48 (bs, 1H), 3.90 (s, 3H), 2.45 (s, 3H). ¹³C NMR (50 MHz, CDCl₃) δ 168.91, 154.55, 131.88, 126.30, 125.69, 122.71, 118.60, 52.22, 12.72.

### methyl 3-acetoxy-2-methylbenzoate (FW-280)

3.84 g (23.1 mmol) FW-279 was dissolved in 45 ml dry THF. 9.66 ml (69.3 mmol, 3.0 eq.) triethylamine, 2.40 ml (25.4 mmol, 1.1 eq.) acetic anhydride and 282 mg (2.31 mmol) 4-dimethylaminopyridine was added. The mixture was stirred overnight at ambient temperature to complete conversion. Brine was added and the aqueous phase was extracted multiple times with EtOAc. Combined organic layers were dried over Na₂SO₄, filtered and solvents removed in vacuo. Yield: 4.48 g (21.5 mmol, 93%) as colorless oil. ESI-MS: 231.3 [M+Na]⁺. ¹H NMR (200 MHz, CDCl₃) δ 7.77 (dd, J=7.6, 1.6 Hz, 1H), 7.25 (t, J=7.8 Hz, 1H), 7.17 (dd, J=8.0, 1.6 Hz, 1H), 3.89 (s, 3H), 2.39 (s, 3H), 2.34 (s, 3H). ¹³C NMR (50 MHz, CDCl₃) δ 169.24, 167.68, 149.99, 132.33, 131.88, 128.32, 126.27, 125.90, 52.17, 20.86, 13.54.

### methyl 3-acetoxy-2-(bromomethyl)benzoate (FW-286)

3.45 g (16.6 mmol) FW-280 was dissolved in 16 ml of CCl₄ and the temperature adjusted to refluxing conditions. 3.00 g (16.9 mmol, 1.02 eq.) NBS and 272 mg (1.66 mmol, 0.1 eq.) of AIBN were added in one portion and the mixture was refluxed overnight. After cooling down to ambient temperature, the solvent was removed in vacuo and the residue precipitated in n-hexane and filtered. Yield of crude product: 4.85 g (quantitative) as pale brown solid. ESI-MS: 309.5, 311.4 [M+Na]⁺. ¹H NMR (400 MHz, DMSO) δ 7.77 (dd, J=7.8, 1.3 Hz, 1H), 7.53 (t, J=8.0 Hz, 1H), 7.45 (dd, J=8.2, 1.3 Hz, 1H), 4.91 (s, 2H), 3.88 (s, 3H), 2.37 (s, 3H). ¹³C NMR (101 MHz, DMSO) δ 168.63, 166.08, 149.47, 130.72, 130.39, 129.48, 128.02, 127.47, 52.51, 24.28, 20.70.

### methyl 3-acetoxy-2-((1,3-dioxoisoindolin-2-yl)methyl)benzoate (FW-289)

4.05 g (14.1 mmol) FW-286 and 2.61 g (14.1 mmol, 1.0 eq.) potassium phthalimide were suspended in DMF and stirred at ambient temperature for 48 h. Iced water was added to the mixture precipitating a white solid, which was filtered and oven dried. Yield of the crude product 4.68 mg (13.3 mmol, 94%) as white solid. ¹H NMR (400 MHz, DMSO) δ 7.85-7.78 (m, 4H), 7.66 (dd, J=7.8, 1.2 Hz, 1H), 7.47 (t, J=7.9 Hz, 1H), 7.32 (dd, J=8.1, 1.1 Hz, 1N), 5.11 (s, 2H), 3.88 (s, 3H), 2.25 (s, 3H). ¹³C NMR (101 MHz, DMSO) δ 169.73, 167.78, 167.72, 150.47, 135.00, 134.36, 131.79, 129.46, 128.34, 127.54, 127.04, 123.51, 52.95, 33.28, 21.35.

### 3-hydroxy-2-((1-oxoisoindolin-2-yl)methyl)benzoic acid (FW-365)

4.13 g (11.7 mmol) FW-289 was suspended in 25 ml acetic acid and 25 ml concentrated hydrochloric acid and warmed up to 80° C. 5.55 g (46.8 mmol, 4.0 eq.) of tin powder was added in one portion and the mixture was stirred overnight at 80° C. The reaction mixture was cooled down to ambient temperature, ice water was added under precipitation of a white solid. Solid was filtered, washed aq 1 N HCl. and oven dried. Yielding 2.41 g (8.54 mmol, 73%) of a white solid. ESI-MS: 306.5 [M+Na]⁺. ¹H NMR (400 MHz, DMSO) δ 13.03 (s, 1H), 10.03 (s, 1H), 7.67 (d, J=7.5 Hz, 1H), 7.56-7.50 (m, 2H), 7.48-7.43 (m, 1H), 7.29-7.23 (m, 2H), 7.07-7.01 (m, 1H), 4.97 (s, 2H), 4.24 (s, 2H). ¹³C NMR (101 MHz, DMSO) δ 169.47, 167.86, 157.24, 142.40, 134.66, 132.67, 131.60, 129.33, 128.23, 123.86, 123.01, 122.41, 120.91, 119.17, 49.87, 38.22.

### 3-acetoxy-2-((1-oxoisoindolin-2-yl)methyl)benzoic acid (FW-300)

2.18 g (7.70 mmol) FW-365 was dissolved in 10 ml dry THF. 3.22 ml (23.1 mmol, 3.0 eq.) triethylamine, 0.76 ml (8.09 mmol, 1.05 eq.) acetic anhydride and 94 mg (0.77 mmol) 4-dimethylaminopyridine was added. The mixture was stirred overnight at ambient temperature to complete conversion. Iced aqueous 0.1 N HCl was added under precipitation of a white solid, which was filtered and oven dried. Yield: 2.40 g (7.38 mmol, 96%) as white solid. ESI-MS: 348.6 [M+Na]⁺. ¹H NMR (200 MHz, DMSO) δ 13.44 (s, 1H), 7.84-7.64 (m, 2H), 7.62-7.39 (m, 4H), 7.32 (d, J=7.7 Hz, 1H), 4.99 (s, 2H), 4.12 (s, 2H), 2.20 (s, 3H). ¹³C NMR (50 MHz, DMSO) δ 169.29, 166.85, 162.39, 150.38, 141.74, 131.86, 131.28, 129.12, 128.65, 127.81, 127.51, 126.60, 123.42, 122.70, 120.20, 48.58, 37.05, 20.75.

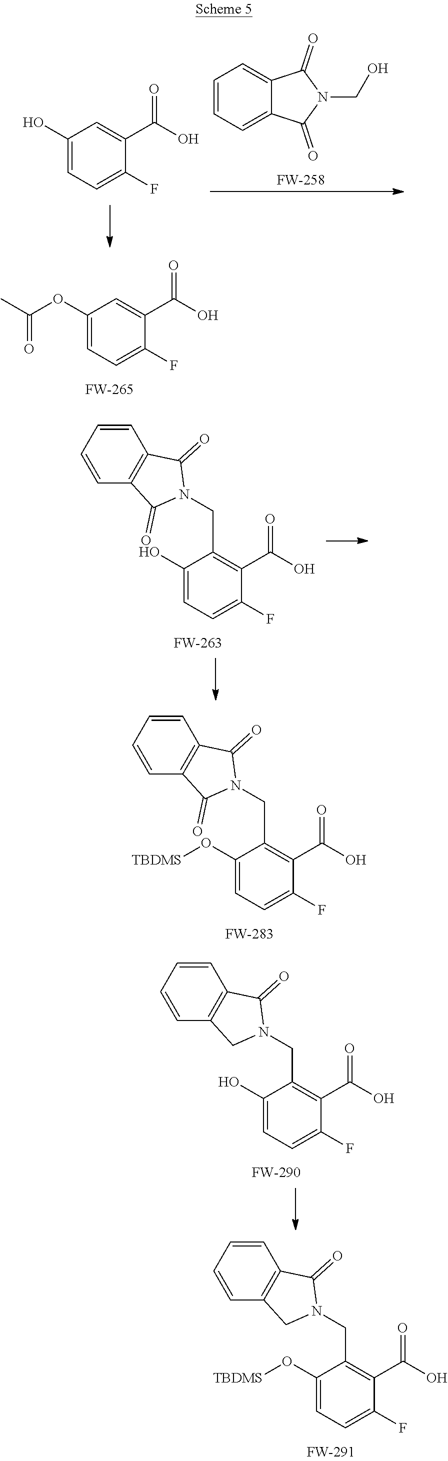

### 2-(Hydroxymethyl)isoindoline-1,3-dione (FW-258)

6.36 g (43 mmol) of phthalimide were suspended in demineralized water and 4.45 ml of formalin solution (35% v/v, 52 mmol) was added. The flask was sealed and heated for 4 h at 95° C. The reaction mixture was cooled down to room temperature and ice was added to the suspension. The precipitate was filtered and washed with cold water, obtaining 96% yield (7.28 g, 41.3 mmol) of white crystals. ¹H NMR (200 MHz, DMSO-d₆) δ 8.03-7.74 (m, 4H), 6.39 (t, J=7.0 Hz, 1H), 4.96 (d, J=7.0 Hz, 2H). C NMR (50 MHz, DMSO-d₆) δ 154.3, 121.7, 118.4, 110.2, 47.1.

### 2-((1,3-Dioxoisoindolin-2-yl)methyl)-6-fluoro-3-hydroxybenzoic acid (FW-263)

3.33 g (21.3 mmol) 2-fluoro-5-hydroxybenzoic acid was dissolved in 50 ml of fresh concentrated sulfuric acid and warmed up to 65° C. 3.78 g (21.3 mol) FW-258 was added portion wise over 30 min to the reaction mixture. The mixture was cooled to room temperature and addition of 300 ml of iced water precipitated a pinkish solid, which was filtered and dried. Recrystallization in 45 ml AcOH yielded 42% (2.85 g, 8.96 mmcl) of a white sold. ¹H NMR (400 MHz DMSO-d₆) δ 13.38 (br s, 1H), 9.86 (s, 1H), 7.82 (s, 4H), 7.02 (t, J=9.1 Hz, 1H), 6.84 (dd, J=9.0, 4.8 Hz, 1H), 4.82 (s, 2H). ¹³C NMR (101 MHz, DMSO-d₆) δ 167.1, 165.8, 151.8 (d, J=1.8 Hz), 151.7 (d, J=237.3 Hz), 134.2, 131.6, 123.5 (d, J=19.5 Hz), 122.9, 120.3 (d, J=2.8 Hz), 116.8 (d, J=8.2 Hz), 115.0 (d, J=23.2 Hz), 34.3. TLC-MS (ESI+): calcd. m/z 315.05 for C₁₆H₁₀FNO₅. Found 315.9 [M+H]⁺.

### 6-Fluoro-3-hydroxy-2-((1-oxoisoindolin-2-yl)methyl)benzoic acid (FW-290)

1.00 g (3.2 mmol) FW-263 was suspended in 15 ml acetic acid and 15 ml concentrated hydrochloric acid and warmed up to 50° C. 2.09 g (17.6 mmol) of tin powder was added portion wise over 3 h under stirring at 50° C. until complete conversion via TLC was indicated. The reaction mixture was cooled down to room temperature the aqueous layer was extracted with EtOAc. The combined organic layers were washed three times with 0.1 N hydrochloric acid and dried over Na₂SO₄. The solvent was removed in vacuo and the residue was purified via flash chromatography (SiO₂; n-hexane/EtOAc/MeOH 50:47.5:2.5+1% acetic acid) to obtain a yellowish solid in 85% yield (0.82 g, 2.7 mmol). ¹H NMR (200 MHz, DMSO-d₆) δ 10.02 (br s, 1H), 7.71-7.64 (m, 1H), 7.56-7.40 (m, 3H), 7.11 (t, J=9.0 Hz, 1H), 6.93 (dd, J=8.9, 4.8 Hz, 1H), 4.73 (s, 2H), 4.22 (s, 2H). ¹³C NMR (101 MHz, DMSO-d₆) δ 167.5, 166.2, 152.6 (d, J=1.2 Hz), 151.7 (d, J=237.7 Hz), 142.0, 132.1, 131.5, 128.0, 124.9 (d, J=19.1 Hz), 123.6, 122.9, 120.6 (d, J=2.4 Hz), 117.4 (d, J=8.1 Hz), 116.2 (d, J=22.9 Hz), 49.4, 38.2. TLC-MS (ESI−): calcd. m/z 301.08 for C₁₆H₁₂FNO₄. Found 300.1 [M−H]⁻.

### 3-Acetoxybenzoic acid (FW-179)

200 mg (1.44 mmol) 3-hydroxybenzoic acid and 4 mg (0.03 mmol, 0.02 eq.) 4-dimethylaminopyridine were suspended in 3 ml acetic anhydride and the mixture was refluxed overnight After cooling down to room temperature a saturated aqueous NH₄Cl solution was added to the reaction mixture and the aqueous layer was extracted with EtOAc. Combined organic layers were dried over Na₂SO₄ and the solvents were removed in vacuo. Yield: 150 mg (58%, 0.83 mmol). The crude product was used directly in the next step without further purification. ESI-MS: 179.0 [M−H]⁻. ¹H NMR (200 MHz, CDCl₃) δ 11.66 (s, 1H), 8.00 (d, J=7.4 Hz, 1H), 7.84 (s, 1H), 7.50 (t, J=7.8 Hz, 1H), 7.36 (d, J=7.3 Hz, 1H), 2.33 (s, 3H). ¹³C NMR (50 MHz, CDCl₃) δ 171.43, 169.38, 150.82, 130.96, 129.72, 127.77, 127.39, 123.58, 21.18.

### 5-Acetoxy-2-fluorobenzoic acid (FW-265)

550 mg (3.52 mmol) 2-fluoro-5-hydroxybenzoic acid and 5 mg (0.04 mmol) 4-dimethylaminopyridine were suspended in 7 ml acetic anhydride and the mixture was refluxed overnight After cooling down to room temperature a saturated aqueous NH₄Cl solution was added to the reaction mixture and the aqueous layer was extracted with EtOAc. Combined organic layers were dried over Na₂SO₄ and the solvents were removed in vacuo. Purification via flash chromatography (SiO₂; DCM/MeOH 90:10+1% formic acid) yielded 75% (530 mg, 2.67 mmol) of a white solid. ¹H NMR (200 MHz, CDCl₃) δ 8.70 (br s, 1H), 7.75 (dd, J=5.9, 2.9 Hz, 1H), 7.39-7.28 (m, 1H), 7.25-7.13 (m, 1H), 2.32 (s, 3H). ¹³C NMR (50 MHz, CDCl₃) δ 169.3, 168.3 (d, J=3.7 Hz), 160.1 (d, J=260.8 Hz), 146.3 (d, J=3.3 Hz), 129.0 (d, J=9.3 Hz), 125.6 (d, J=0.7 Hz), 118.5 (d, J=3.6 Hz), 118.1 (d, J=10.1 Hz), 21.1.

### 3-((tert-Butyldimethylsilyl)oxy)-2-((1,3-dioxoisoindolin-2-yl)methyl)-6-fluorobenzoic acid (FW-283)

2.25 g (7.13 mmol) FW-263 was suspended in 20 ml of ACN and cooled down to 0° C. After slow addition of 2.5 ml (18.0 mmol) triethylamine, 1.29 g (8.56 mmol) TBDMS-Cl was added in one portion. The mixture was warmed up to room temperature and stirred overnight to complete conversion. 10 ml of a 2 N HCl in 1,4-dioxane and 50 ml EtOAc were added. 10 ml of an aqueous 0.01 N hydrochloric acid solution was added under dissolution of the precipitate. Layers were separated, the organic layer was dried with Na₂SO₄ and solvents were removed in vacuo. After precipitation in n-pentane a white solid in 98% yield (3.03 g, 6.98 mmol) was obtained. ¹H NMR (400 MHz, DMSO-d₆) δ 13.41 (s, 1H), 7.83 (s, 4H), 7.11 (t, J=9.0 Hz, 1H), 6.95 (dd, J=9.0, 4.7 Hz, 1H), 4.84 (s, 2H), 0.95 (s, 9H), 0.28 (s, 6H). ¹³C NMR (101 MHz, DMSO-d₆) δ 167.2, 165.6, 152.6 (d, J=239.5 Hz), 49.7 (d, J=2.2 Hz), 134.3, 131.6, 124.5 (d, J=3.3 Hz), 123.6 (d, J=20.4 Hz), 123.0, 120.1 (d, J=8.3 Hz), 115.2 (d, J=23.1 Hz), 34.3, 25.7, −4.4. TLC-MS (ESI+): calcd. m/z 429.14 for C₂₂H₂₄FNO₅Si. Found 430.2 [M+H]⁺.

### 3-((tert-Butyldimethylsilyl)oxy)-6-fluoro-2-((1-oxoisoindolin-2-yl)methyl)benzoic acid (FW-291)

780 mg (2.58 mmol) FW-290 was suspended in 12 ml ACN and cooled down to 0° C. After slow addition of 1.08 ml (7.76 mmol) triethylamine, 507 mg (3.36 mmol) TBDMS-Cl was added portion wise and the reaction mixture was warmed up to room temperature. Stirring over 4 h led to complete conversion, whereupon water was added to the mixture and 0.1 N hydrochloric acid was used to adjust to pH 4. After extraction of the aqueous layer multiple times with EtOAc, the organic layers were dried with Na₂SO₄ and the solvents were removed in vacuo. Purification via flash chromatography (SiO₂; EtOAc/MeOH 85:15) yielded 46% (500 mg, 1.19 mmol) of a yellowish solid. ¹H NMR (200 MHz, DMSO-d₆) δ 7.70-7.60 (m, 1H), 7.57-7.35 (m, 3H), 7.00 (t, J=8.7 Hz, 1H), 6.72 (dd, J=8.8, 4.6 Hz, 1H), 4.66 (s, 2H), 4.14 (s, 2H), 0.82 (s, 9H), 0.17 (s, 6H). TLC-MS (ESI+): calcd. m/z 415.16 for C₂₂H₂₆FNO₄Si. Found 438.6 [M+Na]⁺.

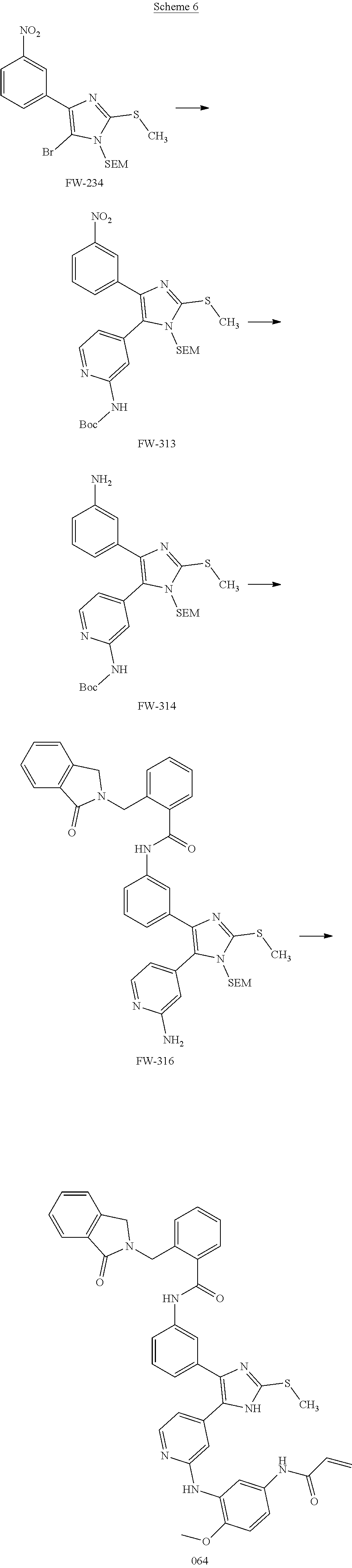 [SMILES: CSc1nc(-c2cccc([N+](=O)[O-])c2)c(Br)n1COCC[Si](C)(C)C ; CSc1nc(-c2cccc([N+](=O)[O-])c2)c(-c2ccnc(NC(=O)OC(C)(C)C)c2)n1COCC[Si](C)(C)C ; CSc1nc(-c2cccc(N)c2)c(-c2ccnc(NC(=O)OC(C)(C)C)c2)n1COCC[Si](C)(C)C ; CSc1nc(-c2cccc(NC(=O)c3ccccc3CN3Cc4ccccc4C3=O)c2)c(-c2ccnc(N)c2)n1COCC[Si](C)(C)C ; C=CC(=O)Nc1ccc(OC)c(Nc2cc(-c3[nH]c(SC)nc3-c3cccc(NC(=O)c4ccccc4CN4Cc5ccccc5C4=O)c3)ccn2)c1]

Covalent inhibitor 064 was synthesized via above shown methodology.

### tert-butyl(4-(2-(methylthio)-4-(3-nitrophenyl)-1-((2-(trimethylsilyl)ethoxy)methyl)-1H-imidazol-5-yl)pyridin-2-yl)carbamate (FW-313)

500 mg (1.12 mmol) FW-234, 432 mg (1.35 mmol, 1.2 eq.) tert-butyl (4-(4,4,5,5-tetramethyl-1,3,2-dioxaborolan-2-yl)pyridin-2-yl)carbamate (previously described procedure DOI:10.1021/acs.jmedchem.7b00316) and 740 mg (3.48 mmol, 3.1 eq.) K₃PO₄ were suspended in 10 ml 1,4-dioxane and 2.5 ml H₂O. The biphasic mixture was degassed several times. 32 mg (0.056 mmol, 0.05 eq.) P(tBu)₃ Pd G3 was added under an atmosphere of argon. The mixture was stirred at 50° C. overnight. The mixture was then cooled to ambient temperature, Brine was added and the aqueous phase was extracted with EtOAc. The combined organic layers were dried over Na₂SO₄, filtered and the volatiles evaporated. The crude product was purified via flash chromatography (SiO₂, Hex→Hex/EA 7:3) to give 500 mg of the pure product as a pale yellow solid in 79% (0.88 mmol) yield. ESI-MS: 580.7 [M+Na]⁺. ¹H NMR (200 MHz, DMSO) δ 10.01 (s, 1H), 8.38 (d, J=4.8 Hz, 1H), 8.27 (s, 1H), 8.06 (d, J=8.4 Hz, 1H), 7.89 (s, 1H), 7.78 (d, J=7.8 Hz, 1H), 7.57 (t, J=8.0 Hz, 1H), 7.08 (d, J=4.9 Hz, 1H), 5.13 (s, 2H), 3.46-3.38 (m, 2H), 2.70 (s, 3H), 1.41 (s, 9H), 0.85-0.75 (m, 2H), −0.08 (s, 9H). ¹³C NMR (50 MHz, DMSO) δ 153.29, 152.47, 148.66, 147.86, 145.69, 138.74, 135.82, 135.15, 132.13, 129.70, 129.18, 121.23, 120.52, 119.22, 113.32, 79.63, 72.70, 65.60, 27.87, 17.14, 15.38, −1.60.

### tert-butyl(4-(4-(3-aminophenyl)-2-(methylthio)-1-((2-(trimethylsilyl)ethoxy)methyl)-1H-imidazol-5-yl)pyridin-2-yl)carbamate (FW-314)

350 mg (0.62 mmol) FW-313 and 205 mg (3.13 mmol, 5.0 eq.) zinc dust were suspended in a mixture of 6 ml of Methanol and 6 ml of THF. Under vigorous stirring 395 mg (6.27 mmol, 10.0 eq.) of ammonium formate was added portion wise. After stirring for 1 h at room temperature, the reaction mixture was diluted with EtOAc and filtered over celite. Solvents of the filtrate were removed in vacuo and the residue was purified via flash column (SiO₂, DCM→DCM/MeOH 2%) Yield: 310 mg (0.58 mmol, 93%) as yellow solid. ESI-MS: 528.7 [M+H]⁺. ¹H NMR (200 MHz, DMSO) δ 9.92 (s, 1H), 8.34-8.25 (m, 1H), 7.85 (s, 1H), 6.99-6.78 (m, 3H), 6.60-6.27 (m, 2H), 5.26-4.80 (m, 4H), 3.44-3.36 (m, 2H), 2.65 (s, 3H), 1.43 (s, 9H), 0.87-0.75 (m, 2H), −0.08 (s, 9H). ¹³C NMR (50 MHz, DMSO) δ 152.98, 152.64, 148.55, 148.25, 144.35, 139.90, 139.12, 134.10, 128.53, 127.49, 119.89, 114.63, 113.59, 112.79, 112.59, 79.71, 72.62, 85.44, 27.99, 17.19, 15.63, −1.48.

### N-(3-(5-(2-aminopyridin-4-yl)-2-(methylthio)-1-((2-(trimethylsilyl)ethoxy)methyl)-1H-imidazol-4-yl)phenyl)-2-((1-oxoisoindolin-2-yl)methyl)benzamide (FW-316)

54 mg (0.20 mmol, 1.2 eq.) FW-259 was dissolved in 4 ml THF and 3 drops DMF were added. 29 μl (0.34 mmol, 2.0 eq.) oxalyl chloride was added drop wise under gas formation and the mixture was stirred for 1 h at 40° C., whereupon the excess of oxalyl chloride was removed in vacuo. 90 mg (0.17 mmol) FW-314 was dissolved in 2 ml THF, 98 μl (0.56 mmol, 3.3. eq.) triethylamine was added and the mixture was cooled down to 0 T. The beforehand prepared acid chloride solved in 2 ml THF was slowly added, whereupon the mixture was warmed to ambient temperature and further stirred for 0.5 h. Sat. aq. NH₄Cl was added and the aqueous layer was extracted with DCM. The combined organic layers were dried over Na₂SO₄ and the solvents were removed in vacuo. The residue was dissolved in a 10% TFA/DCM mixture and stirred for 4 h at ambient temperature. Quenching with sat. aq. NaHCO₃ and extraction with DCM gained a crude product, which was purified via flash chromatography (SiO₂; n-hexane->n-hexane/EtOAc/MeOH 20:75:5). Yield 43% (50 mg, 0.07 mmol) of a yellow solid. ¹H NMR (400 MHz, CDCl₃) δ 9.30 (s, 1H), 7.96 (d, J=4.9 Hz, 1H), 7.86 (s, 1H), 7.79 (d, J=7.4 Hz, 1H), 7.76-7.67 (m, 1H), 7.55-7.46 (m, 2H), 7.42 (t, J=8.2 Hz, 2H), 7.34-7.18 (m, 5H), 6.66 (d, J=4.9 Hz, 1H), 6.56 (s, 1H), 5.11 (s, 2H), 4.85 (s, 2H), 4.70 (s, 2H), 4.51 (s, 2H), 3.58-3.49 (m, 2H), 2.68 (s, 3H), 0.93-0.88 (m, 2H), −0.01 (s, 9H). ¹³C NMR (101 MHz, CDCl₃) δ 169.21, 167.64, 159.03, 148.41, 145.59, 141.81, 139.92, 139.29, 138.39, 136.63, 135.34, 134.64, 132.54, 131.80, 131.59, 130.71, 129.46, 129.07, 128.66, 128.08, 127.80, 127.74, 123.77, 122.94, 119.35, 119.24, 115.41, 110.16, 73.04, 66.63, 51.51, 43.89, 18.04, 16.41, −1.30.

### N-(3-(5-(2-((5-acrylamido-2-methoxyphenyl)amino)pyridin-4-yl)-2-(methylthio)-1H-imidazol-4-yl)phenyl)-2-((1-oxoisoindolin-2-yl)methyl)benzamide (064)

45 mg (0.06 mmol) FW-316, 20 mg (0.08 mmol, 1.2 eq.) N-(3-bromo-4-methoxyphenyl) acrylamide (previously described conditions doi.org/10.1002/anie.201603736) and 108 mg Cs₂CO₃ (0.33 mmol, 5.0 eq.) in 1.5 ml of a mixture of 1,4-dioxane/t-BuOH (4+1) was degassed three times by evacuating and backfilling with argon under stirring. Under an atmosphere of argon, 6 mg Brettphos Pd G3 (10 mol %, 0.007 mmol) was added. The solution was stirred under reflux for 5 h. After cooling down to room temperature, celite was added to the mixture and solvents were removed in vacuo. Purification via flash chromatography (SiO₂; n-hexane/EtOAc 2:8) yielded 25 mg (45%, 0.03 mmol) of a white solid [identification of intermediate via TLC-MS (ESI+): calcd. m/z 851.33. Found 874.9 [M+Na]⁺], which was then dissolved in a mixture of TFA/DCM 5% v/v and stirred overnight at ambient temperature. After quenching the reaction with saturated aqueous NaHCO₃ solution and extraction of the aqueous layer with EtOAc, the combined organic layers were dried over Na₂SO₄, filtered and the solvents removed in vacuo. Residue was again dissolved in 3 ml EtOAc and filtered via syringe filter. To give the product (with 91% purity detected via HPLC) as a yellow solid in 31% yield (15 mg, 0.02 mmol). ESI-MS: 744.3 [M+Na]⁺. As mixture of tautomers: ¹H NMR (600 MHz, DMSO) δ 12.86-12.47 (m, 1H), 10.68-10.43 (m, 1H), 9.97 (s, 1H), 8.38-8.23 (m, 1H), 8.09 (s, 1H), 7.96 (s, 1H), 7.87 (s, 1H), 7.79-7.67 (m, 2H), 7.58-7.54 (m, 2H), 7.54-7.51 (m, 1H), 7.48-7.44 (m, 2H), 7.42-7.39 (m, 2H), 7.36-7.22 (m, 3H), 7.19-7.11 (m, 1H), 6.91 (d, J=8.5 Hz, 1H), 6.74 (s, 1H), 6.54-6.35 (m, 1H), 6.27-6.14 (m, 1H), 5.77-5.63 (m, 1H), 4.88 (s, 2H), 4.38 (s, 2H), 3.76 (s, 3H), 2.63 (s, 3H).

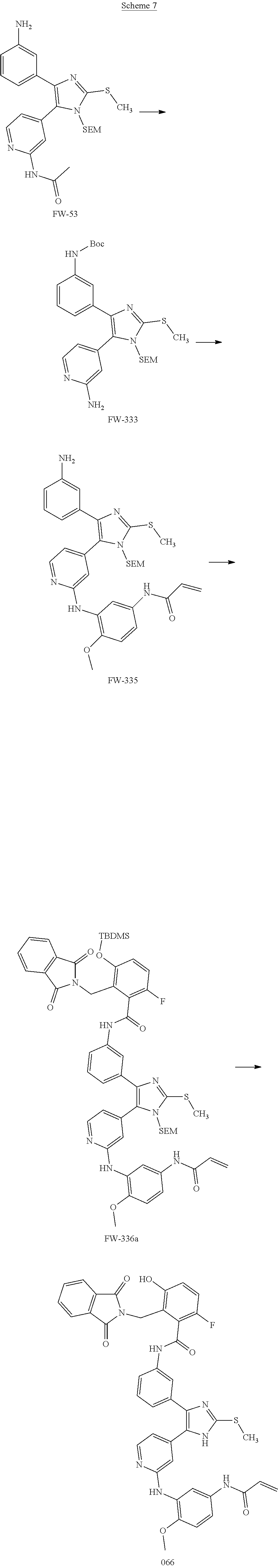

Covalent Inhibitor 66 was synthesized via above shown scheme.

### tert-Butyl(3-(5-(2-aminopyridin-4-yl)-2-(methylthio)-1-((2-(trimethylsilyl)ethoxy)methyl)-1H-imidazol-4-yl)phenyl)carbamate (FW-333)

1.2 g (2.55 mmol) FW-53 was suspended in t-BuOH and 585 mg of di-tert-butyl dicarbonate was added in one portion. The reaction mixture was stirred overnight at 60° C. After complete conversion [identification of intermediate via TLC-MS (ESI+): calcd. m/z 569.25 for C₂₈H₃₉N₅O₄SSi. Found 592.3 [M+Na]⁺] t-BuOH was evaporated and the residue was dissolved in 25 ml of MeOH and 5 ml of a 3 N NaOH solution was added. The mixture was stirred at 60° C. for 5 h, celite was added and the solvents were removed in vacuo. After purification via flash chromatography (SiO²; n-hexane/EtOAc 40:60) a white solid in 70% yield (950 mg, 1.79 mmol) was obtained. ¹H NMR (400 MHz, CDCl₃) δ 8.10-8.01 (m, 1H), 7.47-7.35 (m, 2H), 7.18-7.12 (m, 1H), 7.12-7.07 (m, 1H), 6.72-6.66 (m, 1H), 6.59 (s, 1H), 6.53 (s, 1H), 5.14 (s, 2H), 4.66 (s, 2H), 3.55 (t, J=7.9 Hz, 2H), 2.71 (s, 3H), 1.49 (s, 9H), 0.92 (t, J=7.9 Hz, 2H), −0.00 (s, 9H). ¹³C NMR (101 MHz, CDCl₃) δ 158.8, 152.9, 148.2, 145.6, 140.3, 139.6, 138.5, 134.6, 129.1, 128.5, 122.4, 117.8, 117.7, 115.7, 110.2, 80.6, 73.1, 66.7, 28.5, 18.1, 16.4, −1.3. TLC-MS (ESI+): calcd. m/z 527.24 for C₂₆H₃₇N₅O₃SSi. Found 550.3 [M+Na]⁺.

### N-(3-((4-(4-(3-Aminophenyl)-2-(methylthio)-1-((2-(trimethylsilyl)ethoxy)methyl)-1H-imidazol-5-yl)pyridin-2-yl)amino)-4-methoxyphenyl)acrylamide (FW-335)

600 mg (1.13 mmol) FW-333, 363 mg (1.42 mmol) N-(3-bromo-4-methoxyphenyl) acrylamide (previously described conditions doi.org/10.1002/anie.201603736) and 1.85 g Cs₂CO₃ (5.68 mmol) in 7.5 ml of a mixture of 1,4-dioxane/t-BuOH (4+1) was degassed three times by evacuating and backfilling with argon under stirring. Under an atmosphere of argon, 51 mg Brettphos Pd G3 (5 mol %, 0.05 mmol) was added. The solution was stirred under reflux for 4 h. After cooling down to room temperature, celite was added to the mixture and solvents were removed in vacuo. Purification via flash chromatography (SiO₂; n-hexane/EtOAc 45:55) yielded 500 mg (62%, 0.70 mmol) of a white solid [identification of intermediate via TLC-MS (ESI+): calcd. m/z 702.30 for C₃₈H₄₈N₆O₅SSi. Found 703.3 [M+H]⁺], which was then solved in a mixture of TFA/DCM 5% v/v and stirred overnight at room temperature. After quenching the reaction with saturated aqueous NaHCO₃ solution and extraction of the aqueous layer with EtOAc, the combined organic layers were dried over Na₂SO₄, filtered and the solvents removed in vacuo. The crude product was purified via flash chromatography (SiO₂; n-hexane/EtOAc 15:85) to give the pure product as a white solid in 41% yield (285 mg, 0.46 mmol) over two steps. ¹H NMR (600 MHz, DMSO-d₆) δ 9.97 (s, 1H), 8.39 (d, J=2.4 Hz, 1H), 8.21 (s, 1H), 8.19 (d, J=5.2 Hz, 1H), 7.43 (dd, J=8.8, 2.4 Hz, 1H), 7.02 (s, 1H), 6.95 (d, J=8.9 Hz, 1H), 6.90-6.88 (m, 1H), 6.87-6.84 (m, 1H), 6.69 (dd, J=5.2, 1.2 Hz, 1H), 6.48-6.43 (m, 2H), 6.40 (dd, J=7.9, 1.4 Hz, 1H), 6.22 (dd, J=17.0, 1.9 Hz, 1H), 5.70 (dd, J=10.2, 1.9 Hz, 1H), 5.13 (s, 2H), 5.00 (s, 2H), 3.79 (s, 3H), 3.39-3.35 (m, 2H), 2.65 (s, 3H), 0.78-0.74 (m, 2H), −0.09 (s, 9H). ¹³C NMR (151 MHz, DMSO-d₆) δ 162.7, 156.3, 148.6, 147.6, 145.4, 144.0, 139.2, 138.8, 134.3, 132.2, 131.9, 129.7, 128.5, 127.7, 126.1, 115.9, 114.6, 113.0, 112.7, 112.6, 112.4, 111.8, 110.8, 72.5, 65.6, 55.8, 17.2, 15.6, −1.5. TLC-MS (ESI+): calcd. m/z 602.25 for C₃₁H₃₈N₆O₃SSi. Found 625.5 [M+Na]⁺.

### N-(3-(5-(2-((5-acrylamido-2-methoxyphenyl)amino)pyridin-4-yl)-2-(methylthio)-1-((2-(trimethylsilyl)ethoxy)methyl)-1H-imidazol-4-yl)phenyl)-3-((tert-butyldimethylsilyl)oxy)-2-((1,3-dioxoisoindolin-2-yl)methyl)-6-fluorobenzamide (FW-336a)

138 mg (0.32 mmol) FW-283 was solved in 2 ml THF and 5 drops DMF were added. 32 μl (0.37 mmol) oxalyl chloride was added drop wise under gas formation and the mixture was stirred for 1 h at room temperature, whereupon the excess of oxalyl chloride was removed in vacuo. 130 mg (0.21 mmol) FW-335 was dissolved in 2 ml THF, 90 μl (0.64 mmol) triethylamine was added and the mixture was cooled down to 0°. The beforehand prepared acid chloride solved in 2 ml THF was slowly added, whereupon the mixture was warmed to room temperature and stirred for 1 h. Brine was added and the aqueous layer was extracted with EtOAc. The combined organic layers were dried over Na₂SO₄ and the solvents were removed in vacuo. Purification via flash chromatography (SiO₂; n-hexane/EtOAc 10:90) yielded 70% (154 mg, 0.15 mmol) of a white solid. ¹H NMR (400 MHz. CDCl₃) δ 9.55 (s, 1H), 8.07 (s, 1H), 7.80-7.75 (m, J=2.6 Hz, 2H), 7.73-7.68 (m, 3H), 7.67-7.63 (m, 1H), 7.57-7.53 (m, 1H), 7.53-7.49 (m, 1H), 7.40 (s, 1H), 7.38-7.32 (m, 1H), 7.04 (s, 1H), 6.97-6.69 (m, 6H), 6.46-6.39 (m, 2H), 5.81-5.70 (m, 1H), 5.10 (s, 2H), 4.69 (s, 2H), 3.78 (s, 3H), 3.52 (t, J=8.0 Hz, 2H), 2.69 (s, 3H), 0.90-0.85 (m, 2H), 0.81 (s, 9H), 0.20 (s, 6H), −0.04 (s, 9H). ¹³C NMR (101 MHz, CDCl₃) δ 168.3, 164.0, 163.6, 155.9, 154.5, 152.1, 150.8, 150.8, 146.8, 146.3, 139.9, 139.6, 136.8, 134.8, 134.1, 132.2, 131.8, 131.5, 129.6, 128.8, 127.7, 127.5, 126.9, 125.1, 123.4, 123.4, 122.8, 122.7, 120.7, 120.3, 120.2, 119.7, 116.4, 116.1, 115.6, 111.6, 111.2, 109.2, 73.1, 66.7, 56.0, 36.8, 26.1, 18.9, 18.0, 16.5, −1.3, −3.3. TLC-MS (ESI+): calcd. m/z 1013.38 for C₅₃H₆₀FN₇O₇SSi₂. Found 1037.0 [M+Na]⁺.

### N-(3-(5-(2-((5-acrylamido-2-methoxyphenyl)amino)pyridin-4-yl)-2-(methylthio)-1H-imidazol-4-yl)phenyl)-2-((1,3-dioxoisoindolin-2-yl)methyl)-6-fluoro-3-hydroxybenzamide (066)

50 mg (0.05 mmol) FW-336a was solved in 1.5 ml DCM and 0.5 ml TFA was added. The mixture was stirred at room temperature for 20 h [identification of intermediate via TLC-MS (ESI+): calcd. m/z 883.30 for C₄₇H₄₆FN₇O₆SSi. Found 906.3 [M+Na]⁺]. 4 ml 1,4-dioxane and 1 ml of an aqueous 50% v/v sulfuric acid were added until the complete conversion was indicated via HPLC. A saturated aqueous NaHCO₃ solution was added adjusting to pH 8. The emerging precipitate was filtered, washed with demineralized water and n-pentane and dried via high vacuum pump yielding 89% (34 mg, 0.045 mmol) of an off-white solid. ¹H NMR (400 MHz, DMSO-d₆) 12.92 (br s, 1H), 10.62 (s, 1H), 10.10 (s, 1H), 9.91 (s, 1H), 8.19-7.73 (m, 3H), 7.68-7.60 (m, 5H), 7.54-7.49 (m, 1H), 7.49-7.45 (m, 1H), 7.41-7.23 (m, 2H), 7.15-7.11 (m, 1H), 7.06 (t, J=8.8 Hz, 2H), 6.87 (dd, J=8.9, 4.8 Hz, 1H), 6.82 (s, 1H), 6.43 (dd, J=17.0, 10.1 Hz, 1H), 6.23 (dd, J=17.0, 1.8 Hz, 1H), 5.72 (dd, J=10.3, 1.5 Hz, 1H), 4.82 (s, 2H), 3.76 (s, 3H), 2.65 (s, 3H). HRMS (ESI): exact mass calcd for C₄₁H₃₂FN₇O₆S [M+H]⁺: 770.21916. Found: 770.21941.

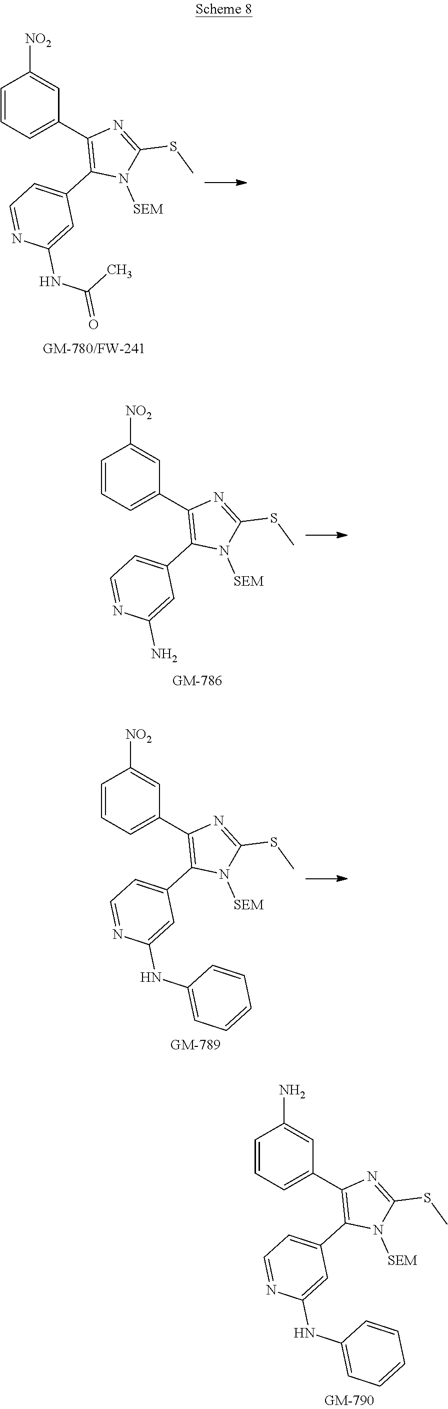 [SMILES: CSc1nc(-c2cccc([N+](=O)[O-])c2)c(-c2ccnc(NC(C)=O)c2)n1COCC[Si](C)(C)C ; CSc1nc(-c2cccc([N+](=O)[O-])c2)c(-c2ccnc(N)c2)n1COCC[Si](C)(C)C ; CSc1nc(-c2cccc([N+](=O)[O-])c2)c(-c2ccnc(Nc3ccccc3)c2)n1COCC[Si](C)(C)C ; CSc1nc(-c2cccc(N)c2)c(-c2ccnc(Nc3ccccc3)c2)n1COCC[Si](C)(C)C]

### 4-(2-(methylthio)-4-(3-nitrophenyl)-1-((2-(trimethylsilyl)ethoxy)methyl)-1H-imidazol-5-yl)pyridin-2-amine (GM-786)

GM-780 (FW-241) (500 mg, 1.0 mmol) was dissolved in 15 ml MeOH. 5 ml of a 5 M aqueous NaOH solution was added under vigorous stirring. The mixture was stirred at 50° C. for 3 hours. Then cooled to ambient temperature and quenched with a saturated aqueous NH₄Cl solution. The aqueous mixture was extracted three times with EtOAc. The combined organics were dried over Na₂SO₄, filtered and the volatiles removed by rotary evaporation. The crude mixture was purified by flash chromatography (SiO₂, Hex→Hex/EtOAc 1:2) to give 420 mg of the pure product as a pale yellow solid in 92% yield. ¹H NMR (200 MHz, Chloroform-d) δ 8.59-8.42 (m, 1H), 8.26-8.12 (m, 1H), 8.10-7.98 (m, 1H), 7.74 (dt, J=7.8, 1.3 Hz, 1H), 7.39 (t, J=8.0 Hz, 1H), 6.81-6.63 (m, 1H), 6.63-6.49 (m, 1H), 5.17 (s, 2H), 4.72 (s, 2H), 3.75-3.48 (m, 2H), 2.79 (s, 3H), 1.06-0.86 (m, 2H), 0.04 (s, 9H).

### 4-(2-(methylthio)-4-(3-nitrophenyl)-1-((2-(trimethylsilyl)ethoxy)methyl)-1H-imidazol-5-yl)-N-phenylpyridin-2-amine (GM-789)

409 mg (0.89 mmol) GM-786, 182 mg (1.16 mmol, 1.3 eq.) bromobenzol, 1450 mg (4.45 mmol, 5.0 eq.) caesium carbonate were suspended in a mixture of 24 ml 1,4-dioxane and 6 ml t-BuOH and the flask was degassed with three cycles of evacuation and backfilling with argon. 41 mg (5 mol %) Brettphos Pd G3 was added and another three cycles of evacuation and argon backfilling were carried out. Refluxing the mixture for 2 h led to complete conversion of the starting material. Mixture was cooled down to ambient temperature and diluted with EtOAc, filtered and volatiles were evaporated in vacuo. The crude product was purified via flash chromatography (SiO₂, Hex→Hex/EtOAc 2:1). Yield: 422 mg (89%) of a pale yellow solid. The product was used in the next step without further characterization.

### 4-(4-(3-aminophenyl)-2-(methylthio)-1-((2-(trimethylsilyl)ethoxy)methyl)-1H-imidazol-5-yl)-N-phenylpyridin-2-amine (GM-790)

GM-789 (420 mg, 0.79 mmol, 1.0 eq.) and zinc powder (258 mg, 3.94 mmd, 5 eq.) was suspended in ca. 8 ml EtOH. Ammonium formate (248 mg, 3.94 mmol, 5 eq.) was added in one portion under vigorous stirring. The mixture was stirred at 50° C. until complete consumption of the starting material. The solvent was removed by rotary evaporation and the residue taken up in EtOAc filtered over celite. The organic layer was washed with a saturated NH₄Cl solution and subjected to flash chromatography (SiO₂, EtOAc) to give 206 mg of the pure product as a yellow solid in 52% yield. (¹H NMR (200 MHz, Chloroform-d) δ 8.25 (d, J=5.3 Hz, 1H), 8.07 (s, 1H), 7.34-7.15 (m, 4H), 7.15-6.97 (m, 4H), 6.87 (d, J=6.3 Hz, 2H), 6.73-6.52 (m, 1H), 5.19 (s, 2H), 3.89 (s, 2H), 3.58 (t, J=8.4 Hz, 2H), 2.74 (s, 3H), 0.92 (t, J=8.4 Hz, 2H), 0.04 (s, 9H). ¹³C NMR (50 MHz, CDCl₃) δ 156.57, 148.59, 146.58, 145.26, 140.32, 140.15, 140.03, 134.68, 129.20, 128.40, 122.79, 120.24, 118.07, 115.87, 114.29, 113.96, 109.54, 72.88, 66.38, 17.89, 16.39, −1.39, −1.43.

### 2,6-difluoro-N-(3-(2-(methylthio)-5-(2-(phenylamino)pyridin-4-yl)-1-((2-(trimethylsilyl)ethoxy)methyl)-1H-imidazol-4-yl)phenyl)benzamide (GM-793)

GM-790 (50 mg, 0.10 mmol, 1.0 eq.) 2,6-difluorobenzoic acid (19 mg, 0.12 mmol, 1.2 eq.), PyBOP (62 mg, 0.12 mmol, 1.2 eq.) and DIPEA (26 μl, 0.15 mmol, 1.5 eq.) was dissolved in 4 ml DMF. The mixture was stirred at ambient temperature overnight. After complete consumption of the starting material, Ether was added and the organic layer washed three times with water. The organic layer was separated, dried over Na₂SO₄, filtered and all volatiles were removed by rotary evaporation. The crude mixture was purified by flash chromatography (SiO₂, Hex→Hex/EA 1:1) to give 45 mg of the pure product as a pale yellow solid in 70% yield. ¹H NMR (200 MHz, Chloroform-d) δ 8.46 (s, 1H), 8.19 (d, J=5.2 Hz, 1H), 7.95 (d, J=7.8 Hz, 1H), 7.70 (d, J=2.3 Hz, 1H), 7.38-7.11 (m, 8H), 7.02 (t, J=7.2 Hz, 1H), 6.93 (d, J=7.2 Hz, 2H), 6.88-6.81 (m, 2H), 5.15 (s, 2H), 3.65-3.53 (m, 2H), 2.72 (s, 3H), 0.97-0.87 (m, 2H), 0.03 (s, 9H). ESI-MS: 643.7 [M+H]⁺.

### 2,6-difluoro-N-(3-(2-(methylthio)-5-(2-(phenylamino)pyridin-4-yl)-1H-imidazol-4-yl)phenyl)benzamide (010)

The title compound was prepared from GM-793 (45 mg, 0.070 mmol) according to general procedure 2B. The crude product was purified by flash chromatography (SiO₂, DCM→DCM/MeOH 10%). 29 mg (0,056 mmol) of the pure were obtained as a white solid in 81% yield. ¹H NMR (200 MHz, DMSO-d₆) δ 12.76 (s, 1H), 10.95 (s, 1H), 9.11 (s, 1H), 8.05 (d, J=5.4 Hz, 1H), 7.89-7.72 (m, 2H), 7.73-7.42 (m, 4H), 7.34-7.20 (m, 5H), 7.12-6.99 (m, 1H), 6.98-6.75 (m, 3H), 2.65 (s, 3H). ¹³C NMR (50 MHz, DMSO) δ 161.70, 161.54, 158.66, 156.76, 156.16, 141.63, 139.25, 132.54, 129.01, 121.20, 119.45, 118.88, 115.75, 112.69, 112.23, 107.56, 15.40.

### 2-fluoro-S-hydroxy-N-(3-(2-(methylthio)-5-(2-(phenylamino)pyridin-4-yl)-1H-imidazol-4-yl)phenyl)benzamide (061)

FW-265 (22 mg, 0.11 mmol, 1.1 eq.) was dissolved in dry THF (3 ml). Oxalylchloride (9.5 μl, 0.11 mmol, 1.1 eq.) and one drop of DMF was added and the mixture stirred for 2 hours at ambient temperature. GM-790 (50 mg, 0.10 mmol, 1.0 eq.) and DIPEA (35 μl, 0.2 mmol, 2 eq.) was dissolved in 2 ml dry THF and added to the carboxylic acid chloride. The mixture was stirred for 2 hours at ambient temperature and then quenched with saturated NH₄Cl solution and extracted with DCM. The organic layer was separated, dried over Na₂SO₄, filtered and evaporated. The residue was dissolved in 2.5 M HCl in EtOH and stirred overnight at ambient temperature. After complete consumption of the intermediate, the mixture was diluted with NaHCO₃ solution and extracted with EtOAc. The organic layer was separated, dried over Na₂SO₄, filtered and evaporated. The crude product was purified by flash chromatography (SiO₂, DCM→DCM/MeOH 2.5%) to give 40 mg of the pure product as a yellow solid in 78% yield over two steps. ¹H NMR (200 MHz, DMSO-d₆) δ 12.63 (s, 1H), 10.50 (s, 1H), 9.73 (s, 1H), 9.00 (s, 1H), 7.95 (d, J=28.2 Hz, 3H), 7.56 (d, J=7.9 Hz, 3H), 7.31-7.04 (m, 4H), 7.04-6.55 (m, 4H), 2.64 (s, 3H).

### 6-fluoro-3-hydroxy-N-(3-(2-(methylthio)-5-(2-(phenylamino)pyridin-4-yl)-1H-Imidazol-4-yl)phenyl)-2-((1-oxoisoindolin-2-yl)methyl)benzamide (062)

FW-291 (50 mg, 0.12 mmol, 1.2 eq.) was dissolved in dry THF (3 ml). Oxalylchloride (10.3 μl, 0.12 mmol, 1.2 eq.) and one drop of DMF was added and the mixture stirred for 2 hours at ambient temperature. GM-790 (50 mg, 0.10 mmol, 1.0 eq.) and DIPEA (35 μl, 0.2 mmol, 2 eq.) was dissolved in 2 ml dry THF and added to the carboxylic acid chloride. The mixture was stirred for 2 hours at ambient temperature and then quenched with saturated NH₄Cl solution and extracted with DCM. The organic layer was separated, dried over Na₂SO₄, filtered and evaporated. The residue was dissolved in 2.5 M HCl in EtOH and stirred overnight at ambient temperature. After complete consumption of the intermediate, the mixture was diluted with NaHCO₃ solution and extracted with EtOAc. The organic layer was separated, dried over Na₂SO₄, filtered and evaporated. The crude product was purified by flash chromatography (SiO₂, DCM→DCM/MeOH 2.5%) to give 30 mg of the pure product as a yellow solid in 46% yield over two steps. ¹H NMR (400 MHz, DMSO-d₆) δ 12.69 (d, J=4.5 Hz, 1H), 10.58 (d, J=41.3 Hz, 1H), 10.01 (s, 1H), 8.99 (d, J=22.5 Hz, 1H), 8.17-7.71 (m, 3H), 7.59-7.50 (m, 3H), 7.49-7.38 (m, 2H), 7.33-7.02 (m, 7H), 6.98-6.91 (m, 1H), 6.88 (s, 2H), 4.82 (s, 2H), 4.22 (s, 2H), 2.63 (s, 3H). ¹³C NMR (101 MHz, DMSO) δ 167.77, 165.40, 156.63, 152.93, 149.02, 147.63, 143.37, 142.52, 142.32, 139.94, 136.76, 135.62, 135.08, 131.02, 129.78, 129.63, 128.92, 128.65, 128.12, 127.75, 124.37, 120.21, 118.37, 116.17, 112.69, 107.57, 48.03, 43.51, 15.57. ESI-MS: 679.7 [M+Na]⁺.

### 2-((1,3-dioxoisoindolin-2-yl)methyl)-N-(3-(2-(methylthio)-5-(2-(phenylamino)pyridin-4-yl)-1H-imidazol-4-yl)phenyl)benzamide (067)

FW-256 (34 mg, 0.12 mmol, 1.2 eq.) was dissolved in dry THF (3 ml). Oxalylchloride (10.3 μl, 0.12 mmol, 1.2 eq.) and one drop of DMF was added and the mixture stirred for 2 hours at ambient temperature. GM-790 (50 mg, 0.10 mmol, 1.0 eq.) and DIPEA (35 μl, 0.2 mmol, 2 eq.) was dissolved in 2 ml dry THF and added to the carboxylic acid chloride. The mixture was stirred for 2 hours at ambient temperature and then quenched with saturated NH₄Cl solution and extracted with DCM. The organic layer was separated, dried over Na₂SO₄, filtered and evaporated. The residue was dissolved in 2.5 M HCl in EtOH and stirred overnight at ambient temperature. After complete consumption of the intermediate, the mixture was diluted with NaHCO₃ solution and extracted with EtOAc. The organic layer was separated, dried over Na₂SO₄, filtered and evaporated. The crude product was purified by flash chromatography (SiO₂, DCM→DCM/MeOH 5%) to give 30 mg of the pure product as a yellow solid in 48% yield over two steps. ¹H NMR (400 MHz, DMSO-d₆) 10.71 (s, 1H), 10.50 (s, 1H), 7.98 (d, J=6.8 Hz, 1H), 7.95 (s, 1H), 7.89-7.78 (m, 5H), 7.57 (d, J=7.1 Hz, 1H), 7.45 (dt, J=17.4, 8.1 Hz, 3H), 7.35 (t, J=7.6 Hz, 2H), 7.29-7.11 (m, 7H), 4.96 (s, 2H), 2.66 (s, 3H). ¹³C NMR (101 MHz, DMSO) δ 168.20, 167.72, 151.65, 148.06, 145.79, 144.82, 140.17, 138.11, 136.87, 135.76, 135.04, 135.00, 132.05, 131.02, 130.93, 130.32, 130.31, 130.16, 130.03, 128.23, 127.71, 127.44, 126.34, 125.53, 124.44, 123.66, 123.28, 120.95, 120.28, 120.27, 112.23, 107.12, 15.35.

### 2-((1,3-dioxoisoindolin-2-yl)methyl)-6-fluoro-3-hydroxy-N-(3-(2-(methylthio)-5-(2-(phenylamino)pyridin-4-yl)-1H-imidazol-4-yl)phenyl)benzamide (068)

FW-281 (39 mg, 0.11 mmol, 1.1 eq.), GM-790 (50 mg, 0.10 mmol, 1.0 eq.), TBTU (35 mg, 0.11 mmol, 1.1 eq.) and DIPEA (35 μl, 0.2 mmol, 2 eq.) was dissolved in dry DMF (3 ml), and the mixture stirred overnight at 50° C. and then quenched with saturated NH₄Cl solution and extracted with DCM. The organic layer was separated, dried over Na₂SO₄, filtered and evaporated. The residue was dissolved in 2.5 M HCl in EtOH and stirred overnight at ambient temperature. After complete consumption of the intermediate, the mixture was diluted with NaHCO₃ solution and extracted with EtOAc. The organic layer was separated, dried over Na₂SO₄, filtered and evaporated. The crude product was purified by flash chromatography (SiO₂, DCM→DCM/MeOH 5%) to give 28 mg of the pure product as a yellow solid in 42% yield over two steps. ¹H NMR (400 MHz, DMSO) δ 12.65 (s, 1H), 10.76-10.41 (m, 1H), 10.00-9.77 (m, 1H), 8.97 (s, 1H), 8.17-7.95 (m, 1H), 7.71-7.63 (m, 4H), 7.60-7.44 (m, 4H), 7.33-7.15 (m, 4H), 7.14-7.01 (m, 2H), 6.91-6.68 (m, 3H), 4.82 (s, 2H), 2.64 (s, 3H).

### N-(3-(2-(methylthio)-5-(2-(phenylamino)pyridin-4-yl)-1H-imidazol-4-yl)phenyl)-2-((1-oxoisoindolin-2-yl)methyl)benzamide (069)

FW-259 (29 mg, 0.11 mmol, 1.1 eq.), GM-790 (50 mg, 0.10 mmol, 1.0 eq.), TBTU (35 mg, 0.11 mmol, 1.1 eq.) and DIPEA (35 μl, 0.2 mmol, 2 eq.) was dissolved in dry DMF (3 ml), and the mixture stirred overnight at 50° C. and then quenched with saturated NH₄Cl solution and extracted with DCM. The organic layer was separated, dried over Na₂SO₄, filtered and evaporated. The residue was dissolved in 2.5 M HCl in EtOH and stirred overnight at ambient temperature. After complete consumption of the intermediate, the mixture was diluted with NaHCO₃ solution and extracted with EtOAc. The organic layer was separated, dried over Na₂SO₄, filtered and evaporated. The crude product was purified by flash chromatography (SiO₂, DCM→DCM/MeOH 5%) to give 41 mg of the pure product as a yellow solid in 66% yield over two steps. ¹H NMR (400 MHz, DMSO-d₆) δ 12.72 (s, 1H), 10.59 (d, J=37.0 Hz, 1H), 8.96 (d, J=21.6 Hz, 1H), 8.20-7.93 (m, 1H), 7.86 (s, 1H), 7.78 (d, J=8.2 Hz, 1H), 7.71-7.65 (m, 1H), 7.61-7.35 (m, 9H), 7.31-7.13 (m, 4H), 7.11 (s, 1H), 6.91-6.75 (m, 2H), 4.86 (s, 2H), 4.35 (s, 2H), 2.62 (s, 3H). ¹³C NMR (101 MHz, DMSO) δ 168.24, 156.58, 147.68, 146.17, 143.34, 142.32, 139.28, 135.69, 135.04, 132.30, 131.91, 130.79, 129.65, 128.95, 128.68, 128.32, 128.09, 127.78, 127.78, 123.88, 123.33, 118.41, 112.82, 107.45, 50.27, 43.59, 15.59. ESI-MS: 621.5 [M−H]⁻.

### 3-(2-(methylthio)-1-((2-(trimethylsilyl)ethoxy)methyl)-1H-imidazol-4-yl)aniline (GM-928)

340 mg (0.93 mmol) of FW-230, 365 mg (5.58 mmol, 6.0 eq.) of zinc powder were suspended in 20 ml of EtOH and 352 mg (5.58 mmol, 6.0 eq.) ammonium formate was added in one portion under vigorous stirring. The mixture was warmed up until complete conversion, volatiles were evaporated and the residue diluted in EtOAc and filtered over celite. The organic layer was washed with sat. aq. NH₄Cl solution, dried over Na₂SO₄, filtered and volatiles removed in vacuo. Purification via flash chromatography (SiO₂, n-hex->EtOAc) yielded 209 mg (67%) of a colorless oil. The product was used directly without further characterization.

### 6-fluoro-3-hydroxy-N-(3-(2-(methylthio)-1H-imidazol-4-yl)phenyl)-2-((1-oxoisoindolin-2-yl)methyl)benzamide (GM-933/1058)

FW-291 (50 mg, 0.120 mmol, 1.0 eq.) was dissolved in 5 ml dry THF and thionylchloride (9 μl, 0.120, 1.0 eq.) was added followed by one drop of DMF. The mixture was stirred for one hour at ambient temperature. GM-928 (40 mg, 0.120 mmol, 1.0 eq.) and triethylamine (33 μl, 0.240 mmol, 2.0 eq.) were dissolved in 5 ml THF and added dropwise to the carboxylic acid chloride in solution. The mixture was stirred at ambient temperature overnight and finally quenched with a saturated NH₄Cl solution and extracted with EtOAc. The organic layer was separated, dried over Na2SO4, filtered and evaporated to dryness. The residue was taken up in 2.5 M HCl in EtOH and stirred at ambient temperature overnight. After complete consumption, the mixture was neutralized with saturated NaHCO₃ solution and extracted with EtOAc. The organic layer was separated, dried over Na₂SO₄, filtered and evaporated. The crude product was purified by flash chromatography (SiO₂, DCM→DCM/MeOH 10%) to give 30 mg of the pure product as white solid in 51% yield over two steps. ¹H NMR (400 MHz, DMSO-d₆) δ 10.61 (s, 1H), 10.23 (s, 1H), 8.18 (d, J=2.0 Hz, 1H), 7.85 (s, 1H), 7.65-7.55 (m, 3H), 7.54-7.37 (m, 3H), 7.32-7.27 (m, 1H), 7.09-6.98 (m, 2H), 4.88 (s, 2H), 4.27 (s, 2H), 2.75 (s, 3H). ESI-MS: 489.7 [M+H]⁺.

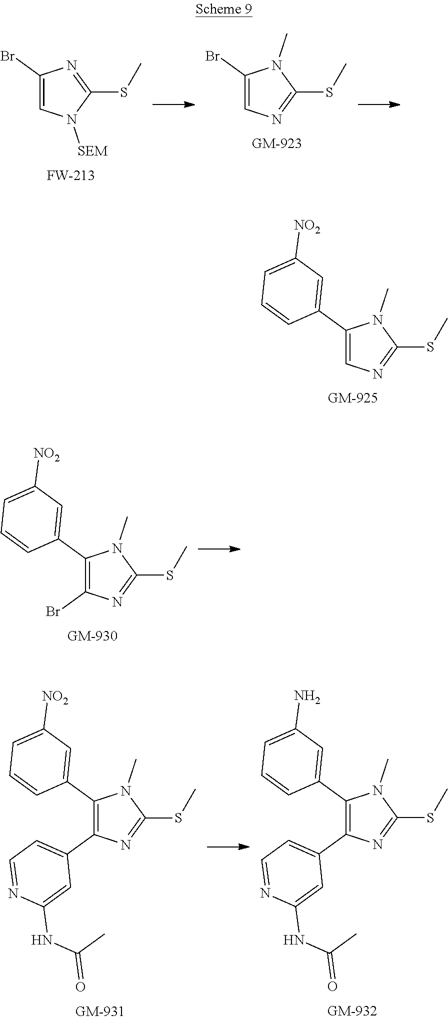

GM-932 was synthesized according above shown methodology. Methylation of the imidazole core ensued according to previously described conditions in combination with the already mentioned procedures to arrange the imidazole scaffold.

### 5-bromo-1-methyl-2-(methylthio)-1H-imidazole (GM-923)

700 mg (2.17 mmol) FW-213 was dissolved in 15 ml of dry toluol. The solution was cooled down to 0° C. and 427 mg (2.60 mmol, 1.2 eq.) methyl trifluoromethanesulfonate was added dropwise. Mixture was warmed up to ambient temperature and stirred for 2 h. Volatiles were evaporated in vacuo and the residue was diluted in 3 ml of TFA+1 drop of H₂O. The mixture was stirred for 1 h at ambient temperature. After complete conversion sat aq. NaHCO₃ was added and the aqueous phase was extracted with EtOAc. Combined organic layers were dried over Na₂SO₄, filtered and solvents removed in vacuo. Purification via flash chromatography (SiO₂, n-hex→n-hex/EtOAc 1:1) ¹H NMR (200 MHz, CDCl₃) δ 7.02 (s, 1H), 3.53 (s, 3H), 2.57 (s, 3H). ¹³C NMR (50 MHz, CDCl₃) δ 143.79, 129.35, 104.17, 32.09, 16.17.

### 1-methyl-2-(methylthio)-5-(3-nitrophenyl)-1H-imidazole (GM-925)

200 mg (0.97 mmol) GM-923, 177 mg (1.07 mmol, 1.1 eq.) 3-nitrophenylboronic acid and 615 mg (2.90 mmol, 3.0 eq.) of K₃PO₄ were dissolved in 8 ml 1,4-dioxane and 2 ml demineralized water. The solution was degassed with three cycles of evacuation and backfilling with argon. 28 mg (5 mol %) P(t-Bu)₃ Pd G3 was added to the solution and another three cycles of evacuation and argon backfilling were carried out. The reaction mixture was warmed up to 50° C. and stirred overnight. After cooling to room temperature, the mixture was diluted with DCM, washed once with brine, dried over Na₂SO₄, filtered and evaporated to dryness. The crude product was purified via flash chromatography (SiO₂; DCM->DCM/MeOH 5%) obtaining a yellow solid in 58% yield (140 mg). The product was used directly without further characterization.

### 4-bromo-1-methyl-2-(methylthio)-5-(3-nitrophenyl)-1H-imidazole (GM-930)

276 mg (1.11 mmol, 1.0 eq.) GM-925 was dissolved in 10 ml MeCN and the solution was cooled to −30° C. 200 mg (1.11 mmol, 1.0 eq.) NBS dissolved in 5 ml MeCN was added dropwise and the mixture was stirred at −30° C. for 1 hour. The volatiles were removed by rotary evaporation, the residue was taken up in DCM and washed with water. The separated organic layer was dried over Na₂SO₄, filtered and evaporated. The crude product was purified by flash chromatography (SiO₂, Hex→Hex/EA 2:1) to give 270 mg of the pure product as a pale yellow solid in 69% yield. ¹H NMR (200 MHz, CDCl₃) δ 8.32-8.18 (m, 2H), 7.79-7.60 (m, 2H), 3.51 (s, 1H), 2.67 (s, 2H). ¹³C NMR (50 MHz, CDCl₃) δ 148.52, 145.57, 135.80, 130.23, 130.00, 129.46, 124.55, 123.38, 115.35, 32.75, 15.81.

### N-(4-(1-ethyl-2-(methylthio)-5-(3-nitrophenyl)-1H-Imidazol-4-yl)pyridin-2-yl)acetamide (GM-931)

270 mg (0.83 mmol, 1.0 eq.) GM-930, 280 mg (1.07 mmol, 1.3 eq.) GM-449 was dissolved in 5 ml degassed 1,4-dioxane. An aqueous solution of K₃PO₄ (1.5 M, 5.0 mmol, 3 eq.) was added ad the biphasic mixture was degassed several times. 24 mg (0.04 mmol, 0.05 eq.) P(tBu)3 Pd G3 was added under an atmosphere of argon. The mixture was stirred at 45° C. for 3 hours and an additional hour at 60° C. After complete consumption of the starting material, the mixture was cooled to ambient temperature, diluted with EtOAc, filtered over Celite and the organic layer was washed with water. The separated organics were dried over Na₂SO₄, filtered and the volatiles evaporated. The crude product was purified by flash chromatography (SiO₂, Hex→Hex/EA 1:2) to give 200 mg of the pure product as a pale yellow solid in 63% yield. ¹H NMR (200 MHz, CDCl₃) δ 9.37 (s, 1H), 8.41-7.92 (m, 4H), 7.79-7.52 (m, 2H), 7.24-7.09 (m, 1H), 3.32 (s, 3H), 2.65 (s, 3H), 1.97 (s, 3H). ¹³C NMR (50 MHz, CDCl₃) δ 168.72, 152.04, 148.81, 147.40, 145.57, 143.81, 136.67, 136.51, 131.54, 130.58, 130.51, 125.48, 124.10, 117.02, 110.88, 31.58, 24.54, 15.64.

### N-(4-(5-(3-aminophenyl)-1-methyl-2-(methylthio)-1H-imidazol-4-yl)pyridin-2-yl)acetamide (GM-932)

200 mg (0.52 mmol) of GM-931, 204 mg (3.13 mmol, 6.0 eq.) of zinc powder were suspended in 20 ml of EtOH and 196 mg (3.13 mmol, 6.0 eq.) ammonium formate was added in one portion under vigorous stirring. The mixture was warmed up to 50° C. until complete conversion, volatiles were evaporated and the residue diluted in EtOAc and filtered over celite. The organic layer was washed with sat. aq. NH₄Cl solution, dried over Na₂SO₄, filtered and volatiles removed in vacuo. Purification via flash chromatography (SiO₂, n-hex->EtOAc) yielded 209 mg (67%) of a colorless oil. The product was used directly without further characterization.

### N-(3-(4-(2-acetamidopyridin-4-yl)-1-methyl-2-(methylthio)-1H-imidazol-5-yl)phenyl)-6-fluoro-3-hydroxy-2-((1-oxoisoindolin-2-yl)methyl)benzamide (059)

FW-291 (50 mg, 0.120 mmol, 1.0 eq.) was dissolved in 5 ml dry THF and thionylchloride (9 μl, 0.120 mmol, 1.0 eq.) was added followed by one drop of DMF. The mixture was stirred for one hour at ambient temperature. GM-932 (42 mg, 0.120 mmol, 1.0 eq.) and triethylamine (33 μl, 0.240 mmol, 2.0 eq.) were dissolved in 5 ml THF and added dropwise to the carboxylic acid chloride in solution. The mixture was stirred at ambient temperature overnight and finally quenched with a saturated NH₄Cl solution and extracted with EtOAc. The organic layer was separated, dried over Na2SO4, filtered and evaporated to dryness. The residue was taken up in 2.5 M HCl in EtOH and stirred at ambient temperature overnight. After complete consumption, the mixture was neutralized with saturated NaHCO3 solution and extracted with EtOAc. The organic layer was separated, dried over Na2SO4, filtered and evaporated. The crude product was purified by flash chromatography (SiO₂, DCM→DCM/MeOH 10%) to give 32 mg of the pure product as white solid in 42% yield over two steps. ¹H NMR (400 MHz, DMSO-d₆) δ 10.62 (s, 1H), 10.29 (s, 1H), 9.96 (s, 1H), 8.38 (s, 1H), 8.05 (d, J=5.3 Hz, 1H), 7.90-7.83 (m, 1H), 7.77 (t, J=1.9 Hz, 1H), 7.60 (dd, J=7.6, 1.6 Hz, 1H), 7.54-7.39 (m, 3H), 7.31 (dd, J=7.7, 1.4 Hz, 1H), 7.14 (dt, J=7.7, 1.3 Hz, 1H), 7.04 (t, J=9.0 Hz, 1H), 6.98-6.88 (m, 2H), 4.86 (s, 2H), 4.25 (s, 2H), 3.37 (s, 3H), 2.67 (s, 3H), 2.03 (s, 3H). ¹³C NMR (101 MHz, DMSO) δ 169.30, 167.77, 152.91, 149.02, 147.89, 143.85, 143.71, 140.20, 136.84, 135.64, 135.05, 133.38, 130.78, 130.37, 130.02, 129.78, 128.89, 128.18, 127.79, 126.08, 120.99, 119.54, 116.26, 47.98, 43.65, 31.82, 24.31, 15.87. ESI-MS: not detected.

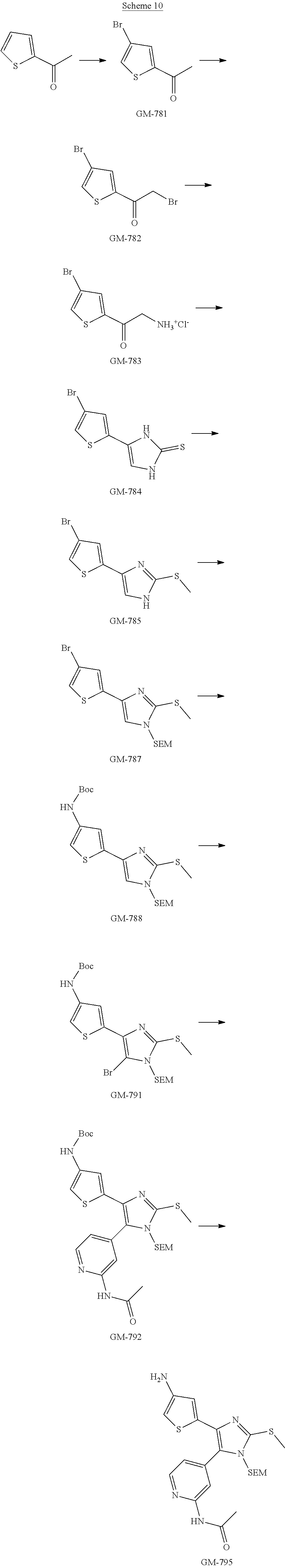

### 1-(4-Bromothiophen-2-yl)ethan-1-one (GM-781)

26.4 g (189.1 mmol, 2.5 eq.) aluminum chloride was suspended under an atmosphere of argon in 5 ml dry DCM. Under vigorous stirring, 10.0 g (79.26 mmol, 1.0 eq.) 2-acetyl thiophene was added. After the evolution gas stopped, Bromine (4.3 ml, 83.2 mmol, 1.05 eq.) dissolved in 680 ml dry DCM was carefully added dropwise. The mixture was stirred at ambient temperature overnight. After complete consumption of the starting material, the mixture was poured on ice water, quenched with sodium thiosulfate. The organic layer was separated, filtered over Na₂SO₄ and the solvents removed by rotary evaporation. The crude mixture was purified by distillation (148° C. at 15 mbar) to give 11.97 g of the product as a colorless oil in 74% yield. ¹H NMR (200 MHz, Chloroform-d) δ 87.55 (d, J=1.4 Hz, 1H), 7.51 (d, J=1.4 Hz, 1H), 2.52 (s, 3H). ¹³C NMR (50 MHz, CDCl₃) δ 189.47, 144.68, 134.38, 131.00, 110.54, 26.56.

### 2-Bromo-1-(4-bromothiophen-2-yl)ethan-1-one (GM-782)

11.9 g (58 mmol, 1.0 eq.) GM-781 was dissolved in 100 ml chloroform. Bromine (3.12 ml, 61 mmol, 1.05 eq.) was dissolved in 100 ml chloroform and added dropwise over 1 hour to well stirred solution at ambient temperature. After complete consumption of the starting material, the mixture was poured on water and quenched with a saturated aqueous sodium thiosulfate solution. The organic layer was separated, dried over Na₂SO₄, filtered and the solvents evaporated to give 16.3 g of the product as an orange oil in 99% yield. ¹H NMR (200 MHz, Chloroform-d) δ 7.77-7.71 (m, 1H), 7.66-7.61 (m, 1H), 4.38-4.34 (m, 2H). ¹³C NMR (50 MHz, CDCl₃) δ 183.46, 140.98, 135.41, 132.39, 111.09, 30.12.

### 2-(4-Bromothiophen-2-yl)-2-oxoethan-1-aminium chloride (GM-783)

16.3 g (57.4 mmol, 1.0 eq.) GM-782 was dissolved in 120 ml chloroform. Urotropine (8.05 g, 57.4 mmol, 1.0 eq.) was added portionwise and the suspension was stirred for 3 hours at 70° C. After cooling to ambient temperature, the solids were collected by filtration and washed with chloroform. The dry solid was resuspended in 90 ml EtOH and treated with 45 ml concentrated hydrochloric acid. The suspension was stirred for 4 hours at ambient temperature. The mixture was cooled to 0° C., filtered and the solids washed with EtOH. The solids were recrystallized with water and washed with EtOH and diethylether to give 4.5 g of the product as a pale pink solid in 31% yield. ¹H NMR (200 MHz, DMSO-d₆) δ 8.26 (dd, J=10.7, 1.4 Hz, 2H), 8.61 (s, 3H), 4.53 (s, 2H). ¹³C NMR (50 MHz, DMSO) δ 185.79, 140.84, 137.03, 134.09, 110.77, 44.89.

### 4-(4-Bromothiophen-2-yl)-1,3-dihydro-2H-imidazole-2-thione (GM-784)

4.5 g (17.54 mmol, 1.0 eq.) GM-783 was suspended in 60 ml glacial acetic acid. 1.88 g (19.30 mmol, 1.1 eq.) KSCN was added in one portion to the well stirred suspension. The mixture was stirred for 1 hour, a color change from dark red to pale yellow was observed. After complete reaction, the mixture was cooled to 0° C. and the solids collected by filtration. The filtrate was poured on water to give a second crop of a crystalline solid. The combined solids were washed with water, dried and resuspended in diethyl ether. The suspension was stirred for 10 minutes at 0° C. and the solids collected by filtration and washed with n-pentane to give 3.65 g of the product as a pale yellow solid in 80% yield. ¹H NMR (200 MHz, DMSO-d₆) δ 12.67 (s, 1H), 12.25 (s, 1H), ¹³C NMR (50 MHz, DMSO) δ 162.40, 132.45, 125.90, 123.14, 122.39, 113.12, 109.47, 7.68-7.53 (m, 1H), 7.44-7.36 (m, 1H), 7.36-7.26 (m, 1H).

### 4-(4-Bromothiophen-2-yl)-2-(methylthio)-1H-imidazole (GM-785)

3.65 g (14 mmol, 1.0 eq.) GM-784 was suspended with 2.32 g (16.77 mmol, 1.2 eq.) K₂CO₃ in 80 ml MeOH. The suspension was vigorously stirred for 30 minutes at ambient temperature. Methyl iodide (915 μl, 14.7 mmol, 1.05 eq.) was added dropwise to the well stirred solution at ambient temperature and stirred for 2 hours. After complete consumption of the starting material, the mixture was poured on water and the solids were collected by filtration and dried under vacuum. The solid was resuspended in n-pentane stirred and filtered to give 3.4 g of the pure product as a white solid in 88% yield. ¹H NMR (400 MHz, DMSO-d₆) δ 12.44 (s, 1H), 7.62 (s, 1H), 7.43 (s, 1H), 7.26 (d, J=1.5 Hz, 1H), 2.55 (s, 3H). ¹³C NMR (101 MHz, DMSO) δ 142.07, 140.47, 135.73, 123.28, 120.96, 115.14, 109.36, 15.93.

### 4-(4-Bromothiophen-2-yl)-2-(methylthio)-1-((2-(trimethylsilyl)ethoxy)methyl)-1H-Imidazole (GM-787)

3.36 g (12.2 mmol, 1.0 eq.) GM-785 was dissolved in 100 ml dry THF and cooled to −15° C. Sodium hydride 60% (586 mg, 14.65 mmol, 1.2 eq.) was added portionwise under vigorous stirring. The mixture was stirred for 10 minutes at −10° C. and SEM-Cl (2267 μl, 12.81 mmol, 1.05 eq.) dissolved in 40 ml dry THF was added dropwise. The mixture was stirred for 2 hours, quenched by the addition of a saturated aqueous NH₄Cl solution. The organic layer was separated, dried over Na₂SO₄, filtered and evaporated. The crude product was purified by flash chromatography (SiO₂, Hex→Hex/EA 6:1) to give 4.45 g of the pure product as a colorless oil in 90% yield.

### tert-Butyl (5-(2-(methylthio)-1-((2-(trimethylsilyl)ethoxy)methyl)-1H-imidazol-4-yl)thiophen-3-yl)carbamate (GM-788)

1.80 g (4.44 mmol, 1.0 eq.) GM-787, 2.70 g (19.98 mmol, 4.5 eq.) K₂CO₃, 624 mg (5.33 mmol, 1.2 eq.) tert-butyl carbamate and 95 μl (0.89 mmol, 0.2 eq.) N,N′-dimethylethylendiamine was dissolved in 12 ml dry 1,4-dioxane. The mixture was degassed and 170 mg (0.89 mmol, 0.2 eq.) was added. The reaction was stirred at 90° C. overnight After cooling to ambient temperature, 100 ml of diethyl ether was added, the suspension filtered over Celite and the filtrate was evaporated. The crude product was purified by flash chromatography (SiO₂, Hex→Hex/EA 6:1) to give 1.50 g of the pure product as a green oil in 77% yield. ¹H NMR (200 MHz, Chloroform-d) δ 7.21 (d, J=8.1 Hz, 2H), 7.04 (s, 1H), 6.94 (dd, J=6.3, 2.2 Hz, 1H), 5.28 (s, 2H), 4.59 (s, OH), 3.65-3.44 (m, 2H), 2.67 (s, 3H), 1.54 (s, 9H), 1.04-0.87 (m, 2H), 0.02 (s, 9H). ¹³C NMR (50 MHz, CDCl₃) δ 152.74, 143.92, 137.13, 136.06, 116.00, 115.75, 105.12, 80.42, 79.54, 75.01, 66.41, 28.26, 28.16, 17.66, 16.57, −1.48. ESI-MS: 403.7 [M−H]⁻.

### tert-Butyl (5(5-bromo-2-(methylthio)-1-((2-(trimethylsilyl)ethoxy)methyl)-1H-imidazol-4-yl)thiophen-3-yl)carbamate (GM-791)

1.37 g (3.11 mmol, 1.0 eq.) GM-788 was dissolved in 60 ml MeCN and the solution was cooled to −30° C. 500 mg (2.80 mmol, 0.9 eq.) NBS dissolved in 10 ml MeCN was added dropwise and the mixture was stirred at −30° C. for 1 hour. The volatiles were removed by rotary evaporation, the residue was taken up in diethyl ether and washed with water. The separated organic layer was dried over Na2SO4, filtered and evaporated. The crude product was purified by flash chromatography (SiO₂, Hex→Hex/EA 4:1) to give 1.0 g of the pure product as a pale yellow solid in 69% yield. ESI-MS: 541.6, 543.6 [M+Na]⁺. ¹H NMR (200 MHz, DMSO-d₆) δ 8.84 (s, 1H), 7.84 (s, 1H), 7.31 (s, 1H), 5.27 (s, 2H), 3.63-3.46 (m, 2H), 2.58 (s, 3H), 1.49 (s, 9H), 0.96-0.79 (m, 2H), −0.01 (s, 9H). ¹³C NMR (50 MHz, DMSO) δ 153.33, 143.89, 136.72, 136.36, 135.50, 118.98, 118.35, 79.81, 75.09, 66.04, 28.42, 17.52, 16.13, −0.97.

### tert-Butyl (5-(5-(2-acetamidopyridin-4-yl)-2-(methylthio)-1-((2-(trimethylsilyl)ethoxy)methyl)-1H-imidazol-4-yl)thiophen-3-yl)carbamate (GM-792)

870 mg (1.68 mmol, 1.0 eq.) GM-791, 659 mg (2.51 mmol, 1.5 eq.) GM-449 was dissolved in 20 ml degassed 1,4-dioxane. An aqueous solution of K₃PO₄ (1.5 M, 5.0 mmol, 3 eq.) was added ad the biphasic mixture was degassed several times. 48 mg (0.084 mmol, 0.05 eq.) P(tBu)₃ Pd G3 was added under an atmosphere of argon. The mixture was stirred at 45° C. for 3 hours and an additional hour at 60° C. After complete consumption of the starting material, the mixture was cooled to ambient temperature, diluted with EtOAc, filtered over Celite and the organic layer was washed with water. The separated organics were dried over Na₂SO₄, filtered and the volatiles evaporated. The crude product was purified by flash chromatography (SiO₂, Hex→Hex/EA 1:2) to give 836 mg of the pure product as a pale yellow solid in 87% yield. ¹H NMR (200 MHz, DMSO-d₆) δ 10.54 (s, 1H), 9.05 (s, 1H), 8.37-8.25 (m, 2H), 7.92 (s, 1H), 7.35-7.21 (m, 2H), 5.29 (s, 2H), 3.65-3.48 (m, 2H), 2.62 (s, 3H), 2.13 (s, 3H), 1.43 (s, 9H), 0.98-0.81 (m, 2H), −0.01 (s, 9H). ¹³C NMR (50 MHz, DMSO) δ 169.67, 153.65, 153.05, 148.53, 144.14, 142.90, 136.38, 135.49, 134.95, 125.08, 121.85, 118.92, 117.00, 110.90, 79.54, 75.11, 66.05, 28.40, 24.37, 17.52, 16.07, −0.97. ESI-MS: 597.7 [M+Na]⁺.

### tert-Butyl (5-(5-(2-acetamidopyridin-4-yl)-2-(methylthio)-1-((2-(trimethylsilyl)ethoxy)methyl)-1H-imidazol-4-yl)thiophen-3-yl)carbamate (GM-795)

GM-792 (800 mg, 1.39 mmol) was dissolved in DCM containing 5% TFA. The reaction was stirred at ambient temperature for ten hours After complete consumption of the starting material, the reaction was quenched by the addition of NH₄Cl solution and extracted three times with EtOAc. The combined organic layers were dried over Na₂SO₄, filtered and the solvents removed by rotary evaporation. The crude product was purified by flash chromatography (SiO₂, Hex→EA) to give 400 mg of the pure product as a pale yellow solid in 61% yield. ESI-MS: 475.9 [M+H]⁺. ¹H NMR (400 MHz. DMSO-d₆) δ 10.40 (a, 1H), 8.21-8.15 (m, 2H), 7.77 (s, 1H), 7.23 (dd, J=5.5, 1.8 Hz, 1H), 6.88 (s, 1H), 5.52 (s, 2H), 5.27 (s, 2H), 3.54 (dd, J=8.6 ¹³C NMR (101 MHz, DMSO) δ 169.72, 153.08, 148.56, 147.05, 144.37, 144.11, 136.96, 135.95, 118.80, 117.89, 115.46, 108.99, 107.45, 75.12, 66.11, 24.46, 17.65, 16.17, −0.89, 7.4 Hz, 2H), 2.59 (s, 3H), 2.11 (s, 3H), 0.92-0.81 (m, 2H), −0.03 (s, 9H).

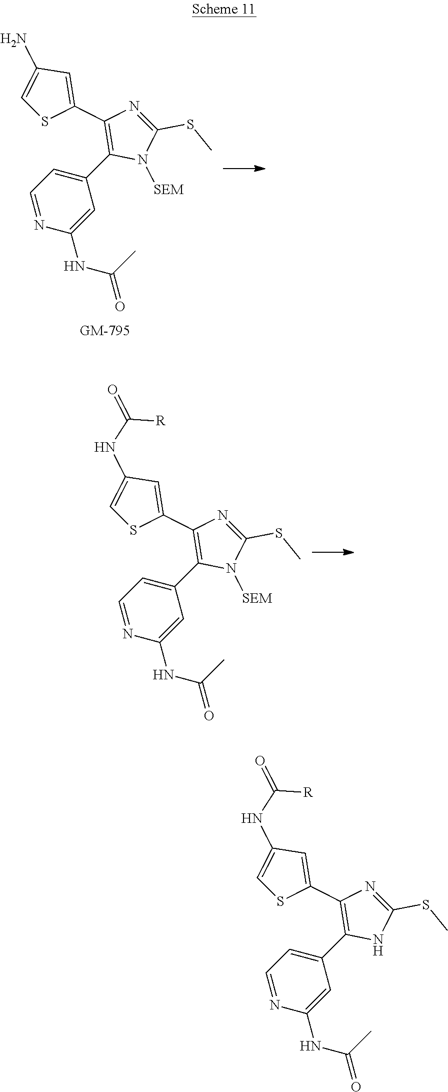

Final compounds of the thiophen derivatives were synthesized by at first amide coupling and subsequent removal of SEM protection group.

### N-(5-(5-(2-acetamidopyridin-4-yl)-2-(methylthio)-14(2-(trimethylsilyl)ethoxy)methyl)-1H-imidazol-4-yl)thiophen-3-yl)-2,6-difluorobenzamide (GM-803)

GM-795 (50 mg, 0.105 mmol, 1.0 eq.) 2,6-difluorobenzoic acid (20 mg, 0.126 mmol, 1.2 eq.), PyBOP (62 mg, 0.126 mmol, 1.2 eq.) and DIPEA (28 μl, 0.158 mmol, 1.5 eq.) was dissolved in 4 ml DMF. The mixture was stirred at ambient temperature overnight. After complete consumption of the starting material, Ether was added and the organic layer washed three times with water. The organic layer was separated, dried over Na₂SO₄, filtered and all volatiles were removed by rotary evaporation. The crude mixture was purified by flash chromatography (SiO₂, Hex→EA) to give 40 mg of the pure product as a red solid in 62% yield. ¹H NMR (200 MHz, Chloroform-d) δ 9.22 (s, 1H), 8.66 (s, 1H), 8.24 (s, 1H), 8.15 (d, J=5.4 Hz, 1H), 7.96 (s, 1H), 7.46-7.19 (m, 3H), 7.03-6.89 (m, 2H), 5.29 (s, 2H), 3.63-3.53 (m, 2H), 2.69 (s, 3H), 2.13 (s, 3H), 1.03-0.93 (m, 2H), 0.03 (s, 9H). ESI-MS: 615.8 [M+H]⁺.

### N-(5-(5-(2-acetamidopyridin-4-yl)-2-(methylthio)-1H-imidazol-4-yl)thiophen-3-yl)-2,6-difluorobenzamide (011)

The title compound was prepared from GM-803 (30 mg, 0.05 mmol) according to general procedure 2A. The crude product was purified by flash chromatography (SiO₂, DCM→DCM/MeOH 10%). 23 mg (0,047 mmol) of the pure were obtained as an off-white solid in 95% yield. ¹H NMR (400 MHz, DMSO-d₆) δ12.49 (s, 1H), 10.69 (s, 1H), 10.54 (s, 1H), 8.30 (d, J=5.3 Hz, 1H), 8.26 (s, 1H), 7.79 (s, 1H), 7.58 (tt, J=8.5, 6.6 Hz, 1H), 7.48 (s, 1H), 7.33 (dd, J=5.4, 1.8 Hz, 1H), 7.28-7.20 (m, 2H), 3.34 (s, 3H), 2.59 (s, 3H), 2.11 (s, 3H). ¹³C NMR (101 MHz, DMSO) δ 169.79, 160.73, 160.66, 159.34, 158.26, 158.18, 153.24, 148.54, 142.41, 142.37, 138.06, 135.97, 133.09, 132.63, 132.53, 132.44, 126.55, 120.96, 117.40, 115.58, 115.44, 112.58, 112.53, 112.39, 112.34, 111.63, 24.40, 15.77. ESI-MS: 485.1 [M+H]⁺.

### N-(5-(5-(2-acetamidopyridin-4-yl)-2-(methylthio)-1H-imidazol-4-yl)thiophen-3-yl)-2-phenylacetamide (012)

GM-795 (68 mg, 0.143 mmol, 1.0 eq.) and NaHCO₃ (24 mg, 0.286 mmol, 2.0 eq.) was dissolved in THF/water (400 μl 1:1) and stirred at ambient temperature. Phenylacetylchloride (23 μl, 0.172 mmol, 1.2 eq.) was added to the well stirred solution. The mixture was stirred at ambient temperature until complete consumption of the starting material (four hours). Then brine was added followed by EtOAc. The organic layer was separated, dried over sodium sulfate, filtered and the solvents removed by rotary evaporation. The residue was dissolved in 5 ml DCM and 2.5 ml TFA. Stirring was continued for 24 hours. The mixture was quenched by the careful addition of saturated NaHCO₃ solution and the product extracted with EtOAc three times. The combined organic layers were dried over sodium sulfate, filtered and the solvents removed by rotary evaporation. The crude product was purified by flash chromatography (SiO₂, EtOAc) to yield the give the final compound in 50% yield (33 mg, 0.071 mmol) over two steps as an off-white solid. ¹H NMR (200 MHz, DMSO-d₆) δ 12.48 (s, 1H), 10.56 (a, 1H), 10.02 (s, 1H), 8.39 (s, 1H), 8.23 (d, J=5.3 Hz, 1H), 7.67 (s, 1H), 7.47-7.25 (m, 6H), 7.14 (d, J=5.4 Hz, 1H), 3.70 (s, 2H), 2.59 (s, 3H), 2.15 (s, 3H). ¹³C NMR (50 MHz, CDCl₃) δ 169.82, 169.61, 153.13, 148.62, 142.45, 136.13, 134.66, 129.71, 128.67, 126.92, 124.75, 121.25, 117.06, 110.91, 42.82, 24.41,

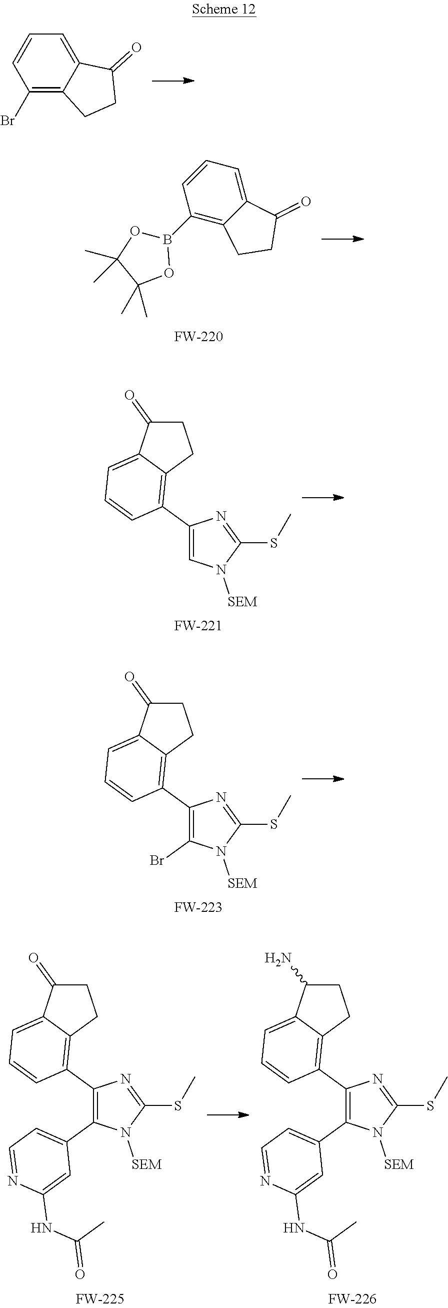 [SMILES: O=C1CCc2c(Br)cccc21 ; CC1(C)OB(c2cccc3c2CCC3=O)OC1(C)C ; CSc1nc(-c2cccc3c2CCC3=O)cn1COCC[Si](C)(C)C ; CSc1nc(-c2cccc3c2CCC3=O)c(Br)n1COCC[Si](C)(C)C ; CSc1nc(-c2cccc3c2CCC3=O)c(-c2ccnc(NC(C)=O)c2)n1COCC[Si](C)(C)C ; CSc1nc(-c2cccc3c2CCC3N)c(-c2ccnc(NC(C)=O)c2)n1COCC[Si](C)(C)C]

### 4-(4,4,5,5-tetramethyl-1,3,2-dioxaborolan-2-yl)-2,3-dihydro-1H-inden-1-one (FW-220)

In an oven dried schlenk flask were combined 767 mg (3.63 mmol) 4-bromo-2,3-dihydro-1H-inden-1-one, 969 mg bis(pinacolato)diboron (3.82 mmol), 1070 mg KOAc (10.9 mmol) and 79 mg Pd(dppf)Cl₂ (0.11 mmol) under inert atmosphere and 10 ml degassed dioxane were added. The mixture was heated to 80° C. oil bath temperature for 20 hours. The brown suspension was diluted with EtOAc and filtered over celite. The filtrate was concentrated under reduced pressure and the residue purified via flash chromatography (SiO₂, Hex→Hex/EtOAc 7:3). Yield: 790 mg (84%) as yellow solid. ¹H NMR (200 MHz, CDCl₃) δ 8.04 (d, J=6.9 Hz, 1H), 7.84 (d, J=7.8 Hz, 1H), 7.37 (t, J=7.4 Hz, 1H), 3.42-3.28 (m, 2H), 2.74-2.61 (m, 2H), 1.36 (s, 12H). ¹³C NMR (50 MHz, CDCl₃) δ 208.00, 162.35, 141.94, 136.58, 126.76, 126.51, 84.03, 36.41, 27.49, 25.09.

### 4-(2-(methylthio)-1-((2-(trimethylsilyl)ethoxy)methyl)-1H-imidazol-4-yl)-2,3-dihydro-1H-inden-1-one (FW-221)

565 mg (1.74 mmol, 1.0 eq.) FW-213, 496 mg (1.92 mmol, 1.1 eq.) FW-220 was dissolved in 17.5 ml degassed 1,4-dioxane. An aqueous solution of K₃PO₄ (1.5 M, 5.2 mmol, 3 eq.) was added ad the biphasic mixture was degassed several times. 39 mg (0.070 mmol, 0.04 eq.) P(tBu)₃ Pd G3 was added under an atmosphere of argon. The mixture was stirred at 60° C. over night After complete consumption of the starting material, the mixture was cooled to ambient temperature and solvents were removed in vacuo. The crude mixture was purified via flash chromatography (SO₂, Hex→Hex/EA 3:2) to give 440 mg (1.17 mmol) of the pure product as a red oil in 67% yield. H NMR (200 MHz, CDCl₃) δ 8.27 (d, J=7.4 Hz, 1H), 7.69 (d, J=7.5 Hz, 1H), 7.45 (t, J=7.6 Hz, 1H), 7.35 (s, 1H), 5.33 (s, 2H), 3.63-3.52 (m, 2H), 3.35-3.22 (m, 2H), 2.79-2.70 (m, 5H), 1.00-0.89 (m, 2H), 0.00 (s, 9H). ¹³C NMR (50 MHz, CDCl₃) δ 207.29, 151.37, 144.54, 139.83, 137.83, 132.25, 131.80, 127.92, 122.35, 118.94, 75.39, 66.81, 36.35, 27.11, 17.88, 16.61, −1.27.

### 4-(5-bromo-2-(methylthio)-1-((2-(trimethylsilyl)ethoxy)methyl)-1H-imidazol-4-yl)-2,3-dihydro-1H-inden-1-one (FW-223)

440 mg (1.17 mmol, 1.0 eq.) FW-221 was dissolved in 11 ml MeCN and the solution was cooled to −30° C. 209 mg (1.17 mmol, 1.0 eq.) NBS dissolved in 5.5 ml MeCN was added dropwise and the mixture was stirred at −30° C. for 0.5 hour. After addition of water and sodium sulfite, the mixture was extracted three times with DCM. The separated organic layers were dried over Na₂SO₄, filtered and evaporated. The crude product was purified via flash chromatography (SiO₂, Hex→Hex/EtOAc 1:1) to give 365 mg of the pure product as a dark red oil in 68% (0.80 mmol). ESI-MS: 506.7, 508.8 [M+Na+MeOH]⁺. ¹H NMR (200 MHz, CDCl₃) δ 7.86 (d, J=7.1 Hz, 1H), 7.74 (d, J=6.7 Hz, 1H), 7.42 (t, J=7.3 Hz, 1H), 5.37 (s, 2H), 3.65 (t, J=7.7 Hz, 2H), 3.43-3.24 (m, 2H), 2.76-2.61 (m, 5H), 1.02-0.87 (m, 2H), 0.00 (s, 9H).

### N-(4-(2-(methylthio)-4-(1-oxo-2,3-dihydro-1H-inden-4-yl)-1-((2-(trimethylsilyl)ethoxy)-methyl)-1H-imidazol-5-yl)pyridin-2-yl)acetamide (FW-225)

430 mg (0.94 mmol, 1.0 eq.) FW-223, 447 mg (1.71 mmol, 1.8 eq.) GM-449 was dissolved in 10 ml degassed 1,4-dioxane. An aqueous solution of K₃PO₄ (1.5 M, 2.84 mmol, 3 eq.) was added ad the biphasic mixture was degassed several times. 28 mg (0.047 mmol, 0.05 eq.) P(tBu)₃ Pd G3 was added under an atmosphere of argon. The mixture was stirred at 40° C. over night. After complete consumption of the starting material, the mixture was cooled to ambient temperature, diluted with EtOAc and the organic layer was washed with water. The separated organic layers were dried over Na₂SO₄, filtered and the volatiles evaporated. The crude product was purified via flash chromatography (SiO₂, Hex→Hex/EtOAc 1:4) to give 430 mg (0.85 mmol) of the pure product as a red oil in 89% yield. ESI-MS: 531.0 [M+Na]⁺. ¹H NMR (200 MHz, CDCl₃) δ 9.11 (s, 1H), 8.38 (s, 1H), 8.10 (d, J=5.5 Hz, 1H), 7.71 (d, J=7.2 Hz, 1H), 7.40 (d, J=6.9 Hz, 1H), 7.33-7.22 (m, 1H), 6.99 (dd, J=5.2, 0.8 Hz, 1H), 5.29 (s, 2H), 3.64 (t, J=8.5 Hz, 2H), 3.19-3.05 (m, 2H), 2.75 (s, 3H), 2.69-2.60 (m, 2H), 2.21 (s, 3H), 1.06-0.94 (m, 2H), 0.00 (s, 9H).

### N-(4-(4-(1-amino-2,3-dihydro-1H-inden-4-yl)-2-(methylthio)-1-((2-(trimethylsilyl)ethoxy)methyl)-1H-imidazol-5-yl)pyridin-2-yl)acetamide (FW-226)

430 mg (0.85 mmol) FW-225 was dissolved in 0.4 ml isopropanol and 1950 mg (25.36 mmol, 30 eq.) of ammonium formate was added. After stirring for 1 h at ambient temperature, 159 mg (2.54 mmol, 3.0 eq.) of NaBH₃CN was added and the mixture was then refluxed for 2 h. After cooling down to ambient temperature, the mixture was diluted with EtOAc and washed with aqeuous 3 N NaOH. Separated organic layers were dried over Na₂SO₄ and solvents evaporated in vacuo. The crude product was purified via flash chromatography (SiO₂, DCM/(2 N NH₃ in MeOH) 1%→10% increasing polar basic proportion). Yield: 260 mg (0.50 mmol, 60%) as a white solid. ESI-MS: 532.0 [M+Na]⁺. ¹H NMR (200 MHz, CDCl₃) δ 8.48-7.92 (m, 3H), 7.45-7.30 (m, 1H), 7.18-7.06 (m, 2H), 7.04-6.94 (m, 1H), 5.29 (s, 2H), 4.44 (t, J=6.5 Hz, 1H), 3.61 (t, J=8.2 Hz, 2H), 3.14-2.79 (m, 3H), 2.75-2.60 (m, 4H), 2.54-2.30 (m, 1H), 2.15 (s, 3H), 1.86-1.65 (m, 1H), 1.02-0.90 (m, 2H), −0.01 (s, 9H).

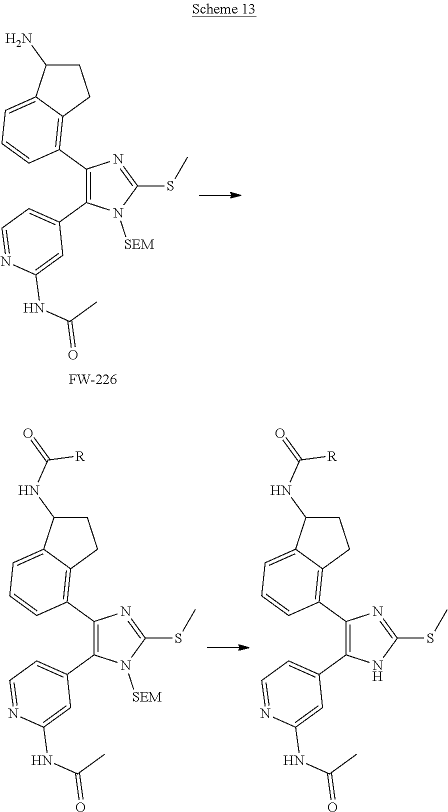

Final compounds of the 2,3-dihydro-1H-inden-4-yl derivatives were synthesized by at first amide coupling and subsequent removal of SEM protection group.

### N-(4-(5-(2-acetamidopyridin-4-yl)-2-(methylthio)-1-((2-(trimethylsilyl)ethoxy)methyl)-1H-imidazol-4-yl)-2,3-dihydro-1H-inden-1-yl)-2,6-difluorobenzamide (FW-228a)

The title compound was synthesized according to general procedure 1A) from 75 mg (0.15 mmol) FW-226, 30 mg (0.19 mmol 1.3 eq.) 2,6-difluorobenzoic acid, 99 mg (0.19 mmol, 1.3 eq.) PyBOP and 61 μl (0.44 mmol, 3.0 eq.) TEA. Flash chromatography (SiO₂, n-hex->n-hex/EtOAc 25:75). The product was directly used in the next step without further characterization. ESI-MS: 672.0 [M+Na]⁺.

### N-(4-(5-(2-acetamidopyridin-4-yl)-2-(methylthio)-1H-imidazol-4-yl)-2,3-dihydro-1H-inden-1-yl)-2,6-difluorobenzamide (055)

The title compound was synthesized according to general procedure 2B) from the product of FW-228a. Flash chromatography (SiO₂, DCM/MeOH 1%->10%) Yield: 44 mg (58%) over two steps as off-white solid. ESI-MS: 542.0 [M+Na]⁺. As mixture of tautomers: ¹H NMR (400 MHz, DMSO) δ 12.92-12.45 (m, 1H), 10.48-10.11 (m, 1H), 9.08 (d, J=7.9 Hz, 1H), 8.22-7.87 (m, 2H), 7.56-7.47 (m, 1H), 7.43-7.25 (m, 3H), 7.19 (t, J=7.9 Hz, 2H), 7.05-6.90 (m, 1H), 5.60-5.51 (m, 1H), 2.70-2.57 (m, 5H), 2.44-2.33 (m, 1H), 1.95-1.80 (m, 4H).

### N-(4-(5-(2-acetamidopyridin-4-yl)-2-(methylthio)-1-((2-(trimethylsilyl)ethoxy)methyl)-1H-imidazol-4-yl)-2,3-dihydro-1H-inden-1-yl)-1H-indole-4-carboxamide (FW-235a)

The title compound was synthesized according to general procedure 1A) from 50 mg (0.10 mmol) FW-226, 20 mg (0.13 mmol 1.3 eq.) 1H-indole-4-carboxylic acid, 66 mg (0.13 mmol, 1.3 eq.) PyBOP and 41 μl (0.29 mmol, 3.0 eq.) TEA. Flash chromatography (SiO₂, n-hex->n-hex/EtOAc 20:80). The product was directly used in the next step without further characterization. ESI-MS: 675.0 [M+Na]⁺.

### N-(4(5-(2-acetamidopyridin-4-yl)-2-(methylthio)-1H-imidazol-4-yl)-2,3-dihydro-1H-inden-1-yl)-1H-indole-4-carboxamide (056)

The product of FW-235a was dissolved in DMF. 72 mg (0.23 mmol, 3.0 eq.) Tetra-n-butylammonium fluoride trihydrate and 30 μl (0.46 mmol, 6.0 eq.) ethylenediamine were added and the mixture was stirred for 48 h at 50° C. Brine was added and the aqueous phase was extracted with EtOAc. Combined organic layers were dried over Na₂SO₄, filtered and volatiles removed in vacuo. Purification via flash chromatography (SiO₂, DCM/MeOH 1%->10%) Yield: 12 mg (30%) over two steps as yellow solid (with 94.5% purity in HPLC at 254 nm, 90.8% at 230 nm). ESI-MS: 522.9 [M+H]⁺. As mixture of tautomers: ¹H NMR (400 MHz, DMSO) δ 12.91-12.54 (m, 1H), 11.29 (s, 1H), 10.68-10.21 (m, 1H), 8.62-8.44 (m, 1H), 8.36-7.90 (m, 2H), 7.54 (d, J=8.0 Hz, 1H), 7.49-7.40 (m, 3H), 7.35 (t, J=7.4 Hz, 1H), 7.26 (d, J=7.5 Hz, 1H), 7.17-6.95 (m, 2H), 6.89-6.79 (m, 1H), 5.73-5.60 (m, 1H), 2.77-2.58 (m, 5H), 2.45-2.33 (m, 1H), 2.02-1.92 (m, 1H), 1.90-1.76 (m, 3H).

### N-(4-(5-(2-acetamidopyridin-4-yl)-2-(methylthio)-1-((2-(trimethylsilyl)ethoxy)methyl)-1H-imidazol-4-yl)-2,3-dihydro-1H-inden-1-yl)-3,5-difluorobenzamide (FW-236a)

The title compound was synthesized according to general procedure 1A) from 50 mg (0.10 mmol) FW-226, 20 mg (0.13 mmol 1.3 eq.) 3,5-difluorobenzoic acid, 66 mg (0.13 mmol, 1.3 eq.) PyBOP and 41 μl (0.29 mmol, 3.0 eq.) TEA. Flash chromatography (SiO₂, n-hex->n-hex/EtOAc 20:80). The product was directly used in the next step without further characterization. ESI-MS: 672.1 [M+Na]⁺.

### N-(4-(5-(2-acetamidopyridin-4-yl)-2-(methylthio)-1H-imidazol-4-yl)-2,3-dihydro-1H-inden-1-yl)-3,5-difluorobenzamide (057)

The title compound was synthesized according to general procedure 2B) from the product of FW-236a. Flash chromatography (SiO₂, DCM/MeOH 1%->10%) Yield: 27 mg (52%) over two steps as off-white solid. ESI-MS: 542.2 [M+Na]⁺. As mixture of tautomers: ¹H NMR (400 MHz, DMSO) δ 12.90-12.53 (m, 1H), 10.49-10.22 (m, 1H), 8.89 (d, J=7.2 Hz, 1H), 8.26-7.98 (m, 2H), 7.68-7.56 (m, 2H), 7.47 (t, J=8.8 Hz, 1H), 7.38-7.19 (m, 3H), 7.02-6.87 (m, 1H), 5.63-5.53 (m, 1H), 2.87-2.59 (m, 5H), 2.44-2.31 (m, 1H), 2.07-1.98 (m, 3H), 1.97-1.85 (m, 1H).

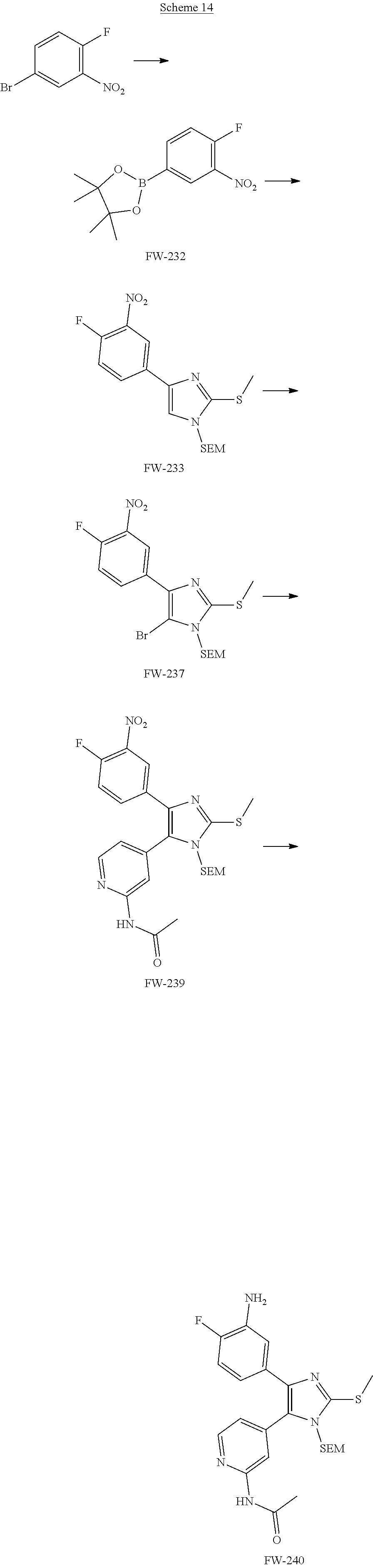 [SMILES: O=[N+]([O-])c1cc(Br)ccc1F ; CC1(C)OB(c2ccc(F)c([N+](=O)[O-])c2)OC1(C)C ; CSc1nc(-c2ccc(F)c([N+](=O)[O-])c2)cn1COCC[Si](C)(C)C ; CSc1nc(-c2ccc(F)c([N+](=O)[O-])c2)c(Br)n1COCC[Si](C)(C)C ; CSc1nc(-c2ccc(F)c([N+](=O)[O-])c2)c(-c2ccnc(NC(C)=O)c2)n1COCC[Si](C)(C)C ; CSc1nc(-c2ccc(F)c(N)c2)c(-c2ccnc(NC(C)=O)c2)n1COCC[Si](C)(C)C]

### 2-(4-fluoro-3-nitrophenyl)-4,4,5,5-tetramethyl-1,3,2-dioxaborolane (FW-232)

In an oven dried schlenk flask were combined 1000 mg (4.54 mmol) 4-bromo-1-fluoro-2-nitrobenzene, 1212 mg bis(pinacolato)diboron (4.77 mmol, 1.05 eq.), 1338 mg KOAc (13.63 mmol, 3.0 eq.) and 99 mg Pd(dppf)Cl₂ (0.14 mmol, 0.03 eq.) under inert atmosphere and 10 ml degassed dioxane were added. The mixture was heated to 90° C. oil bath temperature overnight. The brown suspension was diluted with EtOAc and filtered over celite. The filtrate was concentrated under reduced pressure and the resulting slurry was precipitated in n-hexane and filtered. Yield: 1240 mg (quantitative yield) as yellow solid. ¹H NMR (200 MHz, CDCl₃) δ 8.56-8.42 (m, 1H), 8.13-8.00 (m, 1H), 7.37-7.23 (m, 1H), 1.38 (s, 12H). ¹³C NMR (50 MHz, CDCl₃) δ 160.10, 154.77, 142.00, 141.82, 132.59, 132.54, 118.10, 117.71, 84.87, 24.94.

### 4-(4-fluoro-3-nitrophenyl)-2-(methylthio)-1-((2-(trimethylsilyl)ethoxy)methyl)-1H-imidazole (FW-233)

950 mg (2.94 mmol, 1.0 eq.) FW-213, 1020 mg (3.82 mmol, 1.3 eq.) FW-232 was dissolved in 30 ml degassed 1,4-dioxane. An aqueous solution of K₃PO₄ (1.5 M, 8.81 mmol, 3 eq.) was added ad the biphasic mixture was degassed several times. 67 mg (0.12 mmol, 0.04 eq.) P(tBu)₃ Pd G3 was added under an atmosphere of argon. The mixture was stirred at 50° C. overnight After complete consumption of the starting material, the mixture was cooled to ambient temperature, diluted with EtOAc and the organic layer was washed with water. The separated organic layers were dried over Na₂SO₄, filtered and the volatiles evaporated. The crude product was purified via flash chromatography (SiO₂, Hex→Hex/EtOAc 7:3) to give 940 mg (2.44 mmol) of the pure product as a pale yellow solid in 83% yield. ESI-MS: 383.9 [M+H]⁺. ¹H NMR (200 MHz, CDCl₃) δ 8.40 (dd, J=7.1, 2.1 Hz, 1H), 8.03 (ddd, J=8.6, 4.1, 2.2 Hz, 1H), 7.38 (s, 1H), 7.25 (t, J=9.6 Hz, 1H), 5.28 (s, 2H), 3.60-3.50 (m, 2H), 2.68 (s, 3H), 0.98-0.87 (m, 2H), −0.02 (s, 9H). ¹³C NMR (50 MHz, CDCl₃) δ 156.99, 151.74, 145.34, 139.17, 131.66, 131.50, 131.18, 131.11, 122.01, 121.96, 118.85, 118.43, 117.47, 75.37, 66.84, 17.87, 16.53, −1.30.

### 5-bromo-4-(4-fluoro-3-nitrophenyl)-2-(methylthio)-1-((2-(trimethylsilyl)ethoxy)methyl)-1H-imidazole (FW-237)

900 mg (2.34 mmol) FW-233 was dissolved in 25 ml MeCN and the solution was cooled to −30° C. 418 mg (2.34 mmol, 1.0 eq.) NBS dissolved in 10 ml MeCN was added dropwise and the mixture was stirred at −30° C. for 1 hour. After addition of water and sodium sulfite, the mixture was extracted three times with DCM. The separated organic layers were dried over Na₂SO₄, filtered and evaporated. The crude product was purified via flash chromatography (SiO₂, Hex→Hex/EtOAc 7:3). Yield: 950 mg (2.04 mmol) of the pure product as a pale yellow solid in 87% yield. ¹H NMR (200 MHz, CDCl₃) δ 8.74, 8.73, 8.71, 8.69, 8.33, 8.32, 8.31, 8.29, 8.28, 8.27, 8.26, 8.25, 7.36, 7.32, 7.31, 5.36, 3.67, 3.63, 3.59, 2.69, 1.00, 0.96, 0.92, 0.00. ¹³C NMR (50 MHz, CDCl₃ δ 157.19, 151.92, 146.42, 136.20, 133.39, 133.22, 130.31, 130.23, 123.92, 123.86, 118.59, 118.17, 101.34, 74.08, 67.05, 17.96, 16.04, −1.27.

### N-(4-(4-(4-fluoro-3-nitrophenyl)-2-(methylthio)-1-((2-(trimethylsilyl)ethoxy)methyl)-1H-Imidazol-5-yl)pyridin-2-yl)acetamide (FW-239)

920 mg (1.99 mmol, 1.0 eq.) FW-237, 782 mg (2.98 mmol, 1.5 eq.) GM-449 was dissolved in 20 ml degassed 1,4-dioxane. An aqueous solution of K₃PO₄ (1.5 M, 5.97 mmol, 3 eq.) was added and the biphasic mixture was degassed several times. 58 mg (0.084 mmol, 0.05 eq.) P(tBu)₃ Pd G3 was added under an atmosphere of argon. The mixture was stirred at 50° C. overnight. After complete consumption of the starting material, the mixture was cooled to ambient temperature, diluted with EtOAc and the organic layer was washed with water. The separated organic layer was dried over Na₂SO₄, filtered and the volatiles evaporated. The crude product was purified via flash chromatography (SiO₂, Hex→Hex/EtOAc 2:8) to give 770 mg (1.49 mmol) of the pure product as a yellow solid in 75% yield. ESI-MS: 540.0 [M+Na]+. ¹H NMR (200 MHz, CDCl₃) δ 8.73 (s, 1H), 8.35-8.28 (m, 2H), 8.22 (dd, J=7.2, 2.2 Hz, 1H), 7.70 (ddd, J=8.6, 4.2, 2.3 Hz, 1H), 7.18-7.05 (m, 2H), 5.18 (s, 2H), 3.59-3.48 (m, 2H), 2.74 (s, 3H), 2.20 (s, 3H), 0.97-0.87 (m, 2H), −0.03 (s, 9H). ¹³C NMR (50 MHz, CDCl₃) δ 168.91, 157.13, 152.41, 151.86, 148.42, 147.05, 140.33, 137.51, 137.35, 137.07, 133.99, 133.82, 131.12, 131.04, 128.70, 124.62, 124.57, 121.04, 118.56, 118.15, 115.13, 73.18, 66.81, 24.82, 17.92, 16.17, −1.33.

### N-(4-(4-(3-amino-4-fluorophenyl)-2-(methylthio)-1-((2-(trimethylsilyl)ethoxy)methyl)-1H-imidazol-5-yl)pyridin-2-yl)acetamide (FW-240)

570 mg (1.10 mmol) FW-239 was dissolved in 10 ml dry MeOH under argon atmosphere. 719 mg (11.0 mmol, 10 eq.) Zinc dust and 589 mg (11.0 mmol, 10 eq.) NH₄Cl were added portion wise over 1 h. After complete conversion the mixture was diluted with MeOH and filtered over celite. Solvents were evaporated and the residue was suspended in EtOAc and again filtered over celite. The crude product was used directly in the next step without further purification. Yield: 600 mg (quantitative yield) as yellow solid. ESI-MS: 510.0 [M+Na]⁺. ¹H NMR (200 MHz, CDCl₃) δ 9.48 (s, 1H), 8.28 (d, J=5.5 Hz, 1H), 8.19 (s, 1H), 7.15-7.03 (m, 1H), 6.99-6.88 (m, 1H), 6.86-6.74 (m, 1H), 6.72-6.59 (m, 1H), 5.21 (s, 2H), 3.81-3.37 (m, 4H), 2.70 (s, 3H), 2.23-2.14 (m, 3H), 1.01-0.89 (m, 2H), −0.02 (s, 9H).

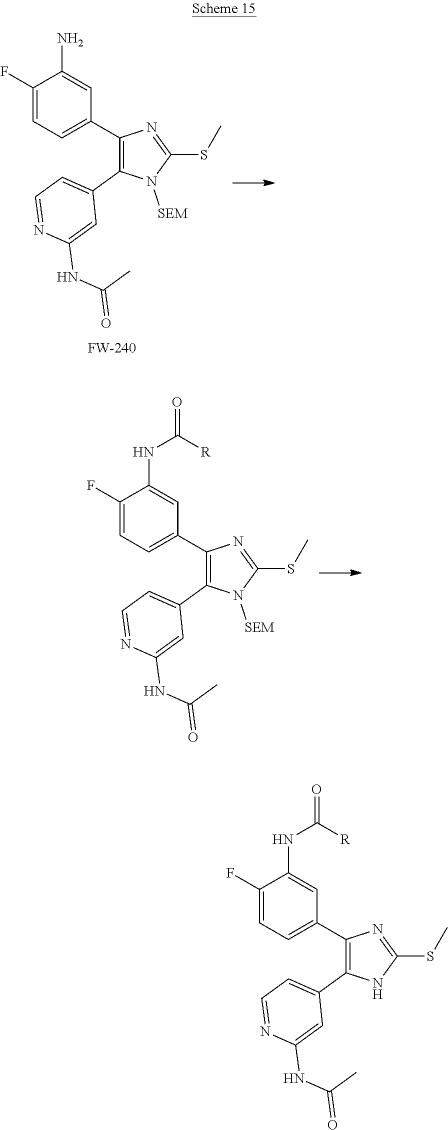

Final compounds of the 4-fluoro-3-anilino derivatives were synthesized by at first amide coupling and subsequent removal of SEM protection group.

### N-(5-(5-(2-acetamidopyridin-4-yl)-2-(methylthio)-1-((2-(trimethylsilyl)ethoxy)methyl)-1H-imidazol-4-yl)-2-fluorophenyl)-2,6-difluorobenzamide (FW-244a)

The title compound was synthesized according to general procedure 1A) from 60 mg (0.12 mmol) FW-240, 24 mg (0.15 mmol 1.25 eq.) 2,6-difluorobenzoic acid, 80 mg (0.15 mmol, 1.25 eq.) PyBOP and 51 μl (0.37 mmol, 3.0 eq.) TEA. Flash chromatography (SiO₂, DCM/MeOH 1%->10%). The product was directly used in the next step without further characterization. ESI-MS: 649.9 [M+Na]⁺.

### N-(5-(5-(2-acetamidopyridin-4-yl)-2-(methylthio)-1H-imidazol-4-yl)-2-fluorophenyl)-2,6-difluorobenzamide (046)

The title compound was synthesized according to general procedure 2A) from the product of FW-244a. Flash chromatography (SiO₂, DCM/MeOH 1%->10%) Yield: 21 mg (35%) over two steps as off-white solid. ESI-MS: 520.0 [M+Na]⁺. As mixture of tautomers: ¹H NMR (400 MHz, DMSO) δ 12.94-12.66 (m, 1H), 10.86-10.57 (m, 1H), 10.52-10.26 (m, 1H), 8.38-8.10 (m, 2H), 8.08-7.96 (m, J=6.2 Hz, 1H), 7.65-7.53 (m, 1H), 7.42-7.19 (m, 4H), 7.13-7.02 (m, J=5.1 Hz, 1H), 2.63 (s, 3H), 2.13-2.00 (m, 3H).

### N-(5-(5-(2-acetamidopyridin-4-yl)-2-(methylthio)-1-((2-(trimethylsilyl)ethoxy)methyl)-1H-imidazol-4-yl)-2-fluorophenyl)-3,5-difluorobenzamide (FW-248a)

The title compound was synthesized according to general procedure 1A) from 60 mg (0.12 mmol) FW-240, 29 mg (0.18 mmol 1.5 eq.) 3,5-difluorobenzoic acid, 79 mg (0.25 mmol, 2.0 eq.) TBTU and 51 μl (0.37 mmol, 3.0 eq.) TEA. Flash chromatography (SiO₂, DCM/MeOH 1%->10%). The product was directly used in the next step without further characterization. ESI-MS: 650.1 [M+Na]⁺.

### N-(5-(5-(2-acetamidopyridin-4-yl)-2-(methylthio)-1H-imidazol-4-yl)-2-fluorophenyl)-3,5-difluorobenzamide (051)

The title compound was synthesized according to general procedure 2A) from the product of FW-248a. Flash chromatography (SiO₂, DCM/MeOH 1%->10%) Yield: 30 mg (50%) over two steps as off-white solid. ESI-MS: 520.0 [M+Na]⁺. As mixture of tautomers: ¹H NMR (200 MHz, DMSO) δ 12.98-12.56 (m, 1H), 10.56-10.25 (m, 2H), 8.37-8.08 (m, 2H), 7.82-7.48 (m, 4H), 7.45-7.19 (m, 2H), 7.13-7.01 (m, 1H), 2.62 (s, 3H), 2.06 (s, 3H).

### N-(5-(5-(2-acetamidopyridin-4-yl)-2-(methylthio)-1-((2-(trimethylsilyl)ethoxy)methyl)-1H-imidazol-4-yl)-2-fluorophenyl)-2-(2,6-difluorophenyl)acetamide (FW-249a)

The title compound was synthesized according to general procedure 1A) from 60 mg (0.12 mmol) FW-240, 32 mg (0.18 mmol 1.5 eq.) 2-(2,6-difluorophenyl)acetic acid, 79 mg (0.25 mmol, 2.0 eq.) TBTU and 51 μl (0.37 mmol, 3.0 eq.) TEA. Flash chromatography (SiO₂, DCM/MeOH 1%->10%). The product was directly used in the next step without further characterization. ESI-MS: 664.2 [M+Na]⁺.

### N-(5-(5-(2-acetamidopyridin-4-yl)-2-(methylthio)-1H-Imidazol-4-yl)-2-fluorophenyl)-2-(2,6-difluorophenyl)acetamide (052)

The title compound was synthesized according to general procedure 2A) from the product of FW-249a. Flash chromatography (SiO₂, DCM/MeOH 1%->10%) Yield: 30 mg (48%) over two steps as off-white solid. ESI-MS: 534.0 [M+Na]⁺. As mixture of tautomers: ¹H NMR (400 MHz, DMSO) δ 12.82-12.64 (m, 1H), 10.52-10.26 (m, 1H), 10.25-10.03 (m, 1H), 8.31-8.00 (m, 3H), 7.42-7.15 (m, 3H), 7.11-6.99 (m, 3H), 3.91-3.80 (m, 2H), 2.59 (s, 3H), 2.07-2.00 (m, 3H).

### N-(5-(5-(2-acetamidopyridin-4-yl)-2-(methylthio)-1-((2-(trimethylsilyl)ethoxy)methyl)-1H-imidazol-4-yl)-2-fluorophenyl)benzamide (FW-250a)

The title compound was synthesized according to general procedure 1A) from 60 mg (0.12 mmol) FW-240, 29 mg (0.18 mmol 1.5 eq.) benzoic acid, 79 mg (0.25 mmol, 2.0 eq.) TBTU and 51 μl (0.37 mmol, 3.0 eq.) TEA. Flash chromatography (SiO₂, DCM/MeOH 1%->10%). The product was directly used in the next step without further characterization. ESI-MS: 614.2 [M+Na]⁺.

### N-(5-(5-(2-acetamidopyridin-4-yl)-2-(methylthio)-1H-imidazol-4-yl)-2-fluorophenyl)benzamide (044)

The title compound was synthesized according to general procedure 2A) from the product of FW-250a. Flash chromatography (SiO₂, DCM/MeOH 1%->10%) Yield: 20 mg (35%) over two steps as off-white solid. ESI-MS: 462.2 [M+H]⁺. As mixture of tautomers: ¹H NMR (400 MHz, DMSO) δ 12.89-12.69 (m, 1H), 10.55-10.32 (m, 1H), 10.26-10.08 (m, 1H), 8.37-8.11 (m, 2H), 8.00-7.93 (m, 2H), 7.82-7.71 (m, J=6.3 Hz, 1H), 7.63-7.50 (m, 3H), 7.42-7.19 (m, 2H), 7.13-7.03 (m, 1H), 2.62 (s, 3H), 2.10-2.03 (m, 3H).

### Example 2: HTRF-Based EGFR Biochemical Assays

EGFR biochemical activity measurements were carried out using the homogeneous time-resolved fluorescence (HTRF) assay (Cisbio). Inhibitors and DMSO normalizations were first dispensed to empty black low-volume 384-well plates (Corning) with D300 digital liquid dispenser (HP). All reactions were carried out at room temperature and solutions were added to plates with a Multidrop Combi Reagent Dispenser (ThermoFisher). The reaction mixture (10 μL final volume) contained 1 μM tyrosine kinase peptide-biotin substrate and mutant EGFR in a reaction buffer (50 mM HEPES pH 7.0, 5 mM MgCl₂, 1 mM MnCl₂, 0.01% BSA, 2 mM TCEP, 0.1 mM NaVO₄). Enzyme concentrations were adjusted to accommodate varying kinase activities (WT 5 nM, L858R 0.1 nM, L858R/T790M 0.02 nM, L858R/T790M/C797S 0.02 nM). Enzyme reaction solution (2× concentrations, 5 μL) was added to 384-well plates containing compounds and incubated for 30 mins. Enzyme reactions were initiated with the addition of 5 μL of ATP to a final concentration of 100 μM and reacted for 20 mins. Reactions were quenched with the addition of 10 μL of phospho-tyrosine antibody-Europium(III) cryptate (1-to-180 volume ratio) and Streptavidin-XL665 (46.7 nM) in EDTA-containing detection buffer, then incubated at room temperature for 1 hour, and read with a PHERAstar plate reader (excitation=337 nm, emission=620 nm and 665 nm). IC₅₀ values (μM) were determined by inhibition curves (11-point curves from 1.0 μM to 0.130 nM or 23-point curves from 1.0 μM to 0.130 μM) in triplicate with non-linear least squares fit in GraphPad Prism 7.0d. The data obtained are shown in Table 2 below.

TABLE 2

| | | | | L858R/ |
|---|---|---|---|---|
| Cmpd. | | | L858R/ | T790M/ |
| No. | WT | L858R | T790M | C797S |
| 001 | ND | 4.195 | 16.670 | ND |
| 002 | ND | 0.805 | 4.194 | ND |
| 003 | ND | 4.235 | 14.550 | ND |
| 004 | ND | 0.097 | 0.731 | ND |
| 005 | ND | 3.597 | 3.707 | ND |
| 006 | ND | 0.133 | 5.100 | ND |
| 007 | ND | 0.434 | 7.707 | ND |
| 008 | ND | 0.660 | 1.753 | ND |
| 009 | ND | 1.226 | 1.511 | ND |
| 010 | 0.015 | 0.032 | 2.380 | ND |
| 011 | ND | >100 | >100 | ND |
| 012 | ND | >100 | >100 | ND |
| 013 | ND | 0.206 | 2.112 | ND |
| 014 | ND | 0.149 | 1.826 | ND |
| 015 | ND | 6.432 | 5.943 | ND |
| 016 | ND | 0.512 | 0.857 | ND |
| 017 | ND | 4.127 | 5.297 | ND |
| 018 | ND | 1.599 | 2.196 | ND |
| 019 | ND | >100 | >100 | ND |
| 020 | ND | >100 | >100 | ND |
| 021 | ND | >100 | >100 | ND |
| 022 | ND | >100 | >100 | ND |
| 023 | ND | >100 | >100 | ND |
| 024 | ND | >100 | >100 | ND |
| 025 | ND | >100 | >100 | ND |
| 026 | ND | >100 | >100 | ND |
| 027 | ND | >100 | >100 | ND |
| 028 | ND | >100 | >100 | ND |
| 029 | ND | >100 | >100 | ND |
| 030 | ND | >100 | >100 | ND |
| 031 | ND | >100 | >100 | ND |
| 032 | ND | >100 | >100 | ND |
| 033 | ND | >100 | >100 | ND |
| 034 | ND | >100 | >100 | ND |
| 035 | ND | 11.200 | >100 | ND |
| 036 | ND | 2.491 | >100 | ND |
| 037 | ND | 1.882 | 7.865 | ND |
| 038 | >100 | 0.416 | 1.605 | 1.563 |
| 039 | 0.060 | 0.001 | 0.054 | 0.01671 |
| 040 | >100 | 0.177 | 0.907 | 0.9017 |
| 042 | ND | 0.012 | 0.098 | ND |
| 043 | 0.0004 | 0.007 | 0.053 | 0.008275 |
| 044 | ND | 0.042 | 1.226 | ND |
| 045 | ND | 0.062 | 0.215 | ND |
| 046 | ND | 0.062 | 1.212 | ND |
| 047 | >100 | 0.096 | 0.759 | 0.6104 |
| 048 | ND | 0.313 | 3.905 | ND |
| 049 | ND | 0.323 | 1.378 | ND |
| 050 | ND | 0.470 | >100 | ND |
| 051 | ND | 0.517 | >100 | ND |
| 052 | ND | 0.969 | 1.887 | ND |
| 053 | ND | 1.302 | 0.799 | ND |
| 054 | ND | 2.532 | >100 | ND |
| 055 | ND | >100 | >100 | ND |
| 056 | ND | >100 | >100 | ND |
| 057 | ND | >100 | >100 | ND |
| 058 | >100 | >100 | >100 | >100 |
| 059 | >100 | 3.072 | >100 | >100 |
| 060 | >100 | >100000 | >100 | >100 |
| 063 | 1.070 | 0.098 | >1.00 | 1.567 |
| 064 | 0.701 | 0.011 | 0.008 | 0.2058 |
| 065 | ND | 0.728 | 1.643 | ND |

### Example 3: Ba/F3 Cell Proliferation Models

The EGFR mutant L858R and L858R/T790M Ba/F3 cells have been previously described (Zhou, W., et al. Nature 462, 2009, 1070-1074). All cell lines were maintained in RPMI 1640 (Cellgro; Mediatech Inc., Herndon, CA) supplemented with 10% FBS, 100 units/mL penicillin. 100 units/mL streptomycin. The EGFR I941R mutation was introduced via site directed mutagenesis using the Quick Change Site-Directed Mutagenesis kit (Stratagene; La Jolla, CA) according to the manufacturer's instructions. All constructs were confirmed by DNA sequencing. The constructs were shuttled into the retroviral vector JP1540 using the Cre-recombination system (Agilent Technologies, Santa Clara, CA). Ba/F3 cells were then infected with retrovirus per standard protocols, as described previously (Zhou, et al, Nature 2009). Stable clones were obtained by selection in puromycin (2 μg/ml).

Growth and inhibition of growth was assessed by the Cell Titer Glo assay (Promega, Madison, WI) and was performed according to the manufacturer's instructions. The Cell Titer Glo assay is a luminescence-based method used to determine the number of viable cells based on quantitation of the ATP present, which is directly proportional to the amount of metabolically active cells present. Ba/F3 cells of different EGFR genotypes were exposed to compounds as a single agent for 72 hours and the number of cells used per experiment was determined empirically as has been previously established (Zhou, et al., Nature 2009). All experimental points were set up in triplicates in 384-well plates and all experiments were repeated at least three times. The luminescent signal was detected using a spectrometer and the data was graphically displayed using GraphPad Prism version 5.0 for Windows, (GraphPad Software; www.graphpad.com). The curves were fitted using a non-linear regression model with a sigmoidal dose response. The results of this assay for the compounds disclosed herein are shown in Table 3 below.

TABLE 3

| | | | | | | | L858R/ | |
|---|---|---|---|---|---|---|---|---|
| Cmpd | | | | | L858R/ | | T790M/ | |
| No. | WT | | L858R | | T790M | | C797S | |
| 035 | >10 | (>10) | >10 | (>10) | >10 | (>10) | >10 | (>10) |
| 038 | >10 | (>10) | >10 | (>10) | >10 | (>10) | >10 | (>10) |
| 039 | >10 | (>10) | >10 | (>10) | >10 | (>10) | >10 | (>10) |
| 041 | >10 | (>4.3) | >4.4 | (>2.9) | >10 | (>10) | 5.7 | (>10) |
| 043 | >10 | (>0.80) | >10 | (>10) | >10 | (>10) | >10 | (>10) |
| 047 | >10 | (>4.3) | >10 | (>10) | >10 | (>10) | >10 | (>10) |
| 064 | >10 | (>4.0) | 0.57 | (0.50) | 3.8 | (2.5) | >10 | (>10) |
| 066 | >10 | (>3.7) | 1.2 | (1.2) | 4.4 | (3.6) | >10 | (>10) |
| 067 | >10 | (>3.3) | >10 | (>8.7) | >10 | (>10) | >10 | (>10) |
| 068 | >10 | (>5.6) | >10 | (>10) | >10 | (>10) | >10 | (>10) |
| 069 | >10 | (>2.1) | >5.7 | (>5.3) | >10 | (>5.6) | >5.2 | (>10) |

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A compound of Formula I: 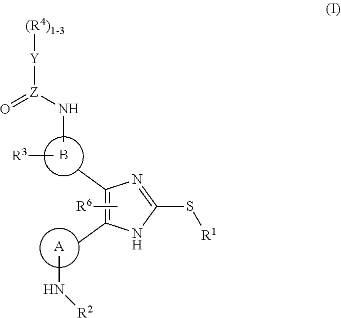
or a pharmaceutically acceptable salt thereof;
wherein:
Z is C or S═O;
Y is selected from the group consisting of NH, C₁-C₆ alkyl, C₆-C₁₀ aryl, 5-10 membered heteroaryl, C₃-C₁₀ cycloalkyl, and 3-10 membered heterocycloalkyl;
alternatively, Z═O and Y are absent;
A is 5-10 membered heteroaryl containing at least one nitrogen atom;
alternatively, A and NHR² are absent;
B is selected from the group consisting of C₆-C₁₀ aryl, 5-10 membered heteroaryl, C₃-C₁₀ cycloalkyl, 3-10 membered heterocycloalkyl, and 5-10 membered bicyclic ring;
R¹ is C₁-C₆ alkyl;
R² is H, CO(C₁-C₆ alkyl), or C₆-C₁₀ aryl, wherein aryl is optionally substituted one or two times with R⁵;
R³ is selected from the group consisting of H, halo, CN, OH, NH₂, and CF₃;
each R⁴ is independently selected from the group consisting of H, OH, halo, C₁-C₆alkyl, C₁-C₆alkoxy, C₆-C₁₀ aryl, 5-10 membered heteroaryl, CH₂-(5-10 membered bicyclic ring), and CH₂NHC(O)(C₆-C₁₀ aryl), wherein aryl is optionally independently substituted one, two, or three times with halo, CO₂H, or C₁-C₆ haloalkyl;
each R⁵ is independently selected from the group consisting of halo, OH, C₁-C₆ alkoxy, and NHC(O)C₂-C₆ alkenyl and
R⁶ is H or C₁-C₆ alkyl.

2. The compound of claim 1, wherein A is pyridine; and B is phenyl, thiophene, or dihydro-indene.

3. (canceled)

4. The compound of claim 1, wherein R¹ is C₁-C₃ alkyl.

5. The compound of claim 1, wherein R² is CO(C₁-C₃ alkyl) or phenyl, wherein phenyl is optionally substituted one or two times with R⁵.

6. The compound of claim 1, wherein Z is C.

7. The compound of claim 1, wherein Z is S═O.

8. The compound of claim 1, wherein Y is selected from the group consisting of NH, C₁-C₃ alkyl, phenyl, naphthalene, pyridine, indole, thiophene, furan, C₃-C₅ cycloalkyl, and 3-5 membered heterocycloalkyl.

9. The compound of claim 1, wherein R³ is H or halo.

10. (canceled)

11. The compound of claim 1 wherein each R⁴ is independently selected from the group consisting of H, OH, halo, C₁-C₃ alkyl, C₁-C₃ alkoxy, phenyl, thiophene, indole, CH₂-(5-10 membered bicyclic ring), and CH₂NHC(O)phenyl, wherein phenyl is optionally substituted one, two, or three times with halo, CO₂H, or C₁-C₃ haloalkyl.

12. The compound of claim 11, wherein each R⁴ is independently selected from the group consisting of H, OH, halo, C₁-C₃ alkyl, C₁-C₃ alkoxy, phenyl, thiophene, indole, 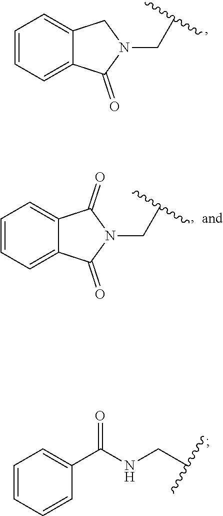 [SMILES: *CN1Cc2ccccc2C1=O ; *CN1C(=O)c2ccccc2C1=O ; *CNC(=O)c1ccccc1]
wherein phenyl is optionally substituted with halo, CO₂H, or C₁-C₃ haloalkyl.

13. The compound of claim 1 wherein R⁶ is H.

14. The compound of claim 1 wherein the compound of Formula I is a compound of Formula IIa or Formula IIb: 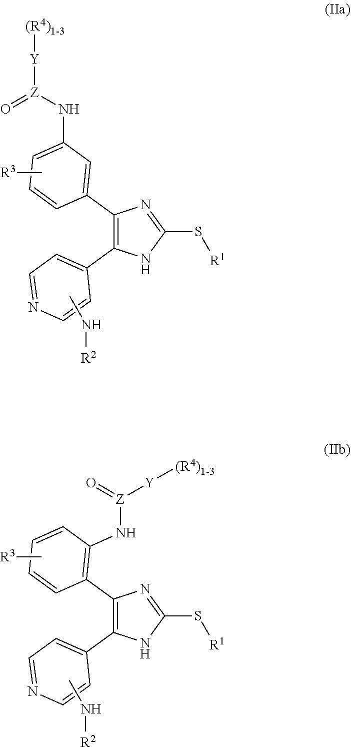
or a pharmaceutically acceptable salt thereof.

15. (canceled)

16. The compound of claim 1, wherein the compound of Formula I is a compound of Formula III: 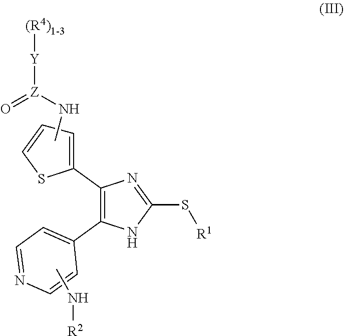
or a pharmaceutically acceptable salt thereof.

17. The compound of claim 1, wherein the compound of Formula I is a compound of Formula IV: 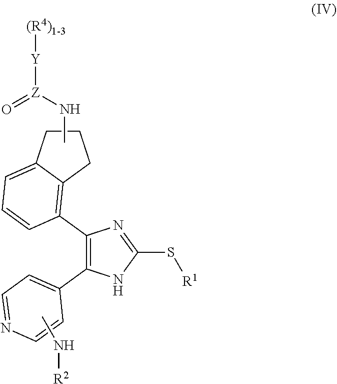
or a pharmaceutically acceptable salt thereof.

18. The compound of claim 1, wherein the compound of Formula I is selected from the group consisting of 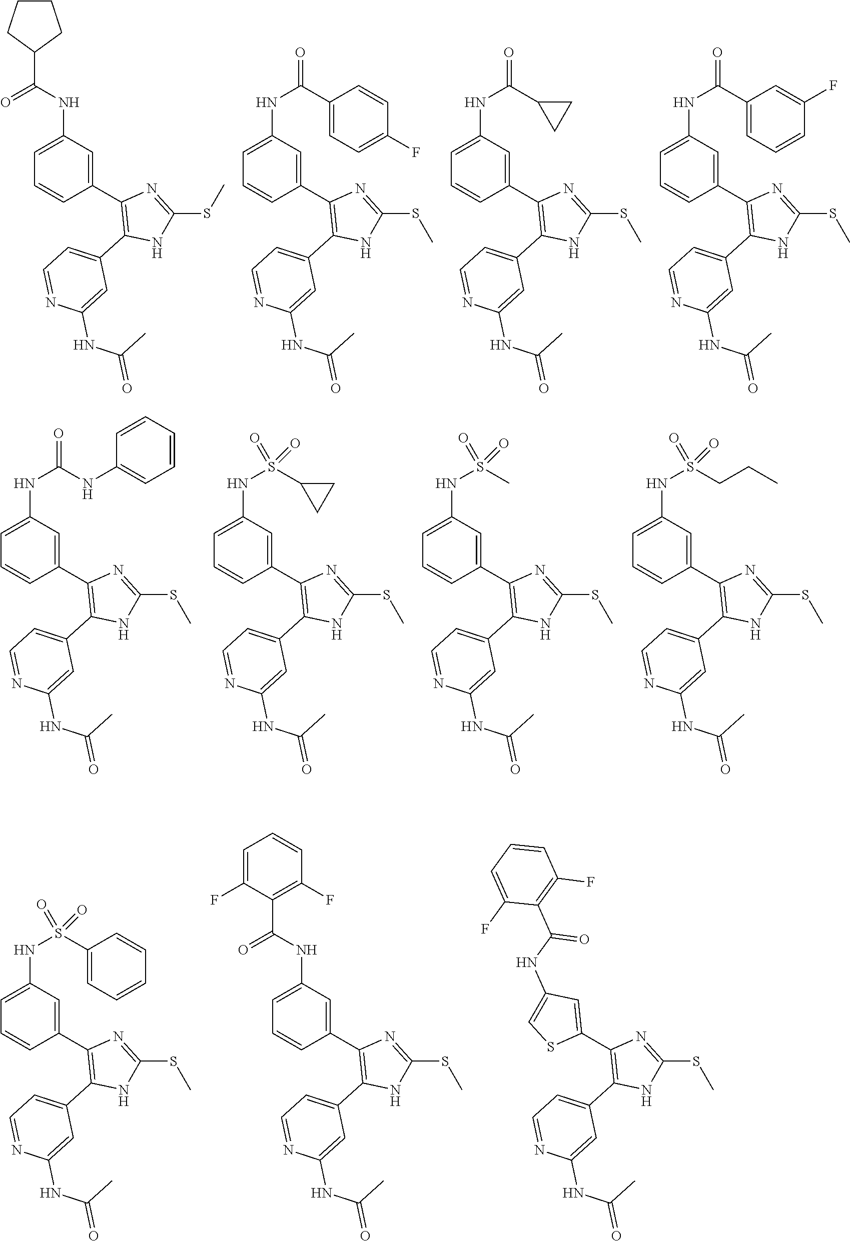 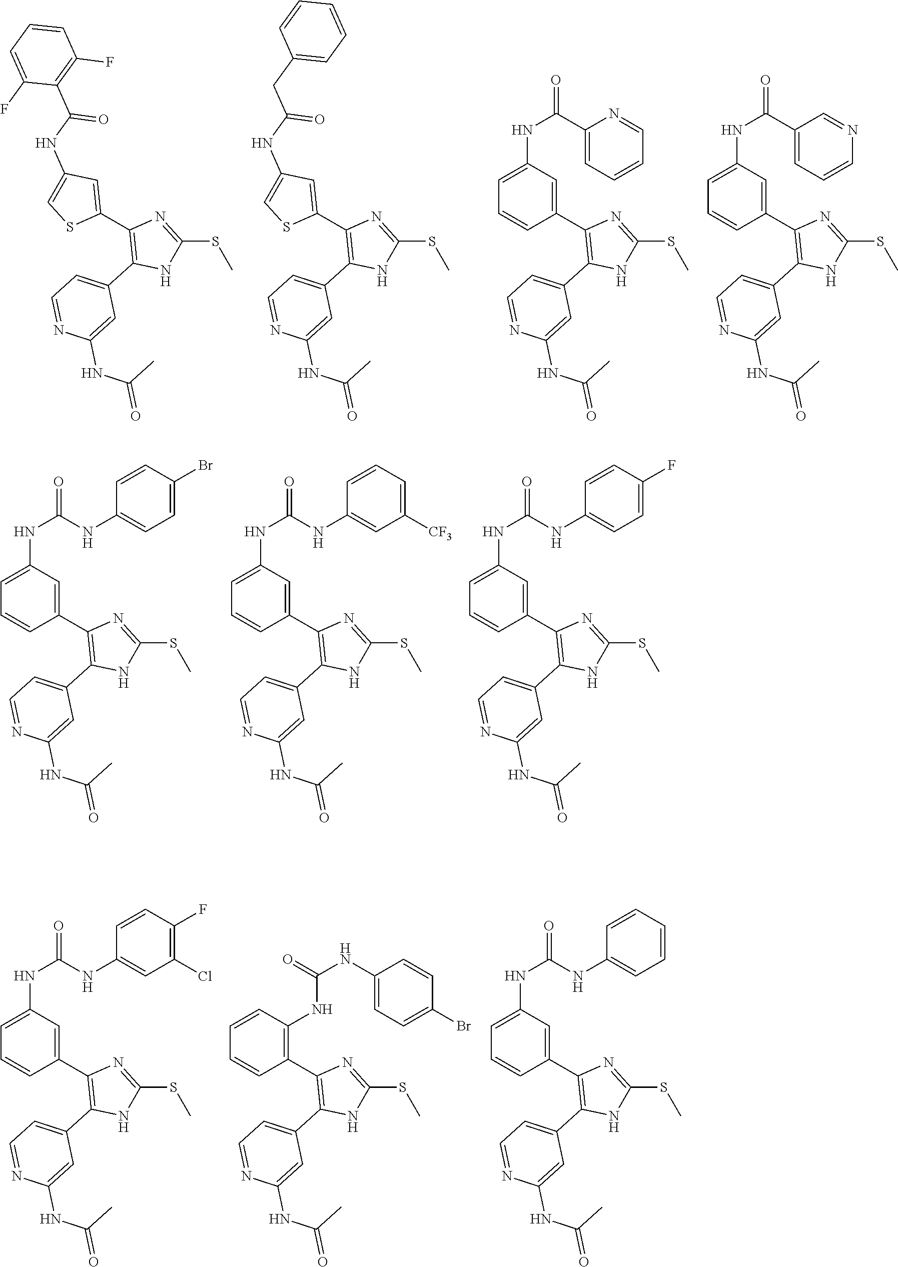 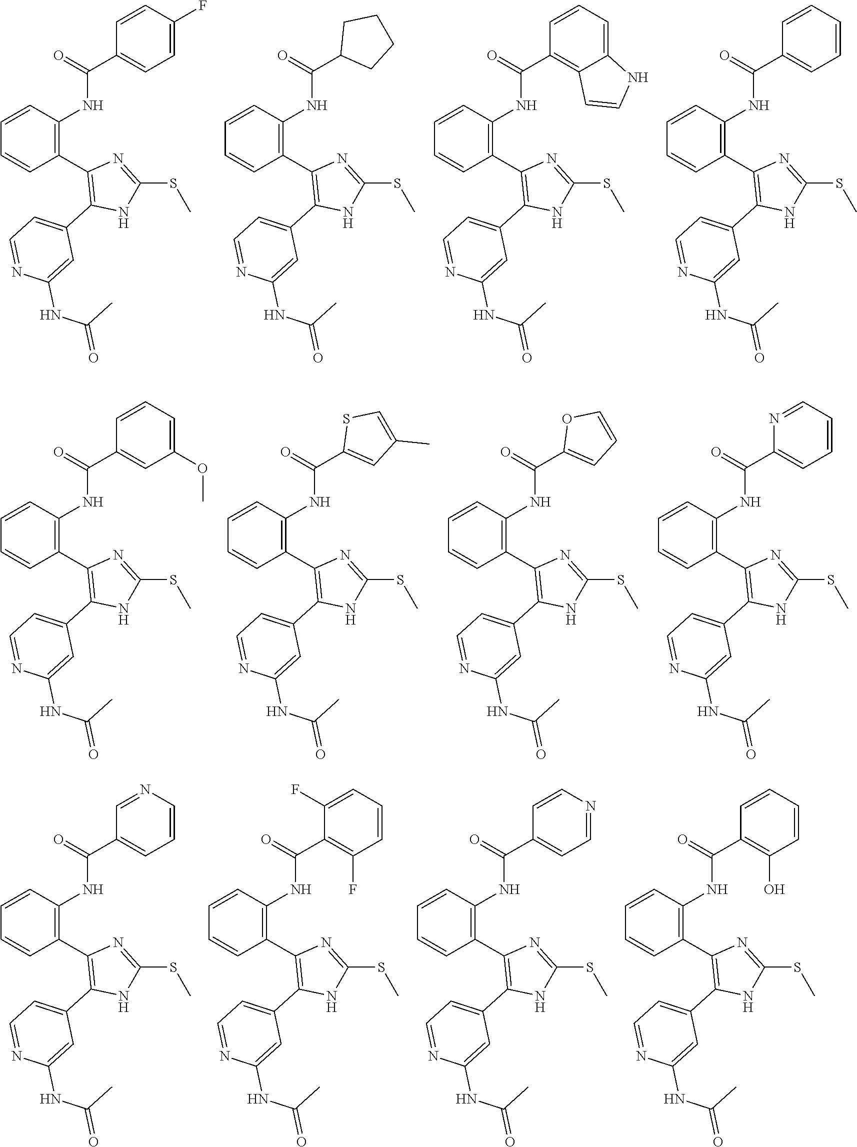 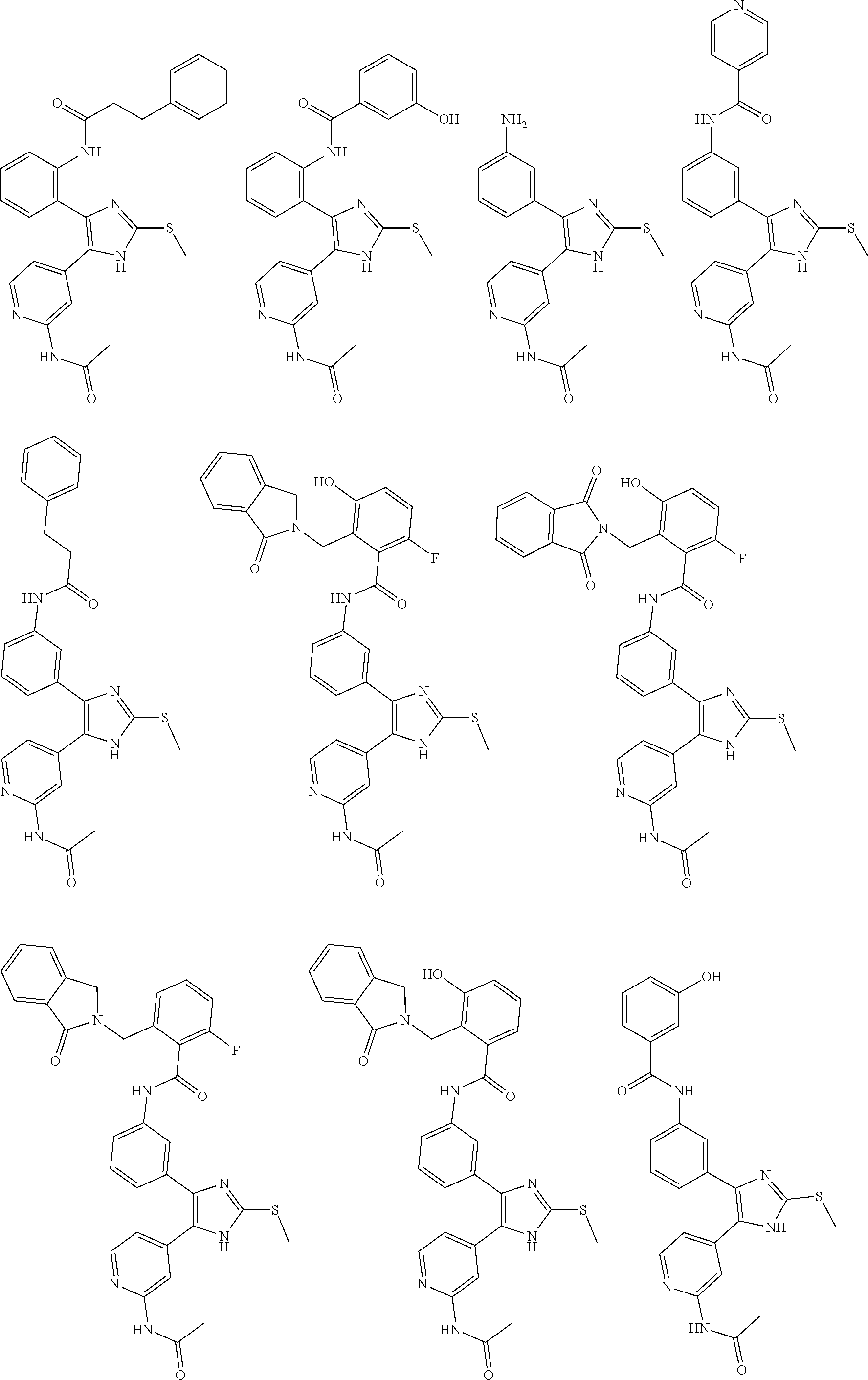 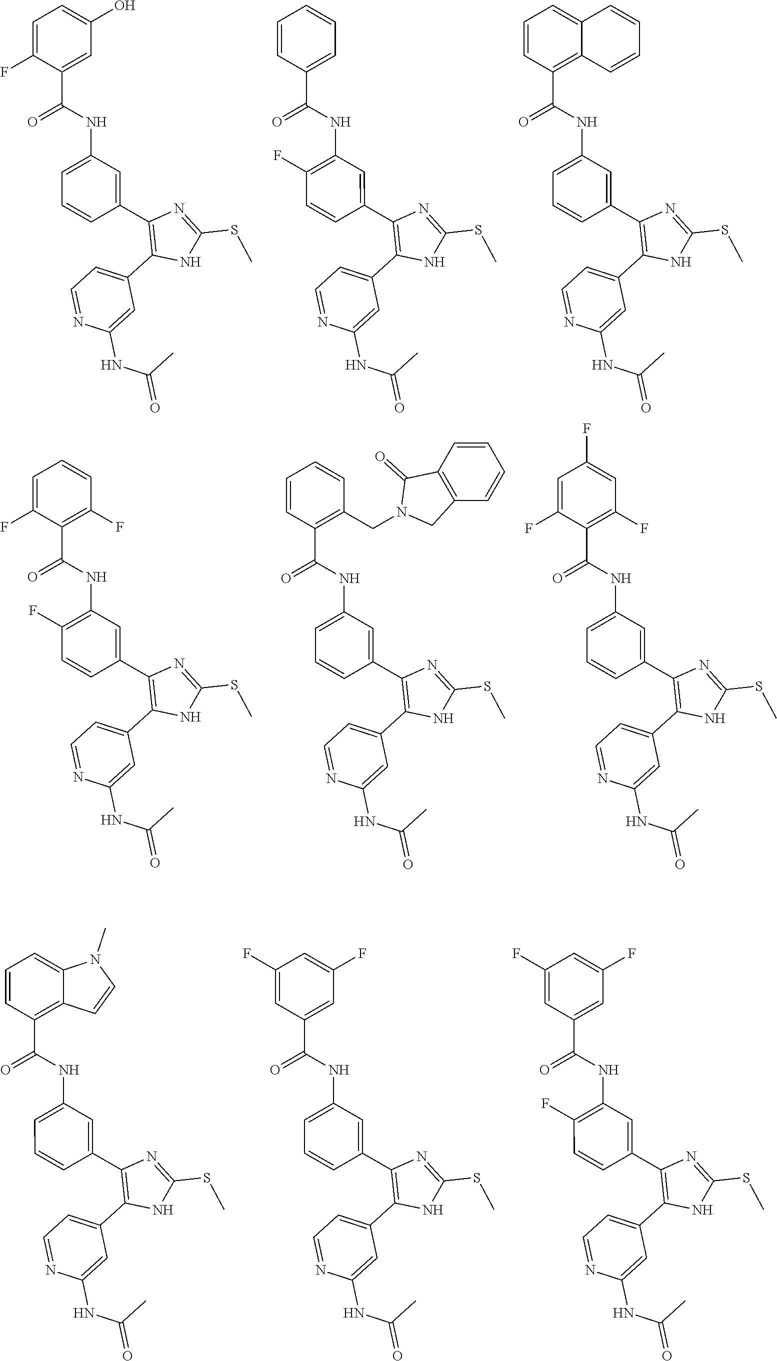 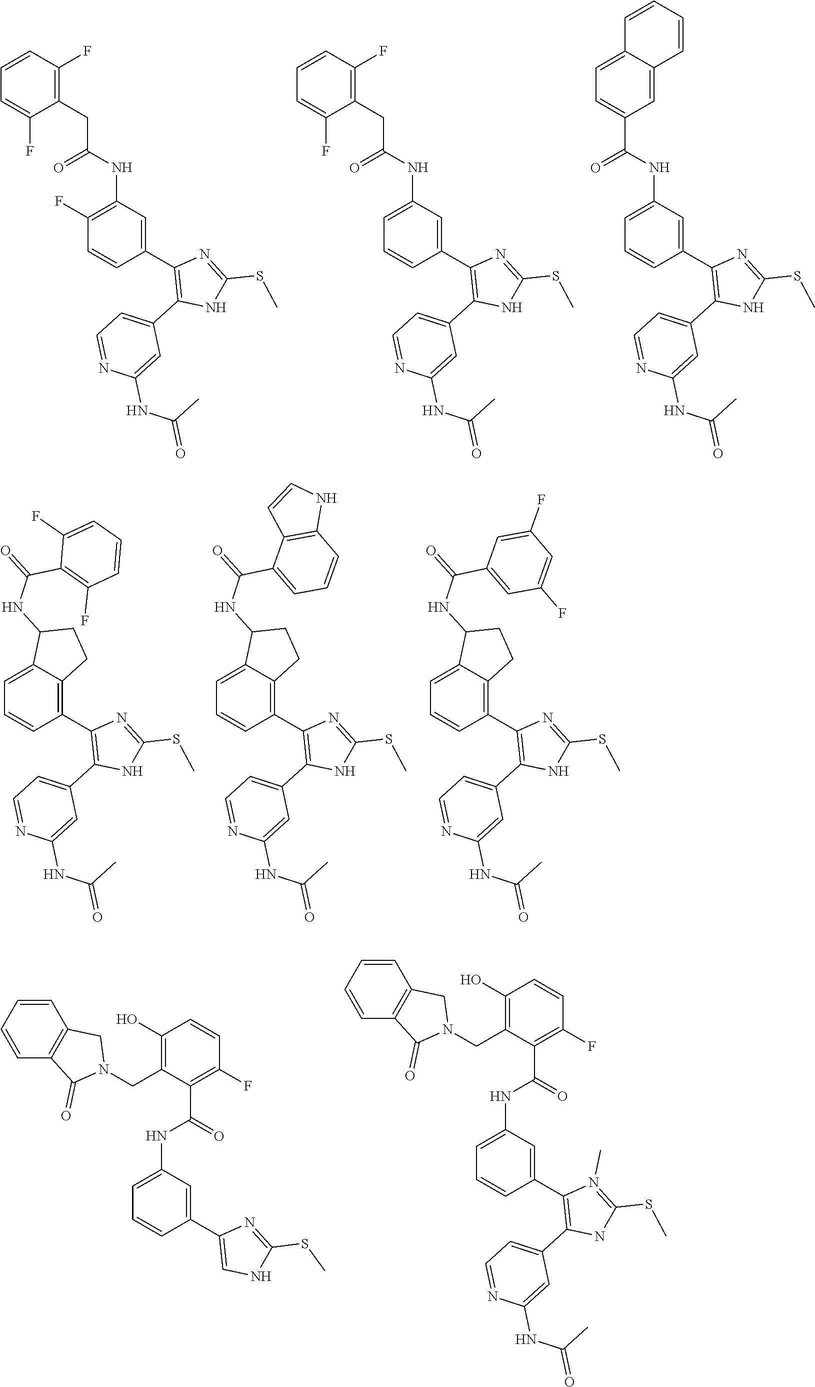 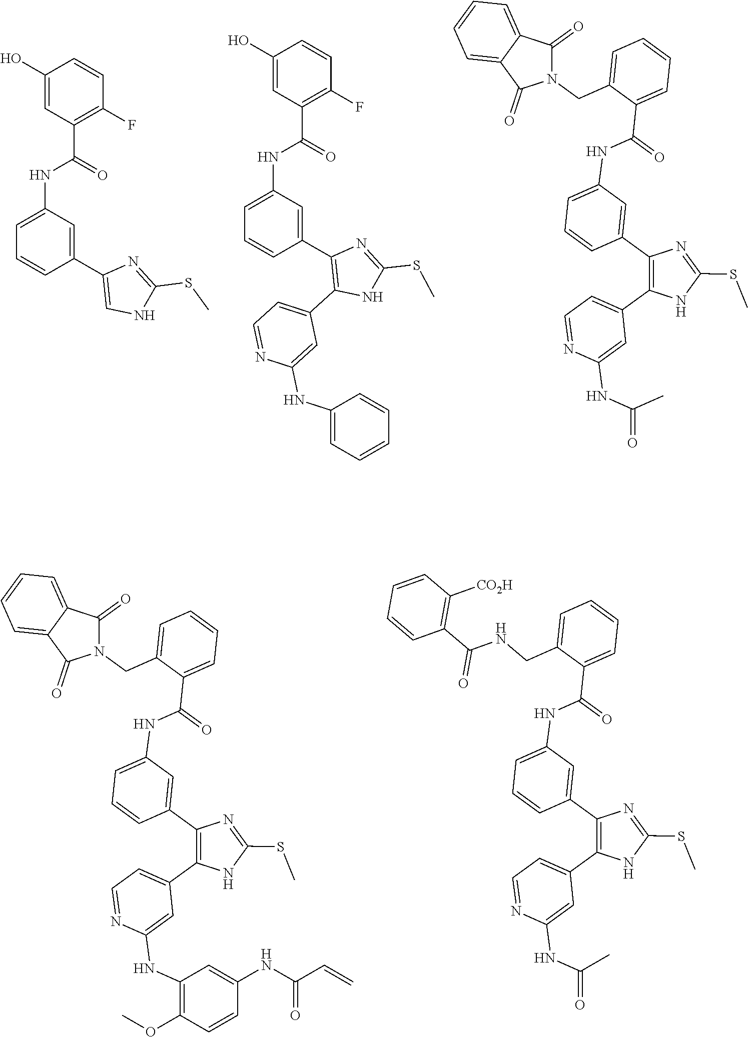 [SMILES: CSc1nc(-c2cccc(NC(=O)c3cc(O)ccc3F)c2)c[nH]1 ; CSc1nc(-c2cccc(NC(=O)c3cc(O)ccc3F)c2)c(-c2ccnc(Nc3ccccc3)c2)[nH]1 ; CSc1nc(-c2cccc(NC(=O)c3ccccc3CN3C(=O)c4ccccc4C3=O)c2)c(-c2ccnc(NC(C)=O)c2)[nH]1 ; C=CC(=O)Nc1ccc(OC)c(Nc2cc(-c3[nH]c(SC)nc3-c3cccc(NC(=O)c4ccccc4CN4C(=O)c5ccccc5C4=O)c3)ccn2)c1 ; CSc1nc(-c2cccc(NC(=O)c3ccccc3CNC(=O)c3ccccc3C(=O)O)c2)c(-c2ccnc(NC(C)=O)c2)[nH]1] 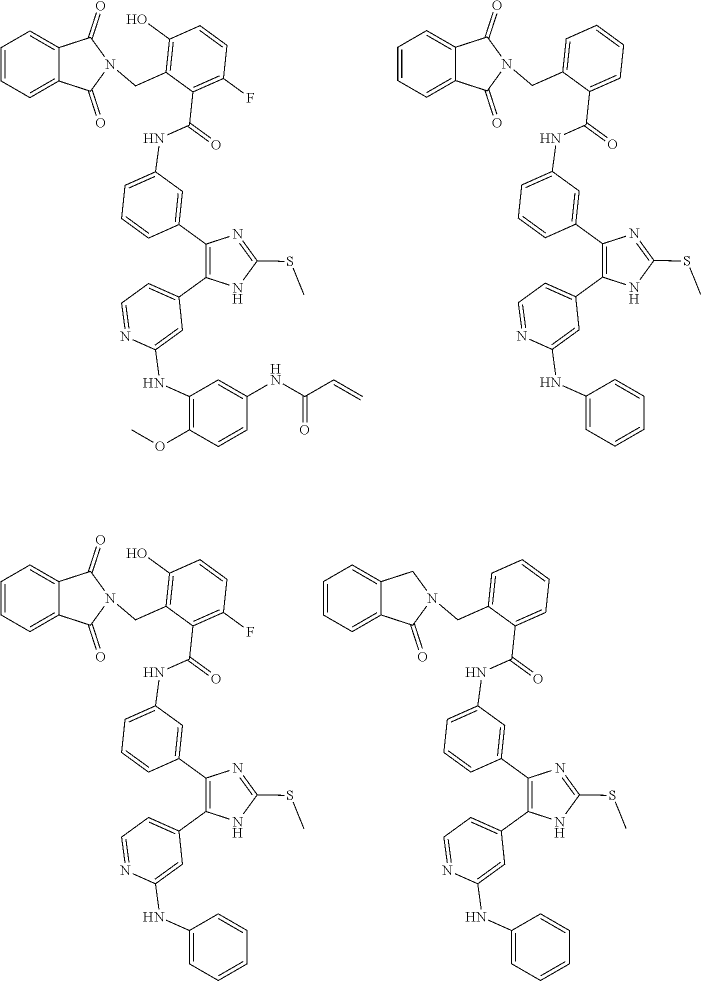 [SMILES: C=CC(=O)Nc1ccc(OC)c(Nc2cc(-c3[nH]c(SC)nc3-c3cccc(NC(=O)c4c(F)ccc(O)c4CN4C(=O)c5ccccc5C4=O)c3)ccn2)c1 ; CSc1nc(-c2cccc(NC(=O)c3ccccc3CN3C(=O)c4ccccc4C3=O)c2)c(-c2ccnc(Nc3ccccc3)c2)[nH]1 ; CSc1nc(-c2cccc(NC(=O)c3c(F)ccc(O)c3CN3C(=O)c4ccccc4C3=O)c2)c(-c2ccnc(Nc3ccccc3)c2)[nH]1 ; CSc1nc(-c2cccc(NC(=O)c3ccccc3CN3Cc4ccccc4C3=O)c2)c(-c2ccnc(Nc3ccccc3)c2)[nH]1]
or a pharmaceutically acceptable salt thereof.

19. The compound of claim 1, wherein
Z is C or S═O;
Y is selected from the group consisting of NH, C₁-C₃ alkyl, phenyl, naphthalene, pyridine, indole, thiophene, furan, C₁-C₅ cycloalkyl, and 3-5 membered heterocycloalkyl;
A is pyridine;
B is phenyl, thiophene, or dihydro-indene;
R¹ is C₁-C₃ alkyl;
R² is CO(C₁-C₃ alkyl) or phenyl, wherein phenyl is optionally substituted one or two times with R⁵;
R³ is H or halo;
each R⁴ is independently selected from the group consisting of H, OH, halo, C₁-C₃ alkyl, C₁-C₃ alkoxy, phenyl, thiophene, indole, CH₂-(5-10 membered bicyclic ring), and CH₂NHC(O)phenyl, wherein phenyl is optionally substituted one, two, or three times with halo, CO₂H, or C₁-C₃ haloalkyl;
each R⁵ is independently selected from the group consisting of halo, OH, C₁-C₃ alkoxy, and NHC(O)C₂-C₃ alkenyl; and
R⁶ is H.

20. A pharmaceutical composition comprising a compound of claim 1, or a pharmaceutically acceptable salt thereof, and a pharmaceutically acceptable carrier.

21. A method of inhibiting the activity of EGFR in a subject in need thereof comprising administering to the subject a therapeutically effective amount of a compound of claim 1.

22. The method of claim 21, wherein the EGFR is characterized by a mutation selected from the group consisting of L858R, T790M, and C797S, or any combination thereof.

23. A method of treating cancer in a subject in need thereof comprising administering to the subject a therapeutically effective amount of a compound of claim 1.

24. The method of claim 23, wherein the cancer is selected from the group consisting of lung cancer, colon cancer, breast cancer, endometrial cancer, thyroid cancer, glioma, squamous cell carcinoma, and prostate cancer.

25. The method according to claim 23, wherein the cancer is non-small cell lung cancer (NSCLC).
